SEQUENCE LISTING (97 sequences)

SEQ ID NO: 1 - DNA, 813 bp, synthetic construct
  source 1..813: mol_type = other DNA
gcggacgtaccgctggaagcgggcaagacttacgtggagctcgccaacccggtcccggtcgccgtgccgggcaaaatcgaagtggtggagctgttctggtacggctgccctcattgctacgcgttcgagcccacgatcaacccctgggccgagaagctgcccaaagacgtcaacttccgtcgcatccccgcgatgttcgggggtccctgggacgcccatggtcagctgtttctcaccttggaagccatgggcgtagagcacaaggtccacaacgccgtgttcgaggccatccaaaagcaaggcaagcggctgaccaagccggacgagatggccgattttgtggccactcaaggcgtggacaaggataagtttctggcgaccttcaatagcttcgcgatccaaggccagatcaagcaagcgaaagaactggcccagaagtatggcgtgcagggcgtccccaccctgatcgtgaacggcaagtaccgcttcgacctgggttcgaccggtgggcccgaggcgaccctgaacgtggcggatcaactcatcgcgaaggaacgcgcagccaagggtggcggtggctcgggcggtggcggtagccaccatcatcaccaccacgggggcgggggtagcgacgatgacgataagggcggcggcggtgccatgggctcggaaggtgacaagacgtgtccggacggctacgagcacacgtgtggctgcatcgggggctgcggctgcaagcggagcgcgtgcattggcgccctctgctgccaggcctccctcggcggctggctgagcgacggtgagacttacacc

SEQ ID NO: 2 - protein, 271 aa, synthetic construct
ADVPLEAGKTYVELANPVPVAVPGKIEVVELFWYGCPHCYAFEPTINPWAEKLPKDVNFRRIPAMFGGPWDAHGQLFLTLEAMGVEHKVHNAVFEAIQKQGKRLTKPDEMADFVATQGVDKDKFLATFNSFAIQGQIKQAKELAQKYGVQGVPTLIVNGKYRFDLGSTGGPEATLNVADQLIAKERAAKGGGGSGGGGSHHHHHHGGGGSDDDDKGGGGAMGSEGDKTCPDGYEHTCGCIGGCGCKRSACIGALCCQASLGGWLSDGETYT

SEQ ID NO: 3 - DNA, 1224 bp, synthetic construct
  source 1..1224: mol_type = other DNA
ctgcccaacatcaccatcctcgcgaccggtggcacgatcgcgggtggcggggatagcgcgaccaagtcgaattacactgcgggcaaggtaggcgtggagaacctcgtcaatgccgtcccgcagctgaaagatattgcgaacgtgaaaggcgagcaagtcgtgaacattggctcccaggatatgaacgacgacgtgtggctgaccctggcgaaaaagatcaacactgactgcgacaagacggatggcttcgtgatcacccatggcacggacaccatggaagaaaccgcctactttctggacctgaccgtgaagtgcgacaagcccgtggtgatggtgggcgcgatgcgccccagcacctcgatgtcggccgacgggcccttcaacctgtacaacgccgtggtcaccgcggccgataaagcaagcgcgaatcggggcgtcctcgtcgtgatgaacgatactgtcctggatggccgcgacgtgaccaagacgaatacgactgacgtcgccaccttcaagagcgtaaactacggtcctctgggctacatccacaacggtaagatcgattaccaacggacccccgcccgcaagcacaccagcgacacgccgttcgatgtcagcaagctgaacgaactgccgaaggtaggcatcgtgtacaactatgccaacgccagcgacctccccgccaaggccctcgtggacgcgggctacgacggcatcgtgtcggcgggtgtgggcaacgggaacctctacaagaccgtctttgacactctggcgaccgccgccaagaacggtaccgcggtagtgcgctcgtcgcgtgtacccactggtgcgacgacgcaagacgccgaagtggacgatgccaagtacggcttcgtggcgtcagggacgctcaacccgcaaaaggcccgggtgctgctgcagctggccctgacccagactaaggatccgcagcagatccaacaaatcttcaaccaatacggcggcggcggctcgggcggtggtggcagccatcatcaccaccaccacggtggcggtggcagcgacgacgacgacaagggcgggggcggtgcgatgggtagcgagggcgacaagacttgcccggacggctacgagcatacctgtggctgcatcggcggctgcggctgtaagcgctccgcatgcatcggtgccctctgctgccaagccagcttgggtggctggctgagtgatggggagacttacacc

SEQ ID NO: 4 - protein, 408 aa, synthetic construct
LPNITILATGGTIAGGGDSATKSNYTAGKVGVENLVNAVPQLKDIANVKGEQVVNIGSQDMNDDVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKTVFDTLATAAKNGTAVVRSSRVPTGATTQDAEVDDAKYGFVASGTLNPQKARVLLQLALTQTKDPQQIQQIFNQYGGGGSGGGGSHHHHHHGGGGSDDDDKGGGGAMGSEGDKTCPDGYEHTCGCIGGCGCKRSACIGALCCQASLGGWLSDGETYT

SEQ ID NO: 5 - DNA, 216 bp, synthetic construct
  source 1..216: mol_type = other DNA
ggcggcggtggcgccatgggcagcgagggcgacaagacttgccccgacggctacgagcatacgtgcgggtgcatcgggggctgcggctgcaagcgcagcgcctgcatcggtgcgctgtgttgccaagcgagcctggggggctggctctcggatggcgaaacctacaccggtggcggtggctcgggtggtggcggctcccaccatcaccaccaccac

SEQ ID NO: 6 - protein, 72 aa, synthetic construct
GGGGAMGSEGDKTCPDGYEHTCGCIGGCGCKRSACIGALCCQASLGGWLSDGETYTGGGGSGGGGSHHHHHH

SEQ ID NO: 7 - DNA, 222 bp, synthetic construct
  source 1..222: mol_type = other DNA
ggtggcggtggcgcgatgggcagcaagactaagaacgcgtgcccggatgacttcgactaccggtgcagctgcatcggtggctgcgggtgtgcccgcaagggctgcgtgggtccgctgtgctgccgcagcgacctgggcggctacctgaccgattcccccgcctacatctacctcggcggcgggggctcgggcggtggcggttcgcaccaccatcaccaccat

SEQ ID NO: 8 - protein, 74 aa, synthetic construct
GGGGAMGSKTKNACPDDFDYRCSCIGGCGCARKGCVGPLCCRSDLGGYLTDSPAYIYLGGGGSGGGGSHHHHHH

SEQ ID NO: 9 - protein, 693 aa, Pseudomonas fluorescens
MKHLFPSTALAFFIGLGFASMSTNTFAANSWDNLQPDRDEVIASLNVVELLKRHHYSKPPLDDARSVIIYDSYLKLLDPSRSYFLASDIAEFDKWKTQFDDFLKSGDLQPGFTIYKRYLDRVKARLDFALGELNKGVDKLDFTQKETLLVDRKDAPWLTSTAALDDLWRKRVKDEVLRLKIAGKEPKAIQELLTKRYKNQLARLDQTRAEDIFQAYINTFAMSYDPHTNYLSPDNAENFDINMSLSLEGIGAVLQSDNDQVKIVRLVPAGPADKTKQVAPADKIIGVAQADKEMVDVVGWRLDEVVKLIRGPKGSVVRLEVIPHTNAPNDQTSKIVSITREAVKLEDQAVQKKVLNLKQDGKDYKLGVIEIPAFYLDFKAFRAGDPDYKSTTRDVKKILTELQKEKVDGVVIDLRNNGGGSLQEATELTSLFIDKGPTVLVRNADGRVDVLEDENPGAFYKGPMALLVNRLSASASEIFAGAMQDYHRALIIGGQTFGKGTVQTIQPLNHGELKLTLAKFYRVSGQSTQHQGVLPDIDFPSIIDTKEIGESALPEAMPWDTIRPAIKPASDPFKPFLAQLKADHDTRSAKDAEFVFIRDKLALAKKLMEEKTVSLNEADRRAQHSSIENQQLVLENTRRKAKGEDPLKELKKEDEDALPTEADKTKPEDDAYLAETGRILLDYLKITKQVAKQ

SEQ ID NO: 10 - protein, 437 aa, Pseudomonas fluorescens
MLHLSRLTSLALTIALVIGAPLAFADQAAPAAPATAATTKAPLPLDELRTFAEVMDRIKAAYVEPVDDKALLENAIKGMLSNLDPHSAYLGPEDFAELQESTSGEFGGLGIEVGSEDGQIKVVSPIDDTPASKAGIQAGDLIVKINGQPTRGQTMTEAVDKMRGKLGQKITLTLVRDGGNPFDVTLARATITVKSVKSQLLESGYGYIRITQFQVKTGDEVAKALAKLRKDNGKKLNGIVLDLRNNPGGVLQSAVEVVDHFVTKGLIVYTKGRIANSELRFSATGNDLSENVPLAVLINGGSASASEIVAGALQDLKRGVLMGTTSFGKGSVQTVLPLNNERALKITTALYYTPNGRSIQAQGIVPDIEVRRAKITNEIDGEYYKEADLQGHLGNGNGGADQPTGSRAKAKPMPQDDDYQLAQALSLLKGLSITRSR

SEQ ID NO: 11 - DNA, 120 bp, synthetic construct
  source 1..120: mol_type = other DNA
ggtagcacttgccccgacggctacgagcacacgtgcgggtgcatcggcggctgcggctgcaagcgctcggcctgcatcggtgcgctgtgctgtcaagcctccctcggcggctggctgagc

SEQ ID NO: 12 - protein, 40 aa, synthetic construct
GSTCPDGYEHTCGCIGGCGCKRSACIGALCCQASLGGWLS

SEQ ID NO: 13 - protein, 468 aa, Pseudomonas fluorescens
MTTEPSKAPPLYPKTHLLAASGIAALLSLALLVFPSSDVEAKRTSLSLDLESPVEQLTQDQDASDAQQATNTATESPFAQIESTPEDTQQAAQEAPAAAKSPQHREVIVGKGDTLSTLFEKVGLPAAAVNDVLASDKQAKQFTQLKRGQKLEFELTPDGQLNNLYTSISDLESISLSKGAKGFAFNRITTKPVMRSAYVHGVINSSLSQSAARAGLSHSMTMDMASVFGYDIDFAQDIRQGDEFDVIYEQKVANGKVVGTGNILSARFTNRGKTYTAVRYTNKQGNSSYYTADGNSMRKAFIRTPVDFARISSRFSMGRKHPILNKIRAHKGVDYAAPRGTPIKAAGDGKVLLAGRRGGYGNTVIIQHGNTYRTLYGHMQGFAKGVKTGGNVKQGQVIGYIGTTGLSTGPHLHYEFQVNGVHVDPLGQKLPMADPIAKAERARFMQQSQPLMARMDQERSTLLASAKR

SEQ ID NO: 14 - protein, 6 aa, synthetic construct
  VARIANT 2: note = Any amino acid
  VARIANT 5: note = Any amino acid
CXTVXQ

SEQ ID NO: 15 - protein, 6 aa, synthetic construct
CTTVHQ

SEQ ID NO: 16 - protein, 509 aa, Pseudomonas fluorescens
MHIPVRQSSYSRPSDKLQPDLSPDEHQVVLWANNKKSFTTDQAAKHITRGGFKFHDRNNDGKIVVGYNFAGGFNAAQKERARQALQYWADVANIEFVENGPNTDGTISIKGVPGSAGVAGLPNKYNSNVQANIGTQGGQNPAMGSHFLGLLIHELGHTLGLSHPGKYDGQGFNYDRAAEYAQDTKARSVMSYWTETHQPGHNFAGRSPGAPMMDDIAAAQRLYGANTKTRNTDTTYGFNSNSGREAYSLKQGSDKPIFTVWDGGGNDTLDFSGFTQNQTINLKAESFSDVGGLRGNVSIAKGVSVENAIGGTGNDTLTGNEGNNRLTGGKGADKLHGGAGADTFVYRRASDSTPQAPDIIQDFQSGSDKIDLTGVVQEAGLKSLSFVEKFSGKAGEAVLGQDAKTGRFTLAVDTTGNGTADLLVASQSQIKQADVIWNGQAPTVTPTPEPTVVPVSDPVPTPTSEPTEPEPTPEPAPLPVPTPRPGGGFIGKIFSSFKGFIKKVWSIFR

SEQ ID NO: 17 - DNA, 138 bp, synthetic construct
  source 1..138: mol_type = other DNA
ggtggttccaacgcgtgccctgatgacttcgactatcgctgttcctgtattggtggttgcggctgcgcccggaaggggtgcgtgggccccctctgctgccgcagcgacctgggcggctacctgaccgattcgccggcc

SEQ ID NO: 18 - protein, 46 aa, synthetic construct
GGSNACPDDFDYRCSCIGGCGCARKGCVGPLCCRSDLGGYLTDSPA

SEQ ID NO: 19 - protein, 478 aa, Pseudomonas fluorescens
MSIPRLKSYLSIVATVLVLGQALPAQAVELPDFTQLVEQASPAVVNISTTQKLPDRKVSNQQMPDLEGLPPMLREFFERGMPQPRSPRGGGGQREAQSLGSGFIISPDGYILTNNHVIADADEILVRLADRSELKAKLIGTDPRSDVALLKIEGKDLPVLKLGKSQDLKAGQWVVAIGSPFGFDHTVTQGIVSAIGRSLPNENYVPFIQTDVPINPGNSGGPLFNLAGEVVGINSQIYTRSGGFMGVSFAIPIDVAMDVSNQLKSGGKVSRGWLGVVIQEVNKDLAESFGLDKPAGALVAQIQDNGPAAKGGLKVGDVILSMNGQPIIMSADLPHLVGALKAGGKAKLEVIRDGKRQNVELTVGAIPEEGATLDALGNAKPGAERSSNRLGIAVVELTAEQKKTFDLQSGVVIKEVQDGPAALIGLQPGDVITHLNNQAIDTTKEFADIAKALPKNRSVSMRVLRQGRASFITFKLAE

SEQ ID NO: 20 - protein, 445 aa, Pseudomonas fluorescens
MSMTPREIVHELNRHIIGQDDAKRAVAIALRNRWRRMQLPEELRVEVTPKNILMIGPTGVGKTEIARRLAKLANAPFIKVEATKFTEVGYVGRDVESIIRDLADAALKMLREQEVTKVSHRAEDAAEERILDALLPPARMGFNEDAAPATDSNTRQLFRKRLREGQLDDKEIEIEVAEVSGVDISAPPGMEEMTSQLQNLFANMGKGKKKSRKLKVKEALKLVRDEEAGRLVNEEELKAKALEAVEQHGIVFIDEIDKVAKRGNSGGVDVSREGVQRDLLPLIEGCTVNTKLGMVKTDHILFIASGAFHLSKPSDLVPELQGRLPIRVELKALTPGDFERILSEPHASLTEQYRELLKTEGLGIEFQADGIKRLAEIAWQVNEKTENIGARRLHTLLERLLEEVSFSAGDMAGAQNGEAIKIDADYVNSHLGELAQNEDLSRYIL

SEQ ID NO: 21-22 - skipped (2 entries)

SEQ ID NO: 23 - DNA, 54 bp, Pseudomonas fluorescens
  source 1..54: mol_type = genomic DNA
atgcgacaactatttttctgtttgatgctgatggtgtcgctcacggcgcacgcc

SEQ ID NO: 24 - protein, 18 aa, Pseudomonas fluorescens
MRQLFFCLMLMVSLTAHA

SEQ ID NO: 25 - DNA, 75 bp, Pseudomonas fluorescens
  source 1..75: mol_type = genomic DNA
atgaaatctgcattgaagaacgttattccgggcgccctggcccttctgctgctattccccgtcgccgcccaggcc

SEQ ID NO: 26 - protein, 25 aa, Pseudomonas fluorescens
MKSALKNVIPGALALLLLFPVAAQA

SEQ ID NO: 27 - DNA, 72 bp, Pseudomonas fluorescens
  source 1..72: mol_type = genomic DNA
atgctttttcgcacattactggcgagccttacctttgctgtcatcgccggcttaccgtccacggcccacgcc

SEQ ID NO: 28 - protein, 24 aa, Pseudomonas fluorescens
MLFRTLLASLTFAVIAGLPSTAHA

SEQ ID NO: 29 - DNA, 63 bp, Pseudomonas fluorescens
  source 1..63: mol_type = genomic DNA
atgaagttcaaacagctgatggcgatggcgcttttgttggccttgagcgctgtggcccaggcc

SEQ ID NO: 30 - protein, 21 aa, Pseudomonas fluorescens
MKFKQLMAMALLLALSAVAQA

SEQ ID NO: 31 - DNA, 93 bp, synthetic construct
  source 1..93: mol_type = other DNA
atgatccgtgacaaccgactcaagacatcccttctgcgcggcctgaccctcaccctactcagcctgaccctgctctcgcccgcggcccatgcc

SEQ ID NO: 32 - protein, 31 aa, synthetic construct
MIRDNRLKTSLLRGLTLTLLSLTLLSPAAHA

SEQ ID NO: 33 - DNA, 69 bp, Pseudomonas fluorescens
  source 1..69: mol_type = genomic DNA
atgcagaactataaaaaattccttctggccgcggccgtctcgatggcgttcagcgccacggccatggcc

SEQ ID NO: 34 - protein, 23 aa, Pseudomonas fluorescens
MQNYKKFLLAAAVSMAFSATAMA

SEQ ID NO: 35 - protein, 56 aa, synthetic construct
GGGGAMGSEGDKTCPDGYEHTCGCIGGCGCKRSACIGALCCQASLGGWLSDGETYT

SEQ ID NO: 36 - protein, 68 aa, synthetic construct
GGGGAMGSKTKNACPDDFDYRCSCIGGCGCARKGCVGPLCCRSDLGGYLTDSPAYIYLGGGGSGGGGS

SEQ ID NO: 37 - DNA, 63 bp, Pseudomonas fluorescens
  source 1..63: mol_type = genomic DNA
atgaagaagtccaccttggctgtggctgtaacgttgggcgcaatcgcccagcaagcaggcgcc

SEQ ID NO: 38 - protein, 21 aa, Pseudomonas fluorescens
MKKSTLAVAVTLGAIAQQAGA

SEQ ID NO: 39 - DNA, 63 bp, Pseudomonas fluorescens
  source 1..63: mol_type = genomic DNA
atgagaaaccttcttcgaggaatgcttgtcgttatttgctgtatggcagggatagcggcggcc

SEQ ID NO: 40 - protein, 21 aa, Pseudomonas fluorescens
MRNLLRGMLVVICCMAGIAAA

SEQ ID NO: 41 - DNA, 477 bp, Pseudomonas fluorescens
  source 1..477: mol_type = genomic DNA
atgactgatcaacagaacaccgaagcagcgcaagaccaaggcccacagttctcgctgcagcggatctatgtgcgtgacctgtcgttcgaagcgccaaaaagcccggccatcttccgtcaggagtggaccccaagcgttgcgctggacctgaacactcgtcagaaatccctggaaggtgacttccacgaagtggtgctgaccctgtcggtcaccgtcaagaatggtgaagaagtcgctttcatcgctgaagtgcaacaggccggtatcttcctgatccagggcctggacgaagcgtccatgagccacaccctgggcgcgttctgcccgaacatcctgttcccgtatgcccgtgagaccctggacagcctggtcacccgtggctcgttcccggcactgatgctggcgccggttaacttcgatgccctgtacgctcaagagctgcagcgcatgcaacaggaaggcgcgccgaccgttcag

SEQ ID NO: 42 - protein, 159 aa, Pseudomonas fluorescens
MTDQQNTEAAQDQGPQFSLQRIYVRDLSFEAPKSPAIFRQEWTPSVALDLNTRQKSLEGDFHEVVLTLSVTVKNGEEVAFIAEVQQAGIFLIQGLDEASMSHTLGAFCPNILFPYARETLDSLVTRGSFPALMLAPVNFDALYAQELQRMQQEGAPTVQ

SEQ ID NO: 43 - DNA, 633 bp, Pseudomonas fluorescens
  source 1..633: mol_type = genomic DNA
atgcgtaatctgatcctcagcgccgctctcgtcactgccagcctcttcggcatgaccgcacaagctgccgatgtgccgcttgaagccggtaaaacctatgttgagctggctaacccggttcccgttgcagtgccgggcaagatcgaagtggtggagctgttctggtacggctgcccgcattgctacgccttcgagccgactatcaacccatgggctgaaaagctgcccaaggacgttaacttccgtcgcattcccgccatgttcggtggcccatgggacgcccacggccagctgttcctgaccctggaagccatgggtgttgagcacaaggtccacaacgctgtcttcgaagcgatccagaaacaaggcaagcgcctgaccaagccggacgaaatggctgacttcgttgccactcagggtgtcgacaaggacaagttcctggcgaccttcaactccttcgctatccagggccagatcaaacaggccaaggaactcgcgcagaagtacggcgtgcaaggcgttccaaccctgatcgtcaacggcaaataccgtttcgacctgggcagcaccggtggtcctgaagcgaccctgaacgttgctgaccagctgattgccaaagaacgcgctgccaag

SEQ ID NO: 44 - protein, 211 aa, Pseudomonas fluorescens
MRNLILSAALVTASLFGMTAQAADVPLEAGKTYVELANPVPVAVPGKIEVVELFWYGCPHCYAFEPTINPWAEKLPKDVNFRRIPAMFGGPWDAHGQLFLTLEAMGVEHKVHNAVFEAIQKQGKRLTKPDEMADFVATQGVDKDKFLATFNSFAIQGQIKQAKELAQKYGVQGVPTLIVNGKYRFDLGSTGGPEATLNVADQLIAKERAAK

SEQ ID NO: 45 - DNA, 729 bp, Pseudomonas fluorescens
  source 1..729: mol_type = genomic DNA
atgcgcttgacccagattattgccgccgcagccattgcgttggtttccacctttgcgctcgccgatgatgcggccgagcagaccatccgcaagagcctggccaacctggcgctcgacacgcctatcgaaagcattagcgccagccccatggccggcctgtacgaagtcaagctcaagggcagccgcgtgctgtacgccagtgccgatggccagtacatcgtccagggctacctgttccagctcaaggacggcaagccggtcaacctgaccgagaaggccgagcgcctgggcgtgtccaagctgatcaacggcatcccggtggctgaaaccgtggtttacccggccattggcgaaaccaagacccacatcaccgtgttcaccgacaccacctgcccgtactgccacaagctgcacgctgaaatcccggcactgaacaagctgggcgtggaagtgcgctacgtcgcgttcccgcgccagggcctgggttcgccgggtgacgagcagttgcaagccgtatggtgttcggccgacaaaaaggcggccatggacaagatggtcgacggcaaggaaatcaaatcggccaaatgcgccaacccggtttccaagcagttcgccctgggccagtccattggtgtgaacggtacaccggccatcgttttggccgacggccaggtgattccgggctaccagccggcgccgcaagttgccaaactggcactgggtgccaag

SEQ ID NO: 46 - protein, 243 aa, Pseudomonas fluorescens
MRLTQIIAAAAIALVSTFALADDAAEQTIRKSLANLALDTPIESISASPMAGLYEVKLKGSRVLYASADGQYIVQGYLFQLKDGKPVNLTEKAERLGVSKLINGIPVAETVVYPAIGETKTHITVFTDTTCPYCHKLHAEIPALNKLGVEVRYVAFPRQGLGSPGDEQLQAVWCSADKKAAMDKMVDGKEIKSAKCANPVSKQFALGQSIGVNGTPAIVLADGQVIPGYQPAPQVAKLALGAK

SEQ ID NO: 47 - DNA, 501 bp, Pseudomonas fluorescens
  source 1..501: mol_type = genomic DNA
atgcgtaagttgactcaattggtcttgctggccactgtgctggtcaccaccccggccttcgccgaaatgaaaatcgccgttctgaactatcagatggccctgctggaatccgatgcggccaagcgatacgccgtggatgccgagaagaagttcggtccgcaactgaccaagctcaagacactggaaagcagcgccaaaggcatccaggaccgcctggtagccggtggcgacaagatgcagcaaggcgagcgcgagcgtctggagcttgaattcaagcaaaaggcccgtgactaccagttccaatccaaggagctgaacgaagccaaggctgtggccgaccgcgaaatgctcaagcagctcaagcctaaattggacagcgctgtggaagaagtcatcaagaagggtgcctttgacctggtgttcgagcgtggcgccgtgatcgacgtcaagcctcaatacgacatcacccgccaggtgatcgagcgcatgaaccagctgaag

SEQ ID NO: 48 - protein, 167 aa, Pseudomonas fluorescens
MRKLTQLVLLATVLVTTPAFAEMKIAVLNYQMALLESDAAKRYAVDAEKKFGPQLTKLKTLESSAKGIQDRLVAGGDKMQQGERERLELEFKQKARDYQFQSKELNEAKAVADREMLKQLKPKLDSAVEEVIKKGAFDLVFERGAVIDVKPQYDITRQVIERMNQLK

SEQ ID NO: 49 - DNA, 738 bp, Pseudomonas fluorescens
  source 1..738: mol_type = genomic DNA
atgaaacagcatcggttggcggcggcggtggccctggttagcctggtacttgcgggttgtgattcgcagaccagcgtagagctgaaaaccccggcgcaaaaggcctcctacggcatcggcctgaacatgggcaagagccttgcccaagaaggcatggacgacctggactccaaagctgttgcccagggcatcgaagatgccgtcggcaagaaagagcagaagctcaaggacgatgagctggttgaagcgtttgccgcactgcaaaagcgtgctgaagaacgcatgaccaaaatgagcgaagagtcggcagccgctggcaagaaattcctcgaagacaacgccaagaaagacggtgtcgtcaccaccgcttccggcctgcagtacaagatcgtgaagaaggccgacggcgcccagcctaagccgaccgacgtggtgactgttcactacaccggcaagctcaccaacggcaccacctttgacagctccgtagatcgcggtagcccgatcgacctgccggtcagcggcgtgatcccgggttgggtcgaaggcctgcaactgatgcacgtgggcgagaaggttgagctgtacatcccgtccgacctggcctacggcgcccagagcccgagcccggcgatcccagcgaactccgtgctggtattcgacctggaactgctgggcatcaaggacccagccaaggcagaagcggctgacgcacctgctgcaccagccgccaagaag

SEQ ID NO: 50 - protein, 246 aa, Pseudomonas fluorescens
MKQHRLAAAVALVSLVLAGCDSQTSVELKTPAQKASYGIGLNMGKSLAQEGMDDLDSKAVAQGIEDAVGKKEQKLKDDELVEAFAALQKRAEERMTKMSEESAAAGKKFLEDNAKKDGVVTTASGLQYKIVKKADGAQPKPTDVVTVHYTGKLTNGTTFDSSVDRGSPIDLPVSGVIPGWVEGLQLMHVGEKVELYIPSDLAYGAQSPSPAIPANSVLVFDLELLGIKDPAKAEAADAPAAPAAKK

SEQ ID NO: 51 - DNA, 1311 bp, Pseudomonas fluorescens
  source 1..1311: mol_type = genomic DNA
atgctgcatttgtcccgcctcacttcgctggccctgacgatcgccctggtgatcggcgcgcctctggcttttgccgaccaggccgcaccggctgcacccgccacggctgcgacgaccaaggcgccattgccgctggacgagctgcgtacctttgccgaggtcatggaccggatcaaggcagcgtatgtcgaacccgtagacgacaaggccctgctggaaaatgccatcaagggcatgctcagcaacctcgacccgcactccgcctacctgggcccggaagatttcgccgagctgcaggaaagcaccagcggtgagttcggcggcctgggcatcgaagtgggctccgaagacggccagatcaaagtggtctcgcctatcgacgacaccccggcgtccaaggccggtatccaggccggcgacctgatcgtgaagatcaacggccagccaacccgcggccagaccatgaccgaagccgtcgacaagatgcgcggcaagctcggccagaagatcaccctgaccctggtacgcgacggcggcaacccgtttgacgtgaccctggcccgcgcgaccatcacggtcaagagcgtgaaaagccagctgctggagtcgggctacggttatatccgtatcacccagttccaggtcaagaccggcgacgaagtggccaaggccctggccaagctgcgcaaagacaacggcaagaagctcaacggcatcgtgcttgacctgcgcaacaacccaggcggcgtgttgcagtcggcggtcgaggtggtcgaccacttcgtcaccaagggcctgatcgtctacaccaagggccgtatcgccaactcagagttgcgcttctcggccaccggcaacgacctcagcgagaacgtgccactggcggtattgatcaacggtggcagcgcctcggcttcggaaatcgtcgccggtgccctgcaagacctcaagcgcggcgtgctgatgggcaccaccagcttcggcaaaggctcggtgcagaccgtattgccgctgaacaacgagcgtgcgctgaagatcaccacggcgctgtactacacgcccaacggccgctcgatccaggcccagggcatcgtgccggacatcgaagtacgccgcgccaagatcaccaacgagatcgacggcgaatactacaaagaggccgacctgcaaggtcacctgggcaatggcaacggcggtgccgaccagccaaccggcagccgcgccaaggccaagccgatgccgcaggacgatgactaccaactggcccaggcactcagcctgctcaagggcttgagcatcacccgcagccgt

SEQ ID NO: 52 - protein, 437 aa, Pseudomonas fluorescens; identical to SEQ ID NO: 10

SEQ ID NO: 53 - DNA, 1449 bp, Pseudomonas fluorescens
  source 1..1449: mol_type = genomic DNA
atgtcattcatcttttccatttcatcttcaaagtcaaaattacttatgaccactgaaccgtctaaagcgccgccgctttacccgaagacccacctgctcgccgcaagtggtatcgccgcccttctcagcctggcactgctggtattcccttccagtgacgttgaagccaaacgaacatccctgagccttgatctggaaagcccagttgaacaactgacacaagatcaagacgcttccgacgctcaacaagccacaaacactgcaactgaatcacctttcgcccagatcgaaagcacacccgaagacacccagcaagccgcccaggaagcacctgcagcagccaagagtccccagcatcgcgaagtcatcgtgggcaaaggcgacacactctcgaccctgttcgaaaaagttgggttgcctgccgccgctgtaaatgacgtgctcgccagcgataagcaagccaagcaattcactcagctcaaacgtggtcaaaagcttgaatttgagctgacgccagacggccagttgaacaacctgtacaccagcatcagtgacttggaaagcatcagcctgagcaaaggcgccaaaggcttcgcattcaacagaatcaccaccaaacccgtcatgcgttccgcctacgtacatggcgtgatcaacagctccctgtcgcagtcggccgcgcgtgcgggcctgtcgcatagcatgaccatggacatggccagcgtatttggctacgacatcgacttcgcccaggacatccgtcaaggcgacgaattcgacgtgatctacgaacagaaagtagccaacggaaaagtggtcggcactggcaacattctttctgcacgcttcacaaaccgtggcaaaacctacaccgccgtgcgctacaccaacaaacaaggcaacagcagctactacacggctgatggcaacagcatgcgtaaggccttcatccgtacacccgttgactttgcccgtattagctcgcgtttctccatgggccgcaagcatccaattctgaacaaaattcgcgcacacaagggcgtcgactatgccgcgccgcgtggcacgccaatcaaagcagcgggcgacggcaaggtcttgttggcggggcgccgtggtggttacggcaatacggtgatcatccagcacggcaacacttaccgcacgctgtacggccacatgcaagggttcgccaagggcgtcaagacaggcggcaacgtgaaacagggccaagtgatcggctacatcggtaccaccggcctctccaccggcccgcacttgcactacgagttccaggtcaacggcgtacacgtcgacccattgggccagaagctgccgatggccgacccgattgccaaggccgaacgcgcgcgcttcatgcaacagagccagccgctgatggcacggatggatcaagagcgctccaccttgctggcttcggcgaagcgt

SEQ ID NO: 54 - protein, 483 aa, Pseudomonas fluorescens
MSFIFSISSSKSKLLMTTEPSKAPPLYPKTHLLAASGIAALLSLALLVFPSSDVEAKRTSLSLDLESPVEQLTQDQDASDAQQATNTATESPFAQIESTPEDTQQAAQEAPAAAKSPQHREVIVGKGDTLSTLFEKVGLPAAAVNDVLASDKQAKQFTQLKRGQKLEFELTPDGQLNNLYTSISDLESISLSKGAKGFAFNRITTKPVMRSAYVHGVINSSLSQSAARAGLSHSMTMDMASVFGYDIDFAQDIRQGDEFDVIYEQKVANGKVVGTGNILSARFTNRGKTYTAVRYTNKQGNSSYYTADGNSMRKAFIRTPVDFARISSRFSMGRKHPILNKIRAHKGVDYAAPRGTPIKAAGDGKVLLAGRRGGYGNTVIIQHGNTYRTLYGHMQGFAKGVKTGGNVKQGQVIGYIGTTGLSTGPHLHYEFQVNGVHVDPLGQKLPMADPIAKAERARFMQQSQPLMARMDQERSTLLASAKR

SEQ ID NO: 55 - DNA, 1335 bp, Pseudomonas fluorescens
  source 1..1335: mol_type = genomic DNA
atgtccatgactccccgcgaaatcgtccatgaactcaatcgccatatcatcggccaggacgatgccaagcgcgccgttgccattgcgctgcgtaaccgctggcgccggatgcaactgccggaagaactgcgcgttgaagtaacgcccaagaacatcctgatgatcggccccaccggcgtgggtaaaaccgagatcgcccggcgcctggccaaactggccaatgcaccgttcatcaaggtcgaagcgaccaagttcaccgaagtcggctatgtgggccgcgatgtcgagtcgatcattcgtgacctggctgacgccgccctgaagatgctgcgcgaacaggaagtaaccaaggtcagccaccgcgccgaagacgccgctgaagagcgcatcctcgacgccctgttgccaccggcacgcatgggtttcaacgaagacgccgcaccggctaccgattccaacactcgccagctgttccgcaagcgcctgcgtgaaggccagctggatgacaaggaaatcgagatcgaagtggctgaagtgtccggcgtggatatttctgccccgcctggcatggaagaaatgaccagccagctgcagaacctgttcgccaacatgggcaagggcaagaagaaaagccgcaagctcaaggtgaaagaggcgctcaagctcgtgcgcgacgaagaagccgggcgcctggtcaatgaggaagaactcaaggccaaggccctggaagcggtcgagcaacatggcatcgtgtttatcgacgagatcgacaaagtggccaagcgaggcaactcaggcggcgtggatgtgtcccgcgaaggcgtgcagcgcgatttgctgccgctgatcgagggctgcacggtcaacaccaagctgggcatggtcaagactgaccacatcctgtttatcgcttccggtgctttccacctgagcaagcccagcgacctggtgcccgagctgcaaggccgcttgccgattcgggtggagctcaaggcgctgacgccgggcgacttcgagcgcatcctcagcgagccgcatgcctcgctcaccgagcagtaccgcgagttgctgaaaaccgaagggctgggtatcgaattccaggcagacgggatcaagcgcctggcggagatcgcctggcaggtcaacgagaagaccgagaacatcggtgcccgtcgcctgcataccttgcttgagcgcctgctggaggaagtgtccttcagtgccggcgacatggccggtgcgcagaatggcgaagcgatcaagatcgatgctgattacgtcaacagccacttgggcgaattggcgcagaacgaagatctgtctcgttatatcctg

SEQ ID NO: 56 - protein, 445 aa, Pseudomonas fluorescens; identical to SEQ ID NO: 20

SEQ ID NO: 57 - DNA, 528 bp, Pseudomonas fluorescens
  source 1..528: mol_type = genomic DNA
ttgaccaccatcgtttcagtacgtcgccacggcaaagttgtcatgggcggcgacggccaggtttccctgggcaacaccgtgatgaaaggcaacgccaagaaagtgcgccgcctgtaccacggccaggtgcttgccggcttcgcaggcgcaaccgccgacgcctttaccctgttcgagcgtttcgaaggccagcttgagaaacaccagggccacctggtgcgcgccgctgtggaactagccaaagaatggcgcaccgaccgctccctcagccgcctggaggccatgctcgcggttgcgaacaaagacgcttccctgatcatcactggcaacggcgacgtggttgaacccgagcatggcctgatcgccatgggttccggcggcggctacgcccaggctgcggccagcgcgctgttgaagaaaaccgacctgtcggcccgtgaaatcgtcgagaccgccctgggtatcgctggcgatatctgcgtgttcaccaaccacaaccagaccattgaggagcaggacctcgccgag

SEQ ID NO: 58 - protein, 176 aa, Pseudomonas fluorescens
MTTIVSVRRHGKVVMGGDGQVSLGNTVMKGNAKKVRRLYHGQVLAGFAGATADAFTLFERFEGQLEKHQGHLVRAAVELAKEWRTDRSLSRLEAMLAVANKDASLIITGNGDVVEPEHGLIAMGSGGGYAQAAASALLKKTDLSAREIVETALGIAGDICVFTNHNQTIEEQDLAE

SEQ ID NO: 59 - DNA, 1434 bp, Pseudomonas fluorescens
  source 1..1434: mol_type = genomic DNA
atgtcgataccacgtttgaagtcttacttatccatagtcgccacagtgctggtgctgggtcaggccttacctgcgcaagcggtcgagttgcctgacttcacccaactggtggagcaggcctcgcctgccgtggtgaacatcagtaccacgcagaagctgccggatcgcaaagtctcgaaccagcagatgcccgacctggaaggcttgccgcccatgctgcgcgagttcttcgaacgagggatgccgcaaccacgctccccccgtggcggcggtggccagcgcgaagcccaatccctgggctccggcttcatcatttcgcctgacggctatatcctcaccaacaaccacgtgattgccgatgccgacgagattctcgtgcgcctggccgaccgcagtgaactcaaggccaagctgattggcaccgatccacgttccgacgtggccttgcttaaaatcgagggcaaggacttgccggtgcttaagctgggcaagtcccaggacctgaaggccggtcagtgggtggtcgcgatcggttcgccgttcggctttgaccacaccgttacccaaggcatcgtcagcgccatcggtcgcagcctgccgaacgaaaactacgtaccgttcatccagaccgacgtgccgatcaacccgggtaactccggtggcccgctgttcaacctggccggcgaagtggtggggatcaactcgcagatctacacccgctccggcggcttcatgggcgtgtctttcgcgatcccaatcgatgtggccatggacgtctccaatcagctcaaaagcggcggcaaggtcagccgcggctggttgggcgtggtaatccaggaagtgaacaaggacctggctgagtccttcggtctcgacaagccggccggtgccctggttgcgcagattcaggacaatggccctgcggccaaaggcggcctgaaagtcggtgacgtcatcctgagcatgaacggccagccgatcatcatgtcggcagacttgcctcatttggtcggcgcgctcaaggccggcggcaaagccaagctggaagtgattcgtgatggcaagcgccagaacgtcgaactgaccgtaggtgccatcccggaagaaggcgcgaccctggatgccctgggcaacgccaagcccggtgccgagcgcagcagtaaccgcctgggtatcgccgtggttgaactgaccgccgagcagaagaaaaccttcgacctgcaaagcggtgtggtgatcaaggaagttcaggacggcccagccgccttgatcggcctgcaaccgggtgacgtgatcactcacttgaacaaccaggcaatcgataccaccaaggaattcgccgacatcgccaaggcgttgccgaagaatcgctcggtgtcgatgcgcgtcctgcgtcaaggccgtgccagcttcattaccttcaagctggctgag

SEQ ID NO: 60 - protein, 478 aa, Pseudomonas fluorescens; identical to SEQ ID NO: 19

SEQ ID NO: 61 - DNA, 1647 bp, Pseudomonas fluorescens
  source 1..1647: mol_type = genomic DNA
atgacggtggtgaaggtcttttcaatgtgggagctttatcgggctgacaacggagcagtcggcatcggtaactcgcatatatggacggttaactttccactgttcagagtatcaaagcacatgcatatccctgttaggcagtcttcttactcgcgtccttcagataagttacagcccgatctttcacccgatgaacaccaagttgttctctgggccaacaataaaaaatctttcaccacggatcaggccgcgaaacacatcacccgcggtggcttcaagtttcatgatcgcaacaatgatggaaaaatcgtcgtgggttataactttgcgggcggcttcaatgcggctcagaaagaacgggccaggcaagcccttcagtactgggcggatgttgctaatatcgaatttgttgaaaatggcccgaacacggatggcacaataagcatcaagggtgttccgggttcggcaggcgtcgcggggttgcccaacaaatataattcgaacgtccaggccaatataggcacccagggtgggcaaaacccggcgatgggcagtcacttcctgggcttattgatccatgaactggggcataccctggggctgagtcatccaggtaaatacgacggccagggtttcaattacgatcgggctgccgaatatgcccaggacaccaaggctcgcagtgtcatgagctattggacggagactcatcagccggggcacaattttgccgggcgcagcccgggtgccccgatgatggacgatatcgccgccgcccagcggctctacggcgccaacaccaaaacccggaataccgacaccacctacggcttcaattccaattcaggccgggaggcttatagcctcaagcaggggagcgacaagccgatcttcaccgtctgggacggtggaggtaatgacacgctcgacttctccgggttcacccagaaccaaaccatcaacctcaaggctgagtcattctcggacgtggggggcttgcgaggaaatgtgtcgattgccaagggtgtgagtgtggaaaacgccattggcggtacaggcaacgataccttgacggggaacgagggcaacaatcggctcacgggcggcaagggggccgataagctgcacggcggagctggagcagacacgtttgtttaccgccgcgccagcgattcaacgccgcaggcaccggacatcatccaggacttccagagcgggagcgacaagatcgacctgaccggtgttgttcaggaggcggggctcaagtcgctgagcttcgtcgagaaattcagcggcaaggcgggcgaggccgtgctcggccaagacgcgaaaaccggccgtttcacgttggcggtggacacaacgggaaatggtacggcggatctactggttgccagccaaagccagatcaaacaggcggatgtgatctggaacggtcaggcgccgacagtgacgccaacgcctgaacccactgtggtgcctgtgtcagatcccgtgccgacccctacttcagagccgactgaacctgaacccacgcctgagcccgcccctttgcccgtcccgactccacggcctggaggagggtttatcgggaaaattttttcatcattcaaggggttcataaaaaaagtgtggtcgatattcagg

SEQ ID NO: 62 - protein, 549 aa, Pseudomonas fluorescens
MTVVKVFSMWELYRADNGAVGIGNSHIWTVNFPLFRVSKHMHIPVRQSSYSRPSDKLQPDLSPDEHQVVLWANNKKSFTTDQAAKHITRGGFKFHDRNNDGKIVVGYNFAGGFNAAQKERARQALQYWADVANIEFVENGPNTDGTISIKGVPGSAGVAGLPNKYNSNVQANIGTQGGQNPAMGSHFLGLLIHELGHTLGLSHPGKYDGQGFNYDRAAEYAQDTKARSVMSYWTETHQPGHNFAGRSPGAPMMDDIAAAQRLYGANTKTRNTDTTYGFNSNSGREAYSLKQGSDKPIFTVWDGGGNDTLDFSGFTQNQTINLKAESFSDVGGLRGNVSIAKGVSVENAIGGTGNDTLTGNEGNNRLTGGKGADKLHGGAGADTFVYRRASDSTPQAPDIIQDFQSGSDKIDLTGVVQEAGLKSLSFVEKFSGKAGEAVLGQDAKTGRFTLAVDTTGNGTADLLVASQSQIKQADVIWNGQAPTVTPTPEPTVVPVSDPVPTPTSEPTEPEPTPEPAPLPVPTPRPGGGFIGKIFSSFKGFIKKVWSIFR

SEQ ID NO: 63 - DNA, 2112 bp, Pseudomonas fluorescens
  source 1..2112: mol_type = genomic DNA
atgcgttatcaattgcccccgcgtcgaatcagcatgaagcatctgttccccagcaccgccctcgcttttttcattggtctcggcttcgcgtcgatgtcgaccaatacgttcgcagccaatagctgggacaaccttcagcctgatcgcgatgaggtgattgccagccttaacgtcgtcgagttgcttaagcgccatcactacagcaagccgccgctggacgacgctcgctcagtgatcatctacgacagctacctcaagctgctggacccgtcgcgcagctacttcctggccagcgatatcgctgagttcgacaagtggaagacgcaattcgacgacttcctcaagagcggcgacctgcagcctggcttcaccatctacaagcgctacctagaccgcgtcaaagcgcgtctggacttcgccctgggtgagctgaacaaaggcgtcgacaagctcgatttcacccagaaagaaacccttctggtggaccgcaaggacgccccttggctgaccagcaccgcagccctagacgacctgtggcgcaaacgcgtcaaggacgaagtgctgcgcttgaagatcgccggcaaagagcccaaggccattcaagagctgttgaccaagcgctacaaaaaccagctggcgcgcctggaccagacccgtgccgaggatatcttccaggcctacatcaacacctttgcgatgtcctacgacccgcacaccaattatctgtcgccagataacgcggaaaatttcgatatcaatatgagtctgtccctggaaggcatcggtgccgtcctgcaaagcgacaatgaccaggtgaagattgtacgtctggtgccggcaggcccggctgacaaaaccaagcaagtggcaccggccgacaagatcatcggcgtggcccaggccgacaaagagatggtcgatgtggtcggctggcgcctggacgaagtggtcaagctgatccgtgggcctaaaggcagcgtggtgcgcctggaagtgattccgcacaccaatgcaccgaacgaccagaccagcaagatcgtgtccatcacccgtgaagcggtgaagctcgaagaccaggccgtgcagaagaaagtcctcaacctcaagcaggatggcaaggactacaagctgggggtgattgaaatcccggccttctacctggacttcaaggcgttccgtgccggtgatccggactacaagtccaccacccgcgacgtgaagaaaatcctcacagaactgcagaaagagaaagtcgacggcgtggtcatcgacctgcgcaacaacggcggcggctccctgcaggaagccaccgagctgaccagcctgtttatcgacaagggcccgaccgtgttggtacgcaacgctgacggccgtgtcgacgtgctcgaagacgagaacccgggggccttctacaaagggccgatggcgctgctggtcaaccgcctctcggcctcggcctcggagattttcgccggtgccatgcaggactaccaccgtgcactgatcatcggcggccagaccttcggcaaaggcaccgtgcagaccatccagccgctgaaccatggcgagcttaagctgacactggccaagttctaccgggtctccgggcagagcacccagcatcagggcgtactgccggatatcgatttcccgtcgatcatcgacaccaaggaaattggcgaaagcgccctgcctgaagccatgccgtgggacaccatccgccctgcgatcaagccggcgtcggatccgttcaagccgttcctggcacagctgaaggctgaccacgacacccgctctgccaaggatgccgagttcgtgtttatccgcgacaagctggccctggccaagaagctgatggaagagaagaccgtcagcctcaacgaagcggatcgccgtgcacagcactccagcatcgagaatcagcaactggtgctggaaaacacccgccgcaaggccaaaggtgaagacccgctcaaagagctgaagaaagaagatgaagacgcgctgccgaccgaggcggataaaaccaagccggaagacgacgcctacttggccgagactggccggatcctgctggattacctgaagatcaccaagcaggtggccaagcag

SEQ ID NO: 64 - protein, 704 aa, Pseudomonas fluorescens
MRYQLPPRRISMKHLFPSTALAFFIGLGFASMSTNTFAANSWDNLQPDRDEVIASLNVVELLKRHHYSKPPLDDARSVIIYDSYLKLLDPSRSYFLASDIAEFDKWKTQFDDFLKSGDLQPGFTIYKRYLDRVKARLDFALGELNKGVDKLDFTQKETLLVDRKDAPWLTSTAALDDLWRKRVKDEVLRLKIAGKEPKAIQELLTKRYKNQLARLDQTRAEDIFQAYINTFAMSYDPHTNYLSPDNAENFDINMSLSLEGIGAVLQSDNDQVKIVRLVPAGPADKTKQVAPADKIIGVAQADKEMVDVVGWRLDEVVKLIRGPKGSVVRLEVIPHTNAPNDQTSKIVSITREAVKLEDQAVQKKVLNLKQDGKDYKLGVIEIPAFYLDFKAFRAGDPDYKSTTRDVKKILTELQKEKVDGVVIDLRNNGGGSLQEATELTSLFIDKGPTVLVRNADGRVDVLEDENPGAFYKGPMALLVNRLSASASEIFAGAMQDYHRALIIGGQTFGKGTVQTIQPLNHGELKLTLAKFYRVSGQSTQHQGVLPDIDFPSIIDTKEIGESALPEAMPWDTIRPAIKPASDPFKPFLAQLKADHDTRSAKDAEFVFIRDKLALAKKLMEEKTVSLNEADRRAQHSSIENQQLVLENTRRKAKGEDPLKELKKEDEDALPTEADKTKPEDDAYLAETGRILLDYLKITKQVAKQ

SEQ ID NO: 65 - DNA, 915 bp, Pseudomonas fluorescens
  source 1..915: mol_type = genomic DNA
gccgtggtgatcaccggcacccggctggtctaccccgcggaccagaaagaaatcaccgtcaagctcaacaacaacggtacgctgccggcactcgtgcaatcatggatcgacactggctcggtcgagagcaccccgactagctccaaggccccattcctgctctcgcctccggtggcccgcatcgaccccaccaaggggcaaagcctgcgtgtactgttcacgggtgcgcccttggcgcaagacaaggaatcggtgttctggctgaatgtcctggaaatcccgccgaagcccgaggccggtgccgatctgaacactctccagatggccttccgctcgcggatcaagctgttctaccgtccggtaggtctgcccggcaacccgaacgaagcggtcgagcaagtgcagtggcaactggtgactgcccgcgacggccagggcctggcgctcaaagcgtacaatcccagcgccttccacgtatcgctcatcgagctggatctggtcgccggcaaccagcgctaccgcagcgaagatggcatggtgggtcccggcgagactcgccagtttgccctccccaccctcaaggcgcggccgagcagccaggcccaagtggagttttcggcgatcaacgactacggtgcgttggtgccgacccggaacacgctgcaaccgggcgggggcgggagtggtggtggcggtagccatcatcaccaccatcacggcggcggtggcagcgacgacgatgacaagggtggcggtggcgcgatggggtccgagggcgacaagacctgtcccgatggctatgagcacacgtgcggctgcatcggcggctgcggctgcaagcgcagcgcctgcattggggccctgtgctgccaagcgagcctcggcggctggctgtcggacggcgaaacctacacc

SEQ ID NO: 66 - protein, 305 aa, Pseudomonas fluorescens
AVVITGTRLVYPADQKEITVKLNNNGTLPALVQSWIDTGSVESTPTSSKAPFLLSPPVARIDPTKGQSLRVLFTGAPLAQDKESVFWLNVLEIPPKPEAGADLNTLQMAFRSRIKLFYRPVGLPGNPNEAVEQVQWQLVTARDGQGLALKAYNPSAFHVSLIELDLVAGNQRYRSEDGMVGPGETRQFALPTLKARPSSQAQVEFSAINDYGALVPTRNTLQPGGGGSGGGGSHHHHHHGGGGSDDDDKGGGGAMGSEGDKTCPDGYEHTCGCIGGCGCKRSACIGALCCQASLGGWLSDGETYT

SEQ ID NO: 67 - protein, 46 aa, synthetic construct
GGSEGDKTCPDGYEHTCGCIGGCGCKRSACIGALCCQASLGGWLSD

SEQ ID NO: 68 - protein, 176 aa, Pseudomonas fluorescens; identical to SEQ ID NO: 58

SEQ ID NO: 69 - DNA, 69 bp, Pseudomonas fluorescens
  source 1..69: mol_type = genomic DNA
atgccgcctcgttctatcgccgcatgtctggggctgctgggcttgctcatggctacccaggccgccgcc

SEQ ID NO: 70 - protein, 23 aa, Pseudomonas fluorescens
MPPRSIAACLGLLGLLMATQAAA

SEQ ID NO: 71 - DNA, 60 bp, Pseudomonas fluorescens
  source 1..60: mol_type = genomic DNA
atgtttgccaaactcgttgctgtttccctgctgactctggcgagcggccagttgcttgct

SEQ ID NO: 72 - protein, 20 aa, Pseudomonas fluorescens
MFAKLVAVSLLTLASGQLLA

SEQ ID NO: 73 - DNA, 72 bp, Pseudomonas fluorescens
  source 1..72: mol_type = genomic DNA
atgaaactgaaacgtttgatggcggcaatgacttttgtcgctgctggcgttgcgaccgccaacgcggtggcc

SEQ ID NO: 74 - protein, 24 aa, Pseudomonas fluorescens
MKLKRLMAAMTFVAAGVATANAVA

SEQ ID NO: 75 - DNA, 72 bp, Pseudomonas fluorescens
  source 1..72: mol_type = genomic DNA
atgaaactgaaacgtttgatggcggcaatgacttttgtcgctgctggcgttgcgaccgtcaacgcggtggcc

SEQ ID NO: 76 - protein, 24 aa, Pseudomonas fluorescens
MKLKRLMAAMTFVAAGVATVNAVA

SEQ ID NO: 77 - DNA, 87 bp, Pseudomonas fluorescens
  source 1..87: mol_type = genomic DNA
atgcaaagcctgccgttctctgcgttacgcctgctcggtgtgctggcagtcatggtctgcgtgctgttgacgacgccagcccgtgcc

SEQ ID NO: 78 - protein, 29 aa, Pseudomonas fluorescens
MQSLPFSALRLLGVLAVMVCVLLTTPARA

SEQ ID NO: 79 - DNA, 93 bp, Pseudomonas fluorescens
  source 1..93: mol_type = genomic DNA
atgatccgtgacaaccgactcaagacatcccttctgcgcggcctgaccctcaccctactcagcctgaccctgctctcgcccgcggcccattct

SEQ ID NO: 80 - protein, 31 aa, Pseudomonas fluorescens
MIRDNRLKTSLLRGLTLTLLSLTLLSPAAHS

SEQ ID NO: 81 - protein, 6 aa, synthetic construct
CATVHQ

SEQ ID NO: 82 - protein, 6 aa, synthetic construct
CAIVQQ

SEQ ID NO: 83 - protein, 6 aa, synthetic construct
CATVDQ

SEQ ID NO: 84 - skipped

SEQ ID NO: 85 - protein, 478 aa, synthetic construct
MSIPRLKSYLSIVATVLVLGQALPAQAVELPDFTQLVEQASPAVVNISTTQKLPDRKVSNQQMPDLEGLPPMLREFFERGMPQPRSPRGGGGQREAQSLGSGFIISPDGYILTNNHVIADADEILVRLADRSELKAKLIGTDPRSDVALLKIEGKDLPVLKLGKSQDLKAGQWVVAIGSPFGFDHTVTQGIVSAIGRSLPNENYVPFIQTDVPINPGNAGGPLFNLAGEVVGINSQIYTRSGGFMGVSFAIPIDVAMDVSNQLKSGGKVSRGWLGVVIQEVNKDLAESFGLDKPAGALVAQIQDNGPAAKGGLKVGDVILSMNGQPIIMSADLPHLVGALKAGGKAKLEVIRDGKRQNVELTVGAIPEEGATLDALGNAKPGAERSSNRLGIAVVELTAEQKKTFDLQSGVVIKEVQDGPAALIGLQPGDVITHLNNQAIDTTKEFADIAKALPKNRSVSMRVLRQGRASFITFKLAE

SEQ ID NO: 86 - DNA, 1434 bp, synthetic construct
  source 1..1434: mol_type = other DNA
atgtcgataccacgtttgaagtcttacttatccatagtcgccacagtgctggtgctgggtcaggccttacctgcgcaagcggtcgagttgcctgacttcacccaactggtggagcaggcctcgcctgccgtggtgaacatcagtaccacgcagaagctgccggatcgcaaagtctcgaaccagcagatgcccgacctggaaggcttgccgcccatgctgcgcgagttcttcgaacgagggatgccgcaaccacgctccccccgtggcggcggtggccagcgcgaagcccaatccctgggctccggcttcatcatttcgcctgacggctatatcctcaccaacaaccacgtgattgccgatgccgacgagattctcgtgcgcctggccgaccgcagtgaactcaaggccaagctgattggcaccgatccacgttccgacgtggccttgcttaaaatcgagggcaaggacttgccggtgcttaagctgggcaagtcccaggacctgaaggccggtcagtgggtggtcgcgatcggttcgccgttcggctttgaccacaccgttacccaaggcatcgtcagcgccatcggtcgcagcctgccgaacgaaaactacgtaccgttcatccagaccgacgtgccgatcaacccgggtaacgccggtggcccgctgttcaacctggccggcgaagtggtggggatcaactcgcagatctacacccgctccggcggcttcatgggcgtgtctttcgcgatcccaatcgatgtggccatggacgtctccaatcagctcaaaagcggcggcaaggtcagccgcggctggttgggcgtggtaatccaggaagtgaacaaggacctggctgagtccttcggtctcgacaagccggccggtgccctggttgcgcagattcaggacaatggccctgcggccaaaggcggcctgaaagtcggtgacgtcatcctgagcatgaacggccagccgatcatcatgtcggcagacttgcctcatttggtcggcgcgctcaaggccggcggcaaagccaagctggaagtgattcgtgatggcaagcgccagaacgtcgaactgaccgtaggtgccatcccggaagaaggcgcgaccctggatgccctgggcaacgccaagcccggtgccgagcgcagcagtaaccgcctgggtatcgccgtggttgaactgaccgccgagcagaagaaaaccttcgacctgcaaagcggtgtggtgatcaaggaagttcaggacggcccagccgccttgatcggcctgcaaccgggtgacgtgatcactcacttgaacaaccaggcaatcgataccaccaaggaattcgccgacatcgccaaggcgttgccgaagaatcgctcggtgtcgatgcgcgtcctgcgtcaaggccgtgccagcttcattaccttcaagctggctgag

SEQ ID NO: 87 - protein, 168 aa, Pseudomonas fluorescens
MIDDMRLGRERRFLVLLGIICLALIGGALYMQVVLGEAPCPLCILQRYALLLIALFAFIGAAMRTKGALTFFEGLVVLSALGGVAAAGHHVYTQFFPQVSCGIDVLQPIVDDLPLAKVFPLGFQVDGFCSTPYPPILGLSLAQWALVAFVLTAILVPLCIYRNRHPKA

SEQ ID NO: 88 - protein, 575 aa, Pseudomonas fluorescens
MRHLFTFLLVLFAGFAQAAPGSPFETKPDFLPVGKAFAFTSERLESGETQLFWQIADGYYLYQQRMKFDGLAEKPVLPEGEAHSDEFFGEQQVYRQGLEVKIPAGTTGQVKLGWQGCADAGLCYPPQSITVDLGGNPAVAATAQAQDQSLASGLQQRSLGWSLLVFFGLGLLLAFAPCSLPMLPILAGLVVGSGASPRRGFALAGSYVVCMALVYAALGVMAALLGANLAALLQTPWILGSFAALFVLLALPMFGFFELQLPAFLRDRLDNVSRQQSGGSLVGAGVLGALSGLLVGPCMTAPLAGALLYIAQSGNALHGGLILFAMGIGIGIPLLLLVTVGNRFLPKPGTWMNVLKGIFGFLFLGTAVLMIRPVVGDSLWIGLWGALALVMAYCGWALARESGLAAKVFGAGSLVLGLWGAVLVVGAAGGSDELWQPLKVYSGSRVADAPSAHDAFTTVSDPAVLQSQLDSAKAQGQWVLLDYYADWCVSCKIMEKQVFGKPEVMDALKDVRLLRLDVTADNAASRELLGRYKVPGPPSFVWIGPDGEERRAQRITGEVDAAAFLQRWTQTRDAR

SEQ ID NO: 89 - protein, 253 aa, Pseudomonas fluorescens
MPRLRHLLTLLPLTLAAALAQAEDLPAPIKQIEAKGAKIIGKFDAPSGLTGYAAQYQNRGMALYLTADGKNVIAGNLYDAQGNDLSTAPLEKLVYAPMAKEVWAKMENSSWIQDGDKNAPRTIYLFSDPNCPYCNMFWEQARPWVKAGKVQLRHIMVGIIREDSPGKSAALLAAKDPQKALQDHEAAGKGSKLKALEKIPAEVEAKLDANMKLMDELELSATPAIFYLDDKGGLQQQQGAPSPDKLVKILGPK

SEQ ID NO: 90 - DNA, 783 bp, synthetic construct
  source 1..783: mol_type = other DNA
gcggacgtaccgctggaagcgggcaagacttacgtggagctcgccaacccggtcccggtcgccgtgccgggcaaaatcgaagtggtggagctgttctggtacggctgccctcattgctacgcgttcgagcccacgatcaacccctgggccgagaagctgcccaaagacgtcaacttccgtcgcatccccgcgatgttcgggggtccctgggacgcccatggtcagctgtttctcaccttggaagccatgggcgtagagcacaaggtccacaacgccgtgttcgaggccatccaaaagcaaggcaagcggctgaccaagccggacgagatggccgattttgtggccactcaaggcgtggacaaggataagtttctggcgaccttcaatagcttcgcgatccaaggccagatcaagcaagcgaaagaactggcccagaagtatggcgtgcagggcgtccccaccctgatcgtgaacggcaagtaccgcttcgacctgggttcgaccggtgggcccgaggcgaccctgaacgtggcggatcaactcatcgcgaaggaacgcgcagccaagggtggcggtggctcgggcggtggcggtagccaccatcatcaccaccacgggggcgggggtagcgacgatgacgataagggtggttccaacgcgtgccctgatgacttcgactatcgctgttcctgtattggtggttgcggctgcgcccggaaggggtgcgtgggccccctctgctgccgcagcgacctgggcggctacctgaccgattcgccggcc

SEQ ID NO: 91 - protein, 261 aa, synthetic construct
ADVPLEAGKTYVELANPVPVAVPGKIEVVELFWYGCPHCYAFEPTINPWAEKLPKDVNFRRIPAMFGGPWDAHGQLFLTLEAMGVEHKVHNAVFEAIQKQGKRLTKPDEMADFVATQGVDKDKFLATFNSFAIQGQIKQAKELAQKYGVQGVPTLIVNGKYRFDLGSTGGPEATLNVADQLIAKERAAKGGGGSGGGGSHHHHHHGGGGSDDDDKGGSNACPDDFDYRCSCIGGCGCARKGCVGPLCCRSDLGGYLTDSPA

SEQ ID NO: 92 - DNA, 765 bp, synthetic construct
  source 1..765: mol_type = other DNA
gcggacgtaccgctggaagcgggcaagacttacgtggagctcgccaacccggtcccggtcgccgtgccgggcaaaatcgaagtggtggagctgttctggtacggctgccctcattgctacgcgttcgagcccacgatcaacccctgggccgagaagctgcccaaagacgtcaacttccgtcgcatccccgcgatgttcgggggtccctgggacgcccatggtcagctgtttctcaccttggaagccatgggcgtagagcacaaggtccacaacgccgtgttcgaggccatccaaaagcaaggcaagcggctgaccaagccggacgagatggccgattttgtggccactcaaggcgtggacaaggataagtttctggcgaccttcaatagcttcgcgatccaaggccagatcaagcaagcgaaagaactggcccagaagtatggcgtgcagggcgtccccaccctgatcgtgaacggcaagtaccgcttcgacctgggttcgaccggtgggcccgaggcgaccctgaacgtggcggatcaactcatcgcgaaggaacgcgcagccaagggtggcggtggctcgggcggtggcggtagccaccatcatcaccaccacgggggcgggggtagcgacgatgacgataagggtagcacttgccccgacggctacgagcacacgtgcgggtgcatcggcggctgcggctgcaagcgctcggcctgcatcggtgcgctgtgctgtcaagcctccctcggcggctggctgagc

SEQ ID NO: 93 - protein, 255 aa, synthetic construct
ADVPLEAGKTYVELANPVPVAVPGKIEVVELFWYGCPHCYAFEPTINPWAEKLPKDVNFRRIPAMFGGPWDAHGQLFLTLEAMGVEHKVHNAVFEAIQKQGKRLTKPDEMADFVATQGVDKDKFLATFNSFAIQGQIKQAKELAQKYGVQGVPTLIVNGKYRFDLGSTGGPEATLNVADQLIAKERAAKGGGGSGGGGSHHHHHHGGGGSDDDDKGSTCPDGYEHTCGCIGGCGCKRSACIGALCCQASLGGWLS

SEQ ID NO: 94 - DNA, 567 bp, Pseudomonas fluorescens
  source 1..567: mol_type = genomic DNA
gcggacgtaccgctggaagcgggcaagacttacgtggagctcgccaacccggtcccggtcgccgtgccgggcaaaatcgaagtggtggagctgttctggtacggctgccctcattgctacgcgttcgagcccacgatcaacccctgggccgagaagctgcccaaagacgtcaacttccgtcgcatccccgcgatgttcgggggtccctgggacgcccatggtcagctgtttctcaccttggaagccatgggcgtagagcacaaggtccacaacgccgtgttcgaggccatccaaaagcaaggcaagcggctgaccaagccggacgagatggccgattttgtggccactcaaggcgtggacaaggataagtttctggcgaccttcaatagcttcgcgatccaaggccagatcaagcaagcgaaagaactggcccagaagtatggcgtgcagggcgtccccaccctgatcgtgaacggcaagtaccgcttcgacctgggttcgaccggtgggcccgaggcgaccctgaacgtggcggatcaactcatcgcgaaggaacgcgcagccaag

SEQ ID NO: 95 - protein, 189 aa, Pseudomonas fluorescens
ADVPLEAGKTYVELANPVPVAVPGKIEVVELFWYGCPHCYAFEPTINPWAEKLPKDVNFRRIPAMFGGPWDAHGQLFLTLEAMGVEHKVHNAVFEAIQKQGKRLTKPDEMADFVATQGVDKDKFLATFNSFAIQGQIKQAKELAQKYGVQGVPTLIVNGKYRFDLGSTGGPEATLNVADQLIAKERAAK

SEQ ID NO: 96 - protein, 5 aa, synthetic construct
DDDDK

SEQ ID NO: 97 - protein, 8 aa, synthetic construct
GGGGAMGS

(19) United States
(12) Patent Application Publication
(10) Pub. No.: US 2026/0201020 A1
(43) Pub. Date: Jul. 16, 2026

(54) METHODS FOR EXPRESSION OF FUSION-FREE BOVINE ULTRALONG CDR3 SCAFFOLD

(71) Applicant: PELICAN TECHNOLOGY HOLDINGS, INC., San Diego, CA (US)

(72) Inventors: Russell COLEMAN, San Diego, CA (US); Yinghui LEE, San Diego, CA (US)

(73) Assignee: PELICAN TECHNOLOGY HOLDINGS, INC., San Diego, CA (US)

(21) Appl. No.: 19/134,680

(22) PCT Filed: Dec. 1, 2023
(86) PCT No.: PCT/US2023/082148

§ 371 Date: May 30, 2025

Related U.S. Application Data
(60) Provisional application No. 63/430,288, filed on Dec. 5, 2022.
(60) Provisional application No. 63/529,931, filed on Jul. 31, 2023.

(51) Int. Cl. C07K 16/104; B01D 15/34; B01D 15/36; C07K 14/415; C12N 1/20; C12N 9/52; C12N 15/78; C12P 21/02; C12R 1/39; G01N 33/68

(52) U.S. Cl. CPC C07K 16/104; B01D 15/34; B01D 15/362; C07K 14/415; C12N 1/20; C12N 9/52; C12N 15/78; C12P 21/02; G01N 33/6857; C07K 2317/14; C07K 2317/20; C07K 2317/565; C07K 2319/034; C12N 2800/22; C12R 2001/39; C12Y 304/2404; G01N 2333/165

(57) ABSTRACT

The present invention provides methods for producing a recombinant ultralong CDR3 knob peptide. Described methods comprise: culturing a Pseudomonadales host cell in a culture medium and expressing the recombinant ultralong CDR3 knob peptide in the periplasm of the Pseudomonadales host cell from an expression construct comprising a nucleic acid encoding the recombinant ultralong CDR3 knob peptide directly and operably linked to a periplasmic secretion leader; wherein the recombinant ultralong CDR3 knob peptide is produced by secretion into the periplasm of the Pseudomonadales host cell, and wherein the produced recombinant ultralong CDR3 knob peptide is present in the periplasm in soluble form, active form, or both.

### BACKGROUND OF THE INVENTION

Ultralong bovine antibodies comprise a very long CDR-H3 sequence that forms a stalk and cysteine-rich knob structure. Due to their unique structure, these antibodies can bind to antigens not accessible to conventional antibodies. Mini-domain peptides containing or derived from such antibodies have been made to specifically target certain antigens, including the SARS-CoV2 RBD. However, due to their small size and folding requirements, production of the small peptides in active form typically requires expression as part of a larger fusion protein which must be cleaved and the resulting components separated. Fast and cost-effective methods for producing pure, soluble, active, ultralong bovine antibody knob domains are needed.

### SUMMARY OF THE INVENTION

In one aspect, a method is provided for producing a recombinant ultralong CDR3 knob peptide, the method comprising: culturing a Pseudomonadales host cell in a culture medium and expressing the recombinant ultralong CDR3 knob peptide in the periplasm of the Pseudomonadales host cell from an expression construct comprising a nucleic acid encoding the recombinant ultralong CDR3 knob peptide directly and operably linked to a periplasmic secretion leader; wherein the recombinant ultralong CDR3 knob peptide is produced by secretion into the periplasm of the Pseudomonadales host cell, and wherein the produced recombinant ultralong CDR3 knob peptide is present in the periplasm in soluble form, active form, or both. In certain embodiments, an amount of the ultralong CDR3 knob peptide secreted into the periplasm is subsequently released to the culture medium. In some embodiments, the expression construct does not comprise a nucleic acid encoding an N-terminal linker, a fusion partner, a C-terminal purification tag, e.g., a His-tag, or any combination thereof. In some embodiments, the expression construct does not comprise any one of: a nucleic acid encoding an N-terminal linker, a fusion partner, and a C-terminal purification tag.

The recombinant ultralong CDR3 knob peptide can be about 1 to about 10 kDa. In certain embodiments, the recombinant ultralong CDR3 knob peptide is about 1 kDa to about 2 kDa, about 1 kDa to about 3 kDa, about 1 kDa to about 4 kDa, about 1 kDa to about 5 kDa, about 1 kDa to about 6 kDa, about 1 kDa to about 7 kDa, about 1 kDa to about 8 kDa, about 1 kDa to about 9 kDa, about 1 kDa to about 10 kDa, about 2 kDa to about 3 kDa, about 2 kDa to about 4 kDa, about 2 kDa to about 5 kDa, about 2 kDa to about 6 kDa, about 2 kDa to about 7 kDa, about 2 kDa to about 8 kDa, about 2 kDa to about 9 kDa, about 2 kDa to about 10 kDa, about 3 kDa to about 4 kDa, about 3 kDa to about 5 kDa, about 3 kDa to about 6 kDa, about 3 kDa to about 7 kDa, about 3 kDa to about 8 kDa, about 3 kDa to about 9 kDa, about 3 kDa to about 10 kDa, about 4 kDa to about 5 kDa, about 4 kDa to about 6 kDa, about 4 kDa to about 7 kDa, about 4 kDa to about 8 kDa, about 4 kDa to about 9 kDa, about 4 kDa to about 10 kDa, about 5 kDa to about 6 kDa, about 5 kDa to about 7 kDa, about 5 kDa to about 8 kDa, about 5 kDa to about 9 kDa, about 5 kDa to about 10 kDa, about 6 kDa to about 7 kDa, about 6 kDa to about 8 kDa, about 6 kDa to about 9 kDa, about 6 kDa to about 10 kDa, about 7 kDa to about 8 kDa, about 7 kDa to about 9 kDa, about 7 kDa to about 10 kDa, about 8 kDa to about 9 kDa, about 8 kDa to about 10 kDa, or about 9 kDa to about 10 kDa. In certain embodiments, the recombinant ultralong CDR3 knob peptide is about 1 kDa, about 2 kDa, about 3 kDa, about 4 kDa, about 5 kDa, about 6 kDa, about 7 kDa, about 8 kDa, about 9 kDa, or about 10 kDa. In certain embodiments, the recombinant ultralong CDR3 knob peptide is about at least about 1 kDa, about 2 kDa, about 3 kDa, about 4 kDa, about 5 kDa, about 6 kDa, about 7 kDa, about 8 kDa, or about 9 kDa. In certain embodiments, the recombinant ultralong CDR3 knob peptide is about at most about 2 kDa, about 3 kDa, about 4 kDa, about 5 kDa, about 6 kDa, about 7 kDa, about 8 kDa, about 9 kDa, or about 10 kDa.

The recombinant ultralong CDR3 knob peptide can be about 10 to about 100 amino acids in length. In certain embodiments, recombinant ultralong CDR3 knob peptide is about 10 amino acids to about 20 amino acids, about 10 amino acids to about 25 amino acids, about 10 amino acids to about 30 amino acids, about 10 amino acids to about 35 amino acids, about 10 amino acids to about 40 amino acids, about 10 amino acids to about 50 amino acids, about 10 amino acids to about 60 amino acids, about 10 amino acids to about 70 amino acids, about 10 amino acids to about 80 amino acids, about 10 amino acids to about 90 amino acids, about 10 amino acids to about 100 amino acids, about 20 amino acids to about 25 amino acids, about 20 amino acids to about 30 amino acids, about 20 amino acids to about 35 amino acids, about 20 amino acids to about 40 amino acids, about 20 amino acids to about 50 amino acids, about 20 amino acids to about 60 amino acids, about 20 amino acids to about 70 amino acids, about 20 amino acids to about 80 amino acids, about 20 amino acids to about 90 amino acids, about 20 amino acids to about 100 amino acids, about 25 amino acids to about 30 amino acids, about 25 amino acids to about 35 amino acids, about 25 amino acids to about 40 amino acids, about 25 amino acids to about 50 amino acids, about 25 amino acids to about 60 amino acids, about 25 amino acids to about 70 amino acids, about 25 amino acids to about 80 amino acids, about 25 amino acids to about 90 amino acids, about 25 amino acids to about 100 amino acids, about 30 amino acids to about 35 amino acids, about 30 amino acids to about 40 amino acids, about 30 amino acids to about 50 amino acids, about 30 amino acids to about 60 amino acids, about 30 amino acids to about 70 amino acids, about 30 amino acids to about 80 amino acids, about 30 amino acids to about 90 amino acids, about 30 amino acids to about 100 amino acids, about 35 amino acids to about 40 amino acids, about 35 amino acids to about 50 amino acids, about 35 amino acids to about 60 amino acids, about 35 amino acids to about 70 amino acids, about 35 amino acids to about 80 amino acids, about 35 amino acids to about 90 amino acids, about 35 amino acids to about 100 amino acids, about 40 amino acids to about 50 amino acids, about 40 amino acids to about 60 amino acids, about 40 amino acids to about 70 amino acids, about 40 amino acids to about 80 amino acids, about 40 amino acids to about 90 amino acids, about 40 amino acids to about 100 amino acids, about 50 amino acids to about 60 amino acids, about 50 amino acids to about 70 amino acids, about 50 amino acids to about 80 amino acids, about 50 amino acids to about 90 amino acids, about 50 amino acids to about 100 amino acids, about 60 amino acids to about 70 amino acids, about 60 amino acids to about 80 amino acids, about 60 amino acids to about 90 amino acids, about 60 amino acids to about 100 amino acids, about 70 amino acids to about 80 amino acids, about 70 amino acids to about 90 amino acids, about 70 amino acids to about 100 amino acids, about 80 amino acids to about 90 amino acids, about 80 amino acids to about 100 amino acids, or about 90 amino acids to about 100 amino acids in length. In certain embodiments, recombinant ultralong CDR3 knob peptide is about 10 amino acids, about 20 amino acids, about 25 amino acids, about 30 amino acids, about 35 amino acids, about 40 amino acids, about 50 amino acids, about 60 amino acids, about 70 amino acids, about 80 amino acids, about 90 amino acids, or about 100 amino acids in length. In certain embodiments, recombinant ultralong CDR3 knob peptide is at least about 10 amino acids, about 20 amino acids, about 25 amino acids, about 30 amino acids, about 35 amino acids, about 40 amino acids, about 50 amino acids, about 60 amino acids, about 70 amino acids, about 80 amino acids, or about 90 amino acids in length. In certain embodiments, recombinant ultralong CDR3 knob peptide is at most about 20 amino acids, about 25 amino acids, about 30 amino acids, about 35 amino acids, about 40 amino acids, about 50 amino acids, about 60 amino acids, about 70 amino acids, about 80 amino acids, about 90 amino acids, or about 100 amino acids in length.

The recombinant ultralong CDR3 knob peptide can include a cysteine motif. In some embodiments, the cysteine motif comprises about 2 cysteine residues to about 20 cysteine residues. In some embodiments, the cysteine motif comprises about 2 cysteine residues to about 4 cysteine residues, about 2 cysteine residues to about 6 cysteine residues, about 2 cysteine residues to about 8 cysteine residues, about 2 cysteine residues to about 10 cysteine residues, about 2 cysteine residues to about 12 cysteine residues, about 2 cysteine residues to about 14 cysteine residues, about 2 cysteine residues to about 16 cysteine residues, about 2 cysteine residues to about 18 cysteine residues, about 2 cysteine residues to about 20 cysteine residues, about 4 cysteine residues to about 6 cysteine residues, about 4 cysteine residues to about 8 cysteine residues, about 4 cysteine residues to about 10 cysteine residues, about 4 cysteine residues to about 12 cysteine residues, about 4 cysteine residues to about 14 cysteine residues, about 4 cysteine residues to about 16 cysteine residues, about 4 cysteine residues to about 18 cysteine residues, about 4 cysteine residues to about 20 cysteine residues, about 6 cysteine residues to about 8 cysteine residues, about 6 cysteine residues to about 10 cysteine residues, about 6 cysteine residues to about 12 cysteine residues, about 6 cysteine residues to about 14 cysteine residues, about 6 cysteine residues to about 16 cysteine residues, about 6 cysteine residues to about 18 cysteine residues, about 6 cysteine residues to about 20 cysteine residues, about 8 cysteine residues to about 10 cysteine residues, about 8 cysteine residues to about 12 cysteine residues, about 8 cysteine residues to about 14 cysteine residues, about 8 cysteine residues to about 16 cysteine residues, about 8 cysteine residues to about 18 cysteine residues, about 8 cysteine residues to about 20 cysteine residues, about 10 cysteine residues to about 12 cysteine residues, about 10 cysteine residues to about 14 cysteine residues, about 10 cysteine residues to about 16 cysteine residues, about 10 cysteine residues to about 18 cysteine residues, about 10 cysteine residues to about 20 cysteine residues, about 12 cysteine residues to about 14 cysteine residues, about 12 cysteine residues to about 16 cysteine residues, about 12 cysteine residues to about 18 cysteine residues, about 12 cysteine residues to about 20 cysteine residues, about 14 cysteine residues to about 16 cysteine residues, about 14 cysteine residues to about 18 cysteine residues, about 14 cysteine residues to about 20 cysteine residues, about 16 cysteine residues to about 18 cysteine residues, about 16 cysteine residues to about 20 cysteine residues, or about 18 cysteine residues to about 20 cysteine residues. In some embodiments, the cysteine motif comprises about 2 cysteine residues, about 4 cysteine residues, about 6 cysteine residues, about 8 cysteine residues, about 10 cysteine residues, about 12 cysteine residues, about 14 cysteine residues, about 16 cysteine residues, about 18 cysteine residues, or about 20 cysteine residues. In some embodiments, the cysteine motif comprises at least about 2 cysteine residues, about 4 cysteine residues, about 6 cysteine residues, about 8 cysteine residues, about 10 cysteine residues, about 12 cysteine residues, about 14 cysteine residues, about 16 cysteine residues, or about 18 cysteine residues. In some embodiments, the cysteine motif comprises at most about 4 cysteine residues, about 6 cysteine residues, about 8 cysteine residues, about 10 cysteine residues, about 12 cysteine residues, about 14 cysteine residues, about 16 cysteine residues, about 18 cysteine residues, or about 20 cysteine residues. The cysteine residues of the cysteine motif can be capable of forming about 1 disulfide bonds to about 10 disulfide bonds. In some embodiments, the cysteine residues of the cysteine motif are capable of forming about 1 disulfide bonds to about 2 disulfide bonds, about 1 disulfide bonds to about 3 disulfide bonds, about 1 disulfide bonds to about 4 disulfide bonds, about 1 disulfide bonds to about 5 disulfide bonds, about 1 disulfide bonds to about 6 disulfide bonds, about 1 disulfide bonds to about 7 disulfide bonds, about 1 disulfide bonds to about 8 disulfide bonds, about 1 disulfide bonds to about 9 disulfide bonds, about 1 disulfide bonds to about 10 disulfide bonds, about 2 disulfide bonds to about 3 disulfide bonds, about 2 disulfide bonds to about 4 disulfide bonds, about 2 disulfide bonds to about 5 disulfide bonds, about 2 disulfide bonds to about 6 disulfide bonds, about 2 disulfide bonds to about 7 disulfide bonds, about 2 disulfide bonds to about 8 disulfide bonds, about 2 disulfide bonds to about 9 disulfide bonds, about 2 disulfide bonds to about 10 disulfide bonds, about 3 disulfide bonds to about 4 disulfide bonds, about 3 disulfide bonds to about 5 disulfide bonds, about 3 disulfide bonds to about 6 disulfide bonds, about 3 disulfide bonds to about 7 disulfide bonds, about 3 disulfide bonds to about 8 disulfide bonds, about 3 disulfide bonds to about 9 disulfide bonds, about 3 disulfide bonds to about 10 disulfide bonds, about 4 disulfide bonds to about 5 disulfide bonds, about 4 disulfide bonds to about 6 disulfide bonds, about 4 disulfide bonds to about 7 disulfide bonds, about 4 disulfide bonds to about 8 disulfide bonds, about 4 disulfide bonds to about 9 disulfide bonds, about 4 disulfide bonds to about 10 disulfide bonds, about 5 disulfide bonds to about 6 disulfide bonds, about 5 disulfide bonds to about 7 disulfide bonds, about 5 disulfide bonds to about 8 disulfide bonds, about 5 disulfide bonds to about 9 disulfide bonds, about 5 disulfide bonds to about 10 disulfide bonds, about 6 disulfide bonds to about 7 disulfide bonds, about 6 disulfide bonds to about 8 disulfide bonds, about 6 disulfide bonds to about 9 disulfide bonds, about 6 disulfide bonds to about 10 disulfide bonds, about 7 disulfide bonds to about 8 disulfide bonds, about 7 disulfide bonds to about 9 disulfide bonds, about 7 disulfide bonds to about 10 disulfide bonds, about 8 disulfide bonds to about 9 disulfide bonds, about 8 disulfide bonds to about 10 disulfide bonds, or about 9 disulfide bonds to about 10 disulfide bonds. In some embodiments, the cysteine residues of the cysteine motif are capable of forming about 1 disulfide bonds, about 2 disulfide bonds, about 3 disulfide bonds, about 4 disulfide bonds, about 5 disulfide bonds, about 6 disulfide bonds, about 7 disulfide bonds, about 8 disulfide bonds, about 9 disulfide bonds, or about 10 disulfide bonds. In some embodiments, the cysteine residues of the cysteine motif are capable of forming at least about 1 disulfide bonds, about 2 disulfide bonds, about 3 disulfide bonds, about 4 disulfide bonds, about 5 disulfide bonds, about 6 disulfide bonds, about 7 disulfide bonds, about 8 disulfide bonds, or about 9 disulfide bonds. In some embodiments, the cysteine residues of the cysteine motif are capable of forming at most about 2 disulfide bonds, about 3 disulfide bonds, about 4 disulfide bonds, about 5 disulfide bonds, about 6 disulfide bonds, about 7 disulfide bonds, about 8 disulfide bonds, about 9 disulfide bonds, or about 10 disulfide bonds. In certain embodiments, the recombinant ultralong CDR3 knob peptide further comprises a first stalk-forming amino acid sequence and a second stalk-forming amino acid sequence, each about 1 to about 15 amino acids in length, wherein the length of the first second stalk-forming amino acid sequences can be the same or different. In some embodiments, each of the first, and second stalk-forming amino acid sequence is about 1 amino acid to about 2 amino acids, about 1 amino acid to about 3 amino acids, about 1 amino acid to about 5 amino acids, about 1 amino acid to about 5 amino acids, about 1 amino acid to about 7 amino acids, about 1 amino acid to about 9 amino acids, about 1 amino acid to about 11 amino acids, about 1 amino acid to about 12 amino acids, about 1 amino acid to about 13 amino acids, about 1 amino acid to about 14 amino acids, about 1 amino acid to about 15 amino acids, about 2 amino acids to about 3 amino acids, about 2 amino acids to about 5 amino acids, about 2 amino acids to about 5 amino acids, about 2 amino acids to about 7 amino acids, about 2 amino acids to about 9 amino acids, about 2 amino acids to about 11 amino acids, about 2 amino acids to about 12 amino acids, about 2 amino acids to about 13 amino acids, about 2 amino acids to about 14 amino acids, about 2 amino acids to about 15 amino acids, about 3 amino acids to about 5 amino acids, about 3 amino acids to about 5 amino acids, about 3 amino acids to about 7 amino acids, about 3 amino acids to about 9 amino acids, about 3 amino acids to about 11 amino acids, about 3 amino acids to about 12 amino acids, about 3 amino acids to about 13 amino acids, about 3 amino acids to about 14 amino acids, about 3 amino acids to about 15 amino acids, about 5 amino acids to about 5 amino acids, about 5 amino acids to about 7 amino acids, about 5 amino acids to about 9 amino acids, about 5 amino acids to about 11 amino acids, about 5 amino acids to about 12 amino acids, about 5 amino acids to about 13 amino acids, about 5 amino acids to about 14 amino acids, about 5 amino acids to about 15 amino acids, about 5 amino acids to about 7 amino acids, about 5 amino acids to about 9 amino acids, about 5 amino acids to about 11 amino acids, about 5 amino acids to about 12 amino acids, about 5 amino acids to about 13 amino acids, about 5 amino acids to about 14 amino acids, about 5 amino acids to about 15 amino acids, about 7 amino acids to about 9 amino acids, about 7 amino acids to about 11 amino acids, about 7 amino acids to about 12 amino acids, about 7 amino acids to about 13 amino acids, about 7 amino acids to about 14 amino acids, about 7 amino acids to about 15 amino acids, about 9 amino acids to about 11 amino acids, about 9 amino acids to about 12 amino acids, about 9 amino acids to about 13 amino acids, about 9 amino acids to about 14 amino acids, about 9 amino acids to about 15 amino acids, about 11 amino acids to about 12 amino acids, about 11 amino acids to about 13 amino acids, about 11 amino acids to about 14 amino acids, about 11 amino acids to about 15 amino acids, about 12 amino acids to about 13 amino acids, about 12 amino acids to about 14 amino acids, about 12 amino acids to about 15 amino acids, about 13 amino acids to about 14 amino acids, about 13 amino acids to about 15 amino acids, or about 14 amino acids to about 15 amino acids, in length, wherein the length the first, and second stalk-forming amino acid sequence can be the same or different. In some embodiments, each of the first, and second stalk-forming amino acid sequence is about 1 amino acid, about 2 amino acids, about 3 amino acids, about 5 amino acids, about 5 amino acids, about 7 amino acids, about 9 amino acids, about 11 amino acids, about 12 amino acids, about 13 amino acids, about 14 amino acids, or about 15 amino acids in length, wherein the length the first, and second stalk-forming amino acid sequence can be the same or different. In some embodiments, each of the first, and second stalk-forming amino acid sequence is at least about 1 amino acid, about 2 amino acids, about 3 amino acids, about 5 amino acids, about 5 amino acids, about 7 amino acids, about 9 amino acids, about 11 amino acids, about 12 amino acids, about 13 amino acids, or about 14 amino acids in length, wherein the length the first, and second stalk-forming amino acid sequence can be the same or different. In some embodiments, each of the first, and second stalk-forming amino acid sequence is at most about 2 amino acids, about 3 amino acids, about 5 amino acids, about 5 amino acids, about 7 amino acids, about 9 amino acids, about 11 amino acids, about 12 amino acids, about 13 amino acids, about 14 amino acids, or about 15 amino acids in length, wherein the length the first, and second stalk-forming amino acid sequence can be the same or different. In certain embodiments, the cysteine motif is positioned between the first and second stalk-forming amino acid sequences. The first stalk-forming sequence can be a first β-strand. The second stalk-forming sequence can be a second β-strand. In certain embodiments, the first and second stalk-forming sequences are the first and second β-strands, respectively, and the first and second β-strands are in anti-parallel configuration. In certain embodiments, the recombinant ultralong CDR3 knob peptide does not include the first stalk-forming amino acid sequence. In certain embodiments, the recombinant ultralong CDR3 knob peptide does not include the second stalk-forming amino acid sequence. In certain embodiments, the recombinant ultralong CDR3 knob peptide does not include the first and second stalk-forming amino acid sequences. The periplasmic secretion leader may be 8484 (SEQ ID NO: 24), AnsB (SEQ ID NO: 26), CupB2 (SEQ ID NO: 28), FlgI (SEQ ID NO: 30), Ibp-S31A (SEQ ID NO: 32), Lao (SEQ ID NO: 34), Leader M, PorE (SEQ ID NO: 38), TolB (SEQ ID NO: 40), CupC2 (SEQ ID NO: 70), Azu (SEQ ID NO: 72), Pbp (SEQ ID NO: 74), PbpA20V (SEQ ID NO: 76), 5193 (SEQ ID NO: 78), or Ibp (SEQ ID NO: 80). In certain embodiments, the periplasmic secretion leader has at least 85% identity, at least 90% identity, or at least 95% identity, to an amino acid sequence selected from SEQ ID NOS: 24, 26, 28, 30, 32, 34, 38, 40, 70, 72, 74, 76, 78, and 80. A secretion leader amino acid sequence described herein may be encoded by any corresponding nucleic acid sequence which may be determined by one of skill in the art in view of the degeneracy of the genetic code. Nucleic acid sequences encoding the secretion leaders set forth in SEQ ID NOS: 24, 26, 28, 30, 32, 34, 38, 40, 70, 72, 74, 76, 78, and 80 are included as examples only in SEQ ID NOS: 23, 25, 27, 29, 31, 33, 35, 37, 39, 60, 71, 73, 75, 77, and 79, respectively. In some embodiments a nucleic acid sequence encoding secretion leader 8484, AnsB, CupB2, FlgI, Ibp-S31A, Lao, Leader M, PorE, TolB, CupC2, Azu, Pbp, PbpA20V, 5193, or Ibp comprises a sequence having at least about 85%, at least about 90%, at least about 95%, at least about 98%, at least about 99%, or 100% identity to SEQ ID NOS: 23, 25, 27, 29, 31, 33, 37, 39, 60, 71, 73, 75, 77, or 79, respectively.

In certain embodiments, the nucleic acid encoding the recombinant ultralong CDR3 knob peptide is operably linked to a ribosome binding site sequence (RBS). The RBS may be a high RBS strength or a medium RBS strength. In certain embodiments, the sequence of the RBS is set forth as SEQ ID NO: 21. In certain embodiments, the sequence of the RBS is set forth as SEQ ID NO: 22.

In certain embodiments, the sequence of the nucleic acid encoding the recombinant ultralong CDR3 knob peptide is not directly and/or operably linked to a nucleic acid sequence encoding a fusion partner, a cleavable linker, or both. A fusion construct as used herein, e.g., as a reference for comparison with a construct of the present invention may include a sequence encoding a cleavable linker. Such a cleavable linker may be any engineered or naturally-occurring cleavable linker known to those of skill in the art. The engineered cleavable linker can be a linker designed to include a unique site specific cleavage site during or following purification of the knob peptide, andcan be cleaved using a suitable recombinant protease. Non limiting examples of the recombinant protease can include enterokinase, factor Xa, trypsin, chymotrypsin, SUMO, Pepsin, TEV protease, Thrombin, HRV3C, and those described in http://dx.doi.org/10.1016/j.chroma.2014.02.02, which is incorporated herein by reference in its entirety. The engineered cleavable linker can be a linker that includes an intein tag which can self cleave. Non-limiting examples of the intein tag can include Chitin binding tag-IMPACT system from New England Biolabs, and those described in doi: 10.1186/1475-2859-4-32, which is incorporated herein by reference in its entirety. The term linker or cleavable linker may not encompass the junction between a periplasmic secretion leader directly and operably linked to the recombinant ultralong CDR3 knob peptide. Accordingly, nucleic acid sequence encoding a linker or cleavable linker may not encompass a nucleic acid sequence encoding the amino acids at or around the junction of a periplasmic secretion leader operably linked to the recombinant ultralong CDR3 knob peptide.

The yield and/or quality of the produced recombinant ultralong CDR3 knob peptide may be measured by any means known to those of skill in the art. The recombinant ultralong CDR3 knob peptide produced by the methods of the invention may be present in the periplasm in soluble form, or having any measured quality as desired and described herein, at a yield of about 0.1 g/L to about 20 g/L (grams per liter). In certain embodiments, the produced recombinant ultralong CDR3 knob peptide is present in the periplasm in soluble form at a yield of about 0.1 g/L to about 0.5 g/L, about 0.1 g/L to about 1 g/L, about 0.1 g/L to about 2 g/L, about 0.1 g/L to about 4 g/L, about 0.1 g/L to about 6 g/L, about 0.1 g/L to about 8 g/L, about 0.1 g/L to about 12 g/L, about 0.1 g/L to about 14 g/L, about 0.1 g/L to about 16 g/L, about 0.1 g/L to about 18 g/L, about 0.1 g/L to about 20 g/L, about 0.5 g/L to about 1 g/L, about 0.5 g/L to about 2 g/L, about 0.5 g/L to about 4 g/L, about 0.5 g/L to about 6 g/L, about 0.5 g/L to about 8 g/L, about 0.5 g/L to about 12 g/L, about 0.5 g/L to about 14 g/L, about 0.5 g/L to about 16 g/L, about 0.5 g/L to about 18 g/L, about 0.5 g/L to about 20 g/L, about 1 g/L to about 2 g/L, about 1 g/L to about 4 g/L, about 1 g/L to about 6 g/L, about 1 g/L to about 8 g/L, about 1 g/L to about 12 g/L, about 1 g/L to about 14 g/L, about 1 g/L to about 16 g/L, about 1 g/L to about 18 g/L, about 1 g/L to about 20 g/L, about 2 g/L to about 4 g/L, about 2 g/L to about 6 g/L, about 2 g/L to about 8 g/L, about 2 g/L to about 12 g/L, about 2 g/L to about 14 g/L, about 2 g/L to about 16 g/L, about 2 g/L to about 18 g/L, about 2 g/L to about 20 g/L, about 4 g/L to about 6 g/L, about 4 g/L to about 8 g/L, about 4 g/L to about 12 g/L, about 4 g/L to about 14 g/L, about 4 g/L to about 16 g/L, about 4 g/L to about 18 g/L, about 4 g/L to about 20 g/L, about 6 g/L to about 8 g/L, about 6 g/L to about 12 g/L, about 6 g/L to about 14 g/L, about 6 g/L to about 16 g/L, about 6 g/L to about 18 g/L, about 6 g/L to about 20 g/L, about 8 g/L to about 12 g/L, about 8 g/L to about 14 g/L, about 8 g/L to about 16 g/L, about 8 g/L to about 18 g/L, about 8 g/L to about 20 g/L, about 12 g/L to about 14 g/L, about 12 g/L to about 16 g/L, about 12 g/L to about 18 g/L, about 12 g/L to about 20 g/L, about 14 g/L to about 16 g/L, about 14 g/L to about 18 g/L, about 14 g/L to about 20 g/L, about 16 g/L to about 18 g/L, about 16 g/L to about 20 g/L, or about 18 g/L to about 20 g/L. In certain embodiments, the produced recombinant ultralong CDR3 knob peptide is present in the periplasm in soluble form at a yield of about 0.1 g/L, about 0.5 g/L, about 1 g/L, about 2 g/L, about 4 g/L, about 6 g/L, about 8 g/L, about 12 g/L, about 14 g/L, about 16 g/L, about 18 g/L, or about 20 g/L. In certain embodiments, the produced recombinant ultralong CDR3 knob peptide is present in the periplasm in soluble form at a yield of at least about 0.1 g/L, about 0.5 g/L, about 1 g/L, about 2 g/L, about 4 g/L, about 6 g/L, about 8 g/L, about 12 g/L, about 14 g/L, about 16 g/L, or about 18 g/L. In certain embodiments, the produced recombinant ultralong CDR3 knob peptide is present in the periplasm in soluble form at a yield of at most about 0.5 g/L, about 1 g/L, about 2 g/L, about 4 g/L, about 6 g/L, about 8 g/L, about 12 g/L, about 14 g/L, about 16 g/L, about 18 g/L, or about 20 g/L.

In certain embodiments, the method further comprises measuring the quality of an amount of the recombinant ultralong CDR3 knob peptide produced using the methods of the invention. The quality may be, e.g., a binding activity of the recombinant ultralong CDR3 knob peptide to a desired target, peptide size, peptide conformation, or peptide sequence. The quality may be the amount of the produced recombinant ultralong CDR3 knob peptide present in the periplasm in properly processed form In some embodiments, the measured yield of the recombinant ultralong CDR3 knob peptide that is present in the periplasm or produced by secretion into the periplasm includes the yield that is released to the culture medium. In some embodiments, the yield of the recombinant ultralong CDR3 knob peptide that is present in the periplasm or produced by secretion into the periplasm excludes the yield that is released to the culture medium. In certain embodiments, the quality is measured by an assay. The quality may be target binding activity, and the activity assay can be a binding assay. Any characteristic of binding of the recombinant ultralong CDR3 knob peptide to its target as known to those of skill in the art may be utilized. In certain embodiments, at least 70% of the recombinant ultralong CDR3 knob peptide produced in the periplasm in soluble form is active. In certain embodiments, about 70% to about 100%, of the recombinant ultralong CDR3 knob peptide present in the periplasm in soluble form is active. In certain embodiments, about 70% to about 100% of the soluble recombinant ultralong CDR3 knob peptide present in the periplasm is active. In certain embodiments, about 70% to about 75%, about 70% to about 80%, about 70% to about 85%, about 70% to about 90%, about 70% to about 92%, about 70% to about 94%, about 70% to about 95%, about 70% to about 96%, about 70% to about 98%, about 70% to about 99%, about 70% to about 100%, about 75% to about 80%, about 75% to about 85%, about 75% to about 90%, about 75% to about 92%, about 75% to about 94%, about 75% to about 95%, about 75% to about 96%, about 75% to about 98%, about 75% to about 99%, about 75% to about 100%, about 80% to about 85%, about 80% to about 90%, about 80% to about 92%, about 80% to about 94%, about 80% to about 95%, about 80% to about 96%, about 80% to about 98%, about 80% to about 99%, about 80% to about 100%, about 85% to about 90%, about 85% to about 92%, about 85% to about 94%, about 85% to about 95%, about 85% to about 96%, about 85% to about 98%, about 85% to about 99%, about 85% to about 100%, about 90% to about 92%, about 90% to about 94%, about 90% to about 95%, about 90% to about 96%, about 90% to about 98%, about 90% to about 99%, about 90% to about 100%, about 92% to about 94%, about 92% to about 95%, about 92% to about 96%, about 92% to about 98%, about 92% to about 99%, about 92% to about 100%, about 94% to about 95%, about 94% to about 96%, about 94% to about 98%, about 94% to about 99%, about 94% to about 100%, about 95% to about 96%, about 95% to about 98%, about 95% to about 99%, about 95% to about 100%, about 96% to about 98%, about 96% to about 99%, about 96% to about 100%, about 98% to about 99%, about 98% to about 100%, or about 99% to about 100%, of the recombinant ultralong CDR3 knob peptide present in the periplasm is active. In certain embodiments, about 70%, about 75%, about 80%, about 85%, about 90%, about 92%, about 94%, about 95%, about 96%, about 98%, about 99%, or about 100%, of the recombinant ultralong CDR3 knob peptide present in the periplasm is active. In certain embodiments, at least about 70%, about 75%, about 80%, about 85%, about 90%, about 92%, about 94%, about 95%, about 96%, about 98%, or about 99%, of the recombinant ultralong CDR3 knob peptide present in the periplasm is active. The activity may be evaluated by comparison with a reference. A reference may be selected as appropriate, and as desired and understood by one of skill in the art. For example, a reference may be a negative control or a corresponding recombinant ultralong CDR3 knob peptide produced and purified from a fusion construct. A fusion construct may comprise a sequence encoding a fusion partner, e.g., a chaperone protein, that when expressed is connected via a linker or other means to the recombinant ultralong CDR3 knob peptide.

The nucleic acid encoding the recombinant ultralong CDR3 knob peptide can be optimized for expression in the host cell. In certain embodiments, the Pseudomonadales host cell is Pseudomonas fluorescens. In certain embodiments, the Pseudomonadales host cell is deficient in expression of one or more proteases, overexpresses one or more folding modulators, overexpresses one or more inactivated proteases, or a combination thereof. In certain embodiments, the one or more protease is selected from: Lon, HslU, HslV, DegP1, DegP2, DegP2 S219A, Prc1, Prc2, MepM1, a serralysin, and AprA. In certain embodiments, the one or more protease comprises DegP2. In certain embodiments, the one or more protease is selected from: Prc1, Prc2, HslU, HslV, MepM1, and a serralysin. In certain embodiments, the one or more protease comprises Prc1, Prc2, HslU, HslV, MepM1, and a serralysin. In some embodiments, the serralysin is RXF04495.2. In certain embodiments, the one or more folding modulator is selected from: SecB, DsbA, DsbC, Skp, and FklB2. In some embodiments, the host cell overexpresses SecB. In some embodiments, the host cell overexpresses DsbA, DsbC and Skp. In some embodiments, the host cell overexpresses DsbA and DsbC. In some embodiments, the host cell overexpresses DsbC and FklB2. In certain embodiments, the host cell is deficient in expression of DegP2, and overexpresses SecB. In certain embodiments, the host cell is deficient in expression of DegP2, and overexpresses DsbA, DsbC, and Skp. In certain embodiments, the host cell is deficient in expression of DegP2, and overexpresses DsbA and DsbC. In certain embodiments, the host cell overexpresses DsbC and FklB2. In certain embodiments, the host strain has a phenotype and genotype as set forth for any host strain in any one of Tables 3, 4, 6, and 9. In certain embodiments, the host strain has a phenotype, genotype, and expression construct sequence elements as set forth for any host strain in any one of Tables 3, 4, 6, and 9. In certain embodiments, the host strain is any as set forth in any one of Tables 3, 4, 6, and 9. In certain embodiments, the periplasmic secretion leader has at least 85% identity to an amino acid sequence SEQ ID NO: 26, and the host cell is deficient in expression of DegP2, and overexpresses SecB. In some embodiments, the periplasmic secretion leader has at least 85% identity to an amino acid sequence SEQ ID NO: 40, and the host cell is deficient in expression of DegP2 and overexpresses SecB. In some embodiments, the periplasmic secretion leader has at least 85% identity to an amino acid sequence SEQ ID NO: 40, and the host cell is deficient in expression of DegP2 and overexpresses DsbA, DsbC and Skp. In some embodiments, the periplasmic secretion leader has at least 85% identity to an amino acid sequence SEQ ID NO: 30, and the host cell is deficient in expression of DegP2 and overexpresses SecB.

In certain embodiments, the invention includes a process for purifying the produced recombinant ultralong CDR3 knob peptide. Purifying the recombinant ultralong CDR3 knob peptide can include separating the cultured Pseudomonadales host cell expressing the recombinant ultralong CDR3 knob peptide from the culture medium. Purifying the recombinant ultralong CDR3 knob peptide include obtaining a cell lysate from the cultured Pseudomonadales host cell expressing the recombinant ultralong CDR3 knob peptide. Separation of the host cell from the culture medium, and obtaining the cell lysate, may be carried out using any method known to those of skill in the art, e.g., as described herein and in U.S. Pat. No. 9,169,304, “Process for Purifying Recombinant Plasmodium falciparum Circumsporozoite Protein,” incorporated by reference herein. In certain embodiments, the process may further comprise performing ultrafiltration of the cell lysate and/or the separated culture medium, to obtain an ultrafiltration permeate and an ultrafiltration concentrate, e.g., using a combination of methods known to those of skill in the art and described herein. The ultrafiltration concentrate may be discarded and chromatographic separation of the ultrafiltration permeate carried out to obtain the purified recombinant ultralong CDR3 knob peptide. Ultrafiltration of the cell lysate, and the separated culture medium can be performed separately or jointly. The ultrafiltration permeate can be the permeate from the ultrafiltration process. Ultrafiltration can include passing the cell lysate and/or the separated culture medium through one or more molecular weight cut offs (MWCO) of about 5 to about 50 kDA, about 5, 8, 10, 15, 20, 25, 30, 35, 40, 50 kDa, or any encompassed ranges or values. The cell lysate and/or the separated culture medium can be passed through the one or more MWCOs in series. In certain embodiments, the ultrafiltration can include passing the cell lysate and/or the separated culture medium through MWCOs of about 25 to about 35 kDA, about 25, 26, 27, 28, 29, 30, 31, 32, 33, 34 to 35 kDa, or any encompassed ranges or values, and MWCOs of about 5 to about 15 kDA, about 5, 6, 7, 8, 9, 10, 11, 12, 13, 14 to 15 kDa MWCO, or any encompassed ranges or values. In certain embodiments, the ultrafiltration can include passing the cell lysate and/or the separated culture medium through a MWCO of about 30 kDa, and a MWCO of about 10 kDa. In some embodiments, the chromatographic separation of the ultrafiltration permeate can include performing cation exchange chromatography on the ultrafiltration permeate, e.g., using a combination of methods as described herein and known to those of skill in the art. The purified recombinant ultralong CDR3 knob peptide can be obtained in an eluate from a cation exchange chromatography column, e.g., using a combination of methods as described herein and known to those of skill in the art.

In certain other embodiments, the process may further comprise performing a first chromatographic separation of the cell lysate and/or the separated culture medium to obtain a first eluate containing the recombinant ultralong CDR3 knob peptide; and performing a second chromatographic separation of the first eluate to obtain a second eluate containing the purified recombinant ultralong CDR3 knob peptide. In some embodiments, the first chromatographic separation of the cell lysate and/or the separated culture medium can include performing cation exchange chromatography on the cell lysate and/or the separated culture medium, e.g., using a combination of methods as described herein and known to those of skill in the art. The first chromatographic separation of the cell lysate and/or the separated culture medium can be performed separately or jointly. The first eluate can be an eluate from the cation exchange chromatography column. In some embodiments, the second chromatographic separation of the first eluate can include performing size exclusion chromatography on the first eluate, e.g., using a combination of methods as described herein and known to those of skill in the art. The purified recombinant ultralong CDR3 knob peptide can be obtained in an eluate from a size exclusion chromatography column.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Expression strategy screening at 96-well scale assessed by binding activity to spike RBD using BLI. Each black closed circle represents the average adjusted binding value of duplicates for each individual strain with a specific ribosome binding site (RBS)+secretion leader expression strategy (expression plasmid screening) in the wild type strain (DC454). The error bar indicates range of binding rate values (n=2, biological replicates). X's indicate wild type strains with empty plasmid (null control) as baseline (dashed line). Open black circles are identified plasmid expression strategies that produced high active fusion knobs and C-terminal affinity tagged knobs binding activity. Adjusted binding rate (raw data) is presented and is proportional to fusion knob and C-terminal affinity tagged knob titer in culture. Adjusted binding rate is measured concentration of a diluted sample multiplied by the dilution factor.

FIG. 2. Host strain screening at 96-well scale by binding activity to spike RBD using BLI. Each closed black circle represents the average binding value of duplicates for each individual strain with specific ribosome binding site (RBS)+secretion leader expression strategy and host strain combinations. The error bar indicates range of binding activity value (biological replicates). Open black circles show wild type host strains (DC454) and X's show the wild-type strain with empty plasmid null control. Dashed line shows the highest value of null controls. Strains having a specific host strain background that produced improved active fusion knob and C-terminal affinity tagged knob expression (open triangles) compared to wild type DC454 strains (open black circles) were identified and selected for 2 L scale up.

FIG. 3. Fusion knob and C-terminal affinity tagged knob expression at 2 L scale. A total of 10 strains were induced at two temperatures and soluble fractions of lysate were assessed by binding activity to spike RBD using BLI. Adjusted binding rate was used to evaluate active knob quantity. Average binding value of 3 sampling replicates for each strain at defined fermentation conditions is presented. Black bars represent whole culture broth samples and hatched bars represent cell-free broth samples. The error bars are sampling replicates.

FIG. 4. Mature knob expression at 2 L scale. Strains were induced under 4 different conditions except PS830-003, which was induced under 2 different conditions (low and high temperature, pH 6). The expression level of mature knobs was assessed by binding to spike RBD using BLI. The average titer of 3 sampling replicates for each strain at defined fermentation conditions is presented. Black solid bars represent whole culture broth and hatched bars represent cell-free broth. The error bars are sampling replicates. Titer at harvest was determined using a truncated R2G3 reference (SEQ ID NO: 67).

FIGS. 5A-5D. Mature knob protein purification using CEX-SEC chromatography. Cell-free-broth-R2G3-mini and R2F12-mini fermentation cultures from lead strains using the mature knob expression strategy were processed. 5A. Chromatogram for R2G3-mini purification after CEX concentration chromatography. 5B. Chromatogram for R2F12-mini purification after CEX concentration chromatography. Solid lines show UV280 traces and dashed lines show conductivity traces. CEX chromatography elution fractions were collected as final product. The bulk of the product elutes in a single peak for both R2G3-mini (FIG. 5A) and R2F12-mini (FIG. 5B) knobs. 5C. A flowchart illustrating the knob protein (picobody) purification using CEX and SEC chromatography steps, according to one example of the disclosure. 5D. SDS-PAGE analysis of chromatography fractions from R2G3-mini and R2F12-mini knob protein (picobody) purification from clarified lysate: clarified extract from cell free broth, eluates from CEX and SEC chromatography, and the final sample following CEX column concentration chromatography. Lanes: 1-MW ladder; 2-5-R2G3 clarified extract, CEX eluate, SEC eluate, and final sample, respectively; 6-9-R2F12 clarified extract, CEX eluate, SEC eluate, and final sample, respectively.

FIGS. 6A-6C. Mature knob protein purification using ultrafiltration (UF)/diafiltration (DF) process. 6A. A flowchart illustrating the knob protein (picobody) purification using UF/DF purification process, according to one example of the disclosure. 6B. SDS-PAGE of the different process solutions/materials (clarified lysate, UF retentate, UF permeate, CEX load, and CEX eluate) obtained during knob protein purification from cell lysate using UF/DF purification process. 6C. SDS-PAGE of the different process materials/solutions (cell free broth, UF retentate, UF permeate, CEX load, and CEX eluate) obtained during knob protein purification from cell free broth using UF/DF purification process.

FIG. 7. Expanded SE-HPLC trace for two knob proteins. Black trace-R2G3-mini with the main peak eluting at 21 minutes; Grey trace-R2F12-mini with the main peak eluting at 23 minutes.

FIG. 8. Expanded reverse phase trace for two knob proteins. Black trace-R2G3-mini with the main peak eluting at 15.3 minutes; Grey trace-R2F12-mini with the main peak eluting at 14.6 minutes.

FIG. 9. Circular dicroism analysis. Top-Circular dichroism analysis of the knob protein samples. Black trace-CD spectrum of the R2G3-mini knob protein. A positive maxima close to 230 nm and negative maxima close to 205 nm are observed; Grey trace-CD spectrum of the R2F12-mini knob protein. A positive maxima close to 230 nm and negative maxima close to 205 nm are observed. Bottom-Expanded regions of the positive and negative maxima of the knob protein CD analysis.

FIG. 10. Variable temperature CD melt analysis. Left: R2F12-mini. Right: R2G3-mini.

FIG. 11. Intrinsic fluorescence analysis. R2G3-mini (black line). R2F12-mini (grey line).

FIG. 12. Binding curves for the association of the knob protein produced through mature knob expression with the SARS-CoV-2 Spike RBD. Left-R2G3-mini; Right-R2F12-mini. Concentrations of the knob protein from the highest to lowest curve were 125, 62.5, 31.3, 15.6, 7.8, and 0 nM for both constructs.

FIG. 13. Reduced SDS-PAGE analysis of the knob protein constructs derived from non-fusion expression. Lane 1-R2G3-mini knob protein; Lane 2-R2F12-mini knob protein, as indicated.

FIG. 14. IC50 determination. Inhibition of SARS-CoV-2 Spike RBD and ACE-2 receptor binding by the R2G3-mini and R2F12-mini knob protein produced from non-fusion expression. SARS-CoV-2 Spike RBD-coated biosensor tips were incubated with a concentration gradient of either R2F12-mini or R2G3-mini followed by incubation with ACE-2 receptor to determine the binding response at each knob protein concentration. Gray: R2F12-mini; Black: R2G3-mini.

FIG. 15. Fusion knob expression at 2 L. Strains were induced under 4 different conditions. The expression level of fusion knobs was assessed by SDS-CGE under reducing conditions. The average titer of 3 sampling replicates for each strain at defined fermentation conditions is presented. The error bars are sampling replicates. Titer at harvest was determined using CGE internal ladder.

FIG. 16. Flowchart illustrating a general, nonlimiting process for purification of knob proteins from knob fusions.

FIG. 17. Polishing CEX Step Chromatogram: processed fusion R2F12-mini load material. At low conductivity (dashed trace), knob protein in load material bound CEX resin and eluted in a sharp A280 peak (solid trace) upon increased conductivity.

FIG. 18. Polishing CEX Step Chromatogram: processed fusion R2G3-mini load material. At low conductivity (dashed trace), knob protein in load material bound CEX resin and eluted in a sharp A280 peak (solid trace) upon increased conductivity.

FIG. 19. SDS-PAGE analysis of fusion knob process intermediates and products. Fusion knob, DsbA fusion partner, and knob are indicated by arrows at right (top to bottom). Lanes, from left to right: 1 and 6-MW ladder; 2-DsbA R2F12-mini IMAC load; 3-DsbA R2F12-mini IMAC elution; 4-DsbA R2F12-mini EK reaction; 5-R2F12-mini Final CEX elution; 7-DsbA R2G3-mini IMAC load; 8-DsbA R2G3-mini IMAC elution; 9-DsbA R2G3-mini EK reaction; 10-R2G3-mini Final CEX elution.

FIG. 20. Expanded reverse phase trace for knob proteins expressed through a fusion expression strategy. Black trace (upper line)-R2G3-mini with the main peak eluting at 15.5 minutes; Grey trace (lower line)-R2F12-mini construct with the main peak eluting at 14.6 minutes.

### DETAILED DESCRIPTION OF THE INVENTION

Provided herein are compositions and methods for producing high quality recombinant proteins at high yield.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed invention. As used herein, the term “consisting” is used to indicate the presence of the recited feature alone. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosure.

Bovine antibodies can contain an ultralong CDR3-H3 that form a stalk and knob structure. This conformation allows binding to antigens typically not well-accessed by conventional antibodies, e.g., those having concave epitopes. The ultralong bovine CDR-H3 knob, which exhibits high sequence diversity, contains a cysteine-rich domain positioned between ascending and descending stalk sequences (“stalk-forming” sequences). The ascending and descending stalk sequences associate in essentially an anti-parallel conformation to form the stalk. See, e.g., Svilenov, H. L., et al., 2021, “Mechanistic principles of an ultra-long bovine CDR reveal strategies for antibody design,” Nat Commun 12, 6737, incorporated herein by reference. Recombinant proteins containing or derived from knob domains, with or without stalk-forming sequences, can be developed to target crevices, pores, or other protein epitopes that are difficult to target with larger antibodies. However, due to their small size and folding requirements, expression of the small cysteine-rich knob domain in active form is challenging. Knob proteins have been expressed as part of cleavable fusion proteins to decrease knob degradation and increase solubility. In such a scenario, a recombinant CDR3 knob protein may be expressed cleavably linked to a fusion partner, such as a bacterial chaperone, in a bacterial host cell. After expression, the protein of interest is cleaved from the fusion partner, e.g., by enzymatic cleavage. These cleavage and purification steps require longer development time, decrease yield, and increase cost.

The present disclosure includes systems and methods for producing a mature recombinant ultralong CDR3 knob peptide, i.e., a knob peptide produced in the absence of a fusion partner or cleavable linker which can be cleaved to separate the fusion partner from the knob peptide.

Thus, a “mature knob protein” or “mature knob peptide” as used herein refers to a knob protein that is produced by a non-fusion-partner strategy. A mature knob protein may be expressed using an expression construct wherein the knob protein coding sequence is not operably linked to a sequence encoding: a fusion partner (as an example only, DsbA), a cleavable linker (e.g., enterokinase-cleavable linker DDDDK (SEQ ID NO: 96)), and a sequence encoding purification tag (e.g., a His-tag). A mature knob protein expression construct may nonetheless encode an operably linked N-terminal secretion leader sequence, e.g., a periplasmic secretion leader that directs the mature knob protein to the periplasm.

A processed, or final, protein (or peptide or polypeptide) may refer to a protein (or peptide or polypeptide) that, while expressed from a construct including operably linked sequences including, e.g., a fusion partner, a cleavable linker, a purification tag, and/or a secretion leader, has been processed to remove any of these operably linked sequences as desired. Such a protein may be produced using a mature (non-fusion) expression strategy to result in a protein that may be referred to as, e.g., a “mature protein,” “mature recombinant protein,” “final mature protein,” “processed mature protein,” or “processed final mature protein,” as appropriate. Here, the term “mature” indicates the absence of a fusion partner coding sequence in the expression construct and does not necessitate maturity in the context of protein processing. Alternatively, a final protein may be produced using a fusion expression strategy to result in a protein that may be referred to as, e.g., a “fusion protein,” “recombinant fusion protein,” “final fusion protein,” “processed fusion protein,” or “processed final fusion protein,” as appropriate. Here, the term “fusion” indicates the presence of a fusion partner coding sequence in the expression construct and does not necessitate the presence of a fusion partner in the produced protein. A “processed” or “final” protein may have the same amino acid sequence, and the same properties, regardless of whether produced by a non-fusion-partner expression strategy (independently produced) or by a fusion partner expression strategy.

A mature protein may be, e.g., a mature knob protein that is an ultralong CDR3-H3 peptide having the amino acid sequence set forth in SEQ ID NO: 12, 18, 35, 36, or 67. In certain embodiments, the method includes expression of a nucleic acid sequence encoding the recombinant ultralong CDR3 knob peptide that is not part of a fusion protein, i.e., the knob peptide coding sequence is not directly and/or operably linked to a nucleic acid sequence encoding a fusion partner and/or cleavable linker. The expressed nucleic acid sequence may comprise a sequence encoding a periplasmic secretion leader operably linked to the sequence encoding the CDR3 knob peptide. The periplasmic secretion leader coding sequence may be 5′ to the sequence encoding the CDR3 knob peptide. The periplasmic secretion leader may be N-terminal to the CDR3 knob peptide sequence. The periplasmic secretion leader may direct the CDR3 knob peptide to the bacterial periplasm. The periplasmic secretion leader's function is to traffic the knob peptide to the periplasm. This secretion leader is removed from the knob peptide by cellular processes, and does not require additional manufacturing steps, e.g., enzymatic cleavage and/or subsequent separation. Once localized to the periplasm, the CDR3 knob peptide may actively or passively be released to the culture medium or cell free broth (CFB). As referred to herein, the term “fusion protein” (as encoded by a fusion construct) does not include a CDR3 knob peptide operably linked to a periplasmic secretion leader in the absence of a fusion partner. Direct linkage of a periplasmic secretion leader to a CDR3 knob peptide indicates that the periplasmic secretion leader is operably linked to the CDR3 knob peptide, with no intervening fusion partner. Direct linkage of a periplasmic secretion leader coding sequence to a CDR3 knob peptide coding sequence indicates that the periplasmic secretion leader coding sequence is operably linked to the CDR3 knob peptide coding sequence, with no intervening fusion partner coding sequence. Accordingly, a “fusion construct” does not include a construct encoding a periplasmic secretion leader. Thus, a CDR3 knob peptide nucleic acid expression construct comprising a secretion leader coding sequence, the CDR3 knob peptide coding sequence, and no fusion partner coding sequence, is not a fusion construct. Similarly, a CDR3 knob peptide comprising a secretion leader, the CDR3 knob peptide, and no fusion partner, is not a fusion protein or peptide.

Any appropriate periplasmic secretion leader may be used in the described methods. As used herein, a “secretion signal,” “secretion leader,” “secretion signal polypeptide,” “signal peptide,” or “leader sequence” is intended to refer to a peptide sequence (or in the context of an expression construct, the polynucleotide encoding the peptide sequence) that is useful for targeting a protein or polypeptide of interest to the periplasm of Gram-negative bacteria or into the extracellular space. In some embodiments, the selected secretion leader, when expressed from a nucleic acid construct operably linked to the CDR3 knob peptide, results in production of soluble CDR3 knob peptide, active CDR3 knob peptide, CDR3 knob peptide having an intact N-terminus, or any combination thereof. A CDR3 knob peptide having an intact N-terminus may include the intact knob peptide sequence desired to be expressed. The intact N-terminus may comprise an N-terminal methionine. The intact N-terminus may comprise no N-terminal methionine.

Thus, methods described herein can produce the mature ultralong CDR3 knob peptide with i) comparable solubility and yield, ii) a faster and simpler purification process, and iii) lower development and manufacturing cost, compared to methods of the producing of a corresponding recombinant ultralong CDR3 knob peptide using a fusion construct.

### Recombinant Ultralong CDR3 Knob Peptide

In one aspect, the present invention provides methods for producing a recombinant protein of interest using a Pseudomonadales host cell. The recombinant protein of interest can be an ultralong CDR3 knob peptide.

The recombinant ultralong CDR3 knob peptide can be about 4 to about 6 kDa. In some embodiments, the recombinant ultralong CDR3 knob peptide is about 4 kDa to about 6 kDa. In some embodiments, the recombinant ultralong CDR3 knob peptide is about 4 kDa to about 4.2 kDa, about 4 kDa to about 4.4 kDa, about 4 kDa to about 4.6 kDa, about 4 kDa to about 4.8 kDa, about 4 kDa to about 5 kDa, about 4 kDa to about 5.2 kDa, about 4 kDa to about 5.4 kDa, about 4 kDa to about 5.6 kDa, about 4 kDa to about 5.8 kDa, about 4 kDa to about 6 kDa, about 4.2 kDa to about 4.4 kDa, about 4.2 kDa to about 4.6 kDa, about 4.2 kDa to about 4.8 kDa, about 4.2 kDa to about 5 kDa, about 4.2 kDa to about 5.2 kDa, about 4.2 kDa to about 5.4 kDa, about 4.2 kDa to about 5.6 kDa, about 4.2 kDa to about 5.8 kDa, about 4.2 kDa to about 6 kDa, about 4.4 kDa to about 4.6 kDa, about 4.4 kDa to about 4.8 kDa, about 4.4 kDa to about 5 kDa, about 4.4 kDa to about 5.2 kDa, about 4.4 kDa to about 5.4 kDa, about 4.4 kDa to about 5.6 kDa, about 4.4 kDa to about 5.8 kDa, about 4.4 kDa to about 6 kDa, about 4.6 kDa to about 4.8 kDa, about 4.6 kDa to about 5 kDa, about 4.6 kDa to about 5.2 kDa, about 4.6 kDa to about 5.4 kDa, about 4.6 kDa to about 5.6 kDa, about 4.6 kDa to about 5.8 kDa, about 4.6 kDa to about 6 kDa, about 4.8 kDa to about 5 kDa, about 4.8 kDa to about 5.2 kDa, about 4.8 kDa to about 5.4 kDa, about 4.8 kDa to about 5.6 kDa, about 4.8 kDa to about 5.8 kDa, about 4.8 kDa to about 6 kDa, about 5 kDa to about 5.2 kDa, about 5 kDa to about 5.4 kDa, about 5 kDa to about 5.6 kDa, about 5 kDa to about 5.8 kDa, about 5 kDa to about 6 kDa, about 5.2 kDa to about 5.4 kDa, about 5.2 kDa to about 5.6 kDa, about 5.2 kDa to about 5.8 kDa, about 5.2 kDa to about 6 kDa, about 5.4 kDa to about 5.6 kDa, about 5.4 kDa to about 5.8 kDa, about 5.4 kDa to about 6 kDa, about 5.6 kDa to about 5.8 kDa, about 5.6 kDa to about 6 kDa, or about 5.8 kDa to about 6 kDa. In some embodiments, the recombinant ultralong CDR3 knob peptide is about 4 kDa, about 4.2 kDa, about 4.4 kDa, about 4.6 kDa, about 4.8 kDa, about 5 kDa, about 5.2 kDa, about 5.4 kDa, about 5.6 kDa, about 5.8 kDa, or about 6 kDa. In some embodiments, the recombinant ultralong CDR3 knob peptide is at least about 4 kDa, about 4.2 kDa, about 4.4 kDa, about 4.6 kDa, about 4.8 kDa, about 5 kDa, about 5.2 kDa, about 5.4 kDa, about 5.6 kDa, or about 5.8 kDa. In some embodiments, the recombinant ultralong CDR3 knob peptide is at most about 4.2 kDa, about 4.4 kDa, about 4.6 kDa, about 4.8 kDa, about 5 kDa, about 5.2 kDa, about 5.4 kDa, about 5.6 kDa, about 5.8 kDa, or about 6 kDa.

The recombinant ultralong CDR3 knob peptide can be about 25 to about 70 amino acids in length. In some embodiments, the recombinant ultralong CDR3 knob peptide is about 25 amino acids to about 30 amino acids, about 25 amino acids to about 35 amino acids, about 25 amino acids to about 40 amino acids, about 25 amino acids to about 45 amino acids, about 25 amino acids to about 50 amino acids, about 25 amino acids to about 55 amino acids, about 25 amino acids to about 60 amino acids, about 25 amino acids to about 65 amino acids, about 25 amino acids to about 70 amino acids, about 30 amino acids to about 35 amino acids, about 30 amino acids to about 40 amino acids, about 30 amino acids to about 45 amino acids, about 30 amino acids to about 50 amino acids, about 30 amino acids to about 55 amino acids, about 30 amino acids to about 60 amino acids, about 30 amino acids to about 65 amino acids, about 30 amino acids to about 70 amino acids, about 35 amino acids to about 40 amino acids, about 35 amino acids to about 45 amino acids, about 35 amino acids to about 50 amino acids, about 35 amino acids to about 55 amino acids, about 35 amino acids to about 60 amino acids, about 35 amino acids to about 65 amino acids, about 35 amino acids to about 70 amino acids, about 40 amino acids to about 45 amino acids, about 40 amino acids to about 50 amino acids, about 40 amino acids to about 55 amino acids, about 40 amino acids to about 60 amino acids, about 40 amino acids to about 65 amino acids, about 40 amino acids to about 70 amino acids, about 45 amino acids to about 50 amino acids, about 45 amino acids to about 55 amino acids, about 45 amino acids to about 60 amino acids, about 45 amino acids to about 65 amino acids, about 45 amino acids to about 70 amino acids, about 50 amino acids to about 55 amino acids, about 50 amino acids to about 60 amino acids, about 50 amino acids to about 65 amino acids, about 50 amino acids to about 70 amino acids, about 55 amino acids to about 60 amino acids, about 55 amino acids to about 65 amino acids, about 55 amino acids to about 70 amino acids, about 60 amino acids to about 65 amino acids, about 60 amino acids to about 70 amino acids, or about 65 amino acids to about 70 amino acids, in length. In some embodiments, the recombinant ultralong CDR3 knob peptide is about 25 amino acids, about 30 amino acids, about 35 amino acids, about 40 amino acids, about 45 amino acids, about 50 amino acids, about 55 amino acids, about 60 amino acids, about 65 amino acids, or about 70 amino acids, in length. In some embodiments, the recombinant ultralong CDR3 knob peptide is about at least about 25 amino acids, about 30 amino acids, about 35 amino acids, about 40 amino acids, about 45 amino acids, about 50 amino acids, about 55 amino acids, about 60 amino acids, or about 65 amino acids, in length. In some embodiments, the recombinant ultralong CDR3 knob peptide is about at most about 30 amino acids, about 35 amino acids, about 40 amino acids, about 45 amino acids, about 50 amino acids, about 55 amino acids, about 60 amino acids, about 65 amino acids, or about 70 amino acids, in length.

### Cysteine Motif

The recombinant ultralong CDR3 knob peptide can comprise a cysteine motif. The cysteine motif can comprise 2-20 cysteine residues capable of forming 1-10 disulfide bonds.

In some embodiments, the cysteine motif comprises about 2 cysteine residues to about 20 cysteine residues. In some embodiments, the cysteine motif comprises about 2 cysteine residues to about 4 cysteine residues, about 2 cysteine residues to about 6 cysteine residues, about 2 cysteine residues to about 8 cysteine residues, about 2 cysteine residues to about 10 cysteine residues, about 2 cysteine residues to about 12 cysteine residues, about 2 cysteine residues to about 14 cysteine residues, about 2 cysteine residues to about 16 cysteine residues, about 2 cysteine residues to about 18 cysteine residues, about 2 cysteine residues to about 20 cysteine residues, about 4 cysteine residues to about 6 cysteine residues, about 4 cysteine residues to about 8 cysteine residues, about 4 cysteine residues to about 10 cysteine residues, about 4 cysteine residues to about 12 cysteine residues, about 4 cysteine residues to about 14 cysteine residues, about 4 cysteine residues to about 16 cysteine residues, about 4 cysteine residues to about 18 cysteine residues, about 4 cysteine residues to about 20 cysteine residues, about 6 cysteine residues to about 8 cysteine residues, about 6 cysteine residues to about 10 cysteine residues, about 6 cysteine residues to about 12 cysteine residues, about 6 cysteine residues to about 14 cysteine residues, about 6 cysteine residues to about 16 cysteine residues, about 6 cysteine residues to about 18 cysteine residues, about 6 cysteine residues to about 20 cysteine residues, about 8 cysteine residues to about 10 cysteine residues, about 8 cysteine residues to about 12 cysteine residues, about 8 cysteine residues to about 14 cysteine residues, about 8 cysteine residues to about 16 cysteine residues, about 8 cysteine residues to about 18 cysteine residues, about 8 cysteine residues to about 20 cysteine residues, about 10 cysteine residues to about 12 cysteine residues, about 10 cysteine residues to about 14 cysteine residues, about 10 cysteine residues to about 16 cysteine residues, about 10 cysteine residues to about 18 cysteine residues, about 10 cysteine residues to about 20 cysteine residues, about 12 cysteine residues to about 14 cysteine residues, about 12 cysteine residues to about 16 cysteine residues, about 12 cysteine residues to about 18 cysteine residues, about 12 cysteine residues to about 20 cysteine residues, about 14 cysteine residues to about 16 cysteine residues, about 14 cysteine residues to about 18 cysteine residues, about 14 cysteine residues to about 20 cysteine residues, about 16 cysteine residues to about 18 cysteine residues, about 16 cysteine residues to about 20 cysteine residues, or about 18 cysteine residues to about 20 cysteine residues. In some embodiments, the cysteine motif comprises about 2 cysteine residues, about 4 cysteine residues, about 6 cysteine residues, about 8 cysteine residues, about 10 cysteine residues, about 12 cysteine residues, about 14 cysteine residues, about 16 cysteine residues, about 18 cysteine residues, or about 20 cysteine residues. In some embodiments, the cysteine motif comprises at least about 2 cysteine residues, about 4 cysteine residues, about 6 cysteine residues, about 8 cysteine residues, about 10 cysteine residues, about 12 cysteine residues, about 14 cysteine residues, about 16 cysteine residues, or about 18 cysteine residues. In some embodiments, the cysteine motif comprises at most about 4 cysteine residues, about 6 cysteine residues, about 8 cysteine residues, about 10 cysteine residues, about 12 cysteine residues, about 14 cysteine residues, about 16 cysteine residues, about 18 cysteine residues, or about 20 cysteine residues.

In some embodiments, the cysteine residues of the cysteine motif are capable of forming about 1 disulfide bonds to about 10 disulfide bonds. In some embodiments, the cysteine residues of the cysteine motif are capable of forming about 1 disulfide bonds to about 2 disulfide bonds, about 1 disulfide bonds to about 3 disulfide bonds, about 1 disulfide bonds to about 4 disulfide bonds, about 1 disulfide bonds to about 5 disulfide bonds, about 1 disulfide bonds to about 6 disulfide bonds, about 1 disulfide bonds to about 7 disulfide bonds, about 1 disulfide bonds to about 8 disulfide bonds, about 1 disulfide bonds to about 9 disulfide bonds, about 1 disulfide bonds to about 10 disulfide bonds, about 2 disulfide bonds to about 3 disulfide bonds, about 2 disulfide bonds to about 4 disulfide bonds, about 2 disulfide bonds to about 5 disulfide bonds, about 2 disulfide bonds to about 6 disulfide bonds, about 2 disulfide bonds to about 7 disulfide bonds, about 2 disulfide bonds to about 8 disulfide bonds, about 2 disulfide bonds to about 9 disulfide bonds, about 2 disulfide bonds to about 10 disulfide bonds, about 3 disulfide bonds to about 4 disulfide bonds, about 3 disulfide bonds to about 5 disulfide bonds, about 3 disulfide bonds to about 6 disulfide bonds, about 3 disulfide bonds to about 7 disulfide bonds, about 3 disulfide bonds to about 8 disulfide bonds, about 3 disulfide bonds to about 9 disulfide bonds, about 3 disulfide bonds to about 10 disulfide bonds, about 4 disulfide bonds to about 5 disulfide bonds, about 4 disulfide bonds to about 6 disulfide bonds, about 4 disulfide bonds to about 7 disulfide bonds, about 4 disulfide bonds to about 8 disulfide bonds, about 4 disulfide bonds to about 9 disulfide bonds, about 4 disulfide bonds to about 10 disulfide bonds, about 5 disulfide bonds to about 6 disulfide bonds, about 5 disulfide bonds to about 7 disulfide bonds, about 5 disulfide bonds to about 8 disulfide bonds, about 5 disulfide bonds to about 9 disulfide bonds, about 5 disulfide bonds to about 10 disulfide bonds, about 6 disulfide bonds to about 7 disulfide bonds, about 6 disulfide bonds to about 8 disulfide bonds, about 6 disulfide bonds to about 9 disulfide bonds, about 6 disulfide bonds to about 10 disulfide bonds, about 7 disulfide bonds to about 8 disulfide bonds, about 7 disulfide bonds to about 9 disulfide bonds, about 7 disulfide bonds to about 10 disulfide bonds, about 8 disulfide bonds to about 9 disulfide bonds, about 8 disulfide bonds to about 10 disulfide bonds, or about 9 disulfide bonds to about 10 disulfide bonds. In some embodiments, the cysteine residues of the cysteine motif are capable of forming about 1 disulfide bonds, about 2 disulfide bonds, about 3 disulfide bonds, about 4 disulfide bonds, about 5 disulfide bonds, about 6 disulfide bonds, about 7 disulfide bonds, about 8 disulfide bonds, about 9 disulfide bonds, or about 10 disulfide bonds. In some embodiments, the cysteine residues of the cysteine motif are capable of forming at least about 1 disulfide bonds, about 2 disulfide bonds, about 3 disulfide bonds, about 4 disulfide bonds, about 5 disulfide bonds, about 6 disulfide bonds, about 7 disulfide bonds, about 8 disulfide bonds, or about 9 disulfide bonds. In some embodiments, the cysteine residues of the cysteine motif are capable of forming at most about 2 disulfide bonds, about 3 disulfide bonds, about 4 disulfide bonds, about 5 disulfide bonds, about 6 disulfide bonds, about 7 disulfide bonds, about 8 disulfide bonds, about 9 disulfide bonds, or about 10 disulfide bonds.

A recombinant ultralong CDR3 knob peptide produced according to the methods of the present invention may be one that is derived from any ultralong CDR3 knob protein/peptide known to those of skill in the art or derived therefrom, including, but not limited to a CDR3 knob peptide described in PCT Pub. No. WO 2022/241057, titled “Binding Proteins against SARS-CoV-2 and uses thereof” or WO 2022/241058, titled “Methods of Screening and Expression of Disulfide-Bonded Binding Peptides,” each incorporated herein by reference in its entirety. The CDR3 knob peptide may have at least 85% sequence identity to any CDR3 knob protein as set forth, e.g., in Table 6 or SEQ ID NO: 112, 114, 116, 117, 119, 121, 123, or 126 of WO 2022/241057. The amino acid sequence of the CDR3 knob protein may have at least 85% identity to an amino acid sequence set forth as SEQ ID NO: 12 or 18 herein. In some embodiments, the amino acid sequence of the cysteine motif has at least 85% identity to an amino acid sequence SEQ ID NO: 12. In some embodiments, the amino acid sequence of the cysteine motif has at least 85% identity to an amino acid sequence SEQ ID NO: 18.

The amino acid sequence identity of the CDR3 knob peptide to any of the above CDR3 knob peptides may be about 85% to about 100%. The sequence identity may be about 85% to about 87%, about 85% to about 89%, about 85% to about 91%, about 85% to about 93%, about 85% to about 94%, about 85% to about 95%, about 85% to about 96%, about 85% to about 97%, about 85% to about 98%, about 85% to about 99%, about 85% to about 100%, about 87% to about 89%, about 87% to about 91%, about 87% to about 93%, about 87% to about 94%, about 87% to about 95%, about 87% to about 96%, about 87% to about 97%, about 87% to about 98%, about 87% to about 99%, about 87% to about 100%, about 89% to about 91%, about 89% to about 93%, about 89% to about 94%, about 89% to about 95%, about 89% to about 96%, about 89% to about 97%, about 89% to about 98%, about 89% to about 99%, about 89% to about 100%, about 91% to about 93%, about 91% to about 94%, about 91% to about 95%, about 91% to about 96%, about 91% to about 97%, about 91% to about 98%, about 91% to about 99%, about 91% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The sequence identity may be about 85%, about 87%, about 89%, about 91%, about 93%, about 94%, about 95%, about 96%, about 97%, about 98%, about 99%, or about 100%. The sequence identity may be at least about 85%, about 87%, about 89%, about 91%, about 93%, about 94%, about 95%, about 96%, about 97%, about 98%, or about 99%. The sequence identity may be at most about 87%, about 89%, about 91%, about 93%, about 94%, about 95%, about 96%, about 97%, about 98%, about 99%, or about 100%.

### Stalk Formation

In some embodiments, the recombinant ultralong CDR3 knob peptide further comprises a first stalk-forming amino acid sequence that is N-terminal to the knob sequence, including any cysteine motif, and a second stalk-forming amino acid sequence that is C-terminal to the knob sequence. The first and second stalk-forming sequences may associate to form the stalk. In some embodiments, the recombinant ultralong CDR3 knob peptide does not comprise a first and/or second stalk-forming sequence. In some embodiments, each first and second stalk-forming amino acid sequence is about 1 to about 15 amino acids in length. The length of the first and second stalk-forming amino acid sequences may be the same or different. In such embodiments, the cysteine motif may be positioned between the first and second stalk-forming amino acid sequences. In some embodiments, the first stalk-forming sequence is a β-strand. In some embodiments, the second stalk-forming sequence is a β-strand. In some embodiments, each of the first and second stalk-forming sequences is a β-strand. In some embodiments, each of the first and second stalk-forming sequences are β-strands, and the first and second β-strand stalk-forming sequences are can associate in anti-parallel configuration resulting in a CDR3 knob peptide having a mushroom-shaped structure as known to those of skill in the art. In some embodiments, the recombinant ultralong CDR3 knob peptide does not comprise a first stalk-forming amino acid sequence. In some embodiments, the recombinant ultralong CDR3 knob peptide does not comprise a second stalk-forming amino acid sequence. In some embodiments, the recombinant ultralong CDR3 knob peptide does not comprise a first or a second stalk-forming amino acid sequence.

In some embodiments, each of the first and second stalk-forming amino acid sequences are independently about 1 amino acid to about 2 amino acids, about 1 amino acid to about 3 amino acids, about 1 amino acid to about 5 amino acids, about 1 amino acid to about 5 amino acids, about 1 amino acid to about 7 amino acids, about 1 amino acid to about 9 amino acids, about 1 amino acid to about 11 amino acids, about 1 amino acid to about 12 amino acids, about 1 amino acid to about 13 amino acids, about 1 amino acid to about 14 amino acids, about 1 amino acid to about 15 amino acids, about 2 amino acids to about 3 amino acids, about 2 amino acids to about 5 amino acids, about 2 amino acids to about 5 amino acids, about 2 amino acids to about 7 amino acids, about 2 amino acids to about 9 amino acids, about 2 amino acids to about 11 amino acids, about 2 amino acids to about 12 amino acids, about 2 amino acids to about 13 amino acids, about 2 amino acids to about 14 amino acids, about 2 amino acids to about 15 amino acids, about 3 amino acids to about 5 amino acids, about 3 amino acids to about 5 amino acids, about 3 amino acids to about 7 amino acids, about 3 amino acids to about 9 amino acids, about 3 amino acids to about 11 amino acids, about 3 amino acids to about 12 amino acids, about 3 amino acids to about 13 amino acids, about 3 amino acids to about 14 amino acids, about 3 amino acids to about 15 amino acids, about 5 amino acids to about 5 amino acids, about 5 amino acids to about 7 amino acids, about 5 amino acids to about 9 amino acids, about 5 amino acids to about 11 amino acids, about 5 amino acids to about 12 amino acids, about 5 amino acids to about 13 amino acids, about 5 amino acids to about 14 amino acids, about 5 amino acids to about 15 amino acids, about 5 amino acids to about 7 amino acids, about 5 amino acids to about 9 amino acids, about 5 amino acids to about 11 amino acids, about 5 amino acids to about 12 amino acids, about 5 amino acids to about 13 amino acids, about 5 amino acids to about 14 amino acids, about 5 amino acids to about 15 amino acids, about 7 amino acids to about 9 amino acids, about 7 amino acids to about 11 amino acids, about 7 amino acids to about 12 amino acids, about 7 amino acids to about 13 amino acids, about 7 amino acids to about 14 amino acids, about 7 amino acids to about 15 amino acids, about 9 amino acids to about 11 amino acids, about 9 amino acids to about 12 amino acids, about 9 amino acids to about 13 amino acids, about 9 amino acids to about 14 amino acids, about 9 amino acids to about 15 amino acids, about 11 amino acids to about 12 amino acids, about 11 amino acids to about 13 amino acids, about 11 amino acids to about 14 amino acids, about 11 amino acids to about 15 amino acids, about 12 amino acids to about 13 amino acids, about 12 amino acids to about 14 amino acids, about 12 amino acids to about 15 amino acids, about 13 amino acids to about 14 amino acids, about 13 amino acids to about 15 amino acids, or about 14 amino acids to about 15 amino acids, in length, wherein the length the first, and second stalk-forming amino acid sequence can be the same or different. In some embodiments, each of the first and second stalk-forming amino acid sequence is about 1 amino acid, about 2 amino acids, about 3 amino acids, about 5 amino acids, about 5 amino acids, about 7 amino acids, about 9 amino acids, about 11 amino acids, about 12 amino acids, about 13 amino acids, about 14 amino acids, or about 15 amino acids in length, wherein the length of the first, and second stalk-forming amino acid sequence can be the same or different. In some embodiments, each of the first and second stalk-forming amino acid sequences is at least about 1 amino acid, about 2 amino acids, about 3 amino acids, about 5 amino acids, about 5 amino acids, about 7 amino acids, about 9 amino acids, about 11 amino acids, about 12 amino acids, about 13 amino acids, or about 14 amino acids in length, wherein the length the first, and second stalk-forming amino acid sequence can be the same or different. In some embodiments, each of the first, and second stalk-forming amino acid sequence is at most about 2 amino acids, about 3 amino acids, about 5 amino acids, about 5 amino acids, about 7 amino acids, about 9 amino acids, about 11 amino acids, about 12 amino acids, about 13 amino acids, about 14 amino acids, or about 15 amino acids in length, wherein the length the first and second stalk-forming amino acid sequence can be the same or different.

Any appropriate stalk-forming amino acid sequences may be used. Stalk-forming sequences are described, e.g., in PCT Pub. No. WO 2022/241057, incorporated by reference above. The sequence of the first stalk-forming amino acid sequence can comprise an amino acid sequence having the formula C-X1-T-V-X2-Q (SEQ ID NO: 14), wherein X1 and X2 are independently selected from any amino acid. In some embodiments, X1 is selected from: Ser, Thr, Gly, Asn, Ala, and Pro. In some embodiments, X1 is selected from: Ser, Thr, and Ala. In some embodiments, X1 is Ser. In some embodiments, X1 is Thr. In some embodiments, X1 is Gly. In some embodiments, X1 is Asn. In some embodiments, X1 is Ala. In some embodiments, X1 is Pro. In some embodiments, X2 is selected from: His, Gln, Arg, Lys, Gly, Thr, Tyr, Phe, Trp, Met, Ile, Val, or Leu. In some embodiments, X2 is His or Tyr. In embodiments, the sequence of the first stalk-forming amino acid sequence comprises an amino acid sequence having the formula CTTVHQ (SEQ ID NO: 15), CATVHQ (SEQ ID NO: 81), CAIVQQ (SEQ ID NO:82), or CATVDQ (SEQ ID NO:83). The sequence of the second stalk-forming amino acid sequence can comprises an amino acid sequence having the formula Y-X3-Y-X4-Y, wherein X3 and X4 are independently any amino acid.

### Target Antigen

The recombinant ultralong CDR3 knob peptide, and/or the cysteine motif is capable of binding to a target antigen. A target antigen may be any desired to be targeted by one of skill in the art. The target antigen can be selected from a cytokine; a chemokine; a drug; a transmembrane protein or a cell-surface protein, e.g., a receptor, cell-surface marker, or pathogen surface-protein; a growth factor; a growth factor receptor; immune checkpoint molecule, and a blood factor. In some embodiments, the target antigen is selected from: carcinoembryonic antigen (CEA); CD22; fibrin II beta chain; TNF-alpha; VEGFR2; ITGB2 (CD18); CA-125; and NCA-90 (granulocyte antigen). A target antigen may comprise a structure that is difficult for a conventional antibody to access, including but not limited to a transmembrane receptor, e.g., an ion channel or a G-protein-coupled receptor (GPCR).

In some embodiments, the target antigen is a pathogen antigen, that is, it is comprised by a pathogen. The pathogen may be, e.g., a virus, bacteria, fungus, parasite, or protozoa. The virus may be a coronavirus, norovirus, immunodeficiency virus (e.g., HIV), varicella zoster virus, influenza virus, herpes virus, human papillomavirus, Epstein-Barr virus, mumps virus, rubeola virus, rotavirus, norovirus, West Nile virus, Ebola virus, respiratory syncytial virus, rhinovirus, parainfluenza virus, or adenovirus. The bacteria may be a species of Eschericia, Salmonella, Helicobacter, Neisseria, Staphylococcus, Streptococcus, Campylobacter, Clostridium, Listeria, Vibrio, Chlamydia, and Treponema. The protozoa may be Giardia, Plasmodium, Trichomonas, or Toxoplasma. In some embodiments, the target antigen is a coronavirus spike protein or nucleocapsid protein. The target antigen may be in the receptor-binding domain (RBD) of a coronavirus spike protein. The coronavirus can be SARS-COV, SARS-CoV2, or MERS. The SARS-CoV2 can be any variant of SARS-CoV2. The variant of SARS-CoV2 can be an alpha, beta, gamma, delta, epsilon, eta, iota, kappa, 1.617.2, mu, zeta, or omicron variant. The variants of SARS-CoV2 are listed in https://www.cdc.gov/coronavirus/2019-ncov/variants/variant-classifications.html, which is incorporated herein by reference by in its entirety. The variant of SARS-CoV2 can be a present or past, variant of interest (VOI), variant of concern (VOC), variant being monitored (VOM) or variant of high consequence (VOHC).

In some embodiments, the recombinant ultralong CDR3 knob peptide binds to a target antigen with a dissociation constant (KD) that is less than or similar to, such as within ±1%, ±2%, ±3%, ±4%, ±5%, ±6%, ±7%, ±8%, ±9%, ±10%, ±15%, ±20%, or ±25%, the dissociation constant (KD) for binding of a corresponding recombinant ultralong CDR3 knob peptide produced from a fusion construct, when measured under similar conditions, e.g., comparably prepared samples are measured by the same or similar means. In some embodiments, the recombinant ultralong CDR3 knob peptide has IC50 less than or similar to, such as within =1%, ±2%, ±3%, ±4%, ±5%, ±6%, ±7%, ±8%, ±9%, ±10%, ±15%, ±20%, or ±25% for the target antigen, compared to the IC50 of a corresponding recombinant ultralong CDR3 knob peptide produced from a fusion construct, when measured under similar conditions. The fusion construct can comprise a sequence encoding a fusion partner, e.g., a chaperone protein, linked to or fused with the recombinant ultralong CDR3 knob peptide. The recombinant ultralong CDR3 knob peptide, and the corresponding recombinant ultralong CDR3 knob peptide produced from a fusion construct and may have the same sequence, excluding the fusion partner of the fusion protein.

### Bacterial Host Cells

The recombinant ultralong CDR3 knob peptide can be produced using a recombinant Pseudomonadales host cell that is genetically engineered to produce high quality (e.g., active, soluble, and/or intact) recombinant ultralong CDR3 knob peptide without a fusion contract. As described herein, the CDR3 knob peptide may be small, e.g., 3 to 7 kD. In some aspects of the invention, a genetically modified Pseudomonadales host cell is used to produce high quality (e.g., active, soluble, and/or intact) recombinant ultralong CDR3 knob peptide from a non-fusion construct. In some aspects, the invention is directed to a method for use of a recombinant Pseudomonadales host cell to produce high quality (e.g., active, soluble, properly processed, and/or intact) ultralong CDR3 knob peptide without a fusion contract, at high yield.

Recombinant proteins expressed in bacterial host cells are subject to degradation by any of several dozen host cell proteases. Degradation lowers protein quality and yield, often making production of useful quantities of proteolytically sensitive proteins impossible. Introduction of protease deficiencies in the host cell can reduce recombinant protein degradation. In some embodiments, in a recombinant Pseudomonadales host cell that is deficient in one or more proteases, the normal activity of the one or more protease is eliminated or substantially reduced. In some embodiments, the normal activity of the one or more protease is eliminated. In some embodiments, the normal activity of the one or more protease is substantially reduced, e.g., the protease activity in the protease-deficient host cell is reduced relative to that of a host cell not deficient in the protease by at least 85%, at least 90%, at least 95%, or 100%. In some embodiments, deficiency in a protease (e.g., of the one or more proteases) activity results from one or more mutation in a gene encoding the protease.

In some embodiments, the one or more protease deficiencies are selected from any known to those of skill in the art, e.g., as described in U.S. Pat. Nos. 9,394,571 and 9,580,719, both titled “Method for Rapidly Screening Microbial Hosts to Identify Certain Strains with Improved Yield and/or Quality in the Expression of Heterologous Proteins,” in U.S. Pat. No. 7,833,752, in U.S. Pat. No. 10,118,956, in U.S. Pat. No. 9,453,251, “Expression of Mammalian Proteins in Pseudomonas fluorescens,” in U.S. Pat. No. 8,603,824, “Process for Improved Protein Expression by Strain Engineering,” in U.S. Pat. No. 8,530,171, “High Level Expression of Recombinant Toxin Proteins.” In some embodiments, the deficiency is in a tail-specific protease, a murein DD-endopeptidase, a serralysin precursor, membrane-localized protease, a murein L,D transpeptidase, a hemolysin precursor, a D-alanyl-D-alanine carboxypeptidase/endopeptidase AmpH precursor, a periplasmic serine endoprotease, a AAA+family proteolytic machine, Lon or AprA.

In some embodiments, the one or more proteases are selected from Prc-1, Prc-2, DegP2, MepM1, HslU, HslV, and a serralysin. In some embodiments, the one or more proteases comprises Prc-1, Prc-2, DegP2, MepM1, HslUV, and serralysin RXF04495.2. In some embodiments, the one or more proteases comprises Prc-1, Prc-2, MepM1, HslUV, and RXF04495.2. In some embodiments, the one or more proteases comprises Prc-1, Prc-2, MepM1, and HslUV. In some embodiments, the one or more proteases comprises Prc-1, Prc-2, Lon, and La1. In some embodiments, the one or more proteases comprises DegP2, Lon, and La1. In some embodiments, the one or more proteases comprises DegP2.

A tail-specific protease can degrade a recombinant protein expressed in a bacterial host cell. Thus, a recombinant host cell that is deficient in tail-specific protease activity can produce a higher quality recombinant protein of interest than a corresponding host cell having a functional tail-specific protease. For example, antibody fragments produced in bacteria deficient in tail-specific protease activity are less degraded. (See, e.g., the Examples herein, and U.S. Pat. No. 9,493,559, “Bacterial host strain expressing recombinant DsbC and having reduced Tsp activity,” each incorporated herein by reference in its entirety). A host cell deficient in tail-specific protease activity can be achieved by mutation of a gene encoding a tail-specific protease, tail-specific protease related protein, and/or a tail-specific protease homologue. In some embodiments, tail-specific protease deficiency results from mutation of a gene encoding a Pseudomonad Prc. In some embodiments, tail-specific protease deficiency results from mutation of a gene encoding Prc1, a Prc1-related protein, or a Prc1 homologue. In some embodiments, Prc1 has the amino acid sequence of SEQ ID NO: 9. In some embodiments, a Prc1-related protein has at least 60% similarity or at least 60% identity to the amino acid sequence of SEQ ID NO: 9. In some embodiments, tail-specific protease deficiency results from mutation of a gene encoding Prc2, a Prc2-related protein, or a Prc2 homologue. In some embodiments, the Prc2 has the amino acid sequence of SEQ ID NO: 10. In some embodiments, a Prc2-related protein has at least 60% similarity or at least 60% identity to the amino acid sequence of SEQ ID NO: 10. In some embodiments, tail-specific protease deficiency results from mutation of a gene encoding both Prc1, a Prc1-related protein, or a Prc1 homologue, and mutation of a gene encoding Prc2, a Prc2-related protein, or a Prc2 homologue.

Murein DD-endopeptidases cleave DD-bonds in the stem peptides of the sacculus glycan strands. See, e.g., Vollmer, W. et al., 2008, “Bacterial peptidoglycan (murein) hydrolases,” FEMS Micro. Rev. 32:259-286, incorporated herein by reference in its entirety. Murein DD-endopeptidases from Pseudomonads, have been described in the literature.

A host cell deficient in Murein DD-endopeptidase activity can be achieved by mutation of one or more gene encoding a murein DD-endopeptidase. In some embodiments, the murein DD-endopeptidase gene encodes a protease having the amino acid sequence of P. fluorescens MepM1 (SEQ ID NO: 13). In some embodiments, murein DD-endopeptidase deficiency results from mutation of a gene encoding a protease having the amino acid sequence of P. fluorescens MepM1 (SEQ ID NO: 13). In some embodiments, a murein DD-endopeptidase-related protein has at least 60% similarity or at least 60% identity to the amino acid sequence of SEQ ID NO: 13.

A protease deficiency can result from any one or more mutation in a host cell gene encoding the protease, e.g, (i) a complete gene deletion, (ii) a partial gene deletion, (iii) a missense mutation, (iv) a nonsense mutation, (v) a frameshift mutation, (vi) an insertion, or (vii) any combination of (ii), (iii), (iv), (v) and (vi). In some embodiments, the protease deficiency results from a mutation that changes an amino acid in a conserved region of the murein DD-endopeptidase having the amino acid sequence set forth as SEQ ID NO: 13 or the analogous conserved region of a murein DD-endopeptidase having at least 60% similarity to the murein DD-endopeptidase amino acid sequence set forth as SEQ ID NO: 13. In some embodiments, the deficiency in any protease described herein results from a mutation that changes or otherwise disrupts (e.g., by substitution, deletion, insertion, or truncation) an amino acid at a conserved position. A conserved position can be identified by one of skill in the art by any known method. In some embodiments, the mutation is a non-conservative amino acid substitution. As described herein, an amino acid substitution can be a conservative or non-conservative substitution. Conservative and non-conservative amino acid substitutions are described in the literature and can readily be identified by methods well-known to those of skill in the art and as described herein (see, e.g., Table A, listing conservative amino acid substitutions).

In some embodiments, the mutation in a gene encoding a protease as described herein changes or otherwise disrupts an allosteric site amino acid. In some embodiments, the mutation in a protease gene changes or otherwise disrupts an amino acid at an active site position. In some embodiments, the active site position corresponds to any position in the regions 319 to 411 of SEQ ID NO: 13.

In some embodiments, a serralysin precursor deficiency results from mutation of a gene encoding an extracellular alkaline metalloprotease, an extracellular alkaline metalloprotease-related protein, or an extracellular alkaline metalloprotease homologue. In some embodiments, serralysin precursor deficiency results from mutation of a gene encoding an autolytic serralysin precursor, an autolytic serralysin precursor-related protein, or an autolytic serralysin precursor homologue. In some embodiments, the extracellular alkaline metalloprotease is RXF04495.2. In some embodiments, the autolytic serralysin precursor is RXF4500. In some embodiments, RXF04495.2 has the amino acid sequence of SEQ ID NO: 16. In some embodiments, RXF04495.2 deficiency results from mutation of a gene encoding RXF04495.2 having the amino acid sequence of SEQ ID NO: 16

In some embodiments, a periplasmic serine endoprotease deficiency results from mutation of a gene encoding DegP2, DegP2-related protein, or DegP2 homologue. In some embodiments, DegP2 has the amino acid sequence of SEQ ID NO: 19. In some embodiments, DegP2 deficiency results from mutation of a gene encoding DegP2 having the amino acid sequence of SEQ ID NO: 19.

In some embodiments, AAA+family proteolytic machine deficiency results from mutation of a gene encoding HslU, HslU-related protein, or HslU homologue. In some embodiments, AAA+family proteolytic machine deficiency results from mutation of a gene encoding HslV, HslV-related protein, or HslV homologue. In some embodiments, AAA+family proteolytic machine deficiency results from mutation of a gene encoding both HslU, a HslU-related protein, or a HslU homologue, and mutation of a gene encoding HslV, a HslV-related protein, or a HslV homologue.

In some embodiments, HslU has the amino acid sequence of SEQ ID NO: 20. In some embodiments, HslU deficiency results from mutation of a gene encoding HslU having the amino acid sequence of SEQ ID NO: 20. In some embodiments, HslV has the amino acid sequence of SEQ ID NO: 68. In some embodiments, HslV deficiency results from mutation of a gene encoding HslV having the amino acid sequence of SEQ ID NO: 68.

Certain proteases have both protease and chaperone-like activity. When these proteases are negatively affecting protein yield and/or quality it is often useful to specifically delete their protease activity, and they are overexpressed when their chaperone activity may positively affect protein yield and/or quality. These proteases include, but are not limited to: Hsp100 (Clp/Hsl) family members RXF04587.1 (clpA), RXF08347.1, RXF04654.2 (clpX), RXF04663.1, RXF01957.2 (hslU), RXF01961.2 (hslV); Peptidyl-prolyl cis-trans isomerase family member RXF05345.2 (ppiB); Metallopeptidase M20 family member RXF04892.1 (aminohydrolase); Metallopeptidase M24 family members RXF04693.1 (methionine aminopeptidase) and RXF03364.1 (methionine aminopeptidase); and Serine Peptidase S26 signal peptidase I family member RXF01181.1 (signal peptidase).

These and other proteases and folding modulators are known in the art and described in the literature, e.g., in U.S. Pat. No. 8,603,824, “Process for improved protein expression by strain engineering,” incorporated by reference in its entirety. For example, Table D of the '824 patent describes Tig (tig, Trigger factor, FKBP type ppiase (ec 5.2.1.8) RXF04655, UniProtKB-POA850 (TIG_ECOLI)). U.S. Pat. Nos. 9,394,571 and 9,580,719, both titled “Method for Rapidly Screening Microbial Hosts to Identify Certain Strains with Improved Yield and/or Quality in the Expression of Heterologous Proteins,” describe Tig (RXF04655.2, SEQ ID NO: 34 therein), LepB (RXF01181.1, SEQ ID NO: 56 therein), DegP1 (RXF01250, SEQ ID NO: 57 therein), AprA (RXF04304.1, SEQ ID NO: 86 therein), Prc1 (RXF06586.1, SEQ ID NO: 120 therein), DegP2, (RXF07210.1, SEQ ID NO: 124 therein), Lon (RXF04653, SEQ ID NO: 92 therein); DsbA (RXF01002.1, SEQ ID NO: 25 therein), and DsbC (RXF03307.1, SEQ ID NO: 26 therein). These sequences and those for other proteases and folding modulators also are set forth in U.S. Pat. No. 9,580,719 (Table of SEQ ID NOS in columns 93-98 therein), incorporated herein by reference in its entirety. For example, U.S. Pat. No. 9,580,719 provides the sequence encoding HslU (RXF01957.2) and HslV (RXF01961.2) as SEQ ID NOS 18 and 19 therein, respectively.

### Overexpressed Proteins

A Pseudomonad host cell useful in the context of the invention may overexpress one or more proteins, e.g., an inactivated protease or a folding modulator, e.g., a chaperone. When co-overexpressed with the recombinant protein of interest in the host cell, the overexpressed protein can improve the quality and/or yield of a recombinant protein of interest produced. In some embodiments, the co-overexpressed protein is expressed from an exogenous expression construct. In some embodiments, the expression construct is in a plasmid or expression vector. In some embodiments, when overexpressed in a host cell that also overexpresses the recombinant protein of interest, the co-overexpressed protein and the recombinant protein of interest are expressed from different plasmids. In some embodiments, the co-overexpressed protein and the recombinant protein of interest are expressed from the same plasmid. In some embodiments, the co-overexpressed protein and the recombinant protein of interest are expressed by transcription from different promoters on the same plasmid. In some embodiments, the co-overexpressed protein and the recombinant protein of interest are co-transcribed, that is, they are expressed by transcription from the same promoter on the same plasmid. In some embodiments, the co-overexpressed protein is not expressed from the bacterial chromosome. In some embodiments, the one or more co-overexpressed protein is an inactivated protease. In some embodiments, the one or more co-overexpressed protein is a chaperone or protein folding modulator. In some embodiments, the recombinant gram-negative host cell overexpresses 1 co-overexpressed protein to 20 different co-overexpressed proteins. In some embodiments, the recombinant gram-negative host cell overexpresses 1 co-overexpressed protein to 2 different co-overexpressed proteins, 1 co-overexpressed protein to 3 different co-overexpressed proteins, 1 co-overexpressed protein to 4 different co-overexpressed proteins, 1 co-overexpressed protein to 5 different co-overexpressed proteins, 1 co-overexpressed protein to 6 different co-overexpressed proteins, 1 co-overexpressed protein to 7 different co-overexpressed proteins, 1 co-overexpressed protein to 8 different co-overexpressed proteins, 1 co-overexpressed protein to 9 different co-overexpressed proteins, 1 co-overexpressed protein to 10 different co-overexpressed proteins, 1 co-overexpressed protein to 15 different co-overexpressed proteins, 1 co-overexpressed protein to 20 different co-overexpressed proteins, 2 different co-overexpressed proteins to 3 different co-overexpressed proteins, 2 different co-overexpressed proteins to 4 different co-overexpressed proteins, 2 different co-overexpressed proteins to 5 different co-overexpressed proteins, 2 different co-overexpressed proteins to 6 different co-overexpressed proteins, 2 different co-overexpressed proteins to 7 different co-overexpressed proteins, 2 different co-overexpressed proteins to 8 different co-overexpressed proteins, 2 different co-overexpressed proteins to 9 different co-overexpressed proteins, 2 different co-overexpressed proteins to 10 different co-overexpressed proteins, 2 different co-overexpressed proteins to 15 different co-overexpressed proteins, 2 different co-overexpressed proteins to 20 different co-overexpressed proteins, 3 different co-overexpressed proteins to 4 different co-overexpressed proteins, 3 different co-overexpressed proteins to 5 different co-overexpressed proteins, 3 different co-overexpressed proteins to 6 different co-overexpressed proteins, 3 different co-overexpressed proteins to 7 different co-overexpressed proteins, 3 different co-overexpressed proteins to 8 different co-overexpressed proteins, 3 different co-overexpressed proteins to 9 different co-overexpressed proteins, 3 different co-overexpressed proteins to 10 different co-overexpressed proteins, 3 different co-overexpressed proteins to 15 different co-overexpressed proteins, 3 different co-overexpressed proteins to 20 different co-overexpressed proteins, 4 different co-overexpressed proteins to 5 different co-overexpressed proteins, 4 different co-overexpressed proteins to 6 different co-overexpressed proteins, 4 different co-overexpressed proteins to 7 different co-overexpressed proteins, 4 different co-overexpressed proteins to 8 different co-overexpressed proteins, 4 different co-overexpressed proteins to 9 different co-overexpressed proteins, 4 different co-overexpressed proteins to 10 different co-overexpressed proteins, 4 different co-overexpressed proteins to 15 different co-overexpressed proteins, 4 different co-overexpressed proteins to 20 different co-overexpressed proteins, 5 different co-overexpressed proteins to 6 different co-overexpressed proteins, 5 different co-overexpressed proteins to 7 different co-overexpressed proteins, 5 different co-overexpressed proteins to 8 different co-overexpressed proteins, 5 different co-overexpressed proteins to 9 different co-overexpressed proteins, 5 different co-overexpressed proteins to 10 different co-overexpressed proteins, 5 different co-overexpressed proteins to 15 different co-overexpressed proteins, 5 different co-overexpressed proteins to 20 different co-overexpressed proteins, 6 different co-overexpressed proteins to 7 different co-overexpressed proteins, 6 different co-overexpressed proteins to 8 different co-overexpressed proteins, 6 different co-overexpressed proteins to 9 different co-overexpressed proteins, 6 different co-overexpressed proteins to 10 different co-overexpressed proteins, 6 different co-overexpressed proteins to 15 different co-overexpressed proteins, 6 different co-overexpressed proteins to 20 different co-overexpressed proteins, 7 different co-overexpressed proteins to 8 different co-overexpressed proteins, 7 different co-overexpressed proteins to 9 different co-overexpressed proteins, 7 different co-overexpressed proteins to 10 different co-overexpressed proteins, 7 different co-overexpressed proteins to 15 different co-overexpressed proteins, 7 different co-overexpressed proteins to 20 different co-overexpressed proteins, 8 different co-overexpressed proteins to 9 different co-overexpressed proteins, 8 different co-overexpressed proteins to 10 different co-overexpressed proteins, 8 different co-overexpressed proteins to 15 different co-overexpressed proteins, 8 different co-overexpressed proteins to 20 different co-overexpressed proteins, 9 different co-overexpressed proteins to 10 different co-overexpressed proteins, 9 different co-overexpressed proteins to 15 different co-overexpressed proteins, 9 different co-overexpressed proteins to 20 different co-overexpressed proteins, 10 different co-overexpressed proteins to 15 different co-overexpressed proteins, 10 different co-overexpressed proteins to 20 different co-overexpressed proteins, or 15 different co-overexpressed proteins to 20 different co-overexpressed proteins. In some embodiments, the recombinant gram-negative host cell overexpresses 1 co-overexpressed protein, 2 different co-overexpressed proteins, 3 different co-overexpressed proteins, 4 different co-overexpressed proteins, 5 different co-overexpressed proteins, 6 different co-overexpressed proteins, 7 different co-overexpressed proteins, 8 different co-overexpressed proteins, 9 different co-overexpressed proteins, 10 different co-overexpressed proteins, 15 different co-overexpressed proteins, or 20 different co-overexpressed proteins. In some embodiments, the recombinant gram-negative host cell overexpresses at least 1 co-overexpressed protein, 2 different co-overexpressed proteins, 3 different co-overexpressed proteins, 4 different co-overexpressed proteins, 5 different co-overexpressed proteins, 6 different co-overexpressed proteins, 7 different co-overexpressed proteins, 8 different co-overexpressed proteins, 9 different co-overexpressed proteins, 10 different co-overexpressed proteins, or 15 different co-overexpressed proteins. In some embodiments, the recombinant gram-negative host cell overexpresses at most 2 different co-overexpressed proteins, 3 different co-overexpressed proteins, 4 different co-overexpressed proteins, 5 different co-overexpressed proteins, 6 different co-overexpressed proteins, 7 different co-overexpressed proteins, 8 different co-overexpressed proteins, 9 different co-overexpressed proteins, 10 different co-overexpressed proteins, 15 different co-overexpressed proteins, or 20 different co-overexpressed proteins.

### Inactivated Proteases

In some embodiments, the one or more co-overexpressed protein is an inactivated protease. An inactivated protease derived from a functional protease present in the host cell can be overexpressed by a host cell to reduce the functional protease activity in a host cell. The inactivated protease mutant can act as dominant negative protease. The overexpressed inactivated protease can be exogenously produced, e.g., from an expression construct on a plasmid. In some embodiments, the recombinant gram-negative host cell overexpresses 1 to 10 different inactivated proteases. In some embodiments, an overexpressed inactivated protease is inactivated by a mutation in a gene encoding the corresponding functional protease.

In some embodiments, an inactivated protease is an inactive form of a gram negative bacterial a serine protease gene from the EC 3.4.21.107 enzyme family. In some embodiments, an inactivated protease is a DegP protease (also known as HtrA). A DegP protease can be, e.g., a DegP2 protease, or a DegP-like protease. DegP proteases are periplasmic serine endoproteases. Their structure is described, e.g., by Pallen, M. J. and Wren, B. W., 1997, “The HtrA family of serine proteases,” Molecular Microbiology 26 (2): 209-221, both incorporated herein by reference. In some embodiments, the DegP protease is inactivated by mutation in a gene encoding P. fluorescens DegP2 (SEQ ID NO: 19).

In some embodiments, an overexpressed inactivated protease is P. fluorescens DegP2 S219A (SEQ ID NO: 85) or an inactivated DegP2 comprising an amino acid substitution or disruption of the amino acid sequence set forth as SEQ ID NO: 19.

In some embodiments, the recombinant gram-negative host cell overexpresses 1 inactivated protease to 10 inactivated proteases.

### Protein Folding Modulators

In some embodiments, the one or more co-overexpressed protein is a protein folding modulator that improves the quality and/or yield of the recombinant protein of interest. Protein folding modulators, including chaperones, disulfide bond isomerases, and peptidyl-prolyl cis-trans isomerases (PPIases) are a class of proteins present in all cells that aid in the folding, unfolding and degradation of nascent polypeptides. An overexpressed protein folding modulator can be exogenously produced, e.g., from an expression construct on a plasmid. In some embodiments, a recombinant gram-negative host cell of the present invention overexpresses any one or more different protein folding modulator. In some embodiments, a recombinant gram-negative host cell of the present invention overexpresses 1 to 10 different protein folding modulators.

In some embodiments, a protein folding modulator is microbial. In some embodiments, a microbial protein folding modulator is from a bacterium, a mammal, a fungus (e.g., a yeast or a filamentous fungus), an arthropod (e.g., an arachnid or an insect), or a Plasmodium. In some embodiments, a bacterial protein folding modulator is from a gram-negative bacteria. In some embodiments, a mammalian protein folding modulator is from a rodent, e.g., a mouse, rat or hamster, e.g., a golden hamster. In some embodiments, a mammalian protein folding modulator is from a pongo, e.g., an orangutan, a human, a horse, a pig, a bird, e.g., a flycatcher. In some embodiments, a gram-negative bacterial protein folding modulator is an E. coli or Pseudomonad folding modulator protein. In some embodiments, a protein folding modulator or chaperone is a P. fluorescens protein folding modulator. An overexpressed protein folding modulator may be any described in, e.g., U.S. Pat. No. 10,118,956, “Fusion Partners for Peptide Production” (e.g., as in Table 1 therein), U.S. Pat. No. 9,580,719 (e.g., providing sequences for each folding modulator by RXF listed in Table 1 of U.S. Pat. No. 10,118,956), and U.S. Pat. No. 8,603,824, (e.g., Tables A to F therein). As used herein, RXF numbers are open reading frame numbers, and PROKKA numbers are designations determined using the Prokka tool as described by, e.g., Seemann, T., 2014, “Prokka: rapid prokaryotic genome annotation,” Bioinformatics 30 (14): 2068-2069, incorporated herein by reference.

In some embodiments, a protein folding modulator is any known to those of skill in the art or described in the literature, e.g., in “Guidebook to Molecular Chaperones and Protein-Folding Catalysts,” 1997, ed. M. Gething, Melbourne University, Australia, incorporated herein by reference. In some embodiments, each one or more protein folding modulator is independently selected from a GroES/EL, DnaKJ, Clp, Hsp90, SecB, Skp, FklB2, HSP70, HSP110/SSE, HSP40 (DnaJ-related), GRPE-like, HSP90, CPN60, CPN10, cytosolic chaperone, HSP100, small HSP, calnexin, calreticulin, protein disulfide isomerase (PDI), thioredoxin-related protein, disulfide bond isomerase, protein disulfide isomerase, peptidyl-prolyl isomerase, cyclophilin PPIase, FK-506 binding protein, parvulin PPIase, individual chaperone, protein specific chaperone, or an intramolecular chaperone.

In some embodiments, an overexpressed folding modulator protein is a disulfide bond isomerase. In some embodiments, a disulfide bond isomerase is a gram-negative bacterial DsbA, DsbB, DsbC, DsbD, or DsbG. In some embodiments, a disulfide bond isomerase is selected from SEQ ID NOS: 46 (DsbC), (putative cytoplasmic disulfide isomerase DsbA), 44 (DsbA), 87 (DsbB), 88 (DsbD), or 89 (DsbG). In some embodiments, an overexpressed folding modulator protein is a protein disulfide isomerase. In some embodiments, a protein disulfide isomerase is a PDIA6. In some embodiments, the one or more protein folding modulators comprise SecB (SEQ ID NO: 42), DsbA (SEQ ID NO: 44), DsbC (SEQ ID NO: 46), Skp (SEQ ID NO: 48), FklB2 (SEQ ID NO: 50) or any combination thereof. In some embodiments, a recombinant Pseudomonadales host cell of the present invention overexpresses i) SecB (SEQ ID NO: 42); ii) DsbA (SEQ ID NO: 44), DsbC (SEQ ID NO: 46), and Skp (SEQ ID NO: 48); iii) DsbA (SEQ ID NO: 44) and DsbC (SEQ ID NO: 46); iv) FklB2 (SEQ ID NO: 50) and DsbC (SEQ ID NO: 46); or v) Ppi.

In some embodiments, the recombinant gram-negative host cell overexpresses 1 protein folding modulator to 10 protein folding modulators. In some embodiments, the recombinant gram-negative host cell overexpresses 1 protein folding modulator to 2 protein folding modulators, 1 protein folding modulator to 3 protein folding modulators, 1 protein folding modulator to 4 protein folding modulators, 1 protein folding modulator to 5 protein folding modulators, 1 protein folding modulator to 6 protein folding modulators, 1 protein folding modulator to 7 protein folding modulators, 1 protein folding modulator to 8 protein folding modulators, 1 protein folding modulator to 9 protein folding modulators, 1 protein folding modulator to 10 protein folding modulators, 2 protein folding modulators to 3 protein folding modulators, 2 protein folding modulators to 4 protein folding modulators, 2 protein folding modulators to 5 protein folding modulators, 2 protein folding modulators to 6 protein folding modulators, 2 protein folding modulators to 7 protein folding modulators, 2 protein folding modulators to 8 protein folding modulators, 2 protein folding modulators to 9 protein folding modulators, 2 protein folding modulators to 10 protein folding modulators, 3 protein folding modulators to 4 protein folding modulators, 3 protein folding modulators to 5 protein folding modulators, 3 protein folding modulators to 6 protein folding modulators, 3 protein folding modulators to 7 protein folding modulators, 3 protein folding modulators to 8 protein folding modulators, 3 protein folding modulators to 9 protein folding modulators, 3 protein folding modulators to 10 protein folding modulators, 4 protein folding modulators to 5 protein folding modulators, 4 protein folding modulators to 6 protein folding modulators, 4 protein folding modulators to 7 protein folding modulators, 4 protein folding modulators to 8 protein folding modulators, 4 protein folding modulators to 9 protein folding modulators, 4 protein folding modulators to 10 protein folding modulators, 5 protein folding modulators to 6 protein folding modulators, 5 protein folding modulators to 7 protein folding modulators, 5 protein folding modulators to 8 protein folding modulators, 5 protein folding modulators to 9 protein folding modulators, 5 protein folding modulators to 10 protein folding modulators, 6 protein folding modulators to 7 protein folding modulators, 6 protein folding modulators to 8 protein folding modulators, 6 protein folding modulators to 9 protein folding modulators, 6 protein folding modulators to 10 protein folding modulators, 7 protein folding modulators to 8 protein folding modulators, 7 protein folding modulators to 9 protein folding modulators, 7 protein folding modulators to 10 protein folding modulators, 8 protein folding modulators to 9 protein folding modulators, 8 protein folding modulators to 10 protein folding modulators, or 9 protein folding modulators to 10 protein folding modulators. In some embodiments, the recombinant gram-negative host cell overexpresses 1 protein folding modulator, 2 protein folding modulators, 3 protein folding modulators, 4 protein folding modulators, 5 protein folding modulators, 6 protein folding modulators, 7 protein folding modulators, 8 protein folding modulators, 9 protein folding modulators, or 10 protein folding modulators. In some embodiments, the recombinant gram-negative host cell overexpresses at least 1 protein folding modulator, 2 protein folding modulators, 3 protein folding modulators, 4 protein folding modulators, 5 protein folding modulators, 6 protein folding modulators, 7 protein folding modulators, 8 protein folding modulators, or 9 protein folding modulators. In some embodiments, the recombinant gram-negative host cell overexpresses at most 2 protein folding modulators, 3 protein folding modulators, 4 protein folding modulators, 5 protein folding modulators, 6 protein folding modulators, 7 protein folding modulators, 8 protein folding modulators, 9 protein folding modulators, or 10 protein folding modulators.

### Protein Sequence Similarity

It is understood by those of skill in the art that the sequences of the proteins and peptides provided in the context of the present invention, e.g., as overexpressed (including the CDR3 knob peptide and folding modulators), or as deficient (e.g., proteases), may vary but retain the same activity. It is understood that any amino acid sequence similarity or identity range provided elsewhere herein may be replaced with a narrower range falling within that range, and that any minimum amino acid sequence similarity or identity provided herein may be replaced with a higher minimum. In some embodiments, a protein may have an amino acid sequence similarity or identity, active/catalytic site amino acid sequence similarity or identity, or allosteric region amino acid sequence similarity or identity, of about 30% to about 100%. Sequence similarity or identity of nucleic acid or amino acid sequences as described herein may be determined by methods known to those of skill in the art. In some embodiments, amino acids are similar with regard to polarity, charge, solubility, hydrophobicity, hydrophilicity and/or the amphipathic nature of the residues. For example, negatively charged amino acids include aspartic acid and glutamic acid; positively charged amino acids include lysine and arginine; amino acids with uncharged polar head groups or nonpolar head groups having similar hydrophilicity values include the following: leucine, isoleucine, valine; glycine, alanine; asparagine, glutamine; serine, threonine; phenylalanine, tyrosine. Thus, a similar amino acid may be an amino acid identified as suitable for a conservative amino acid substitution, e.g., as described in the literature and readily identified by methods known to those of skill in the art, for example, as shown in Table A, listing conservative amino acid substitutions. In some embodiments, a similar amino acid is an amino acid listed in Table A, second column (headed “I. Conservative Substitutions”) in the row corresponding to the original amino acid. In some embodiments, a similar amino acid is an amino acid listed in Table A, third column (headed “II. Alternative Substitutions”) in the row corresponding to the original amino acid.

In some embodiments, related proteins have amino acid sequence similarity or identity, active site amino acid sequence similarity or identity, or allosteric region amino acid sequence similarity or identity, of about 30% to about 35%, about 30% to about 40%, about 30% to about 45%, about 30% to about 50%, about 30% to about 55%, about 30% to about 60%, about 30% to about 65%, about 30% to about 70%, about 30% to about 80%, about 30% to about 90%, about 30% to about 100%, about 35% to about 40%, about 35% to about 45%, about 35% to about 50%, about 35% to about 55%, about 35% to about 60%, about 35% to about 65%, about 35% to about 70%, about 35% to about 80%, about 35% to about 90%, about 35% to about 100%, about 40% to about 45%, about 40% to about 50%, about 40% to about 55%, about 40% to about 60%, about 40% to about 65%, about 40% to about 70%, about 40% to about 80%, about 40% to about 90%, about 40% to about 100%, about 45% to about 50%, about 45% to about 55%, about 45% to about 60%, about 45% to about 65%, about 45% to about 70%, about 45% to about 80%, about 45% to about 90%, about 45% to about 100%, about 50% to about 55%, about 50% to about 60%, about 50% to about 65%, about 50% to about 70%, about 50% to about 80%, about 50% to about 90%, about 50% to about 100%, about 55% to about 60%, about 55% to about 65%, about 55% to about 70%, about 55% to about 80%, about 55% to about 90%, about 55% to about 100%, about 60% to about 65%, about 60% to about 70%, about 60% to about 80%, about 60% to about 90%, about 60% to about 100%, about 65% to about 70%, about 65% to about 80%, about 65% to about 90%, about 65% to about 100%, about 70% to about 80%, about 70% to about 90%, about 70% to about 100%, about 80% to about 90%, about 80% to about 100%, or about 90% to about 100%. In some embodiments, related proteins have amino acid sequence similarity or identity, active site amino acid sequence similarity or identity, or allosteric region amino acid sequence similarity or identity, of about 30%, about 35%, about 40%, about 45%, about 50%, about 55%, about 60%, about 65%, about 70%, about 80%, about 90%, or about 100%. In some embodiments, related proteins have amino acid sequence similarity or identity, active site amino acid sequence similarity or identity, or allosteric region amino acid sequence similarity or identity, of at least about 30%, about 35%, about 40%, about 45%, about 50%, about 55%, about 60%, about 65%, about 70%, about 80%, or about 90%. In some embodiments, related proteins have amino acid sequence similarity or identity, active site amino acid sequence similarity or identity, or allosteric region amino acid sequence similarity or identity, of at most about 35%, about 40%, about 45%, about 50%, about 55%, about 60%, about 65%, about 70%, about 80%, about 90%, or about 100%. In some embodiments, related proteins have amino acid sequence similarity or identity, active site amino acid sequence similarity or identity, or allosteric region amino acid sequence similarity or identity, of about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, related proteins have amino acid sequence similarity or identity, active site amino acid sequence similarity or identity, or allosteric region amino acid sequence similarity or identity, of about 45%, about 50%, about 55%, about 60%, about 65%, about 70%, about 75%, about 80%, about 85%, about 90%, about 95%, or about 100%. In some embodiments, related proteins have amino acid sequence similarity or identity, active site amino acid sequence similarity or identity, or allosteric region amino acid sequence similarity or identity, of at least about 45%, about 50%, about 55%, about 60%, about 65%, about 70%, about 75%, about 80%, about 85%, about 90%, or about 95%. In some embodiments, related proteins have amino acid sequence similarity or identity, active site amino acid sequence similarity or identity, or allosteric region amino acid sequence similarity or identity, of at most about 50%, about 55%, about 60%, about 65%, about 70%, about 75%, about 80%, about 85%, about 90%, about 95%, or about 100%.

Nucleic acid and amino acid sequence similarity identity may be determined according to any suitable method known in the art, including but not limited to those described herein. For example, alignments and searches for similar sequences can be performed using the U.S. National Center for Biotechnology Information (NCBI, Bethesda, MD) program, MegaBLAST. Use of this program with options for percent identity set at, for example, 70% for amino acid sequences, or set at, for example, 90% for nucleotide sequences, will identify those sequences with 70%, or 90%, or greater sequence identity to the query sequence. Other software known in the art is also available for aligning and/or searching for similar sequences, e.g., sequences at least 70% or 90% identical to an information string containing a secretion signal sequence herein. For example, sequence alignments for comparison to identify sequences at least 70% or 90% identical to a query sequence is often performed by use of, e.g., the GAP, BESTFIT, BLAST, FASTA, and TFASTA programs available in the GCG Sequence Analysis Software Package (available from the Genetics Computer Group, University of Wisconsin Biotechnology Center, 1710 University Avenue, Madison, Wis. 53705), with the default parameters as specified therein, plus a parameter for the extent of sequence identity set at the desired percentage. Also, for example, the CLUSTAL program (available in the PC/Gene software package from Intelligenetics, Mountain View, CA.) may be used.

These and other sequence alignment methods are well known in the art and may be conducted by manual alignment, by visual inspection, or by manual or automatic application of a sequence alignment algorithm, such as any of those embodied by the above-described programs. Various useful algorithms include, e.g.: the similarity search method described in W. R. Pearson & D. J. Lipman, Proc. Natl. Acad. Sci. USA 85:2444-48 (April 1988); the local homology method described in T. F. Smith & M. S. Waterman, in Adv. Appl. Math. 2:482-89 (1981) and in J. Molec. Biol. 147:195-97 (1981); the homology alignment method described in S. B. Needleman & C. D. Wunsch, J. Molec. Biol. 48 (3): 443-53 (March 1970); and the various methods described, e.g., by W. R. Pearson, in Genomics 11 (3): 635-50 (November 1991); by W. R. Pearson, in Methods Molec. Biol. 24:307-31 and 25:365-89 (1994); and by D. G. Higgins & P. M. Sharp, in Comp. Appl'ns in Biosci. 5:151-53 (1989) and in Gene 73 (1): 237-44 (15 Dec. 1988).

GAP Version 10, which uses the algorithm of Needleman and Wunsch (1970) supra, can be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent or similar programs may also be used as will be understood by one of skill in the art. For example, a sequence comparison program can be used that, for any two sequences in question, generates an alignment having identical nucleotide residue matches and an identical percent sequence identity when compared to the corresponding alignment generated by GAP Version 10. In some embodiments, the sequence comparison is performed across the entirety of the query or the subject sequence, or both.

### Mutations Resulting in a Deficiency of a Recombinant Host Cell Protein or in an Overexpressed Inactivated Protease

A recombinant bacterial host cell of the invention having a deficient protein activity can be generated by altering one or more genes encoding a protein having the protein activity, by any known method. A “deficient” protein activity or “deficiency” in a protein activity as used throughout this description, may include a partial deficiency, a substantial deficiency, or a complete deficiency. A “deficient” protein activity or “deficiency” in a protein activity as used throughout this description may include a reduction in, or elimination of, the protein activity. In some embodiments, the recombinant host cell protein activity is accordingly deficient in the host cell as compared with a control cell. In some embodiments, a control cell is a corresponding host cell that has wild-type activity of the protein. In some embodiments, a control cell is a corresponding wild-type cell. In some embodiments, a control cell has wild-type activity of the protein but has other differences relative to a wild-type cell. The recombinant host cell of the invention may be modified by any suitable means, e.g., as described herein, to reduce or eliminate the activity of protein. A recombinant bacterial host cell of the invention may also overexpress an inactivated protease, as described herein. In some embodiments, the overexpressed inactivated protease is partially inactivated, substantially inactivated, or fully inactivated with regard to the protease activity. In some embodiments, the overexpressed inactivated protease is partially inactivated, substantially inactivated, or fully inactivated with regard to the protease activity, and active with respect to another property, e.g., a chaperone activity. In some embodiments, the inactivated protease is inactivated by mutation, e.g., by mutation of a gene encoding the active protease (having protease activity).

In some embodiments, the deficient or reduced protein activity of the recombinant host cell results from a mutation that causes an amino acid change or other disruption, e.g., by amino acid substitution, deletion of one or more amino acid, insertion of one or more amino acid, or protein truncation. In some embodiments, the mutation is an inactivating mutation. In some embodiments, the mutation is a partially-inactivating mutation. In some embodiments, a deficiency in the activity of a protein, e.g., a protease or autolytic factor, results from one or more mutation independently selected from (i) a complete gene deletion (gene knockout), (ii) a partial gene deletion, (iii) a missense mutation, (iv) a nonsense mutation, (v) a frameshift mutation, (vi) an insertion, and (vii) any combination of (ii), (iii), (iv), (v) and (vi). In some embodiments, an overexpressed inactivated protease is inactivated by one or more mutation independently selected from (ii) a partial gene deletion, (iii) a missense mutation, (iv) a nonsense mutation, (v) a frameshift mutation, (vi) an insertion, and (vii) any combination of (ii), (iii), (iv), (v) and (vi). In some embodiments, the mutation resulting in a deficient protein activity or an inactivated protease is in a coding region of a gene encoding the protein or inactivated protease. In some embodiments, the mutation resulting in a deficient protein activity is in a non-coding region of the gene encoding the protein. In some embodiments, the non-coding region of the gene is a regulatory region. In some embodiments, the mutation in the regulatory region of the gene disrupts a regulatory element that is required for production of the protein, for example, an element required for transcription of the corresponding RNA, or translation of the mRNA into protein. For example, a noncoding region regulatory element can be a promoter, enhancer, regulatory protein binding site, ribosome binding site, or any other regulatory element as known to those of skill in the art.

In some embodiments, a mutation disrupts a critical site in a protein to result in a deficient protein in the recombinant host cell, or an inactivated overexpressed protease, e.g., by changing or deleting one or more amino acids at a protease active site. In some embodiments, a mutation disrupts an allosteric region of the protein, e.g., by changing one or more amino acids in an allosteric region. An allosteric region may be a region that interacts with another region to form an active protein conformation. In some embodiments, a mutation results in the substitution of an amino acid with any other amino acid. In some embodiments, the substitution is a non-conservative amino acid substitution. A non-conservative amino acid substitution can be readily selected by one of skill in the art. Table A provides examples of conservative amino acid substitutions (column I) and alternative conservative amino acid substitutions (II). In some embodiments, a non-conservative substitution of an original amino acid (e.g., the amino acid in the wild-type protein) is a substitution with any amino acid not listed in (I) for the original amino acid. In some embodiments, a non-conservative substitution of an original amino acid is any amino acid not listed in (II) for the original amino acid. In some embodiments, a non-conservative amino acid substitution is any amino acid not listed in either (I) or (II) for the original amino acid.

TABLE A

| Amino | I. Conservative | II. Alternative |
|---|---|---|
| Acid | Substitutions | Substitutions |
| Ala | Gly, Ile, Leu, Val | any aliphatic amino acid or derivative |
| | | thereof |
| | | (Ala, Gly, Ile, Leu, Val) |
| | | any hydrophobic amino acid or |
| | | derivative thereof |
| | | (Ala, Gly, Ile, Leu, Met, Phe, Pro, Val) |
| Arg | His, Lys | any basic amino acid or derivative |
| | | thereof |
| | | (Arg, His, Lys) |
| | | any charged amino acid or derivative |
| | | thereof |
| | | (Asp, Arg, Glu, Lys) |
| | | any basic amino acid with an |
| | | electrically charged sidechain or |
| | | derivative thereof |
| | | (Arg, His, Lys) |
| Asn | Asp, Gln, Glu | any acidic amino acid or derivative |
| | | thereof, or any amide of any acidic |
| | | amino acid or derivative thereof |
| | | (Asn, Asp, Gln, Glu) |
| | | any polar amino acid or derivative |
| | | thereof |
| | | (Asn, Cys, Gln, His, Ser, Thr, Trp, Tyr) |
| Asp | Asn, Gln, Glu | any acidic amino acid or derivative |
| | | thereof, or any amide of any acidic |
| | | amino acid or derivative thereof |
| | | (Asn, Asp, Gln, Glu) |
| | | any charged amino acid or derivative |
| | | thereof |
| | | (Asp, Arg, Glu, Lys) |
| | | any polar neutral amino acid or |
| | | derivative thereof |
| | | (Asp, Cys, Gln, Ser, Thr) |
| | | any acidic amino acid with an |
| | | electrically charged sidechain or |
| | | derivative thereof |
| | | (Asp, Glu) |
| Cys | Met, Sec, Ser, Thr | any hydroxyl or sulfur/selenium- |
| | | containing amino acid or derivative |
| | | thereof |
| | | (Cys, Sec, Ser, Met, Thr) |
| | | any polar amino acid or derivative |
| | | thereof |
| | | (Asn, Cys, Gln, His, Ser, Thr, Trp, Tyr) |
| | | any polar neutral amino acid or |
| | | derivative thereof |
| | | (Asp, Cys, Gln, Ser, Thr) |
| Gln | Asn, Asp, Glu | any acidic amino acid or derivative |
| | | thereof, or any amide of any acidic |
| | | amino acid or derivative thereof |
| | | (Asn, Asp, Gln, Glu) |
| | | any polar amino acid or derivative |
| | | thereof |
| | | (Asn, Cys, Gln, His, Ser, Thr, Trp, |
| | | Tyr) |
| | | any polar neutral amino acid or |
| | | derivative thereof |
| | | (Asp, Cys, Gln, Ser, Thr) |
| | | any acidic amino acid with an |
| | | electrically charged sidechain or |
| | | derivative thereof |
| | | (Asp, Glu) |
| Glu | Asn, Asp, Gln | any acidic amino acid or derivative |
| | | thereof, or any amide of any acidic |
| | | amino acid or derivative thereof |
| | | (Asn, Asp, Gln, Glu) |
| | | any charged amino acid or derivative |
| | | thereof |
| | | (Asp, Arg, Glu, Lys) |
| Gly | Ala, Ile, Leu, Val | any aliphatic amino acid or derivative |
| | | thereof |
| | | (Ala, Gly, Ile, Leu, Val) |
| | | any hydrophobic amino acid or |
| | | derivative thereof |
| | | (Ala, Gly, Ile, Leu, Met, Phe, Pro, Val) |
| His | Arg, Lys | any basic amino acid or derivative |
| | | thereof |
| | | (Arg, His, Lys) |
| | | any polar amino acid or derivative |
| | | thereof |
| | | (Asn, Cys, Gln, His, Ser, Thr, Trp, Tyr) |
| | | any basic amino acid with an |
| | | electrically charged sidechain or |
| | | derivative thereof |
| | | (Arg, His, Lys) |
| Ile | Ala, Gly, Leu, Val | any aliphatic amino acid or derivative |
| | | thereof |
| | | (Ala, Gly, Ile, Leu, Val) |
| | | any hydrophobic amino acid or |
| | | derivative thereof |
| | | (Ala, Gly, Ile, Leu, Met, Phe, Pro, Val) |
| Leu | Ala, Gly, Ile, Val | any aliphatic amino acid or derivative |
| | | thereof |
| | | (Ala, Gly, Ile, Leu, Val) |
| | | any hydrophobic amino acid or |
| | | derivative thereof |
| | | (Ala, Gly, Ile, Leu, Met, Phe, Pro, Val) |
| Lys | Arg, His | any basic amino acid or derivative |
| | | thereof |
| | | (Arg, His, Lys) |
| | | any charged amino acid or derivative |
| | | thereof |
| | | (Asp, Arg, Glu, Lys) |
| | | any basic amino acid with an |
| | | electrically charged sidechain or |
| | | derivative thereof |
| | | (Arg, His, Lys) |
| Met | Cys, Sec, Ser, Thr | any hydroxyl or sulfur/selenium- |
| | | containing amino acid or derivative |
| | | thereof |
| | | (Cys, Sec, Ser, Met, Thr) |
| | | any hydrophobic amino acid or |
| | | derivative thereof |
| | | (Ala, Gly, Ile, Leu, Met, Phe, Pro, Val) |
| Phe | Trp, Tyr | any aromatic amino acid or derivative |
| | | thereof |
| | | (Phe, Trp, Tyr) |
| | | any hydrophobic amino acid or |
| | | derivative thereof |
| | | (Ala, Gly, Ile, Leu, Met, Phe, Pro, Val) |
| Pro | | any cyclic amino acid or derivative |
| | | thereof |
| | | (Pro) |
| | | any hydrophobic amino acid or |
| | | derivative thereof |
| | | (Ala, Gly, Ile, Leu, Met, Phe, Pro, Val) |
| Ser | Cys, Met, Sec, Thr | any hydroxyl or sulfur/selenium- |
| | | containing amino acid or derivative |
| | | thereof |
| | | (Cys, Sec, Ser, Met, Thr) |
| | | any polar amino acid or derivative |
| | | thereof |
| | | (Asn, Cys, Gln, His, Ser, Thr, Trp, |
| | | Tyr) |
| | | any polar neutral amino acid or |
| | | derivative thereof |
| | | (Asp, Cys, Gln, Ser, Thr) |
| Thr | Cys, Met, Sec, Ser | any hydroxyl or sulfur/selenium- |
| | | containing amino acid or derivative |
| | | thereof |
| | | (Cys, Sec, Ser, Met, Thr) |
| | | any polar amino acid or derivative |
| | | thereof |
| | | (Asn, Cys, Gln, His, Ser, Thr, Trp, |
| | | Tyr) |
| | | any polar neutral amino acid or |
| | | derivative thereof |
| | | (Asp, Cys, Gln, Ser, Thr) |
| Trp | Phe, Tyr | any aromatic amino acid or derivative |
| | | thereof |
| | | (Phe, Trp, Tyr) |
| | | any polar amino acid or derivative |
| | | thereof |
| | | (Asn, Cys, Gln, His, Ser, Thr, Trp, |
| | | Tyr) |
| Tyr | Phe, Trp | any aromatic amino acid or derivative |
| | | thereof |
| | | (Phe, Trp, Tyr) |
| | | any polar amino acid or derivative |
| | | thereof |
| | | (Asn, Cys, Gln, His, Ser, Thr, Trp, |
| | | Tyr) |
| Val | Ala, Gly, Ile, Leu | any aliphatic amino acid or derivative |
| | | thereof |
| | | (Ala, Gly, Ile, Leu, Val) |
| | | any hydrophobic amino acid or |
| | | derivative thereof |
| | | (Ala, Gly, Ile, Leu, Met, Phe, Pro, Val) |

### Methods for Producing a Recombinant Ultralong CDR3 Knob Peptide

The present invention includes methods for producing a recombinant protein of interest, e.g., recombinant ultralong CDR3 knob peptide using the recombinant gram-negative bacterial host cells described herein. The compositions and methods of the invention can be used to produce a recombinant protein of interest of high quality, at high yield, or both. A high quality recombinant protein of interest can be soluble, active, intact, or any combination thereof. In some embodiments, the compositions and methods of the invention are used to produce a recombinant protein that is soluble, active, intact, present at high yield, or any combination thereof.

In some embodiments, a method for producing a recombinant protein of interest comprises: recovering the recombinant protein of interest from a recombinant gram-negative bacterial host cell as set forth herein, wherein the recombinant gram-negative host cell has been cultured under suitable fermentation conditions, wherein the recombinant gram-negative host cell has been transformed with at least one expression vector encoding the recombinant protein of interest. In some embodiments, recovery of the recombinant protein of interest from the recombinant gram-negative bacterial host cell comprises at least one purification step. In some embodiments, the yield and/or quality of the recovered recombinant protein of interest is measured. In some embodiments, the yield and/or quality of the recovered recombinant protein of interest is compared with that recovered from a control cell.

Production and evaluation of a recombinant protein of interest using the inventive gram-negative bacterial host cells as described herein may carried out as set forth herein, in combination with known tools and methods for producing recombinant proteins in bacterial host cells.

### Gram-Negative Bacterial Host Cells

Gram-negative bacterial host cells of the present invention include Pseudomonads (i.e., host cells in the order Pseudomonadales) and related bacterial organisms known in the art, e.g., Escherichia, Erwinia, Salmonella, Shigella, Moraxella, Helicobacter, Legionella, Neisseria, Haemophilus, Acinetobacter, Xylella, Bacteroides, Citrobacter, Enterobacter, Klebsiella, Proteus, Serratia, Shigella, Yersinia and Vibrio, and including any species or subspecies, including but not limited to P. fluorescens, P. aeruginosa, P. putida, E. coli, E. chrysanthemi, S. typhimurium, Helicobacter pylori, L. pneumophila, N. meningitidis, N. gonorrhoeae, Haemophilus influenzae, V. cholerae, X. fastidiosa, and A. baylyi.

In some embodiments, the Pseudomonad host cell is Pseudomonas fluorescens.

In some embodiments, the host cell is of the order Pseudomonadales (referred to herein as a “Pseudomonad.” Where the host cell is of the order Pseudomonadales, it may be a member of the family Pseudomonadaceae, including the genus Pseudomonas.

Gamma Proteobacterial hosts include members of the species Escherichia coli and members of the species Pseudomonas fluorescens. Other Pseudomonas organisms may also be useful. Pseudomonads and closely related species include Gram-negative Proteobacteria Subgroup 1, which include the group of Proteobacteria belonging to the families and/or genera described as “Gram-Negative Aerobic Rods and Cocci” by R. E. Buchanan and N. E. Gibbons (eds.), Bergey's Manual of Determinative Bacteriology, pp. 217-289 (8th ed., 1974) (The Williams & Wilkins Co., Baltimore, Md., USA), all are incorporated by reference herein in its entirety. Table B presents these families and genera of organisms.

TABLE B
Families and Genera (“Gram-Negative Aerobic
Rods and Cocci.” Bergey's, 1974)

| | |
|---|---|
| Family I. Pseudomonaceae Gluconobacter | Pseudomonas |
| | Xanthomonas |
| | Zoogloea |
| Family II. Azotobacteraceae Azomonas | Azotobacter |
| | Beijerinckia |
| | Derxia |
| Family III. Rhizobiaceae Agrobacterium | Rhizobium |
| Family IV. Methylomonadaceae Methylococcus | Methylomonas |
| Family V. Halobacteriaceae Halobacterium | Halococcus |
| Other Genera Acetobacter | Alcaligenes |
| | Bordetella |
| | Brucella |
| | Francisella |
| | Thermus |

Pseudomonas and closely related bacteria are generally part of the group defined as “Gram (−) Proteobacteria Subgroup 1” or “Gram-Negative Aerobic Rods and Cocci” (Buchanan and Gibbons (eds.) (1974) Bergey's Manual of Determinative Bacteriology, pp. 217-289). Pseudomonas host strains are described in the literature, e.g., in U.S. Pat. Nos. 9,458,487 and 9,453,251, both entitled “Expression of mammalian proteins in Pseudomonas fluorescens,” and U.S. Pat. No. 10,118,956, “Fusion Partners for Peptide Production,” each incorporated by reference herein.

“Gram-negative Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition, hosts can include cells from the genus Pseudomonas, Pseudomonas enalia (ATCC 14393), Pseudomonas nigrifaciensi (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram-negative Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram-negative Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

The host cell can be selected from “Gram-negative Proteobacteria Subgroup 16.” “Gram-negative Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beyerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas cedrina; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginate (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas filva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis. In one embodiment, the host cell is Pseudomonas fluorescens.

The host cell can also be selected from “Gram-negative Proteobacteria Subgroup 17.” “Gram-negative Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

### Host Strain Backgrounds

Host cells, strains and expression constructs useful in practicing the methods of the invention can be identified or made using reagents and methods known to those of skill in the art and described in the literature. For example, U.S. Pat. No. 8,288,127, “Protein Expression Systems,” incorporated herein by reference in its entirety, describes production of a recombinant polypeptide by introduction of a nucleic acid construct into an auxotrophic Pseudomonas fluorescens host cell comprising a chromosomal lacI gene insert (e.g., lsc::lacI^{Q1}). The nucleic acid construct comprises a nucleotide sequence encoding the recombinant polypeptide operably linked to a promoter capable of directing expression of the nucleic acid in the host cell, and also comprises a nucleotide sequence encoding an auxotrophic selection marker. The auxotrophic selection marker is a polypeptide that restores prototrophy to the auxotrophic host cell. In some embodiments, the cell is auxotrophic for proline, uracil, or combinations thereof. In some embodiments, the host cell is derived from MB101 (ATCC deposit PTA-7841). U.S. Pat. No. 8,288,127, “Protein Expression Systems,” and Schneider, et al., 2005, “Auxotrophic markers pyrF and proC can replace antibiotic markers on protein production plasmids in high-cell-density Pseudomonas fluorescens fermentation,” Biotechnol. Progress 21 (2): 343-8, both incorporated herein by reference in their entirety, describe a production host strain auxotrophic for uracil that was constructed by deleting the pyrF gene in strain MB101. The pyrF gene can be cloned from strain MB214 (ATCC deposit PTA-7840) to generate a plasmid that complements the pyrF deletion to restore prototrophy. In particular embodiments, a dual pyrF-proC dual auxotrophic selection marker system in a P. fluorescens host cell is used. Given the published literature, a pyrF deleted production host strain as described can be produced by one of skill in the art using known methods and used as the background for introducing other desired genomic changes, including those described herein as useful in practicing the methods of the invention. It would be understood by one of skill in the art that a production host strain useful in the methods of the present invention can be generated using a publicly available host cell, for example, P. fluorescens MB101, e.g., by inactivating and/or introducing any genes as needed, using any of many suitable methods known in the art and described in the literature. It is also understood that a prototrophy restoring plasmid can be transformed into the strain, e.g., a plasmid carrying the pyrF gene from strain MB214, using any suitable method known in the art and described in the literature. Additionally, in such strains inactivated protease and folding modulator overexpression constructs may be introduced, using methods well known in the art.

In some embodiments, a P. fluorescens host strain used in the methods of the invention is DC454 (ΔpyrF, lsc::lacI^{Q1}), a derivative of deposited strain MB101 in which the gene pyrFis deleted, and the E. coli lacI transcriptional repressor is inserted and fused with the levansucrase gene (lsc). Sequences for these genes and methods for their use are known in the art and described in the literature, e.g., in U.S. Pat. Nos. 8,288,127, 8,017,355, “Mannitol induced promoter systems in bacterial host cells,” and 7,794,972, “Benzoate- and anthranilate-inducible promoters,” each incorporated by reference herein.

Host cell DC454 is described by Schneider, et al., 2005, where it is referred to as DC206, and in U.S. Pat. No. 8,569,015, “rPA Optimization,” incorporated herein by reference in its entirety. DC206 is the same strain as DC454; it was renamed DC454 after passage three times in animal-free media.

In some embodiments, a host cell genomic deletion or mutation (e.g., an inactivating or debilitating mutation) can be made by, e.g., allele exchange, using a deletion plasmid carrying regions that flank the gene to be deleted, which does not replicate in P. fluorescens. The deletion plasmid can be constructed by PCR amplifying the gene to be deleted, including the upstream and downstream regions of the gene to be deleted. The deletion can be verified by sequencing a PCR product amplified from genomic DNA using analytical primers, observed after separation by electrophoresis in an agarose slab gel, followed by DNA sequencing of the fragment. In some embodiments, a gene is inactivated by complete deletion, partial deletion, or mutation, e.g., frameshift, point, or insertion mutation.

In some embodiments, a strain used in the context of the present invention has been transformed with an FMO plasmid according to methods known in the art. In some embodiments, a host cell equivalent to any host cell described in any one of Tables 3, 4, 6 or 9 is transformed with an expression vector as described herein, to obtain an expression strain equivalent to one described herein for expressing a recombinant protein of interest using the methods of the invention. As described, appropriate expression strains can be similarly derived according to methods set forth herein and in the literature.

Expression strains useful for practicing the methods of the invention can be constructed using methods described herein and in the published literature. In some embodiments, an expression strain useful in the methods of the invention comprises a plasmid overexpressing one or more P. fluorescens chaperone or folding modulator protein. For example, SecB, DsbA, DsbC, Skp, FklB2, or any combination thereof can be overexpressed in the expression strain. In some embodiments, one or more overexpression plasmid encodes SecB, DsbA, DsbC, Skp, FklB2 or any combination thereof.

A recombinant Pseudomonad host cell useful in the methods of the invention, for producing a recombinant ultralong CDR3 knob peptide, may: be deficient in a protease selected from Prc1, Prc2, DegP2, HslUV, MepM1, Lon, La1, and a serralysin (e.g., serralysin RXF04495.2); overexpress a folding modulator selected from: SecB, DsbA, DsbB, DsbC, Skp, FklB2, and Ppi; or any combination thereof.

The recombinant Pseudomonad host cell may be deficient in proteases HslUV, Prc 1, Prc2, MepM1, and serralysin RXF04495.2, and comprise an expression construct comprising sequences encoding a recombinant ultralong CDR3 knob peptide directly and operably linked to a secretion leader sequence selected from AnsB, CupB2, Leader M, and LAO.

The recombinant Pseudomonad host cell may overexpress folding modulators DsbC and DsbA, and comprise an expression construct comprising sequences encoding a recombinant ultralong CDR3 knob peptide directly and operably linked to a FlgI secretion leader sequence.

The recombinant Pseudomonad host cell may overexpress folding modulators FklB2 and DsbC, and comprise an expression construct comprising sequences encoding a recombinant ultralong CDR3 knob peptide directly and operably linked to a FlgI secretion leader sequence.

The recombinant Pseudomonad host cell may overexpress folding modulator Ppi, and comprise an expression construct comprising sequences encoding a recombinant ultralong CDR3 knob peptide directly and operably linked to a Leader M secretion leader sequence. The Ppi may be PpiB, RXF05345.2.

Soluble Lysate The recombinant Pseudomonad host cell may be deficient in proteases HslUV, Prc 1, Prc2, and MepM1, and comprise an expression construct comprising sequences encoding a recombinant ultralong CDR3 knob peptide directly and operably linked to a secretion leader sequence selected from Leader M, CupB2, and AnsB.

The recombinant Pseudomonad host cell may be deficient in proteases Lon, La1, Prc 1, and Prc2, and comprise an expression construct comprising sequences encoding a recombinant ultralong CDR3 knob peptide directly and operably linked to a secretion leader sequence selected from Leader M, CupB2, AnsB and FlgI.

The recombinant Pseudomonad host cell may overexpress folding modulator DsbA and DsbC, and comprise an expression construct comprising sequences encoding a recombinant ultralong CDR3 knob peptide directly and operably linked to an AnsB secretion leader sequence.

The recombinant Pseudomonad host cell may overexpress folding modulators FklB3 and DsbC, and comprise an expression construct comprising sequences encoding a recombinant ultralong CDR3 knob peptide directly and operably linked to an AnsB or FlgI secretion leader sequence.

The recombinant Pseudomonad host cell may be deficient in proteases Lon, La1, and DegP2, and Prc2, and comprise an expression construct comprising sequences encoding a recombinant ultralong CDR3 knob peptide directly and operably linked to a FlgI secretion leader sequence.

The recombinant Pseudomonad host cell may be deficient in protease DegP2, overexpress folding modulator SecB, and comprise an expression construct comprising sequences encoding a recombinant ultralong CDR3 knob peptide directly and operably linked to an AnsB, FlgI, or TolB secretion leader sequence. The expression construct comprising the AnsB secretion leader sequence may also comprise an RBS Hi. The expression construct comprising the FlgI secretion leader sequence may also comprise an RBS Hi. The expression construct comprising the TolB secretion leader sequence may also comprise an RBS Med.

The recombinant Pseudomonad host cell may be deficient in protease DegP2, overexpress folding modulators DsbA, DsbC, and Skp, and comprise an expression construct comprising sequences encoding a recombinant ultralong CDR3 knob peptide directly and operably linked to an AnsB, FlgI or TolB secretion leader sequence. The expression construct comprising the TolB secretion leader sequence may also comprise an RBS Med.

In some embodiments, the host strain has a phenotype and genotype as set forth for any host strain in any one of Tables 3, 4, 6, and 9. In some embodiments, the host strain has a phenotype, genotype, and expression construct sequence elements as set forth for any bacterial strain in any one of Tables 3, 4, 6, and 9. In some embodiments, the host strain is any as set forth in any one of Tables 3, 4, 6, and 9.

### Expression Systems

An appropriate bacterial expression system useful for producing the recombinant protein of interest according to the present methods can be identified by one of skill in the art based on the teachings herein. In some embodiments, an expression construct comprising a nucleotide sequence encoding a recombinant protein of interest is provided as part of an inducible expression vector. In some embodiments, a host cell that has been transformed with the expression vector is cultured, and expression of the recombinant protein of interest from the expression vector is induced. The expression vector can be, for example, a plasmid. In some embodiments, the expression vector is a plasmid encoding a recombinant protein coding sequence further comprising a selection marker, and the host cells are grown under selective conditions that allow maintenance of the plasmid. In some embodiments, the expression construct is integrated into the host cell genome. In some embodiments, the expression construct encodes a recombinant protein of interest fused to a secretory signal that can direct the recombinant protein of interest to the periplasm.

Methods for expressing heterologous proteins, including useful regulatory sequences (e.g., promoters, secretion signals, and ribosome binding sites), in host cells useful in the methods of the present invention, are described in the literature, e.g., in U.S. Pat. No. 7,618,799, “Bacterial leader sequences for increased expression,” in U.S. Pat. No. 7,985,564, “Expression systems with Sec-system secretion,” in U.S. Pat. Nos. 9,394,571 and 9,580,719, 9,458,487 and 9,453,251, 8,603,824, 8,530,171, “High level expression of recombinant toxin proteins,” U.S. Pat. Nos. 10,118,956, 5,888,808, Bacterial polypeptide expression employing tryptophan promoter-operator,” U.S. Pat. No. 9,534,217, “Method of creating a library of bacterial clones with varying levels of gene expression,” and Vellanoweth, R. L., and Rabinowitz, J. C., May 1992, “The influence of ribosome-binding-site elements on translational efficiency in Bacillus subtilis and Escherichia coli in vivo,” Molecular Microbiology 6 (9): 1105-1114, each incorporated herein by reference in its entirety. In some embodiments, a secretion leader used in the context of the present invention is a secretion leader as disclosed in any of U.S. Pat. Nos. 7,618,799, 7,985,564, 9,394,571, 9,580,719, 9,453,251, 8,603,824, 8,530,171, and 10,118,956. These patents also describe bacterial host strains useful in practicing the methods herein, that have been engineered to overexpress folding modulators or wherein protease mutations have been introduced, in order to increase heterologous protein expression.

Promoters used in accordance with the present invention may be constitutive promoters or regulated promoters. Examples of inducible promoters include those of the family derived from the lac promoter (i.e. the lacZ promoter), e.g., the tac and trc promoters described in U.S. Pat. No. 4,551,433, “Microbial Hybrid Promoters,” incorporated herein by reference, as well as Ptac16, Ptac17, PtacII, PlacUV5, and the T7lac promoter. In some embodiments, the promoter is not derived from the host cell organism. In some embodiments, the promoter is derived from an E. coli organism. In some embodiments, a lac promoter is used to regulate expression of a recombinant protein of interest from a plasmid. In the case of the lac promoter derivatives or family members, e.g., the tac promoter, an inducer is IPTG (isopropyl-β-D-1-thiogalactopyranoside, “isopropylthiogalactoside”). In some embodiments, IPTG is added to the host cell culture to induce expression of the recombinant protein of interest from a lac promoter in a Pseudomonas host cell according to methods known in the art and described in the literature, e.g., in U.S. Pat. Nos. 9,458,487 and 9,453,251.

Examples of non-lac promoters useful in expression systems according to the present invention include, PR (induced by high temperature), PL (induced by high temperature), Pₘ (induced by Alkyl- or halo-benzoates), Pu (induced by alkyl- or halo-toluenes), or Pₛₐₗ (induced by salicylates), described in, e.g. J. Sanchez-Romero & V. De Lorenzo (1999) Manual of Industrial Microbiology and Biotechnology (A. Demain & J. Davies, eds.) pp. 460-74 (ASM Press, Washington, D.C.); H. Schweizer (2001) Current Opinion in Biotechnology, 12:439-445; and R. Slater & R. Williams (2000 Molecular Biology and Biotechnology (J. Walker & R. Rapley, eds.) pp. 125-54 (The Royal Society of Chemistry, Cambridge, UK). A promoter having the nucleotide sequence of a promoter native to the selected bacterial host cell also may be used to control expression of the expression construct encoding the polypeptide of interest, e.g, a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter, derived from the same or different organisms. In some embodiments, the promoter is Pmtl, as described in, e.g., U.S. Pat. Nos. 7,476,532, and 8,017,355, both titled “Mannitol induced promoter systems in bacterial host cells,” incorporated by reference herein in their entirety.

Regulated (inducible) promoters utilize promoter regulatory proteins in order to control transcription of the gene of which the promoter is a part. Where a regulated promoter is used herein, a corresponding promoter regulatory protein will also be part of an expression system according to the present invention. Examples of promoter regulatory proteins include: activator proteins, e.g., E. coli catabolite activator protein, MalT protein; AraC family transcriptional activators; repressor proteins, e.g., E. coli Lad proteins; and dual-function regulatory proteins, e.g., E. coli NagC protein. Many regulated-promoter/promoter-regulatory-protein pairs are known in the art. In some embodiments, a promoter used to transcribe a gene encoding a recombinant protein of interest produced using the present compositions and methods is selected from: a tac promoter, a mannitol promoter, a Pben, a T7 promoter, a lac promoter, a T5 promoter, a xylose promoter, a Trp promoter, and an arabinose promoter. When more than one expression construct is used to produce the recombinant protein of interest, more than one different promoter may be used.

Promoter regulatory proteins interact with an effector compound, i.e., a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a preferred embodiment in which a regulated promoter is used, after growth of a desired quantity or density of host cell biomass, an appropriate effector compound is added to the culture to directly or indirectly result in expression of the desired gene(s) encoding the recombinant protein of interest.

In some embodiments wherein a lac family promoter is utilized, a lacI gene can also be present in the system. The lac gene, which is normally a constitutively expressed gene, encodes the Lac repressor protein LacI protein, which binds to the lac operator of lac family promoters. Thus, where a lac family promoter is utilized, the lac gene can also be included and expressed in the expression system.

### Expression Vectors

At least one nucleic acid sequence encoding a recombinant protein of interest e.g., a recombinant ultralong CDR3 knob peptide, can be introduced into a suitable expression vector(s) to produce either the recombinant protein of interest, an overexpressed protein, e.g., a chaperone, folding modulator, or inactivated protease as described herein. The expression vector can be a plasmid. An expression vector may be selected for use in the context of the present invention by one of skill in the art as desired and appropriate, from commercially available expression vectors. In some embodiments, a plasmid encoding a recombinant protein of interest can comprise a selection marker, and host cells maintaining the plasmid can be grown under selective conditions. In some embodiments, the plasmid does not comprise a selection marker. In some embodiments, the expression vector is integrated into the host cell genome. In some embodiments, the expression vector encodes a recombinant protein of interest fused to a secretion signal that can direct the expressed recombinant protein of interest to the periplasm. In some embodiments, the expression vector encodes a recombinant protein of interest fused to a secretion signal that can direct the expressed recombinant protein of interest to the cytoplasm. In some embodiments, an expression vector encodes a recombinant ultralong CDR3 knob peptide, fused to a periplasmic secretion signal that can direct the expressed recombinant ultralong CDR3 knob peptide to the periplasm.

Recombinant proteins of interest that can be produced using the present compositions and methods are described herein. Amino acid sequences of recombinant proteins of interest, and potential coding sequences, are described herein and/or may readily be obtained by those of skill in the art.

### Other Regulatory Elements

In some embodiments, other regulatory elements are present in the expression construct encoding the recombinant protein of interest. In some embodiments, the soluble recombinant protein of interest is present in either the cytoplasm or periplasm of the cell during production. Secretion leaders useful for targeting a recombinant protein of interest to either compartment are described herein. In some embodiments, an expression construct of the present invention encodes a recombinant protein of interest fused to a secretion signal that can transport the recombinant protein of interest to the cytoplasm of a Pseudomonad cell. In some embodiments, an expression construct encodes a recombinant protein of interest fused to a secretion leader that can transport a recombinant protein of interest to the periplasm of a Pseudomonad cell. In some embodiments, the secretion leader is cleaved from the recombinant protein of interest.

Other elements include, but are not limited to, transcriptional enhancer sequences, translational enhancer sequences, other promoters, activators, translational start and stop signals, transcription terminators, cistronic regulators, polycistronic regulators, tag sequences, such as nucleotide sequence tags and tag polypeptide coding sequences, which facilitate identification, separation, purification, and/or isolation of an expressed polypeptide, as previously described. In some embodiments, the expression construct includes, in addition to the protein coding sequence, any of the following regulatory elements operably linked thereto: a promoter, a ribosome binding site (RBS), a transcription terminator, and translational start and stop signals. Useful RBSs can be obtained from any of the species useful as host cells in expression systems according to, e.g., U.S. Pat. Nos. 10,118,956 and 9,580,719, previously referenced. Many RBSs are known, e.g., those described in and referenced by D. Frishman et al., Gene 234 (2): 257-65 (8 Jul. 1999); and B. E. Suzek et al., Bioinformatics 17 (12): 1123-30 (December 2001), incorporated herein by reference. In addition, either native or synthetic RBSs may be used, e.g., those described in: EP 0207459 (synthetic RBSs); O. Ikehata et al., Eur. J. Biochem. 181 (3): 563-70 (1989). In some embodiments, a “Hi” ribosome binding site, aggaggt, is used in the construct. Ribosome binding sites, including the optimization of spacing between the RBS and translation initiation codon, are described in the literature, e.g., by Chen, et al., 1994, “Determination of the optimal aligned spacing between the Shine-Dalgarno sequence and the translation initiation codon of Escherichia coli mRNAs,” Nucleic Acids Research 22 (23): 4953-4957, and Ma, et al., 2002, “Correlations between Shine-Dalgarno Sequences and Gene Features Such as Predicted Expression Levels and Operon Structures,” J. Bact. 184 (20): 5733-45, incorporated herein by reference.

Further examples of methods, vectors, and translation and transcription elements, and other elements useful in the present invention are well known in the art and described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox, all incorporated herein by reference, as well as in other publications incorporated herein by reference.

### Secretion Leader Sequences

In some embodiments, a secretion signal or leader coding sequence is fused to the N-terminus of the sequence encoding the recombinant protein of interest. Use of secretion signal sequences can increase production of recombinant proteins in bacteria. Additionally, many types of proteins require secondary modifications that are inefficiently achieved using known methods. Secretion leader utilization can increase the harvest of properly folded proteins by secreting the protein from the intracellular environment. In gram-negative bacteria, a protein secreted from the cytoplasm can end up in the periplasmic space, attached to the outer membrane, or in the extracellular broth. These methods may avoid formation of inclusion bodies. Secretion of proteins into the periplasmic space also has the effect of facilitating proper disulfide bond formation (Bardwell et al., 1994, Phosphate Microorg, Chapter 45, 270-5, and Manoil, 2000, Methods in Enzymol. 326:35-47). Other benefits of secretion of recombinant protein include more efficient isolation of the protein, proper folding and disulfide bond formation of the protein leading to an increase in yield represented by, e.g., the percentage of the protein in active form, reduced formation of inclusion bodies and reduced toxicity to the host cell, and an increased percentage of the recombinant protein in soluble form. The potential for excretion of the protein of interest into the culture medium can also potentially promote continuous, rather than batch, culture for protein production. Secretion signals are described, e.g., in U.S. Pat. No. 7,618,799,” U.S. Pat. Nos. 7,985,564, 7,833,752, and U.S. Pat. App. Pub. No. 2019/0127744, “Bacterial leader sequences for periplasmic protein expression,” each incorporated herein by reference in its entirety, as well as by U.S. Pat. No. 10,118,956. In some embodiments, the secretion leader can be selected from 8484 (SEQ ID NO: 24), AnsB (SEQ ID NO: 26), CupB2 (SEQ ID NO: 28), FlgI (SEQ ID NO: 30), Ibp-S31A (SEQ ID NO: 32), Lao (SEQ ID NO: 34), Leader M, PorE (SEQ ID NO: 38), TolB (SEQ ID NO: 40), CupC2 (SEQ ID NO: 70), Azu (SEQ ID NO: 72), Pbp (SEQ ID NO: 74), PbpA20V (SEQ ID NO: 76), 5193 (SEQ ID NO: 78), or Ibp (SEQ ID NO: 80). In certain embodiments, the secretion leader has at least 85% identity, at least 90% identity, or at least 95% identity, to an amino acid sequence selected from SEQ ID NOS: 24, 26, 28, 30, 32, 34, 38, 40, 70, 72, 74, 76, 78, and 80.

In some embodiments, the recombinant protein of interest is targeted to the periplasm of the host cell or into the extracellular space. In some embodiments, the expression vector further comprises a nucleotide sequence encoding a secretion signal polypeptide operably linked to the nucleotide sequence encoding the recombinant protein of interest.

Compositions and methods for producing high levels of properly processed recombinant proteins or polypeptides in a host cell are provided. In some aspects, a novel secretion signal that promotes the targeting of the recombinant protein or polypeptide of interest to the periplasm of Gram-negative bacteria or into the extracellular environment is provided. The periplasmic secretion signal peptide disclosed herein enables transport of proteins across the inner membrane to the periplasmic space in Gram negative bacteria. In some aspects, periplasmic secretion signal peptide provided herein promotes the targeting of the recombinant protein or polypeptide of interest to the extracellular space in Gram-positive bacteria. Periplasmic protein expression allows for proper formation of disulfide bonds in the periplasm and can result in high level recombinant protein expression. Expression to the periplasmic space may enable more efficient recovery/purification of the recombinant protein. For the purposes of the present disclosure, a “secretion signal,” “secretion leader,” “secretion signal polypeptide,” “signal peptide,” “leader peptide” or “leader sequence” are intended to refer to a peptide sequence (or the polynucleotide encoding the peptide sequence) that is useful for targeting a protein or polypeptide of interest to a cell compartment, e.g., the periplasm of Gram-negative bacteria or into the extracellular space. The secretion signal sequence may be selected from: an amino acid sequence set forth as SEQ ID NO: 24, 26, 28, 30, 32, 34, 38, 40, 70, 72, 74, 76, 78, and 80; or a fragment or variant thereof. Nucleotide sequences encoding SEQ ID NOS: 24, 26, 28, 30, 32, 34, 38, 40, 70, 72, 74, 76, 78, and 80 are provided in SEQ ID NOS: 23, 25, 27, 29, 31, 33, 35, 37, 39, 69, 71, 73, 75, 77, and 79, respectively. As known to those of skill in the art, an amino acid sequence can be encoded by different nucleotide sequences due to the redundancy in the genetic code. The compositions and methods of the present invention thus may include the same secretion signal amino acid sequence whilst encoded by different nucleotide sequences. Also provided herein are fragments and variants of the secretion signal peptide sequence that can direct periplasmic expression of an operably linked recombinant protein or polypeptide of interest.

A secretion signal coding sequence that encodes the amino acid sequence as set forth in SEQ ID NOS: 24, 26, 28, 30, 32, 34, 38, or 40 may be fused to the N-terminus of a sequence encoding a heterologous recombinant protein or polypeptide of interest, e.g., an ultralong CDR3 knob peptide, to be expressed and targeted to the host cell periplasm or into the extracellular space. As used herein with regard to a heterologous secretion signal and protein or polypeptide of interest, a “heterologous” secretion signal peptide is not native to the protein or polypeptide of interest. Conversely, with regard to a secretion signal peptide, a “heterologous” protein or polypeptide of interest is not native to the secretion signal. In the context of the host cell, the term heterologous may refer to a protein or polypeptide of interest that is not native to a particular host cell.

The invention includes a method of producing a protein or polypeptide of interest in a prokaryotic host cell, comprising producing the protein or polypeptide of interest in the periplasm of a prokaryotic host cell cultured in a cell culture growth medium, wherein the prokaryotic host cell comprises an expression construct comprising a nucleic acid encoding a recombinant polypeptide comprising the protein or polypeptide of interest operably linked to a secretion signal peptide that directs expression of the protein or polypeptide of interest to the periplasm of the prokaryotic host cell, wherein the secretion signal peptide comprises the amino acid sequence of SEQ ID NOS: 24, 26, 28, 30, 32, 34, 38, 40, 70, 72, 74, 76, 78, or 80, and wherein the secretion signal peptide is not native to the protein or polypeptide of interest.

In some embodiments, the protein or polypeptide of interest is expressed in the periplasm properly cleaved from the secretion signal peptide, e.g., SEQ ID NO: 24, 26, 28, 30, 32, 34, 38, 40, 70, 72, 74, 76, 78, or 80. In some embodiments, the secretion signal peptide directs expression of the protein or polypeptide of interest to the periplasm or the extracellular space of a prokaryotic host cell in properly cleaved form, soluble form, active form, or any combination thereof. A correctly or properly cleaved or processed protein or polypeptide of interest may have an intact or substantially intact N-terminus. In some embodiments, an intact N-terminus comprises the full N-terminus of the recombinant protein or polypeptide construct, and/or the N-terminal methionine. In some embodiments, an intact N-terminus does not comprise any residual secretion signal peptide sequence. A substantially intact N-terminus may be truncated relative to the full N-terminus of the recombinant protein or polypeptide by 1, 2, 3, or more amino acids. This number may vary as determined by one of skill in the art based on the known influence of the N-terminal amino acids required for activity or solubility of the particular recombinant protein or polypeptide. A properly cleaved, soluble, and/or active, recombinant protein or polypeptide of interest expressed in the periplasm or extracellular space according to the present methods may comprise about 85% to about 100% recombinant protein or polypeptide having an intact or substantially intact N-terminus. In some embodiments, the amount of expressed recombinant protein or polypeptide having an intact or substantially intact N-terminus is about 85% to about 100%. In some embodiments, the amount of expressed recombinant protein or polypeptide having an intact or substantially intact N-terminus is about 85% to about 100%. In some embodiments, the amount of expressed recombinant protein or polypeptide having an intact or substantially intact N-terminus is about 85% to about 90%, about 85% to about 91%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the amount of expressed recombinant protein or polypeptide having an intact or substantially intact N-terminus is about 85%, about 90%, about 91%, about 92%, about 93%, about 94%, about 95%, about 96%, about 97%, about 98%, about 99%, or about 100%. In some embodiments, the amount of expressed recombinant protein or polypeptide having an intact or substantially intact N-terminus is at least about 85%, about 90%, about 91%, about 92%, about 93%, about 94%, about 95%, about 96%, about 97%, about 98%, or about 99%. In some embodiments, the amount of expressed recombinant protein or polypeptide having an intact or substantially intact N-terminus is at most about 90%, about 91%, about 92%, about 93%, about 94%, about 95%, about 96%, about 97%, about 98%, about 99%, or about 100%.

In some embodiments, the properly cleaved protein or polypeptide of interest having an intact or substantially intact N-terminus comprises the N-terminal methionine. In some embodiments, the properly cleaved protein or polypeptide of interest having an intact or substantially intact N-terminus does not comprise the N-terminal methionine, even when encoded. A protein or polypeptide of interest may require a substantially intact N-terminus for activity, solubility, or both. In some embodiments, a protein or polypeptide of interest has about 80-100% activity when compared to a control. In some embodiments, the control is the same protein or polypeptide of interest that comprises an N-terminal methionine. In some embodiments, the control is the same protein or polypeptide of interest that does not comprise an N-terminal methionine. In some embodiments, the control is the same protein or polypeptide of interest that has a substantially intact N-terminus. In some embodiments, the expressed or produced protein or polypeptide of interest has an activity relative to a control of about 80% to about 100%. In some embodiments, a protein or polypeptide of interest having a substantially intact N-terminus has an activity relative to a control of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 100%, about 90% to about 92%, about 90% to about 94%, about 90% to about 95%, about 90% to about 96%, about 90% to about 97%, about 90% to about 98%, about 90% to about 99%, about 90% to about 100%, about 92% to about 94%, about 92% to about 95%, about 92% to about 96%, about 92% to about 97%, about 92% to about 98%, about 92% to about 99%, about 92% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, a protein or polypeptide of interest having a substantially intact N-terminus has an activity relative to a control of about 80%, about 85%, about 90%, about 92%, about 94%, about 95%, about 96%, about 97%, about 98%, about 99%, or about 100%. In some embodiments, a protein or polypeptide of interest having a substantially intact N-terminus has an activity relative to a control of at least about 80%, about 85%, about 90%, about 92%, about 94%, about 95%, about 96%, about 97%, about 98%, or about 99%. In some embodiments, a protein or polypeptide of interest having a substantially intact N-terminus has an activity relative to a control of at most about 85%, about 90%, about 92%, about 94%, about 95%, about 96%, about 97%, about 98%, about 99%, or about 100%.

In some embodiments, the process produces correctly processed periplasmic or extracellular protein at about 0.1 g/L to about 50 g/L. In some embodiments, the process produces correctly processed periplasmic or extracellular protein at about 0.1 g/L to about 3 g/L. In some embodiments, the process produces correctly processed periplasmic or extracellular protein at about 0.1 g/L to about 0.2 g/L, about 0.1 g/L to about 0.3 g/L, about 0.1 g/L to about 0.4 g/L, about 0.1 g/L to about 0.5 g/L, about 0.1 g/L to about 0.6 g/L, about 0.1 g/L to about 0.7 g/L, about 0.1 g/L to about 0.8 g/L, about 0.1 g/L to about 0.9 g/L, about 0.1 g/L to about 1 g/L, about 0.1 g/L to about 2 g/L, about 0.1 g/L to about 3 g/L, about 0.2 g/L to about 0.3 g/L, about 0.2 g/L to about 0.4 g/L, about 0.2 g/L to about 0.5 g/L, about 0.2 g/L to about 0.6 g/L, about 0.2 g/L to about 0.7 g/L, about 0.2 g/L to about 0.8 g/L, about 0.2 g/L to about 0.9 g/L, about 0.2 g/L to about 1 g/L, about 0.2 g/L to about 2 g/L, about 0.2 g/L to about 3 g/L, about 0.3 g/L to about 0.4 g/L, about 0.3 g/L to about 0.5 g/L, about 0.3 g/L to about 0.6 g/L, about 0.3 g/L to about 0.7 g/L, about 0.3 g/L to about 0.8 g/L, about 0.3 g/L to about 0.9 g/L, about 0.3 g/L to about 1 g/L, about 0.3 g/L to about 2 g/L, about 0.3 g/L to about 3 g/L, about 0.4 g/L to about 0.5 g/L, about 0.4 g/L to about 0.6 g/L, about 0.4 g/L to about 0.7 g/L, about 0.4 g/L to about 0.8 g/L, about 0.4 g/L to about 0.9 g/L, about 0.4 g/L to about 1 g/L, about 0.4 g/L to about 2 g/L, about 0.4 g/L to about 3 g/L, about 0.5 g/L to about 0.6 g/L, about 0.5 g/L to about 0.7 g/L, about 0.5 g/L to about 0.8 g/L, about 0.5 g/L to about 0.9 g/L, about 0.5 g/L to about 1 g/L, about 0.5 g/L to about 2 g/L, about 0.5 g/L to about 3 g/L, about 0.6 g/L to about 0.7 g/L, about 0.6 g/L to about 0.8 g/L, about 0.6 g/L to about 0.9 g/L, about 0.6 g/L to about 1 g/L, about 0.6 g/L to about 2 g/L, about 0.6 g/L to about 3 g/L, about 0.7 g/L to about 0.8 g/L, about 0.7 g/L to about 0.9 g/L, about 0.7 g/L to about 1 g/L, about 0.7 g/L to about 2 g/L, about 0.7 g/L to about 3 g/L, about 0.8 g/L to about 0.9 g/L, about 0.8 g/L to about 1 g/L, about 0.8 g/L to about 2 g/L, about 0.8 g/L to about 3 g/L, about 0.9 g/L to about 1 g/L, about 0.9 g/L to about 2 g/L, about 0.9 g/L to about 3 g/L, about 1 g/L to about 2 g/L, about 1 g/L to about 3 g/L, or about 2 g/L to about 3 g/L. In some embodiments, the process produces correctly processed periplasmic or extracellular protein at about 0.1 g/L, about 0.2 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, or about 3 g/L. In some embodiments, the process produces correctly processed periplasmic or extracellular protein at at least about 0.1 g/L, about 0.2 g/L, about 0.3 g/L, about 0.4 g/L, about 0.5 g/L, about 0.6 g/L, about 0.7 g/L, about 0.8 g/L, about 0.9 g/L, about 1 g/L, or about 2 g/L. In some embodiments, the process produces correctly processed periplasmic or extracellular protein at at most about 0.2 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, or about 3 g/L. In some embodiments, the process produces correctly processed periplasmic or extracellular protein at about 0.1 g/L to about 50 g/L. In some embodiments, the process produces correctly processed periplasmic or extracellular protein at about 0.1 g/L to about 0.5 g/L, about 0.1 g/L to about 1 g/L, about 0.1 g/L to about 2 g/L, about 0.1 g/L to about 5 g/L, about 0.1 g/L to about 10 g/L, about 0.1 g/L to about 15 g/L, about 0.1 g/L to about 20 g/L, about 0.1 g/L to about 25 g/L, about 0.1 g/L to about 30 g/L, about 0.1 g/L to about 40 g/L, about 0.1 g/L to about 50 g/L, about 0.5 g/L to about 1 g/L, about 0.5 g/L to about 2 g/L, about 0.5 g/L to about 5 g/L, about 0.5 g/L to about 10 g/L, about 0.5 g/L to about 15 g/L, about 0.5 g/L to about 20 g/L, about 0.5 g/L to about 25 g/L, about 0.5 g/L to about 30 g/L, about 0.5 g/L to about 40 g/L, about 0.5 g/L to about 50 g/L, about 1 g/L to about 2 g/L, about 1 g/L to about 5 g/L, about 1 g/L to about 10 g/L, about 1 g/L to about 15 g/L, about 1 g/L to about 20 g/L, about 1 g/L to about 25 g/L, about 1 g/L to about 30 g/L, about 1 g/L to about 40 g/L, about 1 g/L to about 50 g/L, about 2 g/L to about 5 g/L, about 2 g/L to about 10 g/L, about 2 g/L to about 15 g/L, about 2 g/L to about 20 g/L, about 2 g/L to about 25 g/L, about 2 g/L to about 30 g/L, about 2 g/L to about 40 g/L, about 2 g/L to about 50 g/L, about 5 g/L to about 10 g/L, about 5 g/L to about 15 g/L, about 5 g/L to about 20 g/L, about 5 g/L to about 25 g/L, about 5 g/L to about 30 g/L, about 5 g/L to about 40 g/L, about 5 g/L to about 50 g/L, about 10 g/L to about 15 g/L, about 10 g/L to about 20 g/L, about 10 g/L to about 25 g/L, about 10 g/L to about 30 g/L, about 10 g/L to about 40 g/L, about 10 g/L to about 50 g/L, about 15 g/L to about 20 g/L, about 15 g/L to about 25 g/L, about 15 g/L to about 30 g/L, about 15 g/L to about 40 g/L, about 15 g/L to about 50 g/L, about 20 g/L to about 25 g/L, about 20 g/L to about 30 g/L, about 20 g/L to about 40 g/L, about 20 g/L to about 50 g/L, about 25 g/L to about 30 g/L, about 25 g/L to about 40 g/L, about 25 g/L to about 50 g/L, about 30 g/L to about 40 g/L, about 30 g/L to about 50 g/L, or about 40 g/L to about 50 g/L. In some embodiments, the process produces correctly processed periplasmic or extracellular protein at about 0.1 g/L, about 0.5 g/L, about 1 g/L, about 2 g/L, about 5 g/L, about 10 g/L, about 15 g/L, about 20 g/L, about 25 g/L, about 30 g/L, about 40 g/L, or about 50 g/L. In some embodiments, the process produces correctly processed periplasmic or extracellular protein at at least about 0.1 g/L, about 0.5 g/L, about 1 g/L, about 2 g/L, about 5 g/L, about 10 g/L, about 15 g/L, about 20 g/L, about 25 g/L, about 30 g/L, or about 40 g/L. In some embodiments, the process produces correctly processed periplasmic or extracellular protein at at most about 0.5 g/L, about 1 g/L, about 2 g/L, about 5 g/L, about 10 g/L, about 15 g/L, about 20 g/L, about 25 g/L, about 30 g/L, about 40 g/L, or about 50 g/L.

In some embodiments, the % of total recombinant protein or polypeptide that is produced in correctly processed form is about 5 to about 100. In some embodiments, the % of total recombinant protein or polypeptide that is produced in correctly processed form is about 5 to about 10, about 5 to about 20, about 5 to about 30, about 5 to about 40, about 5 to about 50, about 5 to about 60, about 5 to about 70, about 5 to about 80, about 5 to about 90, about 5 to about 95, about 5 to about 100, about 10 to about 20, about 10 to about 30, about 10 to about 40, about 10 to about 50, about 10 to about 60, about 10 to about 70, about 10 to about 80, about 10 to about 90, about 10 to about 95, about 10 to about 100, about 20 to about 30, about 20 to about 40, about 20 to about 50, about 20 to about 60, about 20 to about 70, about 20 to about 80, about 20 to about 90, about 20 to about 95, about 20 to about 100, about 30 to about 40, about 30 to about 50, about 30 to about 60, about 30 to about 70, about 30 to about 80, about 30 to about 90, about 30 to about 95, about 30 to about 100, about 40 to about 50, about 40 to about 60, about 40 to about 70, about 40 to about 80, about 40 to about 90, about 40 to about 95, about 40 to about 100, about 50 to about 60, about 50 to about 70, about 50 to about 80, about 50 to about 90, about 50 to about 95, about 50 to about 100, about 60 to about 70, about 60 to about 80, about 60 to about 90, about 60 to about 95, about 60 to about 100, about 70 to about 80, about 70 to about 90, about 70 to about 95, about 70 to about 100, about 80 to about 90, about 80 to about 95, about 80 to about 100, about 90 to about 95, about 90 to about 100, or about 95 to about 100. In some embodiments, the % of total recombinant protein or polypeptide that is produced in correctly processed form is about 5, about 10, about 20, about 30, about 40, about 50, about 60, about 70, about 80, about 90, about 95, or about 100. In some embodiments, the % of total recombinant protein or polypeptide that is produced in correctly processed form is at least about 5, about 10, about 20, about 30, about 40, about 50, about 60, about 70, about 80, about 90, or about 95. In some embodiments, the % of total recombinant protein or polypeptide that is produced in correctly processed form is at most about 10, about 20, about 30, about 40, about 50, about 60, about 70, about 80, about 90, about 95, or about 100.

The following patents and patent applications are incorporated herein by reference in their entirety, including for their disclosure on processing/cleavage and periplasmic expression of recombinant proteins and polypeptides, e.g., as fused to secretion signal peptides: U.S. Pat. No. 7,618,799, “Bacterial leader sequences for increased expression,” in U.S. Pat. No. 7,985,564, “Expression systems with Sec-system secretion,” in U.S. Pat. Nos. 9,394,571 and 9,580,719, both titled “Method for Rapidly Screening Microbial Hosts to Identify Certain Strains with Improved Yield and/or Quality in the Expression of Heterologous Proteins,” in U.S. Pat. No. 7,833,752, in U.S. Pat. No. 10,118,956, in U.S. Pat. No. 9,453,251, “Expression of Mammalian Proteins in Pseudomonas fluorescens,” in U.S. Pat. No. 8,603,824, “Process for Improved Protein Expression by Strain Engineering,” in U.S. Pat. No. 8,530,171, “High Level Expression of Recombinant Toxin Proteins,” and in U.S. Pat. Pub. No. 2019/0127744, “Bacterial Leader Sequences for Periplasmic Protein Expression.” In some embodiments, the secretion leader is selected from 8484 (SEQ ID NO: 24), AnsB (SEQ ID NO: 26), CupB2 (SEQ ID NO: 28), FlgI (SEQ ID NO: 30), Ibp-S31A (SEQ ID NO: 32), Lao (SEQ ID NO: 34), Leader M, PorE (SEQ ID NO: 38), TolB (SEQ ID NO: 40), CupC2 (SEQ ID NO: 70), Azu (SEQ ID NO: 72), Pbp (SEQ ID NO: 74), PbpA20V (SEQ ID NO: 76), 5193 (SEQ ID NO: 78), or Ibp (SEQ ID NO: 80). In certain embodiments, the secretion leader has at least 85% identity, at least 90% identity, or at least 95% identity, to an amino acid sequence selected from SEQ ID NOS: 24, 26, 28, 30, 32, 34, 38, 40, 70, 72, 74, 76, 78, and 80.

In some embodiments, the secretion signal sequence is identical to or substantially identical to a secretion signal peptide set forth in SEQ ID NO: 24, and/or is encoded by a polynucleotide sequence set forth in SEQ ID NO: 23. In some embodiments, the secretion signal sequence is identical to or substantially identical to a secretion signal peptide set forth in SEQ ID NO: 26, and/or is encoded by a polynucleotide sequence set forth in SEQ ID NO: 25. In some embodiments, the secretion signal sequence is identical to or substantially identical to a secretion signal peptide set forth in SEQ ID NO: 28, and/or is encoded by a polynucleotide sequence set forth in SEQ ID NO: 27. In some embodiments, the secretion signal sequence is identical to or substantially identical to a secretion signal peptide set forth in SEQ ID NO: 30, and/or is encoded by a polynucleotide sequence set forth in SEQ ID NO: 29. In some embodiments, the secretion signal sequence is identical to or substantially identical to a secretion signal peptide set forth in SEQ ID NO: 32, and/or is encoded by a polynucleotide sequence set forth in SEQ ID NO: 31. In some embodiments, the secretion signal sequence is identical to or substantially identical to a secretion signal peptide set forth in SEQ ID NO: 34, and/or is encoded by a polynucleotide sequence set forth in SEQ ID NO: 33. In some embodiments, the secretion signal sequence is identical to or substantially identical to a secretion signal peptide set forth in SEQ ID NO: 38, and/or is encoded by a polynucleotide sequence set forth in SEQ ID NO: 37. In some embodiments, the secretion signal sequence is identical to or substantially identical to a secretion signal peptide set forth in SEQ ID NO: 40, and/or is encoded by a polynucleotide sequence set forth in SEQ ID NO: 39. In another embodiment, the secretion signal sequence comprises at least amino acids 2-29 of SEQ ID NOS: 24, 26, 28, 30, 32, 34, 38, or 40. In yet another embodiment, the secretion signal sequence comprises a fragment of SEQ ID NOS: 24, 26, 28, 30, 32, 34, 38, or 40, which is truncated by 1, 2, 3, 4, 5, 6, 7, 8, 9, or 10 amino acids from the amino terminus but retains biological activity, i.e., secretion signal activity.

In one embodiment the amino acid sequence of the peptide is a variant of a given original peptide, wherein the sequence of the variant is obtainable by replacing up to or about 30% of the original peptide's amino acid residues with other amino acid residue(s), including up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%, provided that the variant retains the desired function of the original peptide. A variant amino acid with substantial homology will be at least about 70%, at least about 75%, at least about 80%, about 85%, about 90%, about 95%, about 96%, about 97%, about 98%, or at least about 99% homologous to the original peptide. A variant amino acid sequence may be obtained in various ways including amino acid substitutions, deletions, truncations, and insertions of one or more amino acids of SEQ ID NOS: 24, 26, 28, 30, 32, 34, 38, or 40. In some embodiments, a variant amino acid sequence comprises 1-9 amino acid substitutions, deletions, insertions, or any combination thereof. In some embodiments, the number of amino acid substitutions, deletions, insertions, or any combination thereof, in a variant of SEQ ID NOS: 24, 26, 28, 30, 32, 34, 38, or 40, is 1 to 10. In some embodiments, the number of amino acid substitutions, deletions, insertions, or any combination thereof, in a variant of SEQ ID NOS: 24, 26, 28, 30, 32, 34, 38, or 40, is 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In some embodiments, the number of amino acid substitutions, deletions, insertions, or any combination thereof, in a variant of SEQ ID NOS: 24, 26, 28, 30, 32, 34, 38, or 40, is 1, 2, 3, 4, 5, 6, 7, 8, 9, or 10. In some embodiments, the number of amino acid substitutions, deletions, insertions, or any combination thereof, in a variant of SEQ ID NOS: 24, 26, 28, 30, 32, 34, 38, or 40, is at least 1, 2, 3, 4, 5, 6, 7, 8, or 9. In some embodiments, the number of amino acid substitutions, deletions, insertions, or any combination thereof, in a variant of SEQ ID NOS: 24, 26, 28, 30, 32, 34, 38, or 40, is at most 2, 3, 4, 5, 6, 7, 8, 9, or 10.

By “substantially homologous,” “substantially identical,” or “substantially similar” is intended an amino acid or nucleotide sequence that has about or at least about 60%, about or at least about 65%, about or at least about 70%, about or at least about 75%, about or at least about 80%, about or at least about 85%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98% or about or at least about 99%, or greater sequence identity as compared to a reference sequence using a suitable alignment program described herein or known in the art using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

In some embodiments, a secretion signal peptide used in the present invention may include one or more modifications of a “non-essential” amino acid residue. In this context, a “non-essential” amino acid residue is a residue that can be altered, e.g., deleted, substituted, or derivatized, in the novel amino acid sequence without abolishing or substantially reducing the activity (e.g., the agonist activity) of the original secretion signal peptide (also referred to as the “analog” or “reference” peptide). In some embodiments, a secretion signal peptide may include one or more modifications of an “essential” amino acid residue. In this context, an “essential” amino acid residue is a residue that when altered, e.g., deleted, substituted, or derivatized, in the novel amino acid sequence the activity of the reference peptide is substantially reduced or abolished. In such embodiments where an essential amino acid residue is altered, the modified secretion signal peptide may possess an activity of the original secretion signal. The substitutions, insertions and deletions may be at the N-terminal or C-terminal end, or may be at internal portions of the secretion signal. By way of example, the secretion signal peptide may include 1, 2, 3, 4, 5, 6, 7, 8, 9, 10, or more substitutions, both in a consecutive manner or spaced throughout the secretion signal peptide. Alone or in combination with the substitutions, the secretion signal peptide may include 1, 2, 3, 4, 5, 6, 7, 8, 9, 10, or more insertions, again either in consecutive manner or spaced throughout the secretion signal peptide. The secretion signal peptide, alone or in combination with the substitutions and/or insertions, may also include 1, 2, 3, 4, 5, 6, 7, 8, 9, 10, or more deletions, again either in consecutive manner or spaced throughout the peptide. The secretion signal peptide, alone or in combination with the substitutions, insertions and/or deletions, may also include 1, 2, 3, 4, 5, 6, 7, 8, 9, 10, or more amino acid additions.

Substitutions include conservative amino acid substitutions. A “conservative amino acid substitution” is one in which the amino acid residue is replaced with an amino acid residue having a similar side chain, or physicochemical characteristics (e.g., electrostatic, hydrogen bonding, isosteric, hydrophobic features). The amino acids may be naturally occurring or unnatural. Families of amino acid residues having similar side chains are known in the art. These families include amino acids with basic side chains (e.g. lysine, arginine, histidine), acidic side chains (e.g., aspartic acid, glutamic acid), uncharged polar side chains (e.g., glycine, asparagine, glutamine, serine, threonine, tyrosine, methionine, cysteine), nonpolar side chains (e.g., alanine, valine, leucine, isoleucine, proline, phenylalanine, tryptophan), β-branched side chains (e.g., threonine, valine, isoleucine) and aromatic side chains (e.g., tyrosine, phenylalanine, tryptophan, histidine). Substitutions may also include non-conservative changes.

Variant proteins or polypeptide of interest encompassed herein are biologically active, that is they continue to possess the desired biological activity of the original protein or polypeptide of interest; for example, a variant secretion leader peptide retains secretion signal activity. By “retains activity” is intended that the variant will have about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98% or about or at least about 99%, about or at least about 100%, about or at least about 110%, about or at least about 125%, about or at least about 150%, about or at least about 200% or greater activity, e.g., secretion signal activity, of the original peptide, protein, or polypeptide.

### Codon Optimization

The present invention contemplates the use of any appropriate coding sequence for the recombinant protein of interest, including any sequence that has been optimized for expression in the host cell being used. A nucleic acid sequence encoding the recombinant protein of interest may be codon-optimized to improve expression in the recombinant gram-negative bacterial host cell, as understood by one of skill in the art. For example, optimization of codons for expression in a Pseudomonas host strain is described, e.g., in U.S. Pat. App. Pub. No. 2007/0292918, “Codon Optimization Method,” incorporated herein by reference in its entirety. Codon optimization for expression in E. coli is described, e.g., by Welch, et al., 2009, PLOS One, “Design Parameters to Control Synthetic Gene Expression in Escherichia coli, 4 (9): e7002, incorporated by reference herein. It is understood that any suitable sequence encoding a recombinant protein of interest can be generated as desired according to methods well known by those of skill in the art.

### Expression Constructs

An appropriate expression construct for producing a recombinant protein of interest according to the methods of the invention may be selected by one of skill in the art in view of the present disclosure.

In some embodiments, a recombinant protein of interest produced in a recombinant gram-negative host cell of the present invention is encoded by an expression vector comprising at least one expression construct encoding the recombinant protein of interest, wherein the expression construct comprises at least one nucleic acid sequence encoding the recombinant protein of interest. In certain embodiments, the expression construct comprises a nucleic acid encoding the recombinant ultralong CDR3 knob peptide. The nucleic acid can encode a sequences of recombinant ultralong CDR3 knob peptides, and sequences of the nucleic acid encoding the sequences of recombinant ultralong CDR3 knob peptides, are described herein and/or may readily be obtained by those of skill in the art. In some embodiments, the sequence of the nucleic acid encoding the recombinant ultralong CDR3 knob peptide has at least 85% identity to a nucleic acid sequence selected from SEQ ID NOS: 11 or 17, and encodes an amino acid sequence selected from SEQ ID NOS: 12, or 18, respectively. In certain embodiments, the nucleic acid does not include a fusion construct, wherein the fusion construct comprises a sequence encoding a fusion partner, e.g., a chaperone protein, linked to or fused with the recombinant ultralong CDR3 knob peptide.

In some embodiments, at least two nucleic acid sequences encoding the recombinant protein of interest are transcribed from the same promoter (co-transcribed). In some embodiments at least two nucleic acid sequences encoding the recombinant protein of interest are transcribed from different promoters (not co-transcribed). When not co-transcribed, each of the least two nucleic acid sequences encoding the at least two nucleic acid sequences encoding the recombinant protein of interest may be produced from the same expression vector or separate expression vectors. In some embodiments, a nucleic acid sequence encoding a recombinant protein of interest is operably linked to a nucleic acid sequence encoding a secretion signal. In some embodiments, each of at least two nucleic acid sequences encoding a recombinant protein of interest is individually operably linked to a nucleic acid sequence encoding the same or different secretion signal. In some embodiments, each nucleic acid sequence encoding a recombinant protein of interest in a host cell is individually operably linked to a nucleic acid sequence independently selected from the periplasmic secretion signals having the amino acid sequence set forth as: SEQ ID NOS: 24, 26, 28, 30, 32, 34, 38, or 40.

In some embodiments, a recombinant Pseudomonad host cell of the present invention is transformed with expression vector(s) comprising the at least one expression construct encoding the recombinant protein of interest. In some embodiments, the transformed recombinant gram-negative bacterial host cell is further: optionally deficient in one or more protease activity, optionally deficient in at least one autolytic factor activity, optionally overexpresses one or more inactivated protease, and optionally overexpresses one or more chaperone or folding modulator, each as described elsewhere herein in detail. In some embodiments, the transformed Pseudomonad host cell is a Pseudomonas host cell. In some embodiments, the Pseudomonas host cell is P. fluorescens, P. putida, or P. aeruginosa.

In some embodiments, the recombinant Pseudomonad host cell transformed with an expression vector(s) comprising the at least one expression construct encoding the recombinant CDR3 knob peptide is: (i) lsc::lacI^{Q1}; (ii) Prc1 deficient; (ii) Prc2 deficient; (iii) HslU deficient; (iv) HslV deficient; (v) MepM1 deficient; and (vi) PyrF deficient; wherein the host cell is optionally deficient in a serralysin precursor that is: a serralysin precursor having the amino acid sequence set forth as SEQ ID NO: 16; a homologue of the serralysin precursor having the amino acid sequence set forth as SEQ ID NO: 16; or a serralysin precursor related protein having at least 60% similarity or at least 60% identity to the amino acid sequence set forth as SEQ ID NO: 16.

In some embodiments, the recombinant Pseudomonad host cell transformed with an expression vector(s) comprising the at least one expression construct encoding the recombinant CDR3 knob peptide is: DegP2 deficient; and overexpress SecB, DsbA, DsbC, Skp, FklB2 or any combination thereof. In some embodiments, the recombinant gram-negative bacterial host cell transformed with expression vector(s) comprising the at least one expression construct encoding the recombinant protein of interest is: DegP2 deficient; and overexpresses: i) SecB; ii) DsbA, DsbC and Skp; iii) DsbA and DsbC; iv) DsbC and FklB2.

In some embodiments, the Pseudomonad bacterial host cell transformed with expression vector(s) comprising the at least one expression construct encoding the CDR3 knob peptide, has a phenotype and genotype as set forth for any host strain in any one of Tables 3, 4, 6, and 9. In some embodiments, the recombinant gram-negative bacterial host cell transformed with expression vector(s) comprising the at least one expression construct encoding the recombinant protein of interest, has a phenotype, genotype, and expression construct sequence elements as set forth for any bacterial strain in any one of Tables 3, 4, 6, and 9. In some embodiments, the recombinant gram-negative bacterial host cell transformed with expression vector(s) comprising the at least one expression construct encoding the recombinant protein of interest, is a bacterial strain as set forth in any one of Tables 3, 4, 6, and 9.

In some embodiments, a recombinant protein or polypeptide of interest is produced in a recombinant gram-negative bacterial host cell that is any one of strains STR92557, STR87639, STR92567, STR94974, STR94975, STR94976, and STR94977. In some embodiments, the recombinant protein or polypeptide of interest is produced in a recombinant gram-negative bacterial host cell that has the genotype (genomic modifications) of, and/or has the protease deficiency, inactivated protease, and folding modulator overexpression profile of, any one of strains STR92557, STR87639, STR92567, STR94974, STR94975, STR94976, and STR94977. In some embodiments, the recombinant protein or polypeptide of interest is produced in a recombinant gram-negative bacterial host cell that has the genotype of, and/or has the protease deficiency, inactivated protease, and folding modulator overexpression profile of, STR94975, STR94976, or STR94977.

In some embodiments, the Pseudomonad host cell is a Pseudomonas host cell. In some embodiments, the Pseudomonas host cell is P. fluorescens, P. putida, or P. aeruginosa. In some embodiments, the transformed recombinant gram-negative bacterial host cell is not an E. coli host cell.

In some embodiments, the recombinant gram-negative bacterial host cell transformed with expression vector(s) comprising the at least one expression construct encoding the antibody is: (i) lsc: lacI^{Q1}; (ii) Prc1 deficient; (ii) Prc2 deficient; (iii) HslU deficient; (iv) HslV deficient; (v) MepM1 deficient; (vi) PyrF deficient; wherein the host cell is optionally deficient in a serralysin precursor that is: a serralysin precursor having the amino acid sequence set forth as SEQ ID NO: 16; a homologue of the serralysin precursor having the amino acid sequence set forth as SEQ ID NO: 16; or a serralysin precursor related protein having at least 60% similarity or at least 60% identity to the amino acid sequence set forth as SEQ ID NO: 16.

In some embodiments, an antibody is produced in a recombinant gram-negative bacterial host cell that is any one of strains STR92557, STR87639, STR92567, STR94974, STR94975, STR94976, and STR94977. In some embodiments, the antibody is produced in a recombinant gram-negative bacterial host cell that has the genotype of, and/or has the protease deficiency, inactivated protease, and folding modulator overexpression profile of, any one of strains STR92557, STR87639, STR92567, STR94974, STR94975, STR94976, and STR94977. In some embodiments, the antibody is produced in a recombinant gram-negative bacterial host cell that has the genotype of, and/or has the protease deficiency, inactivated protease, and folding modulator overexpression profile of, STR94975, STR94976, and STR94977.

In some embodiments, the recombinant protein of interest is produced in a recombinant gram-negative bacterial host cell that has the following genotype: Δprc1, Δprc2, Δhs1U, Δhs1V, ΔmepM1, ΔRXF04495.2, ΔpyrF, and Isc::lacI^{Q1}. In some embodiments, the recombinant protein of interest is produced in a recombinant gram-negative bacterial host cell, that is mtlDYZ knock-out mutant ΔpyrF ΔproC ΔbenAB Isc::lacI^{Q1}, a derivative of deposited strain in which the genes pyrF, proC, benA, benB, and mtIDYZ from the mannitol (mtl) operon are deleted, and the E. coli lacI transcriptional repressor is inserted and fused with the levansucrase gene (lsc). In some embodiments, the recombinant protein of interest is produced in a recombinant a P. fluorescens host strain, that is mtlDYZ knock-out mutant ΔpyrF ΔproC ΔbenAB lsc: lacI^{Q1}, a derivative of deposited strain in which the genes pyrF, proC, benA, benB, and mtIDYZ from the mannitol (mtl) operon are deleted, and the E. coli lacI transcriptional repressor is inserted and fused with the levansucrase gene (lsc).

In some embodiments, the recombinant gram-negative bacterial host cell is any one of strains STR92557, STR87639, STR92567, STR94974, STR94975, STR94976, and STR94977. In some embodiments, the recombinant gram-negative bacterial host cell that the genotype (genomic modifications) of, and/or has the protease deficiency, inactivated protease, and folding modulator overexpression profile of, any one of strains STR92557, STR87639, STR92567, STR94974, STR94975, STR94976, and STR94977. In some embodiments, the recombinant gram-negative bacterial host cell that has the genotype of, and/or has the protease deficiency, inactivated protease, and folding modulator overexpression profile of, STR94975, STR94976, or STR94977.

### Fermentation Format

A recombinant protein of interest may be produced using the methods as described herein, by culturing the recombinant gram-negative bacterial host cells transformed with a plasmid encoding the recombinant protein of interest (an expression strain) under suitable fermentation conditions. Any fermentation format, e.g., a batch, fed-batch, semi-continuous, or continuous fermentation mode, may be employed.

The fermentation medium may be selected from rich media, minimal media, and mineral salts media. In some embodiments, a minimal medium or a mineral salts medium is selected. In some embodiments, a mineral salts medium is selected.

Mineral salts media consists of mineral salts and a carbon source such as, e.g., glucose, sucrose, or glycerol. Examples of mineral salts media include, e.g., M9 medium, Pseudomonas medium (ATCC 179), and Davis and Mingioli medium (see, Davis, B. D., and Mingioli, E. S., 1950, J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. Typically, no organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. A mineral salts medium will typically contain glucose or glycerol as the carbon source. In comparison to mineral salts media, minimal media can also contain mineral salts and a carbon source, but can be supplemented with, e.g., low levels of amino acids, vitamins, peptones, or other ingredients, though these are added at very minimal levels. Suitable media for use in the methods of the present invention can be prepared using methods described in the literature, e.g., in U.S. Pat. Nos. 9,458,487 and 9,453,251. Details of cultivation procedures and mineral salts media useful in the methods of the present invention are described by Riesenberg, D et al., 1991, “High cell density cultivation of Escherichia coli at controlled specific growth rate,” J. Biotechnol. 20 (1): 17-27, incorporated by reference herein.

In some embodiments, production can be achieved in bioreactor cultures. Cultures can be grown in, e.g., up to 2 L bioreactors containing a mineral salts medium, and maintained at 32° C. and pH 6.5 through the addition of ammonia. Dissolved oxygen can be maintained in excess through increases in agitation and flow of sparged air and oxygen into the fermentor. Glycerol can be delivered to the culture throughout the fermentation to maintain excess levels. In some embodiments, these conditions are maintained until a target culture cell density, e.g., an optical density of 575 nm (A575), for induction is reached and IPTG is added to initiate the target protein production. It is understood that the cell density at induction, the concentration of IPTG, pH, temperature, CaCl₂) concentration, dissolved oxygen flow rate, each can be varied to determine optimal conditions for expression. In some embodiments, cell density at induction can be varied from A575 of 40 to 200 absorbance units (AU). IPTG concentrations can be varied in the range from 0.02 to 1.0 mM, pH from 5 to 7.5, temperature from 20 to 35° C., CaCl₂) concentration from 0 to 0.5 g/L, and the dissolved oxygen flow rate from 1 LPM (liters per minute) to 10 LPM. After 6-96 hours, the culture from each bioreactor can be harvested by centrifugation and the cell pellet frozen at −80° C. Samples can then be analyzed, e.g., by SDS-CGE, for product formation.

Fermentation may be performed at any scale. The expression systems according to the present invention are useful for recombinant protein expression at any scale. Thus, e.g., microliter-scale, milliliter scale, centiliter scale, and deciliter scale fermentation volumes may be used, and 1 Liter scale and larger fermentation volumes can be used.

In some embodiments, the fermentation volume is at or above about 1 Liter. In some embodiments, the fermentation volume is about 1 Liter to about 100 Liters. In some embodiments, the fermentation volume is about 1 Liter, about 2 Liters, about 3 Liters about 4 Liters, about 5 Liters, about 6 Liters, about 7 Liters, about 8 Liters, about 9 Liters, or about 10 Liters. In some embodiments, the fermentation volume is about 1 Liter to about 5 Liters, about 1 Liter to about 10 Liters, about 1 Liter to about 25 Liters, about 1 Liter to about 50 Liters, about 1 Liter to about 75 Liters, about 10 Liters to about 25 Liters, about 25 Liters to about 50 Liters, or about 50 Liters to about 100 Liters. In other embodiments, the fermentation volume is at or above 5 Liters, 10 Liters, 15 Liters, 20 Liters, 25 Liters, 50 Liters, 75 Liters, 100 Liters, 200 Liters, 250 Liters, 300 Liters, 500 Liters, 1,000 Liters, 2,000 Liters, 5,000 Liters, 10,000 Liters, or 50,000 Liters.

In general, the amount of a recombinant protein yielded by a larger culture volume, e.g., a 50 mL shake-flask culture, a 1 liter culture, or greater, is increased relative to that observed in a smaller culture volume, e.g, a 0.5 mL high-throughput screening culture. This can be due to not only the increase in culture size but, e.g., the ability to grow cells to a higher density in large-scale fermentation (e.g., as reflected by culture absorbance). For example, the volumetric yield from the same strain can increase up to ten-fold from HTP scale to large-scale fermentation. In some embodiments, the volumetric yield observed for the same expression strain is 2-fold to 10-fold greater following large-scale fermentation than HTP scale growth. In some embodiments, the yield observed for the same expression strain is 2-fold, 3-fold, 4-fold, 5-fold, 6-fold, 7-fold, 8-fold, 9-fold, 2-fold to 10-fold, 2-fold to 9-fold, 2-fold to 8-fold, 2-fold to 7-fold, 2-fold to 6-fold, 2-fold to 5-fold, 2-fold to 4-fold, 2-fold to 3-fold, 3-fold to 10-fold, 3-fold to 9-fold, 3-fold to 8-fold, 3-fold to 7-fold, 3-fold to 6-fold, 3-fold to 5-fold, 3-fold to 4-fold, 4-fold to 10-fold, 4-fold to 9-fold, 4-fold to 8-fold, 4-fold to 7-fold, 4-fold to 6-fold, 4-fold to 5-fold, 5-fold to 10-fold, 5-fold to 9-fold, 5-fold to 8-fold, 5-fold to 7-fold, 5-fold to 6-fold, 6-fold to 10-fold, 6-fold to 9-fold, 6-fold to 8-fold, 6-fold to 7-fold, 7-fold to 10-fold, 7-fold to 9-fold, 7-fold to 8-fold, 8-fold to 10-fold, 8-fold to 9-fold, 9-fold to 10-fold, greater following large-scale fermentation than following HTP-scale growth. See, e.g., Retallack, et al., 2012, “Reliable protein production in a Pseudomonas fluorescens expression system,” Prot. Exp. and Purif. 81:157-165, incorporated herein by reference in its entirety.

### Bacterial Growth Conditions

Suitable fermentation conditions useful in the methods of the provided invention can comprise growth at a temperature of about 4 deg C. to about 42 deg C. and a pH of about 5.7 to about 8.8. When an expression construct with a lacZ promoter is used, expression can be induced by adding IPTG to a culture at a final concentration of about 0.01 mM to about 1.0 mM. In some embodiments, the fermentation conditions comprise induction of the inducible promoter at: an OD575 of about 40 to about 200, a culture pH of about 5.5 to about 7.2, and a temperature of about 20 to about 34 deg C., fed batch. In some embodiments, the fermentation conditions comprise induction of the inducible promoter at: an OD575 of about 80 to about 160, a culture pH of about 5.8 to about 7.0, a temperature of about 28 to about 33 deg C., fed batch. In some embodiments, the resulting recombinant protein titer is about 0.2 to about 5 g/L of cell culture.

The pH of the culture can be maintained using pH buffers and methods known to those of skill in the art. Control of pH during culturing also can be achieved using aqueous ammonia. In some embodiments, the pH of the culture during growth, induction, and/or production phase is about 5 to about 8.8. In some embodiments, the culture pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, or any range therein. In some embodiments, the culture pH is about 5 to about 8.8. In some embodiments, the culture pH is about 5 to about 5.5, about 5 to about 6, about 5 to about 6.5, about 5 to about 7, about 5 to about 7.5, about 5 to about 8, about 5 to about 8.5, about 5 to about 8.8, about 5.5 to about 6, about 5.5 to about 6.5, about 5.5 to about 7, about 5.5 to about 7.5, about 5.5 to about 8, about 5.5 to about 8.5, about 5.5 to about 8.8, about 6 to about 6.5, about 6 to about 7, about 6 to about 7.5, about 6 to about 8, about 6 to about 8.5, about 6 to about 8.8, about 6.5 to about 7, about 6.5 to about 7.5, about 6.5 to about 8, about 6.5 to about 8.5, about 6.5 to about 8.8, about 7 to about 7.5, about 7 to about 8, about 7 to about 8.5, about 7 to about 8.8, about 7.5 to about 8, about 7.5 to about 8.5, about 7.5 to about 8.8, about 8 to about 8.5, about 8 to about 8.8, or about 8.5 to about 8.8. In some embodiments, the culture pH is about 5, about 5.5, about 6, about 6.5, about 7, about 7.5, about 8, about 8.5, or about 8.8. In some embodiments, the culture pH is at least about 5, about 5.5, about 6, about 6.5, about 7, about 7.5, about 8, or about 8.5. In some embodiments, the culture pH is at most about 5.5, about 6, about 6.5, about 7, about 7.5, about 8, about 8.5, or about 8.8. In some embodiments, the culture pH is about 5.8 to about 7. In some embodiments, the culture pH is about 5.8 to about 5.9, about 5.8 to about 6, about 5.8 to about 6.1, about 5.8 to about 6.2, about 5.8 to about 6.2, about 5.8 to about 6.4, about 5.8 to about 6.5, about 5.8 to about 6.6, about 5.8 to about 6.7, about 5.8 to about 6.8, about 5.8 to about 7, about 5.9 to about 6, about 5.9 to about 6.1, about 5.9 to about 6.2, about 5.9 to about 6.2, about 5.9 to about 6.4, about 5.9 to about 6.5, about 5.9 to about 6.6, about 5.9 to about 6.7, about 5.9 to about 6.8, about 5.9 to about 7, about 6 to about 6.1, about 6 to about 6.2, about 6 to about 6.2, about 6 to about 6.4, about 6 to about 6.5, about 6 to about 6.6, about 6 to about 6.7, about 6 to about 6.8, about 6 to about 7, about 6.1 to about 6.2, about 6.1 to about 6.2, about 6.1 to about 6.4, about 6.1 to about 6.5, about 6.1 to about 6.6, about 6.1 to about 6.7, about 6.1 to about 6.8, about 6.1 to about 7, about 6.2 to about 6.2, about 6.2 to about 6.4, about 6.2 to about 6.5, about 6.2 to about 6.6, about 6.2 to about 6.7, about 6.2 to about 6.8, about 6.2 to about 7, about 6.2 to about 6.4, about 6.2 to about 6.5, about 6.2 to about 6.6, about 6.2 to about 6.7, about 6.2 to about 6.8, about 6.2 to about 7, about 6.4 to about 6.5, about 6.4 to about 6.6, about 6.4 to about 6.7, about 6.4 to about 6.8, about 6.4 to about 7, about 6.5 to about 6.6, about 6.5 to about 6.7, about 6.5 to about 6.8, about 6.5 to about 7, about 6.6 to about 6.7, about 6.6 to about 6.8, about 6.6 to about 7, about 6.7 to about 6.8, about 6.7 to about 7, or about 6.8 to about 7. In some embodiments, the culture pH is about 5.8, about 5.9, about 6, about 6.1, about 6.2, about 6.2, about 6.4, about 6.5, about 6.6, about 6.7, about 6.8, or about 7. In some embodiments, the culture pH is at least about 5.8, about 5.9, about 6, about 6.1, about 6.2, about 6.2, about 6.4, about 6.5, about 6.6, about 6.7, or about 6.8. In some embodiments, the culture pH is at most about 5.9, about 6, about 6.1, about 6.2, about 6.2, about 6.4, about 6.5, about 6.6, about 6.7, about 6.8, or about 7. In some embodiments, the pH is about 6 to about 6.5. In some embodiments, the culture pH is about 6 to about 6.1, about 6 to about 6.2, about 6 to about 6.3, about 6 to about 6.4, about 6 to about 6.5, about 6.1 to about 6.2, about 6.1 to about 6.3, about 6.1 to about 6.4, about 6.1 to about 6.5, about 6.2 to about 6.3, about 6.2 to about 6.4, about 6.2 to about 6.5, about 6.3 to about 6.4, about 6.3 to about 6.5, or about 6.4 to about 6.5. In some embodiments, the culture pH is about 6, about 6.1, about 6.2, about 6.3, about 6.4, or about 6.5. In some embodiments, the culture pH is at least about 6, about 6.1, about 6.2, about 6.3, or about 6.4. In some embodiments, the culture pH is at most about 6.1, about 6.2, about 6.3, about 6.4, or about 6.5.

In some embodiments, the growth temperature of the culture during growth, induction, and/or production phase is maintained at about 4° C. to about 42° C. In some embodiments, the growth temperature is about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., or any range therein. In some embodiments, the growth temperature is about 25° C. to about 35° C. In some embodiments, the growth temperature is about 25° C. to about 35° C. In some embodiments, the growth temperature is about 25° C. to about 26° C., about 25° C. to about 27° C., about 25° C. to about 28° C., about 25° C. to about 29° C., about 25° C. to about 30° C., about 25° C. to about 31° C., about 25° C. to about 32° C., about 25° C. to about 33° C., about 25° C. to about 34° C., about 25° C. to about 35° C., about 26° C. to about 27° C., about 26° C. to about 28° C., about 26° C. to about 29° C., about 26° C. to about 30° C., about 26° C. to about 31° C., about 26° C. to about 32° C., about 26° C. to about 33° C., about 26° C. to about 34° C., about 26° C. to about 35° C., about 27° C. to about 28° C., about 27° C. to about 29° C., about 27° C. to about 30° C., about 27° C. to about 31° C., about 27° C. to about 32° C., about 27° C. to about 33° C., about 27° C. to about 34° C., about 27° C. to about 35° C., about 28° C. to about 29° C., about 28° C. to about 30° C., about 28° C. to about 31° C., about 28° C. to about 32° C., about 28° C. to about 33° C., about 28° C. to about 34° C., about 28° C. to about 35° C., about 29° C. to about 30° C., about 29° C. to about 31° C., about 29° C. to about 32° C., about 29° C. to about 33° C., about 29° C. to about 34° C., about 29° C. to about 35° C., about 30° C. to about 31° C., about 30° C. to about 32° C., about 30° C. to about 33° C., about 30° C. to about 34° C., about 30° C. to about 35° C., about 31° C. to about 32° C., about 31° C. to about 33° C., about 31° C. to about 34° C., about 31° C. to about 35° C., about 32° C. to about 33° C., about 32° C. to about 34° C., about 32° C. to about 35° C., about 33° C. to about 34° C., about 33° C. to about 35° C., or about 34° C. to about 35° C. In some embodiments, the growth temperature is about 25° C., about 26° C., about 27° C., about 28° C., about 29° C., about 30° C., about 31° C., about 32° C., about 33° C., about 34° C., or about 35° C. In some embodiments, the growth temperature is at least about 25° C., about 26° C., about 27° C., about 28° C., about 29° C., about 30° C., about 31° C., about 32° C., about 33° C., or about 34° C. In some embodiments, the growth temperature is at most about 26° C., about 27° C., about 28° C., about 29° C., about 30° C., about 31° C., about 32° C., about 33° C., about 34° C., or about 35° C.

In some embodiments, the temperature is changed during culturing. In some embodiments, the temperature is maintained at about 30° C. to about 32° C. before an agent, e.g., IPTG, is added to the culture to induce expression from the construct, and after adding the induction agent, the temperature is reduced to about 25° C. to about 28° C. In some embodiments, the temperature is maintained at about 30° C. before an agent, e.g., IPTG, is added to the culture to induce expression from the construct, and after adding the induction agent, the temperature is reduced to about 25° C.

As described elsewhere herein, inducible promoters can be used in the expression construct to control expression of the recombinant protein of interest, e.g., a lac promoter. In the case of the lac promoter derivatives or family members, e.g., the tac promoter, the effector compound is an inducer, such as a gratuitous inducer like IPTG. In some embodiments, a lac promoter derivative is used, and recombinant protein expression is induced by the addition of IPTG to a final concentration of about 0.01 mM to about 1.0 mM, when the cell density has reached a level identified by an OD575 of about 80 to about 300. In some embodiments, the OD575 at the time of culture induction for the recombinant protein is about 80 to about 300. In some embodiments, the OD575 at the time of culture induction for the recombinant protein is about 80 to about 100, about 80 to about 120, about 80 to about 140, about 80 to about 160, about 80 to about 180, about 80 to about 200, about 80 to about 220, about 80 to about 240, about 80 to about 260, about 80 to about 280, about 80 to about 300, about 100 to about 120, about 100 to about 140, about 100 to about 160, about 100 to about 180, about 100 to about 200, about 100 to about 220, about 100 to about 240, about 100 to about 260, about 100 to about 280, about 100 to about 300, about 120 to about 140, about 120 to about 160, about 120 to about 180, about 120 to about 200, about 120 to about 220, about 120 to about 240, about 120 to about 260, about 120 to about 280, about 120 to about 300, about 140 to about 160, about 140 to about 180, about 140 to about 200, about 140 to about 220, about 140 to about 240, about 140 to about 260, about 140 to about 280, about 140 to about 300, about 160 to about 180, about 160 to about 200, about 160 to about 220, about 160 to about 240, about 160 to about 260, about 160 to about 280, about 160 to about 300, about 180 to about 200, about 180 to about 220, about 180 to about 240, about 180 to about 260, about 180 to about 280, about 180 to about 300, about 200 to about 220, about 200 to about 240, about 200 to about 260, about 200 to about 280, about 200 to about 300, about 220 to about 240, about 220 to about 260, about 220 to about 280, about 220 to about 300, about 240 to about 260, about 240 to about 280, about 240 to about 300, about 260 to about 280, about 260 to about 300, or about 280 to about 300. In some embodiments, the OD575 at the time of culture induction for the recombinant protein is about 80, about 100, about 120, about 140, about 160, about 180, about 200, about 220, about 240, about 260, about 280, or about 300. In some embodiments, the OD575 at the time of culture induction for the recombinant protein is at least about 80, about 100, about 120, about 140, about 160, about 180, about 200, about 220, about 240, about 260, or about 280. In some embodiments, the OD575 at the time of culture induction for the recombinant protein is at most about 100, about 120, about 140, about 160, about 180, about 200, about 220, about 240, about 260, about 280, or about 300. In some embodiments, the induction OD575 is about 80-160. In some embodiments, the OD575 at the time of culture induction for the recombinant protein is about 80 to about 160. In some embodiments, the OD575 at the time of culture induction for the recombinant protein is about 80 to about 90, about 80 to about 100, about 80 to about 110, about 80 to about 120, about 80 to about 130, about 80 to about 140, about 80 to about 150, about 80 to about 160, about 90 to about 100, about 90 to about 110, about 90 to about 120, about 90 to about 130, about 90 to about 140, about 90 to about 150, about 90 to about 160, about 100 to about 110, about 100 to about 120, about 100 to about 130, about 100 to about 140, about 100 to about 150, about 100 to about 160, about 110 to about 120, about 110 to about 130, about 110 to about 140, about 110 to about 150, about 110 to about 160, about 120 to about 130, about 120 to about 140, about 120 to about 150, about 120 to about 160, about 130 to about 140, about 130 to about 150, about 130 to about 160, about 140 to about 150, about 140 to about 160, or about 150 to about 160. In some embodiments, the OD575 at the time of culture induction for the recombinant protein is about 80, about 90, about 100, about 110, about 120, about 130, about 140, about 150, or about 160. In some embodiments, the OD575 at the time of culture induction for the recombinant protein is at least about 80, about 90, about 100, about 110, about 120, about 130, about 140, or about 150. In some embodiments, the OD575 at the time of culture induction for the recombinant protein is at most about 90, about 100, about 110, about 120, about 130, about 140, about 150, or about 160.

The cell density can be measured by other methods and expressed in other units, e.g., in cells per unit volume. For example, an OD575 of about 40 to about 160 of a P. fluorescens culture is equivalent to approximately 4×10¹⁰ to about 1.6×10¹¹ colony forming units per mL or 17.5 to 70 g/L dry cell weight. In some embodiments, the cell density at the time of culture induction is equivalent to the cell density as specified herein by the absorbance at OD575, regardless of the method used for determining cell density or the units of measurement. One of skill in the art will know how to make the appropriate conversion for any cell culture.

In some embodiments, the final IPTG concentration of the culture is about 0.01 mM to about 1 mM. In some embodiments, the final IPTG concentration of the culture is about 0.01 mM to about 0.02 mM, about 0.01 mM to about 0.03 mM, about 0.01 mM to about 0.05 mM, about 0.01 mM to about 0.06 mM, about 0.01 mM to about 0.07 mM, about 0.01 mM to about 0.08 mM, about 0.01 mM to about 0.09 mM, about 0.01 mM to about 0.1 mM, about 0.01 mM to about 0.2 mM, about 0.01 mM to about 0.5 mM, about 0.01 mM to about 1 mM, about 0.02 mM to about 0.03 mM, about 0.02 mM to about 0.05 mM, about 0.02 mM to about 0.06 mM, about 0.02 mM to about 0.07 mM, about 0.02 mM to about 0.08 mM, about 0.02 mM to about 0.09 mM, about 0.02 mM to about 0.1 mM, about 0.02 mM to about 0.2 mM, about 0.02 mM to about 0.5 mM, about 0.02 mM to about 1 mM, about 0.03 mM to about 0.05 mM, about 0.03 mM to about 0.06 mM, about 0.03 mM to about 0.07 mM, about 0.03 mM to about 0.08 mM, about 0.03 mM to about 0.09 mM, about 0.03 mM to about 0.1 mM, about 0.03 mM to about 0.2 mM, about 0.03 mM to about 0.5 mM, about 0.03 mM to about 1 mM, about 0.05 mM to about 0.06 mM, about 0.05 mM to about 0.07 mM, about 0.05 mM to about 0.08 mM, about 0.05 mM to about 0.09 mM, about 0.05 mM to about 0.1 mM, about 0.05 mM to about 0.2 mM, about 0.05 mM to about 0.5 mM, about 0.05 mM to about 1 mM, about 0.06 mM to about 0.07 mM, about 0.06 mM to about 0.08 mM, about 0.06 mM to about 0.09 mM, about 0.06 mM to about 0.1 mM, about 0.06 mM to about 0.2 mM, about 0.06 mM to about 0.5 mM, about 0.06 mM to about 1 mM, about 0.07 mM to about 0.08 mM, about 0.07 mM to about 0.09 mM, about 0.07 mM to about 0.1 mM, about 0.07 mM to about 0.2 mM, about 0.07 mM to about 0.5 mM, about 0.07 mM to about 1 mM, about 0.08 mM to about 0.09 mM, about 0.08 mM to about 0.1 mM, about 0.08 mM to about 0.2 mM, about 0.08 mM to about 0.5 mM, about 0.08 mM to about 1 mM, about 0.09 mM to about 0.1 mM, about 0.09 mM to about 0.2 mM, about 0.09 mM to about 0.5 mM, about 0.09 mM to about 1 mM, about 0.1 mM to about 0.2 mM, about 0.1 mM to about 0.5 mM, about 0.1 mM to about 1 mM, about 0.2 mM to about 0.5 mM, about 0.2 mM to about 1 mM, or about 0.5 mM to about 1 mM. In some embodiments, the final IPTG concentration of the culture is about 0.01 mM, about 0.02 mM, about 0.03 mM, about 0.05 mM, about 0.06 mM, about 0.07 mM, about 0.08 mM, about 0.09 mM, about 0.1 mM, about 0.2 mM, about 0.5 mM, or about 1 mM. In some embodiments, the final IPTG concentration of the culture is at least about 0.01 mM, about 0.02 mM, about 0.03 mM, about 0.05 mM, about 0.06 mM, about 0.07 mM, about 0.08 mM, about 0.09 mM, about 0.1 mM, about 0.2 mM, or about 0.5 mM. In some embodiments, the final IPTG concentration of the culture is at most about 0.02 mM, about 0.03 mM, about 0.05 mM, about 0.06 mM, about 0.07 mM, about 0.08 mM, about 0.09 mM, about 0.1 mM, about 0.2 mM, about 0.5 mM, or about 1 mM. In some embodiments, the final IPTG concentration of the culture is about 0.08 mM to about 0.3 mM. In some embodiments, the final IPTG concentration of the culture is about 0.08 mM to about 0.09 mM, about 0.08 mM to about 0.1 mM, about 0.08 mM to about 0.125 mM, about 0.08 mM to about 0.15 mM, about 0.08 mM to about 0.175 mM, about 0.08 mM to about 0.2 mM, about 0.08 mM to about 0.225 mM, about 0.08 mM to about 0.25 mM, about 0.08 mM to about 0.275 mM, about 0.08 mM to about 0.3 mM, about 0.09 mM to about 0.1 mM, about 0.09 mM to about 0.125 mM, about 0.09 mM to about 0.15 mM, about 0.09 mM to about 0.175 mM, about 0.09 mM to about 0.2 mM, about 0.09 mM to about 0.225 mM, about 0.09 mM to about 0.25 mM, about 0.09 mM to about 0.275 mM, about 0.09 mM to about 0.3 mM, about 0.1 mM to about 0.125 mM, about 0.1 mM to about 0.15 mM, about 0.1 mM to about 0.175 mM, about 0.1 mM to about 0.2 mM, about 0.1 mM to about 0.225 mM, about 0.1 mM to about 0.25 mM, about 0.1 mM to about 0.275 mM, about 0.1 mM to about 0.3 mM, about 0.125 mM to about 0.15 mM, about 0.125 mM to about 0.175 mM, about 0.125 mM to about 0.2 mM, about 0.125 mM to about 0.225 mM, about 0.125 mM to about 0.25 mM, about 0.125 mM to about 0.275 mM, about 0.125 mM to about 0.3 mM, about 0.15 mM to about 0.175 mM, about 0.15 mM to about 0.2 mM, about 0.15 mM to about 0.225 mM, about 0.15 mM to about 0.25 mM, about 0.15 mM to about 0.275 mM, about 0.15 mM to about 0.3 mM, about 0.175 mM to about 0.2 mM, about 0.175 mM to about 0.225 mM, about 0.175 mM to about 0.25 mM, about 0.175 mM to about 0.275 mM, about 0.175 mM to about 0.3 mM, about 0.2 mM to about 0.225 mM, about 0.2 mM to about 0.25 mM, about 0.2 mM to about 0.275 mM, about 0.2 mM to about 0.3 mM, about 0.225 mM to about 0.25 mM, about 0.225 mM to about 0.275 mM, about 0.225 mM to about 0.3 mM, about 0.25 mM to about 0.275 mM, about 0.25 mM to about 0.3 mM, or about 0.275 mM to about 0.3 mM. In some embodiments, the final IPTG concentration of the culture is about 0.08 mM, about 0.09 mM, about 0.1 mM, about 0.125 mM, about 0.15 mM, about 0.175 mM, about 0.2 mM, about 0.225 mM, about 0.25 mM, about 0.275 mM, or about 0.3 mM. In some embodiments, the final IPTG concentration of the culture is at least about 0.08 mM, about 0.09 mM, about 0.1 mM, about 0.125 mM, about 0.15 mM, about 0.175 mM, about 0.2 mM, about 0.225 mM, about 0.25 mM, or about 0.275 mM. In some embodiments, the final IPTG concentration of the culture is at most about 0.09 mM, about 0.1 mM, about 0.125 mM, about 0.15 mM, about 0.175 mM, about 0.2 mM, about 0.225 mM, about 0.25 mM, about 0.275 mM, or about 0.3 mM.

In some embodiments wherein a non-lac type promoter is used, as described herein and in the literature, other inducers or effectors can be used. In one embodiment, the promoter is a constitutive promoter.

After adding and inducing agent, cultures can be grown for a period of time, for example about 24 hours, during which time the recombinant protein is expressed (production phase). After adding an inducing agent, a culture can be grown for about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, about 36 hr, or about 48 hr. After an inducing agent is added to a culture, the culture can be grown for about 1 to 48 hr, about 1 to 24 hr, about 1 to 8 hr, about 10 to 24 hr, about 15 to 24 hr, or about 20 to 24 hr. Cell cultures can be concentrated by centrifugation, and the culture pellet resuspended in a buffer or solution appropriate for the subsequent lysis procedure.

In some embodiments a constant feed is used. In some embodiments, a fed-batch format is used. In some embodiments, the feed is glycerol or glucose. In some embodiments the feed bolus is about 10 g/L to about 50 g/L. In some embodiments the feed bolus is about 10 g/L to about 15 g/L, about 10 g/L to about 20 g/L, about 10 g/L to about 25 g/L, about 10 g/L to about 30 g/L, about 10 g/L to about 35 g/L, about 10 g/L to about 40 g/L, about 10 g/L to about 45 g/L, about 10 g/L to about 50 g/L, about 15 g/L to about 20 g/L, about 15 g/L to about 25 g/L, about 15 g/L to about 30 g/L, about 15 g/L to about 35 g/L, about 15 g/L to about 40 g/L, about 15 g/L to about 45 g/L, about 15 g/L to about 50 g/L, about 20 g/L to about 25 g/L, about 20 g/L to about 30 g/L, about 20 g/L to about 35 g/L, about 20 g/L to about 40 g/L, about 20 g/L to about 45 g/L, about 20 g/L to about 50 g/L, about 25 g/L to about 30 g/L, about 25 g/L to about 35 g/L, about 25 g/L to about 40 g/L, about 25 g/L to about 45 g/L, about 25 g/L to about 50 g/L, about 30 g/L to about 35 g/L, about 30 g/L to about 40 g/L, about 30 g/L to about 45 g/L, about 30 g/L to about 50 g/L, about 35 g/L to about 40 g/L, about 35 g/L to about 45 g/L, about 35 g/L to about 50 g/L, about 40 g/L to about 45 g/L, about 40 g/L to about 50 g/L, or about 45 g/L to about 50 g/L. In some embodiments the feed bolus is about 10 g/L, about 15 g/L, about 20 g/L, about 25 g/L, about 30 g/L, about 35 g/L, about 40 g/L, about 45 g/L, or about 50 g/L. In some embodiments the feed bolus is at least about 10 g/L, about 15 g/L, about 20 g/L, about 25 g/L, about 30 g/L, about 35 g/L, about 40 g/L, or about 45 g/L. In some embodiments the feed bolus is at most about 15 g/L, about 20 g/L, about 25 g/L, about 30 g/L, about 35 g/L, about 40 g/L, about 45 g/L, or about 50 g/L.

In some embodiments, cells are disrupted using equipment for high pressure mechanical cell disruption (which are available commercially, e.g., Microfluidics Micro fluidizer, Constant Cell Disruptor, Niro-Soavi homogenizer or APV-Gaulin homogenizer). Cells expressing the recombinant protein can be disrupted, for example, using sonication. Any appropriate method known in the art for lysing cells can be used to release the soluble fraction. For example, In some embodiments, chemical and/or enzymatic cell lysis reagents, such as cell-wall lytic enzyme and EDTA, can be used. Use of frozen or previously stored cultures is also contemplated in the methods of the invention. Cultures can be OD-normalized prior to lysis. For example, cells can be normalized to an OD600 of about 10, about 11, about 12, about 13, about 14, about 15, about 16, about 17, about 18, about 19, or about 20.

Centrifugation can be performed using any appropriate equipment and method. Centrifugation of cell culture or lysate for the purposes of separating a soluble fraction from an insoluble fraction is well-known in the art. For example, lysed cells can be centrifuged at 20,800×g for 20 minutes (at 4° C.), and the supernatants removed using manual or automated liquid handling. The cell pellet obtained by centrifugation of cell culture, or the insoluble fraction obtained by centrifugation of cell lysate, can be resuspended in a buffered solution. Resuspension of the cell pellet or insoluble fraction can be carried out using, e.g., equipment such as impellers connected to an overhead mixer, magnetic stir-bars, rocking shakers, etc.

A “soluble fraction,” i.e., the soluble supernatant obtained after centrifugation of a lysate, and an “insoluble fraction,” i.e., the pellet obtained after centrifugation of a lysate, result from lysing and centrifuging the cultures.

### High Throughput Screens

In some embodiments, a high throughput screen is conducted to determine optimal conditions for expressing a recombinant protein of interest. Conditions that can be varied in the screen include, for example, the host cell, genetic background of the host cell (e.g., as described in detail herein), type of promoter in an expression construct, type of secretion leader fused to the encoded polypeptide or protein of interest, temperature of growth, OD of induction when an inducible promoter is used, amount of inducer added (e.g. amount of IPTG used for induction when a lacZ promoter or derivative thereof is used), duration of protein induction, temperature of growth following addition of an inducing agent to a culture, rate of agitation of culture, method of selection for plasmid maintenance, volume of culture in a vessel, and method of cell lysing.

In some embodiments, a library (or “array”) of host strains is provided, wherein each strain (or “population of host cells”) in the library has been genetically modified to modulate the expression of one or more target genes in the host cell. An “optimal host strain” or “optimal expression system” may be identified or selected based on the quantity, quality, and/or location of the expressed protein of interest compared to other populations of phenotypically distinct host cells in the array. Thus, an optimal host strain is the strain that produces the recombinant protein of interest according to a desired specification. While the desired specification will vary depending on the polypeptide being produced, the specification includes the quality and/or quantity of protein, whether the protein is sequestered or secreted, protein folding, and the like. For example, the optimal host strain or optimal expression system produces a yield, characterized by the amount of soluble recombinant protein, the amount of recoverable recombinant protein, the amount of properly processed recombinant protein, the amount of properly folded recombinant protein, the amount of active recombinant protein, and/or the total amount of the recombinant protein of interest, of a certain absolute level or a certain level relative to that produced by a control or indicator strain, i.e., a strain used for comparison.

In certain embodiments, the expression plasmids were transformed into the P. fluorescens host strains in an array format. The transformation reaction was initiated by mixing P. fluorescens competent cells and plasmid DNA. A 25 μL aliquot of the mixture was transferred to a 96-multi-well Nucleovette® plate (Lonza). Electroporation was carried out using the Nucleofector™ 96-well Shuttle™ system (Lonza AG), and the electroporated cells were subsequently transferred to a fresh 96-well deep well plate, containing 500 μL M9 salts supplemented with 1% glucose medium, and trace elements. The plates were incubated at 30° C. with shaking for 48 hours, to generate seed cultures.

Ten μL aliquots of the seed cultures were transferred in duplicate into 96-well deep well plates. Each well contained 500 μL of HTP-YE medium (Teknova), supplemented with trace elements and 5% glycerol. The seed cultures, plated in the glycerol supplemented HTP media, were incubated for 24 hours, in a shaker, at 30° C. Isopropyl-β-D-1-thiogalactopyranoside (IPTG) was added to each well at a final concentration of 0.3 mM to induce expression of the Fab′. After 24 hours of induction, cell density was calculated by measuring the optical density at 600 nm (OD600). The cells were subsequently harvested, diluted 1:3 with 1× Phosphate Buffered Saline (PBS) to a final volume of 400 μL, and frozen for later processing.

Soluble Lysate Sample Preparation for Analytical Characterization: The harvested cell samples were diluted and lysed by sonication with a Cell Lysis Automated Sonication System (CLASS, Scinomix) using a 24 probe tip horn. The lysates were centrifuged at 5,500×g for 15 minutes at 8° C. The supernatant was collected and labeled as the soluble fraction. The pellets were collected, resuspended in 400 μL of 1×PBS pH 7.4 by another round of sonication, and labeled as the insoluble fraction.

Methods of screening microbial hosts to identify strains with improved yield and/or quality in the expression of recombinant proteins are described, for example, in U.S. Pat. Nos. 9,394,571 and 9,580,719, both titled “Method for rapidly screening microbial hosts to identify certain strains with improved yield and/or quality in the expression of heterologous proteins” each encorporated herein by reference.

### Protein Analysis

A recombinant protein of interest produced according to the methods of the present invention may be of high quality, e.g., active, soluble, and/or intact; produced at a high yield or titer; or any combination thereof. In some embodiments, a recombinant protein of interest is produced by a recombinant gram-negative bacterial host cell according to the methods of the present invention at higher quality and/or higher yield when compared to those observed with a control host cell. In some embodiments, a recombinant gram-negative bacterial host cell of the present invention grows to a higher cell density than a control host cell.

In some embodiments, recombinant proteins of interest produced by the methods provided herein are analyzed with regard to yield, solubility, activity, and degradation (e.g., by measuring intact protein). A recombinant protein of interest can be analyzed by any appropriate method known to those of skill in the art. The “solubility” and “activity” of a protein, though related qualities, are generally determined by different means. Solubility of a protein, particularly a hydrophobic protein, indicates that hydrophobic amino acid residues are properly located on the inside of the folded protein. Protein activity, which is often evaluated using different methods, e.g., as described below, is another indicator of proper protein conformation.

In some embodiments, a recombinant protein of interest is analyzed by biolayer interferometry, SDS-PAGE, Western blot, Far Western blot, ELISA, absorbance, or mass spectrometry (e.g., tandem mass spectrometry). In some embodiments, the concentration and/or amounts of polypeptides or proteins of interest generated are determined, for example, by Bradford assay, absorbance, Coomassie staining, mass spectrometry, hydrogen-deuterium exchange (HDX), etc. Protein yield and fragmentation in the insoluble and soluble fractions can be analyzed by methods known to those of skill in the art, for example, by capillary gel electrophoresis (CGE), SDS-PAGE, and Western blot analysis. Soluble fractions also can be evaluated, for example, using biolayer interferometry. Protein activity may be measured by any known method as appropriate for the recombinant protein of interest. For a recombinant protein of interest that is a binding protein, this may comprise measuring its binding to a target ligand, e.g., TNF-alpha, or any other target, by any known method. A recombinant protein of interest, e.g., a knob protein, produced using the methods of the present invention may be characterized and evaluated using any method known to one of skill in the art, including but not limited to those described herein in the Examples. Evaluation may include comparison with a reference, e.g., a knob protein produced according to a different method, e.g., a previously used method or any other method known to those of skill in the art. A previously used method may be, e.g., a fusion method as used in Example 1 herein.

Useful measures of protein yield include any as described, or known to those of skill in the art, e.g., the amount of recombinant protein per culture volume (e.g., concentration, which may be expressed in grams or milligrams of protein/liter of culture), percent or fraction of recombinant protein measured in the insoluble pellet obtained after lysis (e.g., amount of recombinant protein in extract supernatant/amount of protein in insoluble fraction), percent or fraction of active protein (e.g., amount of active protein/amount protein used in the assay), percent or fraction of total cell protein (tcp), amount of protein/cell, and percent dry biomass. A measure as used herein may refer to that determined for a large-scale fermentation culture.

In some embodiments, a recombinant gram-negative bacterial host cell of the invention grows to an increased cell density in culture than a control cell, under substantially the same growth conditions. In some embodiments, the increase in cell density relative to the control cell is about 2-fold to about 15-fold. In some embodiments, the increase in cell density relative to the control cell is about 2 fold to about 3 fold, about 2 fold to about 4 fold, about 2 fold to about 5 fold, about 2 fold to about 6 fold, about 2 fold to about 7 fold, about 2 fold to about 8 fold, about 2 fold to about 9 fold, about 2 fold to about 10 fold, about 2 fold to about 11 fold, about 2 fold to about 12 fold, about 2 fold to about 15 fold, about 3 fold to about 4 fold, about 3 fold to about 5 fold, about 3 fold to about 6 fold, about 3 fold to about 7 fold, about 3 fold to about 8 fold, about 3 fold to about 9 fold, about 3 fold to about 10 fold, about 3 fold to about 11 fold, about 3 fold to about 12 fold, about 3 fold to about 15 fold, about 4 fold to about 5 fold, about 4 fold to about 6 fold, about 4 fold to about 7 fold, about 4 fold to about 8 fold, about 4 fold to about 9 fold, about 4 fold to about 10 fold, about 4 fold to about 11 fold, about 4 fold to about 12 fold, about 4 fold to about 15 fold, about 5 fold to about 6 fold, about 5 fold to about 7 fold, about 5 fold to about 8 fold, about 5 fold to about 9 fold, about 5 fold to about 10 fold, about 5 fold to about 11 fold, about 5 fold to about 12 fold, about 5 fold to about 15 fold, about 6 fold to about 7 fold, about 6 fold to about 8 fold, about 6 fold to about 9 fold, about 6 fold to about 10 fold, about 6 fold to about 11 fold, about 6 fold to about 12 fold, about 6 fold to about 15 fold, about 7 fold to about 8 fold, about 7 fold to about 9 fold, about 7 fold to about 10 fold, about 7 fold to about 11 fold, about 7 fold to about 12 fold, about 7 fold to about 15 fold, about 8 fold to about 9 fold, about 8 fold to about 10 fold, about 8 fold to about 11 fold, about 8 fold to about 12 fold, about 8 fold to about 15 fold, about 9 fold to about 10 fold, about 9 fold to about 11 fold, about 9 fold to about 12 fold, about 9 fold to about 15 fold, about 10 fold to about 11 fold, about 10 fold to about 12 fold, about 10 fold to about 15 fold, about 11 fold to about 12 fold, about 11 fold to about 15 fold, or about 12 fold to about 15 fold. In some embodiments, the increase in cell density relative to the control cell is about 2 fold, about 3 fold, about 4 fold, about 5 fold, about 6 fold, about 7 fold, about 8 fold, about 9 fold, about 10 fold, about 11 fold, about 12 fold, or about 15 fold. In some embodiments, the increase in cell density relative to the control cell is at least about 2 fold, about 3 fold, about 4 fold, about 5 fold, about 6 fold, about 7 fold, about 8 fold, about 9 fold, about 10 fold, about 11 fold, or about 12 fold. In some embodiments, the increase in cell density relative to the control cell is at most about 3 fold, about 4 fold, about 5 fold, about 6 fold, about 7 fold, about 8 fold, about 9 fold, about 10 fold, about 11 fold, about 12 fold, or about 15 fold.

In some embodiments, a recombinant gram-negative bacterial host cell of the invention produces an increased yield of high-quality recombinant protein relative to a control cell. In some embodiments, the increased yield relative to the control cell is about 2-fold to about 100-fold. In some embodiments, the increased yield relative to the control cell is about 2 fold to about 5 fold, about 2 fold to about 10 fold, about 2 fold to about 20 fold, about 2 fold to about 30 fold, about 2 fold to about 40 fold, about 2 fold to about 50 fold, about 2 fold to about 60 fold, about 2 fold to about 70 fold, about 2 fold to about 80 fold, about 2 fold to about 90 fold, about 2 fold to about 100 fold, about 5 fold to about 10 fold, about 5 fold to about 20 fold, about 5 fold to about 30 fold, about 5 fold to about 40 fold, about 5 fold to about 50 fold, about 5 fold to about 60 fold, about 5 fold to about 70 fold, about 5 fold to about 80 fold, about 5 fold to about 90 fold, about 5 fold to about 100 fold, about 10 fold to about 20 fold, about 10 fold to about 30 fold, about 10 fold to about 40 fold, about 10 fold to about 50 fold, about 10 fold to about 60 fold, about 10 fold to about 70 fold, about 10 fold to about 80 fold, about 10 fold to about 90 fold, about 10 fold to about 100 fold, about 20 fold to about 30 fold, about 20 fold to about 40 fold, about 20 fold to about 50 fold, about 20 fold to about 60 fold, about 20 fold to about 70 fold, about 20 fold to about 80 fold, about 20 fold to about 90 fold, about 20 fold to about 100 fold, about 30 fold to about 40 fold, about 30 fold to about 50 fold, about 30 fold to about 60 fold, about 30 fold to about 70 fold, about 30 fold to about 80 fold, about 30 fold to about 90 fold, about 30 fold to about 100 fold, about 40 fold to about 50 fold, about 40 fold to about 60 fold, about 40 fold to about 70 fold, about 40 fold to about 80 fold, about 40 fold to about 90 fold, about 40 fold to about 100 fold, about 50 fold to about 60 fold, about 50 fold to about 70 fold, about 50 fold to about 80 fold, about 50 fold to about 90 fold, about 50 fold to about 100 fold, about 60 fold to about 70 fold, about 60 fold to about 80 fold, about 60 fold to about 90 fold, about 60 fold to about 100 fold, about 70 fold to about 80 fold, about 70 fold to about 90 fold, about 70 fold to about 100 fold, about 80 fold to about 90 fold, about 80 fold to about 100 fold, or about 90 fold to about 100 fold. In some embodiments, the increased yield relative to the control cell is about 2 fold, about 5 fold, about 10 fold, about 20 fold, about 30 fold, about 40 fold, about 50 fold, about 60 fold, about 70 fold, about 80 fold, about 90 fold, or about 100 fold. In some embodiments, the increased yield relative to the control cell is at least about 2 fold, about 5 fold, about 10 fold, about 20 fold, about 30 fold, about 40 fold, about 50 fold, about 60 fold, about 70 fold, about 80 fold, or about 90 fold. In some embodiments, the increased yield relative to the control cell is at most about 5 fold, about 10 fold, about 20 fold, about 30 fold, about 40 fold, about 50 fold, about 60 fold, about 70 fold, about 80 fold, about 90 fold, or about 100 fold.

In some embodiments, the methods herein are used to obtain a yield of active, soluble, and/or intact recombinant protein of interest of about 20% to about 90% total cell protein. In some embodiments, the yield of active, soluble, and/or intact polypeptide or protein of interest is about 20% total cell protein, about 25% total cell protein, about 30% total cell protein, about 31% total cell protein, about 32% total cell protein, about 33% total cell protein, about 34% total cell protein, about 35% total cell protein, about 36% total cell protein, about 37% total cell protein, about 38% total cell protein, about 39% total cell protein, about 40% total cell protein, about 41% total cell protein, about 42% total cell protein, about 43% total cell protein, about 44% total cell protein, about 45% total cell protein, about 46% total cell protein, about 47% total cell protein, about 48% total cell protein, about 49% total cell protein, about 50% total cell protein, about 51% total cell protein, about 52% total cell protein, about 53% total cell protein, about 54% total cell protein, about 55% total cell protein, about 56% total cell protein, about 57% total cell protein, about 58% total cell protein, about 59% total cell protein, about 60% total cell protein, about 65% total cell protein, about 70% total cell protein, about 75% total cell protein, about 80% total cell protein, about 85% total cell protein, or about 90% total cell protein. In some embodiments, the yield of active, soluble, and/or intact recombinant protein of interest is about 20% to about 25% total cell protein, about 20% to about 30% total cell protein, about 20% to about 35% total cell protein, about 20% to about 40% total cell protein, about 20% to about 45% total cell protein, about 20% to about 50% total cell protein, about 20% to about 55% total cell protein, about 20% to about 60% total cell protein, about 20% to about 65% total cell protein, about 20% to about 70% total cell protein, about 20% to about 75% total cell protein, about 20% to about 80% total cell protein, about 20% to about 85% total cell protein, about 20% to about 90% total cell protein, about 25% to about 90% total cell protein, about 30% to about 90% total cell protein, about 35% to about 90% total cell protein, about 40% to about 90% total cell protein, about 45% to about 90% total cell protein, about 50% to about 90% total cell protein, about 55% to about 90% total cell protein, about 60% to about 90% total cell protein, about 65% to about 90% total cell protein, about 70% to about 90% total cell protein, about 75% to about 90% total cell protein, about 80% to about 90% total cell protein, about 85% to about 90% total cell protein, about 31% to about 60% total cell protein, about 35% to about 60% total cell protein, about 40% to about 60% total cell protein, about 45% to about 60% total cell protein, about 50% to about 60% total cell protein, about 55% to about 60% total cell protein, about 31% to about 55% total cell protein, about 31% to about 50% total cell protein, about 31% to about 45% total cell protein, about 31% to about 40% total cell protein, about 31% to about 35% total cell protein, about 35% to about 55% total cell protein, or about 40% to about 50% total cell protein.

In some embodiments, the methods herein are used to obtain a yield (which may be referred to as a titer when expressed as a concentration) of active, soluble, and/or intact recombinant protein of interest of about 1 gram per liter to about 50 grams per liter. In some embodiments, the methods herein are used to obtain a yield of active, soluble, and/or intact recombinant protein of interest of about 0.1 g/L to about 50 g/L. In some embodiments, the methods herein are used to obtain a yield of active, soluble, and/or intact recombinant protein of interest of about 0.1 g/L to about 1 g/L, about 0.1 g/L to about 5 g/L, about 0.1 g/L to about 10 g/L, about 0.1 g/L to about 15 g/L, about 0.1 g/L to about 20 g/L, about 0.1 g/L to about 25 g/L, about 0.1 g/L to about 30 g/L, about 0.1 g/L to about 35 g/L, about 0.1 g/L to about 40 g/L, about 0.1 g/L to about 45 g/L, about 0.1 g/L to about 50 g/L, about 1 g/L to about 5 g/L, about 1 g/L to about 10 g/L, about 1 g/L to about 15 g/L, about 1 g/L to about 20 g/L, about 1 g/L to about 25 g/L, about 1 g/L to about 30 g/L, about 1 g/L to about 35 g/L, about 1 g/L to about 40 g/L, about 1 g/L to about 45 g/L, about 1 g/L to about 50 g/L, about 5 g/L to about 10 g/L, about 5 g/L to about 15 g/L, about 5 g/L to about 20 g/L, about 5 g/L to about 25 g/L, about 5 g/L to about 30 g/L, about 5 g/L to about 35 g/L, about 5 g/L to about 40 g/L, about 5 g/L to about 45 g/L, about 5 g/L to about 50 g/L, about 10 g/L to about 15 g/L, about 10 g/L to about 20 g/L, about 10 g/L to about 25 g/L, about 10 g/L to about 30 g/L, about 10 g/L to about 35 g/L, about 10 g/L to about 40 g/L, about 10 g/L to about 45 g/L, about 10 g/L to about 50 g/L, about 15 g/L to about 20 g/L, about 15 g/L to about 25 g/L, about 15 g/L to about 30 g/L, about 15 g/L to about 35 g/L, about 15 g/L to about 40 g/L, about 15 g/L to about 45 g/L, about 15 g/L to about 50 g/L, about 20 g/L to about 25 g/L, about 20 g/L to about 30 g/L, about 20 g/L to about 35 g/L, about 20 g/L to about 40 g/L, about 20 g/L to about 45 g/L, about 20 g/L to about 50 g/L, about 25 g/L to about 30 g/L, about 25 g/L to about 35 g/L, about 25 g/L to about 40 g/L, about 25 g/L to about 45 g/L, about 25 g/L to about 50 g/L, about 30 g/L to about 35 g/L, about 30 g/L to about 40 g/L, about 30 g/L to about 45 g/L, about 30 g/L to about 50 g/L, about 35 g/L to about 40 g/L, about 35 g/L to about 45 g/L, about 35 g/L to about 50 g/L, about 40 g/L to about 45 g/L, about 40 g/L to about 50 g/L, or about 45 g/L to about 50 g/L. In some embodiments, the methods herein are used to obtain a yield of active, soluble, and/or intact recombinant protein of interest of about 0.1 g/L, about 1 g/L, about 5 g/L, about 10 g/L, about 15 g/L, about 20 g/L, about 25 g/L, about 30 g/L, about 35 g/L, about 40 g/L, about 45 g/L, or about 50 g/L. In some embodiments, the methods herein are used to obtain a yield of active, soluble, and/or intact recombinant protein of interest of at least about 0.1 g/L, about 1 g/L, about 5 g/L, about 10 g/L, about 15 g/L, about 20 g/L, about 25 g/L, about 30 g/L, about 35 g/L, about 40 g/L, or about 45 g/L. In some embodiments, the methods herein are used to obtain a yield of active, soluble, and/or intact recombinant protein of interest of at most about 1 g/L, about 5 g/L, about 10 g/L, about 15 g/L, about 20 g/L, about 25 g/L, about 30 g/L, about 35 g/L, about 40 g/L, about 45 g/L, or about 50 g/L. In some embodiments, the methods herein are used to obtain a yield of active, soluble, and/or intact recombinant protein of interest of about 0.1 g/L to about 10 g/L. In some embodiments, the methods herein are used to obtain a yield of active, soluble, and/or intact recombinant protein of interest of about 0.1 g/L to about 0.5 g/L, about 0.1 g/L to about 1 g/L, about 0.1 g/L to about 2 g/L, about 0.1 g/L to about 3 g/L, about 0.1 g/L to about 4 g/L, about 0.1 g/L to about 5 g/L, about 0.1 g/L to about 6 g/L, about 0.1 g/L to about 7 g/L, about 0.1 g/L to about 8 g/L, about 0.1 g/L to about 9 g/L, about 0.1 g/L to about 10 g/L, about 0.5 g/L to about 1 g/L, about 0.5 g/L to about 2 g/L, about 0.5 g/L to about 3 g/L, about 0.5 g/L to about 4 g/L, about 0.5 g/L to about 5 g/L, about 0.5 g/L to about 6 g/L, about 0.5 g/L to about 7 g/L, about 0.5 g/L to about 8 g/L, about 0.5 g/L to about 9 g/L, about 0.5 g/L to about 10 g/L, about 1 g/L to about 2 g/L, about 1 g/L to about 3 g/L, about 1 g/L to about 4 g/L, about 1 g/L to about 5 g/L, about 1 g/L to about 6 g/L, about 1 g/L to about 7 g/L, about 1 g/L to about 8 g/L, about 1 g/L to about 9 g/L, about 1 g/L to about 10 g/L, about 2 g/L to about 3 g/L, about 2 g/L to about 4 g/L, about 2 g/L to about 5 g/L, about 2 g/L to about 6 g/L, about 2 g/L to about 7 g/L, about 2 g/L to about 8 g/L, about 2 g/L to about 9 g/L, about 2 g/L to about 10 g/L, about 3 g/L to about 4 g/L, about 3 g/L to about 5 g/L, about 3 g/L to about 6 g/L, about 3 g/L to about 7 g/L, about 3 g/L to about 8 g/L, about 3 g/L to about 9 g/L, about 3 g/L to about 10 g/L, about 4 g/L to about 5 g/L, about 4 g/L to about 6 g/L, about 4 g/L to about 7 g/L, about 4 g/L to about 8 g/L, about 4 g/L to about 9 g/L, about 4 g/L to about 10 g/L, about 5 g/L to about 6 g/L, about 5 g/L to about 7 g/L, about 5 g/L to about 8 g/L, about 5 g/L to about 9 g/L, about 5 g/L to about 10 g/L, about 6 g/L to about 7 g/L, about 6 g/L to about 8 g/L, about 6 g/L to about 9 g/L, about 6 g/L to about 10 g/L, about 7 g/L to about 8 g/L, about 7 g/L to about 9 g/L, about 7 g/L to about 10 g/L, about 8 g/L to about 9 g/L, about 8 g/L to about 10 g/L, or about 9 g/L to about 10 g/L. In some embodiments, the methods herein are used to obtain a yield of active, soluble, and/or intact recombinant protein of interest of about 0.1 g/L, about 0.5 g/L, about 1 g/L, about 2 g/L, about 3 g/L, about 4 g/L, about 5 g/L, about 6 g/L, about 7 g/L, about 8 g/L, about 9 g/L, or about 10 g/L. In some embodiments, the methods herein are used to obtain a yield of active, soluble, and/or intact recombinant protein of interest of at least about 0.1 g/L, about 0.5 g/L, about 1 g/L, about 2 g/L, about 3 g/L, about 4 g/L, about 5 g/L, about 6 g/L, about 7 g/L, about 8 g/L, or about 9 g/L. In some embodiments, the methods herein are used to obtain a yield of active, soluble, and/or intact recombinant protein of interest of at most about 0.5 g/L, about 1 g/L, about 2 g/L, about 3 g/L, about 4 g/L, about 5 g/L, about 6 g/L, about 7 g/L, about 8 g/L, about 9 g/L, or about 10 g/L. In some embodiments, the methods herein are used to obtain a yield of active, soluble, and/or intact recombinant protein of interest of about 0.2 to about 5 g/L. In some embodiments, the methods herein are used to obtain a yield of active, soluble, and/or intact recombinant protein of interest of about 0.2 g/L to about 5 g/L. In some embodiments, the methods herein are used to obtain a yield of active, soluble, and/or intact recombinant protein of interest of about 0.2 g/L to about 0.3 g/L, about 0.2 g/L to about 0.4 g/L, about 0.2 g/L to about 0.5 g/L, about 0.2 g/L to about 0.75 g/L, about 0.2 g/L to about 1 g/L, about 0.2 g/L to about 1.25 g/L, about 0.2 g/L to about 1.5 g/L, about 0.2 g/L to about 2 g/L, about 0.2 g/L to about 3 g/L, about 0.2 g/L to about 4 g/L, about 0.2 g/L to about 5 g/L, about 0.3 g/L to about 0.4 g/L, about 0.3 g/L to about 0.5 g/L, about 0.3 g/L to about 0.75 g/L, about 0.3 g/L to about 1 g/L, about 0.3 g/L to about 1.25 g/L, about 0.3 g/L to about 1.5 g/L, about 0.3 g/L to about 2 g/L, about 0.3 g/L to about 3 g/L, about 0.3 g/L to about 4 g/L, about 0.3 g/L to about 5 g/L, about 0.4 g/L to about 0.5 g/L, about 0.4 g/L to about 0.75 g/L, about 0.4 g/L to about 1 g/L, about 0.4 g/L to about 1.25 g/L, about 0.4 g/L to about 1.5 g/L, about 0.4 g/L to about 2 g/L, about 0.4 g/L to about 3 g/L, about 0.4 g/L to about 4 g/L, about 0.4 g/L to about 5 g/L, about 0.5 g/L to about 0.75 g/L, about 0.5 g/L to about 1 g/L, about 0.5 g/L to about 1.25 g/L, about 0.5 g/L to about 1.5 g/L, about 0.5 g/L to about 2 g/L, about 0.5 g/L to about 3 g/L, about 0.5 g/L to about 4 g/L, about 0.5 g/L to about 5 g/L, about 0.75 g/L to about 1 g/L, about 0.75 g/L to about 1.25 g/L, about 0.75 g/L to about 1.5 g/L, about 0.75 g/L to about 2 g/L, about 0.75 g/L to about 3 g/L, about 0.75 g/L to about 4 g/L, about 0.75 g/L to about 5 g/L, about 1 g/L to about 1.25 g/L, about 1 g/L to about 1.5 g/L, about 1 g/L to about 2 g/L, about 1 g/L to about 3 g/L, about 1 g/L to about 4 g/L, about 1 g/L to about 5 g/L, about 1.25 g/L to about 1.5 g/L, about 1.25 g/L to about 2 g/L, about 1.25 g/L to about 3 g/L, about 1.25 g/L to about 4 g/L, about 1.25 g/L to about 5 g/L, about 1.5 g/L to about 2 g/L, about 1.5 g/L to about 3 g/L, about 1.5 g/L to about 4 g/L, about 1.5 g/L to about 5 g/L, about 2 g/L to about 3 g/L, about 2 g/L to about 4 g/L, about 2 g/L to about 5 g/L, about 3 g/L to about 4 g/L, about 3 g/L to about 5 g/L, or about 4 g/L to about 5 g/L. In some embodiments, the methods herein are used to obtain a yield of active, soluble, and/or intact recombinant protein of interest of about 0.2 g/L, about 0.3 g/L, about 0.4 g/L, about 0.5 g/L, about 0.75 g/L, about 1 g/L, about 1.25 g/L, about 1.5 g/L, about 2 g/L, about 3 g/L, about 4 g/L, or about 5 g/L. In some embodiments, the methods herein are used to obtain a yield of active, soluble, and/or intact recombinant protein of interest of at least about 0.2 g/L, about 0.3 g/L, about 0.4 g/L, about 0.5 g/L, about 0.75 g/L, about 1 g/L, about 1.25 g/L, about 1.5 g/L, about 2 g/L, about 3 g/L, or about 4 g/L. In some embodiments, the methods herein are used to obtain a yield of active, soluble, and/or intact recombinant protein of interest of at most about 0.3 g/L, about 0.4 g/L, about 0.5 g/L, about 0.75 g/L, about 1 g/L, about 1.25 g/L, about 1.5 g/L, about 2 g/L, about 3 g/L, about 4 g/L, or about 5 g/L.

In some embodiments, the amount of active, soluble, and/or intact recombinant protein of interest is about 10% to about 100% of the amount of the total active, soluble, and/or intact recombinant protein of interest produced. In some embodiments, this amount is about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99%, or about 100% of the amount of the active, soluble, and/or intact recombinant protein of interest produced. In some embodiments, this amount is about 10% to about 20%, 20% to about 50%, about 25% to about 50%, about 25% to about 50%, about 25% to about 95%, about 30% to about 50%, about 30% to about 40%, about 30% to about 60%, about 30% to about 70%, about 35% to about 50%, about 35% to about 70%, about 35% to about 75%, about 35% to about 95%, about 40% to about 50%, about 40% to about 95%, about 50% to about 75%, about 50% to about 95%, about 70% to about 95%, or about 80 to about 100% of the amount of the active, soluble, and/or intact recombinant protein of interest produced.

In some embodiments, the amount of active, soluble, and/or intact recombinant protein of interest is expressed as a percentage of the total active, soluble, and/or intact protein produced in a culture. Data expressed in terms of active, soluble, and/or intact recombinant protein of interest weight/volume of cell culture at a given cell density can be converted to data expressed as percent recombinant protein of total cell protein. It is within the capabilities of a skilled artisan to convert volumetric protein yield to % total cell protein, for example, knowing the amount of total cell protein per volume of cell culture at the given cell density. This number can be determined if one knows 1) the cell weight/volume of culture at the given cell density, and 2) the percent of cell weight comprised by total protein. For example, at an OD550 of 1.0, the dry cell weight of E. coli is reported to be 0.5 grams/liter (“Production of Heterologous Proteins from Recombinant DNA Escherichia coli in Bench Fermentors,” Lin, N. S., and Swartz, J. R., 1992, METHODS: A Companion to Methods in Enzymology 4:159-168). A bacterial cell is comprised of polysaccharides, lipids, and nucleic acids, as well as proteins. An E. coli cell is reported to be about 52.4 to 55% protein by references including, but not limited to, Da Silva, N. A., et al., 1986, “Theoretical Growth Yield Estimates for Recombinant Cells,” Biotechnology and Bioengineering, Vol. XXVIII: 741-746, estimating protein to make up 52.4% by weight of E. coli cells, and “Escherichia coli and Salmonella typhimurium Cellular and Molecular Biology,” 1987, Ed. in Chief Frederick C. Neidhardt, Vol. 1, pp. 3-6, reporting protein content in E. coli as 55% dry cell weight. Using the measurements above (i.e., a dry cell weight of 0.5 grams/liter, and protein as 55% cell weight), the amount of total cell protein per volume of cell culture at an A550 of 1.0 for E. coli is calculated as 275 μg total cell protein/ml/A550. A calculation of total cell protein per volume of cell culture based on wet cell weight can use, e.g., the determination by Glazyrina, et al. (Microbial Cell Factories 2010, 9:42, incorporated herein by reference) that an A600 of 1.0 for E. coli resulted in a wet cell weight of 1.7 grams/liter and a dry cell weight of 0.39 grams/liter. For example, using this wet cell weight to dry cell weight comparison, and protein as 55% dry cell weight as described above, the amount of total cell protein per volume of cell culture at an A600 of 1.0 for E. coli can be calculated as 215 μg total cell protein/ml/A600. For Pseudomonas fluorescens, the amount of total cell protein per volume of cell culture at a given cell density is similar to that found for E. coli. P. fluorescens, like E. coli, is a gram-negative, rod-shaped bacterium. The dry cell weight of P. fluorescens ATCC 11150 as reported by Edwards, et al., 1972, “Continuous Culture of Pseudomonas fluorescens with Sodium Maleate as a Carbon Source,” Biotechnology and Bioengineering, Vol. XIV, pages 123-147, is 0.5 grams/liter/A500. This is the same weight reported by Lin, et al., for E. coli at an A550 of 1.0. Light scattering measurements made at 500 nm and at 550 nm are expected to be very similar. The percent of cell weight comprised by total cell protein for P. fluorescens HK44 is described as 55% by, e.g., Yarwood, et al., July 2002, “Noninvasive Quantitative Measurement of Bacterial Growth in Porous Media under Unsaturated-Flow Conditions,” Applied and Environmental Microbiology 68 (7): 3597-3605. This percentage is similar to or the same as those given for E. coli by the references described above.

In some embodiments, the amount of active, soluble, and/or intact recombinant protein of interest produced is about 0.1% to about 95% of the total active, soluble, and/or intact protein produced in a culture. In some embodiments, this amount is more than about 0.1%, 0.5%, 1%, 5%, 10%, 15%, 20%, 25%, 30%, 40%, 45%, 50%, 55%, 60%, 65%, 70%, 75%, 80%, 85%, 90%, or 95% of the total active, soluble, and/or intact protein produced in a culture. In some embodiments, this amount is about 0.1%, 0.5%, 1%, 5%, 10%, 15%, 20%, 25%, 30%, 40%, 45%, 50%, 55%, 60%, 65%, 70%, 75%, 80%, 85%, 90%, or 95% of the total active, soluble, and/or intact protein produced in a culture. In some embodiments, this amount is about 5% to about 95%, about 10% to about 85%, about 20% to about 75%, about 30% to about 65%, about 40% to about 55%, about 1% to about 95%, about 5% to about 30%, about 1% to about 10%, about 10% to about 20%, about 20% to about 30%, about 30% to about 40%, about 40% to about 50%, about 50 to about 60%, about 60% to about 70%, or about 80% to about 90% of the total active, soluble, and/or intact protein produced in a culture.

In some embodiments, the amount of active, soluble, and/or intact recombinant protein of interest produced is about 0.1% to about 50% of the dry cell weight (DCW). In some embodiments, this amount is more than about 0.1%, 0.5%, 1%, 5%, 10%, 15%, 20%, 25%, 30%, 40%, 45%, or 50% of DCW. In some embodiments, this amount is about 0.1%, 0.5%, 1%, 5%, 10%, 15%, 20%, 25%, 30%, 40%, 45%, or 50% of DCW. In some embodiments, this amount is about 5% to about 50%, about 10% to about 40%, about 20% to about 30%, about 1% to about 20%, about 5% to about 25%, about 1% to about 10%, about 10% to about 20%, about 20% to about 30%, about 30% to about 40%, or about 40% to about 50% of the total active, soluble, and/or intact protein produced in a culture.

In some embodiments, the amount of an active, soluble, and/or intact recombinant protein of interest produced using the methods of the invention is greater than the amount of the protein produced by a control host cell under substantially similar conditions, e.g., the same growth conditions. A control host cell may be a host cell that is the same in all respects to the recombinant gram-negative host cell, but that (a) is not deficient in one or more activities deficient in the recombinant gram-negative host cell, (b) does not overexpress one or more chaperones, folding modulators, or inactivated proteases that are overexpressed in the recombinant gram-negative host cell, or (c) any combination of (a) and (b). A control host cell may be a host cell that has the wild-type background of the recombinant gram-negative host cell, but that (a) is not deficient in one or more activities deficient in the recombinant gram-negative host cell, (b) does not overexpress one or more chaperones, folding modulators, or inactivated proteases that are overexpressed in the recombinant gram-negative host cell, or (c) any combination of (a) and (b). In some embodiments, an active, soluble, and/or intact recombinant protein of interest produced according to the present methods using a recombinant gram-negative host cell of the invention, is produced in an amount greater than the amount of the protein produced by a control host cell. In some embodiments, an active, soluble, and/or intact recombinant protein of interest produced by a recombinant gram-negative host cell of the invention is produced at a yield that is about 1.5 fold to about 10 fold. In some embodiments, an active, soluble, and/or intact recombinant protein of interest produced by a recombinant gram-negative host cell of the invention is produced at a yield that is about 1.5 fold to about 2 fold, about 1.5 fold to about 2.5 fold, about 1.5 fold to about 3 fold, about 1.5 fold to about 3.5 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 9 fold, about 1.5 fold to about 10 fold, about 2 fold to about 2.5 fold, about 2 fold to about 3 fold, about 2 fold to about 3.5 fold, about 2 fold to about 4 fold, about 2 fold to about 5 fold, about 2 fold to about 6 fold, about 2 fold to about 7 fold, about 2 fold to about 8 fold, about 2 fold to about 9 fold, about 2 fold to about 10 fold, about 2.5 fold to about 3 fold, about 2.5 fold to about 3.5 fold, about 2.5 fold to about 4 fold, about 2.5 fold to about 5 fold, about 2.5 fold to about 6 fold, about 2.5 fold to about 7 fold, about 2.5 fold to about 8 fold, about 2.5 fold to about 9 fold, about 2.5 fold to about 10 fold, about 3 fold to about 3.5 fold, about 3 fold to about 4 fold, about 3 fold to about 5 fold, about 3 fold to about 6 fold, about 3 fold to about 7 fold, about 3 fold to about 8 fold, about 3 fold to about 9 fold, about 3 fold to about 10 fold, about 3.5 fold to about 4 fold, about 3.5 fold to about 5 fold, about 3.5 fold to about 6 fold, about 3.5 fold to about 7 fold, about 3.5 fold to about 8 fold, about 3.5 fold to about 9 fold, about 3.5 fold to about 10 fold, about 4 fold to about 5 fold, about 4 fold to about 6 fold, about 4 fold to about 7 fold, about 4 fold to about 8 fold, about 4 fold to about 9 fold, about 4 fold to about 10 fold, about 5 fold to about 6 fold, about 5 fold to about 7 fold, about 5 fold to about 8 fold, about 5 fold to about 9 fold, about 5 fold to about 10 fold, about 6 fold to about 7 fold, about 6 fold to about 8 fold, about 6 fold to about 9 fold, about 6 fold to about 10 fold, about 7 fold to about 8 fold, about 7 fold to about 9 fold, about 7 fold to about 10 fold, about 8 fold to about 9 fold, about 8 fold to about 10 fold, or about 9 fold to about 10 fold greater than the amount of the protein produced by a control host cell. In some embodiments, an active, soluble, and/or intact recombinant protein of interest produced by a recombinant gram-negative host cell of the invention is produced at a yield that is about 1.5 fold, about 2 fold, about 2.5 fold, about 3 fold, about 3.5 fold, about 4 fold, about 5 fold, about 6 fold, about 7 fold, about 8 fold, about 9 fold, or about 10 fold greater than the amount of the protein produced by a control host cell. In some embodiments, active, soluble, and/or intact recombinant protein of interest produced by a recombinant gram-negative host cell of the invention is produced at a yield that is at least about 1.5 fold, about 2 fold, about 2.5 fold, about 3 fold, about 3.5 fold, about 4 fold, about 5 fold, about 6 fold, about 7 fold, about 8 fold, or about 9 fold greater than the amount of the protein produced by a control host cell. In some embodiments, active, soluble, and/or intact recombinant protein of interest produced by a recombinant gram-negative host cell of the invention is produced at a yield that is at most about 2 fold, about 2.5 fold, about 3 fold, about 3.5 fold, about 4 fold, about 5 fold, about 6 fold, about 7 fold, about 8 fold, about 9 fold, or about 10 fold greater than the amount of the protein produced by a control host cell.

### Activity Assays

Assays for evaluating the activity of a recombinant protein of interest are known in the art and include but are not limited to fluorometric, colorometric, chemiluminescent, spectrophotometric, and other enzyme assays available to one of skill in the art. A binding protein such as an antibody, antibody fragment, or derivative thereof may be evaluated by any appropriate assay, e.g., a target binding assay, known in the art. These assays may be used to compare activity of a preparation of a recombinant protein of interest to a commercial or other preparation of the recombinant protein. As understood by those of skill in the art, an activity assay may comprise evaluation of the effect of target binding by the recombinant protein or polypeptide, e.g., target neutralization, target inactivation, target activation (e.g., binding to the target to induce signaling), or any alteration of target activity as desired. Any appropriate in vitro or in vivo assay known in the art appropriate for evaluating the effect on the particular target may be used.

In some embodiments, activity is represented by the percent active protein in the extract supernatant as compared with the total amount assayed. This is based on the amount of protein determined to be active by the assay relative to the total amount of protein used in assay. In other embodiments, activity is represented by the % activity level of the protein compared to a standard, e.g., native protein. This is based on the amount of active protein in supernatant extract sample relative to the amount of active protein in a standard sample (where the same amount of protein from each sample is used in assay).

In some embodiments, about 40% to about 100% of the peptide, polypeptide or protein of interest, is determined to be active. In some embodiments, about 40%, about 50%, about 60%, about 70%, about 80%, about 90%, or about 100% of the peptide, polypeptide or protein of interest is determined to be active. In some embodiments, about 40% to about 50%, about 50% to about 60%, about 60% to about 70%, about 70% to about 80%, about 80% to about 90%, about 90% to about 100%, about 50% to about 100%, about 60% to about 100%, about 70% to about 100%, about 80% to about 100%, about 40% to about 90%, about 40% to about 95%, about 50% to about 90%, about 50% to about 95%, about 50% to about 100%, about 60% to about 90%, about 60% to about 95%, about 60% to about 100%, about 70% to about 90%, about 70% to about 95%, about 70% to about 100%, or about 70% to about 100% of the peptide, polypeptide or protein of interest is determined to be active.

In other embodiments, about 75% to about 100% of the peptide, polypeptide or protein of interest is determined to be active. In some embodiments, about 75% to about 80%, about 75% to about 85%, about 75% to about 90%, about 75% to about 95%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100% of the peptide, polypeptide or protein of interest is determined to be active.

### Exemplary Embodiments

1. A method for producing a recombinant ultralong CDR3 knob peptide, the method comprising: culturing a Pseudomonadales host cell in a culture medium and expressing the recombinant ultralong CDR3 knob peptide in the periplasm of the Pseudomonadales host cell from an expression construct comprising a nucleic acid encoding the recombinant ultralong CDR3 knob peptide directly and operably linked to a periplasmic secretion leader; wherein the recombinant ultralong CDR3 knob peptide is produced by secretion into the periplasm of the Pseudomonadales host cell, and wherein the secreted recombinant ultralong CDR3 knob peptide is present in soluble form, active form, or both.
2. The method of embodiment 1, wherein the recombinant ultralong CDR3 knob peptide is about 3 to about 8 kDa.
3. The method of embodiment 1 or 2, wherein the recombinant ultralong CDR3 knob peptide is about 25 to about 90 amino acids in length and comprises a cysteine motif, wherein the cysteine motif comprises 2-20 cysteine residues capable of forming 1-10 disulfide bonds.
4. The method of embodiment 3, wherein the recombinant ultralong CDR3 knob peptide further comprises a first stalk-forming amino acid sequence and a second stalk-forming amino acid sequence, each about 1 to about 15 amino acids in length.
5. The method of embodiment 4, wherein the cysteine motif is positioned between the first and second stalk-forming amino acid sequences.
6. The method of embodiment 4 or 5, wherein the first and second stalk-forming sequences are first and second β-strands, respectively, wherein the first and second β-strands are in anti-parallel configuration.
7. The method of any one of embodiments 1 to 6, wherein the periplasmic secretion leader has at least 85% identity to an amino acid sequence selected from SEQ ID NOS: 24, 26, 28, 30, 32, 34, 38, or 40.
8. The method of any one of embodiments 1 to 7, wherein the nucleic acid encoding the recombinant ultralong CDR3 knob peptide is operably linked to a ribosome binding site sequence (RBS).
9. The method of embodiment 8, wherein the sequence of the RBS is aggaggt or ggagcgt.
10. The method of any one of embodiments 1 to 9, wherein the sequence of the nucleic acid encoding the recombinant ultralong CDR3 knob peptide is not directly and/or operably linked to a nucleic acid sequence encoding a fusion partner, a cleavable linker, or both.
11. The method of any one of embodiments 1 to 10, wherein the produced recombinant ultralong CDR3 knob peptide is present in the periplasm in soluble form at a yield of about 0.5 g/L to about 8 g/L.
12. The method of any one of embodiments 1 to 11, wherein the produced recombinant ultralong CDR3 knob peptide is present in the periplasm in properly processed form.
13. The method of any one of embodiments 1 to 12, further comprising: measuring the quality of an amount of the recombinant ultralong CDR3 knob peptide produced.
14. The method of embodiment 13, wherein the quality is measured by an activity assay.
15. The method of embodiment 14, wherein the activity is target binding, and the activity assay is a binding assay.
16. The method of any one of embodiments 1 to 15, wherein at least 80% of the produced recombinant ultralong CDR3 knob peptide present in the periplasm in soluble form is active.
17. The method of any one of embodiments 13 to 16, wherein the activity is evaluated by comparison to a reference.
18. The method of embodiment 17, wherein the reference is selected from: a negative control or a corresponding recombinant ultralong CDR3 knob peptide produced from a fusion construct.
19. The method of any one of embodiments 1 to 18, wherein the nucleic acid encoding the recombinant ultralong CDR3 knob peptide is optimized for expression in the host cell.
20. The method of any one of embodiments 1 to 19, wherein the Pseudomonadales host cell is Pseudomonas fluorescens.
21. The method of any one of embodiments 1 to 21, wherein the Pseudomonadales host cell is deficient in expression of one or more proteases, overexpresses one or more folding modulators, overexpresses one or more inactivated proteases, or a combination thereof.
22. The method of embodiment 21, wherein the one or more protease is selected from: Lon, HslU, HslV, DegP1, DegP2, DegP2 S219A, Prc1, Prc2, MepM1, a serralysin, and AprA.
23. The method of embodiment 22, wherein the one or more protease comprises DegP2.
24. The method of embodiment 21, wherein the one or more protease is selected from: Prc1, Prc2, HslU, HslV, MepM1, and a serralysin.
25. The method of embodiment 24, wherein the serralysin is RXF04495.2.
26. The method of any one of embodiments 21 to 25, wherein the one or more folding modulator is selected from: SecB, DsbA, DsbC, Skp, and FklB2.
27. The method of any one of embodiments 1 to 26, wherein the host cell overexpresses: i) SecB; ii) DsbA, DsbC and Skp; iii) DsbA and DsbC; or iv) DsbC and FklB2.
28. The method of any one of embodiments 1 to 23, wherein the host cell is deficient in expression of DegP2, and overexpresses SecB.
29. The method of any one of embodiments 1 to 23, wherein the host cell is deficient in expression of DegP2, and overexpresses DsbA, DsbC, and Skp.
30. The method of any one of embodiments 1 to 23, wherein the host cell is deficient in expression of DegP2, and overexpresses DsbA and DsbC.
31. The method of any one of embodiments 1 to 23, wherein the host cell overexpresses DsbC and FklB2.
32. The method of any one of embodiments 1 to 21, wherein the host strain has a phenotype and genotype as set forth for any host strain in any one of Tables 3, 4, 6, and 9.
33. The method of any one of embodiments 1 to 21, wherein the host strain has a phenotype, genotype, and expression construct sequence elements as set forth for any host strain in any one of Tables 3, 4, 6, and 9.
34. The method of any one of embodiments 1 to 21, wherein the host strain is any as set forth in any one of Tables 3, 4, 6, and 9.
35. The method of any one of embodiments 1 to 23, wherein
   a) the periplasmic secretion leader has at least 85% identity to an amino acid sequence SEQ ID NO: 26;
   b) the host cell is deficient in expression of DegP2; and overexpresses SecB.
36. The method of any one of embodiments 1 to 23, wherein
   a) the periplasmic secretion leader has at least 85% identity to an amino acid sequence SEQ ID NO: 40;
   b) the host cell is deficient in expression of DegP2; and overexpresses i) SecB, or ii) DsbA, DsbC and Skp.
37. The method of any one of embodiments 1 to 23, wherein
   a) the periplasmic secretion leader has at least 85% identity to an amino acid sequence SEQ ID NO: 30;
   b) the host cell is deficient in expression of DegP2; and overexpresses SecB.
38. The method of any one of embodiments 1 to 37 wherein an amount of the ultralong CDR3 knob peptide secreted into the periplasm is released to the culture medium.
39. The method of any one of embodiments 1 to 38, further comprising purifying the produced recombinant ultralong CDR3 knob peptide.
40. The method of embodiment 39, wherein purifying the produced recombinant ultralong CDR3 knob peptide comprises: separating the cultured Pseudomonadales host cell expressing the recombinant ultralong CDR3 knob peptide from the culture medium to obtain separated Pseudomonadales host cell and separated culture medium; obtaining a cell lysate from the separated Pseudomonadales host cell; performing ultrafiltration of the cell lysate and/or the separated culture medium, to obtain an ultrafiltration permeate and an ultrafiltration concentrate; and performing chromatographic separation of the ultrafiltration permeate to obtain the purified recombinant ultralong CDR3 knob peptide.
41. The method of embodiment 40, wherein the ultrafiltration comprises passing the cell lysate and/or the separated culture medium through one or more molecular weight cut offs (MWCO) of about 5 to about 50 kDA.
42. The method of embodiment 40 or 41, wherein performing chromatographic separation of the ultrafiltration permeate comprises performing cation exchange chromatography on the ultrafiltration permeate.
43. The method of embodiment 39, wherein purifying the recombinant ultralong CDR3 knob peptide comprises: separating the cultured Pseudomonadales host cell expressing the recombinant ultralong CDR3 knob peptide from the culture medium to obtain separated Pseudomonadales host cell and separated culture medium; obtaining a cell lysate from the separated Pseudomonadales host cell; performing a first chromatographic separation of the cell lysate and/or the separated culture medium, to obtain a first eluate containing the recombinant ultralong CDR3 knob peptide; and performing a second chromatographic separation of the first eluate to obtain the purified recombinant ultralong CDR3 knob peptide.
44. The method of embodiment 43, wherein performing the first chromatographic separation of the cell lysate and/or the separated culture medium comprises performing cation exchange chromatography on the cell lysate and/or the separated culture medium.
45. The method of embodiment 43 or 44, wherein performing the second chromatographic separation of the first eluate comprises performing size exclusion chromatography on the first eluate.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the disclosure and are not meant to limit the present disclosure in any fashion. The present examples, along with the methods described herein are presently representative embodiments, are exemplary, and are not intended as limitations on the scope. Changes therein and other uses which are encompassed within the spirit of the disclosure as defined by the scope of the claims will occur to those skilled in the art.

### Example 1. Expression of Recombinant Fusion-Knob Proteins in a Pseudomonad Host Cell

### High Throughput Screening

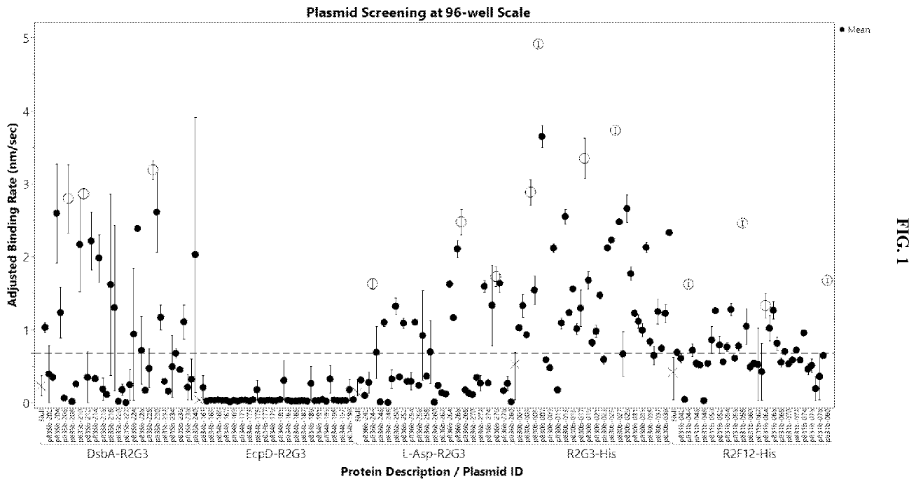

Screening of protease-deficient host cell strains, secretion leaders, and ribosome binding site (RBS) sequences for production of fusion proteins containing an anti-SARS-CoV-2 bovine knob protein, R2G3 or R2F12, was carried out. Nucleic acid constructs for 5 fusion-knob or affinity tagged knob proteins containing R2G3 or R2F12 were designed, and each construct was cloned into one of 40 plasmids to represent a unique combination of construct, secretion leader and RBS sequence at 96-well scale. The nucleic acid constructs and fusion proteins encoded by the constructs are shown in Table 1. Plasmids promoting high active fusion knob or C-terminal affinity tagged-knob expression in a wild-type P. fluorescens strain (DC454) were identified by assessing binding activity to receptor-binding domain (RBD) of the SARS-CoV-2 spike protein (“spike RBD”). Cultures were harvested 24 hours post-induction and sonicated to generate cell lysates. Soluble fractions of lysates were analyzed by biolayer interferometry (BLI) binding assay. The binding results are shown in FIG. 1. Adjusted binding rate, raw data from BLI readout, was used to evaluate amounts of active fusion knobs or C-terminal affinity tagged knobs in culture. The readout may be impacted by size of fusion/tag-knob and its orientation once bound to spike RBD. A larger fusion knob could potentially generate a higher binding rate compared to a smaller C-terminal affinity tagged-knob protein. Adjusted binding rate is proportional to fusion knob or C-terminal affinity tagged knob titer in culture. Adjusted binding rate is the measured concentration of diluted sample multiplied by the dilution factor. Plasmids promoting soluble active DsbA-R2G3, L-Asp-R2G3, R2G3-His, R2F12-His production are shown in Table 2 and indicated by open circles in FIG. 1.

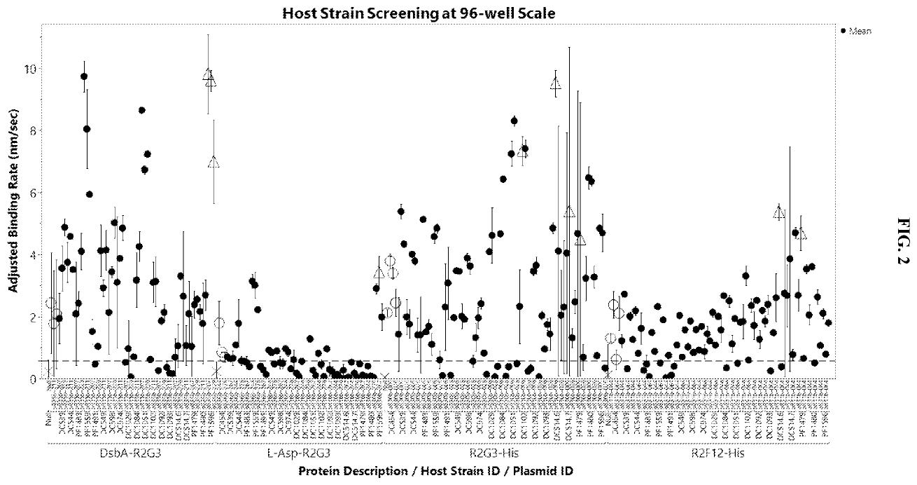

Host cell screening: The identified plasmids listed in Table 2 were transformed into 20 host strains for screening at 96-well scale. Soluble fractions of lysates were analyzed by BLI binding assay and SDS-capillary gel electrophoresis (CGE) for protein identification and titer estimation. The binding results are shown in FIG. 2. Host strains that produced high soluble active fusion knobs or C-terminal affinity tagged knobs are shown in Table 3. As can be seen from FIG. 2, multiple modified host strains significantly improved active DsbA-R2G3, R2G3-His, and R2F12-His production, compared to wild type DC454 strains. Based on binding results and the potential size impact on binding readout, top R2G3-His and top DsbA-R2G3 strains produced 8-10 nm/sec active fusion-knobs that suggested a higher amount of R2G3-His was produced compared to larger size of DsbA-R2G3. Fusion-knobs from host strain screening were also analyzed using SDS-CGE and titers were estimated based on CGE system ladder. High soluble fusion knob titers for larger DsbA and L-Asp fusion constructs were detected; however, the titer by SDS-CGE was not correlated to binding results. For example, 840 mcg/mL of DsbA-R2G3 from PS835-081 strain was determined by SDS-CGE but with low binding activity that suggested the protein quality from that fusion knob strain was poor. Similarly, L-Asp-R2G3 from PS836-111 produced 1061 mcg/mL fusion knob but had low/no binding activity. Thus, the criteria for host strain selection for fusion protein selection were based on the amount of soluble and active knobs determined by binding assay. Modified host strains producing improved active fusion knob or C-terminal affinity tagged knob expression (open triangles in FIG. 2) compared to wild type DC454 strains (open black circles), were selected for 2 L fermentation scale up.

### Large-Scale Expression

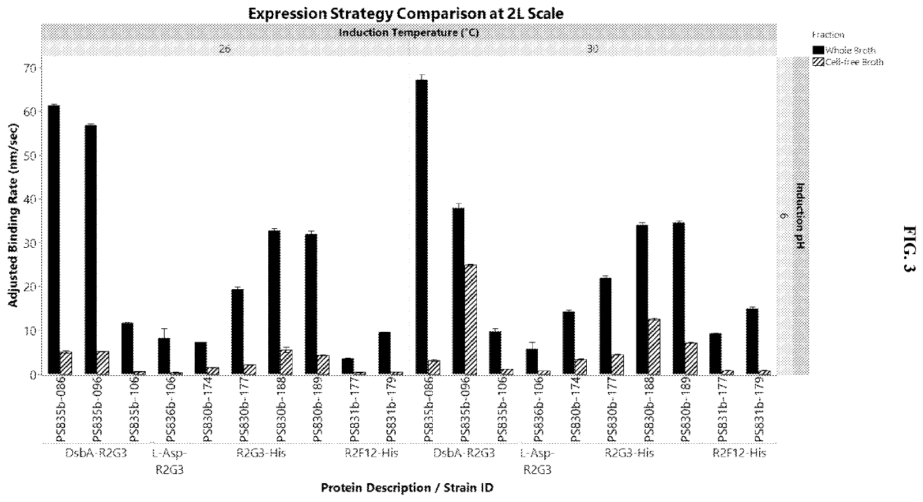

Selected host strains as shown in Table 4 were scaled up to 2 L fermentation, and active fusion knob and C-terminal affinity tagged knob titers were assessed by BLI as well as SDS-CGE for protein identification and titer estimation. FIG. 3 shows higher amounts of active DsbA-R2G3 and R2G3-His were produced compared to the other fusion constructs tested. As shown in Table 5, DsbA-R2G3 from PS835b-086 strain had approximately 2-fold higher adjusted binding rate (61-67 nm/sec) than that from PS830b-188 and PS830b-189 strains (32-35 nm/sec). In consideration of protein size impact on BLI readout, R2G3-His could have comparable or higher active titer compared to DsbA-R2G3 since R2G3-His, which is 25% smaller than DsbA-R2G3, had 50% binding rate of DsbA-R2G3. In addition to the binding assay, fusion-knob soluble fractions were examined by SDS-CGE under reducing conditions. Soluble titers of DsbA-R2G3 and L-Asp-R2G3 were estimated using internal CGE ladder and results showed up to 6 g/L of DsbA-R2G3 and 3 g/L of L-Asp-R2G3 were produced; this is correlated to high binding activity by BLI. Variability between culture sample replicates of PS835b-086, which expresses DsbA-R2G3, under the 30° C. induction temperature condition (5.94-9.23 g/L) was observed, potentially due to CGE sample preparation variability since low variability of the culture sample replicates was observed in the binding assay. With that consideration, the highest titer estimated by SDS-CGE was considered to be 6 g/L from DsbA-R2G3 strains. Expression strains used are listed in Table 9.

Conclusion: Using the optimized fusion protein expression strategy, approximately 6 g/L of R2G3 DsbA-R2G3 and 3 g/L of L-Asp-R2G3 knob-L-Asp fusion proteins was produced at 2 L scale, translating to 1 and 0.4 g/L of knobs, respectively, assuming 100% recovery after enzymatic cleavage. Recovery after enzymatic cleavage of fusion partner was in high single digit % at first attempt. Enzyme reaction optimization will improve cleavage efficiency and overall yield.

TABLE 1
Description of fusion knobs and C-terminal affinity tagged knobs.

| | Nucleic acid | Protein | No. of amino | No. of | |
|---|---|---|---|---|---|
| Protein | SEQ ID NO | SEQ ID NO | acids | Cysteine | MW (kDa) |
| DsbA-R2G3 | 1 | 2 | 271 | 10 | 28.576 |
| EcpD-R2G3 | 65 | 66 | 305 | 8 | 32.026 |
| L-Asp-R2G3 | 3 | 4 | 408 | 10 | 42.386 |
| R2G3-His | 5 | 6 | 72 | 8 | 6.906 |
| R2F12-His | 7 | 8 | 58 | 8 | 7.359 |

TABLE 2
Plasmids promoting high active fusion knob and C-terminal affinity tagged knob expression
in wild type strain (DC454) at 96-well scale. See open circles in FIG. 1. Adjusted
binding rate correlated to active knob titer in culture is presented.

| | | | % Knob | | | | Adjusted |
|---|---|---|---|---|---|---|---|
| | Number | Molecular | by | | | | Binding |
| | of | Weight | Molecular | | Secretion | | Rate |
| Protein | Cysteine | (kDa) | Weight | Plasmid ID | Leader | RBS | (nm/sec) |
| DsbA- | 10 | 28.576 | 19% | p835b-207 | AnsB | Me | 2.80 |
| R2G3 | | | | p835b-211 | CupB2 | Hi | 2.86 |
| | | | | p835b-229 | Leader M | Hi | 3.19 |
| L-Asp- | 10 | 42.386 | 13% | p836b-244 | 8484 | Hi | 1.63 |
| R2G3 | | | | p836b-267 | Lao | Me | 2.48 |
| | | | | p836b-276 | PorE | Me | 1.73 |
| R2G3-His | 8 | 6.906 | 79% | p830b-004 | 8484 | Hi | 2.88 |
| | | | | p830b-006 | AnsB | Hi | 4.91 |
| | | | | p830b-018 | FlgI | Hi | 3.35 |
| | | | | p830b-026 | Lao | Hi | 3.73 |
| R2F12-His | 8 | 7.359 | 80% | p831b-044 | 8484 | Hi | 1.62 |
| | | | | p831b-058 | FlgI | Hi | 2.46 |
| | | | | p831b-064 | Ibp-S31A | Hi | 1.33 |
| | | | | p831b-080 | TolB | Me | 1.67 |

TABLE 3
Top strains and wild type (DC454) strains from host strain screening at 96-well scale.
Top strains were determined based on binding activity to spike RBD. For each construct,
the strains are listed based on binding activity, from low to high.

| | | | | | | Adjusted | |
|---|---|---|---|---|---|---|---|
| | | | | | | Binding | Titer by |
| | | | | Secretion | | Rate | CGE |
| Protein | Strain ID | Host Strain** | Plasmid ID | Leader | RBS | (nm/sec) | (mcg/mL) |
| DsbA- | PS835b-091 | DC454 | p835b-211 | CupB2 | Hi | 1.77 | 404 |
| R2G3 | PS835b-101 | WT | p835b-229 | Leader M | Hi | 2.09 | 812 |
| | PS835b-081 | | p835b-207 | AnsB | Me | 2.44 | 840 |
| | PS835b-109 | DC598 | p835b-229 | Leader M | Hi | 5.03 | 773 |
| | | FMO: Ppi | | | | | |
| | PS835b-105 | PF1559 | p835b-229 | Leader M | Hi | 5.94 | 143 |
| | | PD: prc1, | | | | | |
| | | prc2, hslUV, | | | | | |
| | | mepM1 | | | | | |
| | PS835b-153 | DC1051 | p835b-211 | CupB2 | Hi | 6.73 | 656 |
| | | PD: lon, la1, | | | | | |
| | | prc1, prc2 | | | | | |
| | PS835b-106* | PF1596 | p835b-229 | Leader M | Hi | 6.99 | 490 |
| | PS835b-163 | DC1051 | p835b-229 | Leader M | Hi | 7.24 | 434 |
| | PS835b-095 | PF1559 | p835b-211 | CupB2 | Hi | 8.04 | 793 |
| | PS835b-143 | DC1051 | p835b-207 | AnsB | Me | 8.65 | 932 |
| | PS835b-096* | PF1596 | p835b-211 | CupB2 | Hi | 9.60 | 767 |
| | PS835b-085 | PF1559 | p835b-207 | AnsB | Me | 9.74 | 951 |
| | PS835b-086* | PF1596 | p835b-207 | AnsB | Me | 9.81 | 774 |
| L-Asp- | PS836b-111 | DC454 | p836b-276 | PorE | Me | 0.6915 | 1061 |
| R2G3 | PS836b-101 | | p836b-267 | Lao | Me | 0.8595 | 772 |
| | PS836b-081 | | p836b-244 | 8484 | Hi | 1.797 | 683 |
| | PS836b-106* | PF1596 | p836b-267 | Lao | Me | 3.4305 | 705 |
| R2G3- | PS830b-081 | DC454 | p830b-004 | 8484 | Hi | 2.12 | NA† |
| His | PS830b-111 | | p830b-026 | Lao | Hi | 2.44 | |
| | PS830b-101 | | p830b-018 | FlgI | Hi | 3.39 | |
| | PS830b-091 | | p830b-006 | AnsB | Hi | 3.79 | |
| | PS830b-092 | DC539 | p830b-006 | AnsB | Hi | 5.39 | |
| | | FMO: dsbAC | | | | | |
| | PS830b-188* | DC514.7 | p830b-018 | FlgI | Hi | 5.39 | |
| | PS830b-190 | PF1480 | p830b-018 | FlgI | Hi | 6.36 | |
| | | FMO: FklB3, | | | | | |
| | | DsbC | | | | | |
| | PS830b-182 | DC1084 | p830b-018 | FlgI | Hi | 6.42 | |
| | PS830b-180 | PF1480 | p830b-006 | AnsB | Hi | 6.47 | |
| | | PD: degP2, lon, | | | | | |
| | | la1 | | | | | |
| | PS830b-173 | DC1051 | p830b-006 | AnsB | Hi | 7.24 | |
| | PS830b-174* | DC1102 | p830b-006 | AnsB | Hi | 7.34 | |
| | PS830b-184 | DC1102 | p830b-018 | FlgI | Hi | 7.41 | |
| | PS830b-183 | DC1051 | p830b-018 | FlgI | Hi | 8.31 | |
| | PS830b-177* | DC514.6 | p830b-006 | AnsB | Hi | 9.51 | |
| R2F12- | PS831b-101 | DC454 | p831b-064 | Ibp-S31A | Hi | 0.62 | |
| His | PS831b-081 | | p831b-044 | 8484 | Hi | 1.30 | |
| | PS831b-111 | | p831b-080 | TolB | Me | 2.11 | |
| | PS831b-091 | | p831b-058 | FlgI | Hi | 2.38 | |
| | PS831b-168 | DC514.7 | p831b-044 | 8484 | Hi | 2.68 | |
| | PS831b-169 | PF1479 | p831b-044 | 8484 | Hi | 2.69 | |
| | PS831b-197 | DC514.6 | p831b-080 | TolB | Me | 2.76 | |
| | PS831b-174 | DC1102 | p831b-058 | FlgI | Hi | 3.32 | |
| | PS831b-199 | PF1479 | p831b-080 | TolB | Me | 3.54 | |
| | PS831b-180 | PF1480 | p831b-058 | FlgI | Hi | 3.61 | |
| | PS831b-178 | DC514.7 | p831b-058 | FlgI | Hi | 3.86 | |
| | PS831b-179* | PF1479 | p831b-058 | FlgI | Hi | 4.69 | |
| | PS831b-198 | DC514.7 | p831b-080 | TolB | Me | 4.70 | |
| | PS831b-177* | DC514.6 | p831b-058 | FlgI | Hi | 5.37 | |

*Selected strains for 2 L scale-up.
**PD = Protease Deletion (listed proteases are deleted); FMO = Folding Modulator Overexpressor (listed folding modulators are overexpressed.
†His-tagged knob cannot be resolved on SDS-CGE due to its small size.

TABLE 4
Identified host strains with improved active fusion knob and C-terminal
affinity tagged knob expression, selected for 2 L scale-up.

| | | | | | | | Genomic |
|---|---|---|---|---|---|---|---|
| | Strain | Plasmid | Secretion | Host | | | Knockout |
| Protein | ID | ID | Leader | Strain | Phenotype | FMO | (KO) |
| DsbA- | PS835b- | p835b- | AnsB | PF1596 | Protease | N/A | hslUV |
| R2G3 | 086 | 207 | | | deletion (PD) | | prc 1 |
| | PS835b- | p835b- | CupB2 | | | | prc2 |
| | 096 | 211 | | | | | MepM1 |
| | PS835b- | p835b- | Leader M | | | | serralysin |
| | 106 | 229 | | | | | |
| L-Asp- | PS836b- | p836b- | Lao | | | | |
| R2G3 | 106 | 267 | | | | | |
| R2G3-His | PS830b- | p830b- | AnsB | DC1102 | PD/Folding | secB | degP2 |
| | 174 | 006 | | | modulators | | |
| | | | | | overexpression | | |
| | | | | | (FMO) | | |
| | PS830b- | p830b- | AnsB | DC514.6 | PD/FMO | DsbA, DsbC, | degP2 |
| | 177 | 006 | | | | Skp | |
| | PS830b- | p830b- | FlgI | DC514.7 | PD/FMO | DsbC, DsbA | degP2 |
| | 188 | 018 | | | | | |
| | PS830b- | p830b- | FlgI | PF1479 | FMO | FklB2, DsbC | N/A |
| | 189 | 018 | | | | | |
| R2F12- | PS831b- | p831b- | FlgI | DC514.6 | PD/FMO | DsbA, DsbC, | degP2 |
| His | 177 | 058 | | | | Skp | |
| | PS831b- | p831b- | FlgI | PF1479 | FMO | FklB2, DsbC | N/A |
| | 179 | 058 | | | | | |

TABLE 5
Fusion knob and C-terminal affinity tagged knobs expression at 2 L scale. Adjusted
binding rate (active fusion knobs and C-terminal affinity tagged knobs) and soluble
titer estimation of fusion-knob strains by SDS-CGE under reducing conditions.

| | | Induction | | Adjusted | |
|---|---|---|---|---|---|
| | | Temperature | | Binding Rate | Soluble Titer by |
| Protein | Strain ID | (° C.) | Fraction | (nm/sec) | CGE (g/L)^{†} |
| DsbA-R2G3 | PS835b- | 26 | Whole Broth | 61.4 | 5.84 (5.75-5.94) |
| | 086 | | Cell-free Broth | 5.1 | 0.25 (0.23-0.26) |
| | | 30 | Whole Broth | 67.2 | 7.59 (5.94-9.23) |
| | | | Cell-free Broth | 3.2 | 0.20 (0.17-0.22) |
| | PS835b- | 26 | Whole Broth | 56.8 | 5.27 (4.90-5.64) |
| | 096 | | Cell-free Broth | 5.2 | 0.26 (0.21-0.30) |
| | | 30 | Whole Broth | 37.9 | 3.85 (3.62-4.08) |
| | | | Cell-free Broth | 24.9 | 1.98 (1.93-2.02) |
| | PS835b- | 26 | Whole Broth | 11.7 | 0.38 (0.34-0.41) |
| | 106 | | Cell-free Broth | 0.5 | too low for |
| | | | | | detection |
| | | 30 | Whole Broth | 9.7 | 0.43 (0.38-0.48) |
| | | | Cell-free Broth | 1.1 | 0.03 (0.03-0.03) |
| L-Asp- | PS836b- | 26 | Whole Broth | 8.2 | 2.96 (1.78-4.14) |
| R2G3 | 106 | | Cell-free Broth | 0.3 | too low for |
| | | | | | detection |
| | | 30 | Whole Broth | 5.7 | 3.17 (1.42-4.92) |
| | | | Cell-free Broth | 0.7 | 0.14 (0.14-0.14) |
| R2G3-His | PS830b- | 26 | Whole Broth | 7.3 | NA^{‡} |
| | 174 | | Cell-free Broth | 1.5 | |
| | | 30 | Whole Broth | 14.3 | |
| | | | Cell-free Broth | 3.4 | |
| | PS830b- | 26 | Whole Broth | 19.4 | |
| | 177 | | Cell-free Broth | 2.1 | |
| | | 30 | Whole Broth | 22.0 | |
| | | | Cell-free Broth | 4.4 | |
| | PS830b- | 26 | Whole Broth | 32.8 | |
| | 188 | | Cell-free Broth | 5.5 | |
| | | 30 | Whole Broth | 34.0 | |
| | | | Cell-free Broth | 12.5 | |
| | PS830b- | 26 | Whole Broth | 31.9 | |
| | 189 | | Cell-free Broth | 4.3 | |
| | | 30 | Whole Broth | 34.6 | |
| | | | Cell-free Broth | 7.1 | |
| R2F12-His | PS831b- | 26 | Whole Broth | 3.6 | |
| | 177 | | Cell-free Broth | 0.4 | |
| | | 30 | Whole Broth | 9.3 | |
| | | | Cell-free Broth | 0.8 | |
| | PS831b- | 26 | Whole Broth | 9.6 | |
| | 179 | | Cell-free Broth | 0.5 | |
| | | 30 | Whole Broth | 14.9 | |
| | | | Cell-free Broth | 0.8 | |

^{†}Soluble titer estimated by SDS-CGE was presented as mean with range of sampling duplicates.
^{‡}His-tagged knobs cannot be resolved on SDS-CGE due to its small size.

### Example 2. Mature Expression Strategy: Recombinant Mature Knob Protein Expression in a Pseudomonad Host Cell

Screening of protease-deficient P. fluorescens host cell strains, secretion leaders, and RBS sequences for production of two anti-SARS-CoV-2 bovine knob proteins, R2G3 and R2F12, using a mature expression strategy rather than a fusion-partner expression strategy, were carried out.

The expression of “mature” knob proteins from constructs encoding R2G3-mini (nucleic acid: SEQ ID NO: 11; amino acid: SEQ ID NO: 12), and R2F12-mini (nucleic acid: SEQ ID NO: 17; amino acid: SEQ ID NO: 18), was evaluated. R2G3-mini and R2F12-mini are truncated versions of R2G3 and R2F12, respectively, and maintain binding activity to spike RBD. This expression strategy contrasts with the fusion strategy described in Example 1, for example it does not exploit an N-terminal linker, such as GGGGAMGS (SEQ ID NO: 97), a fusion partner, or a C-terminal His tag.

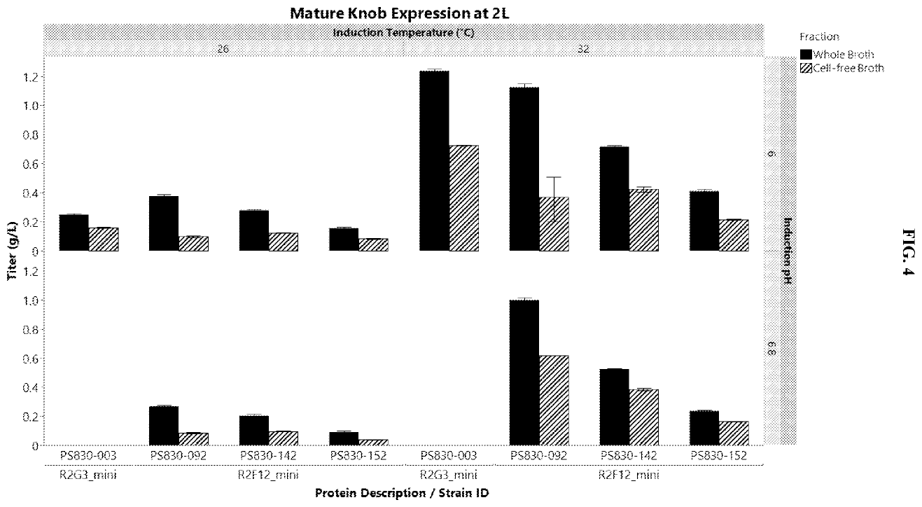

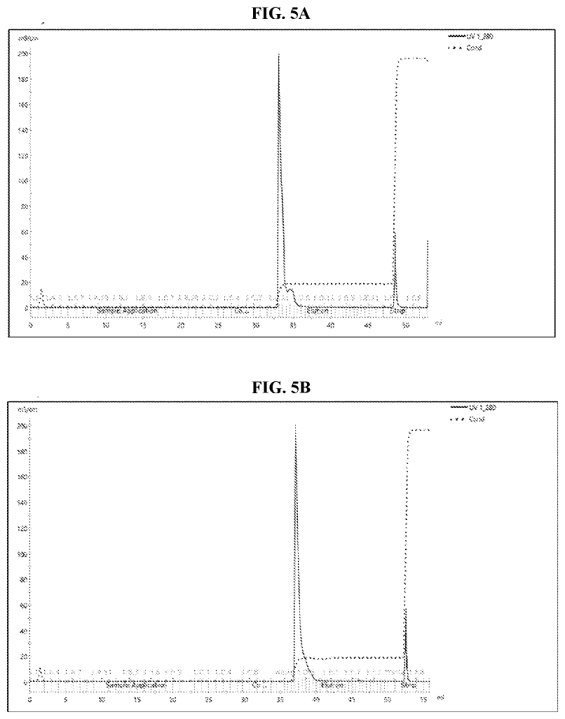

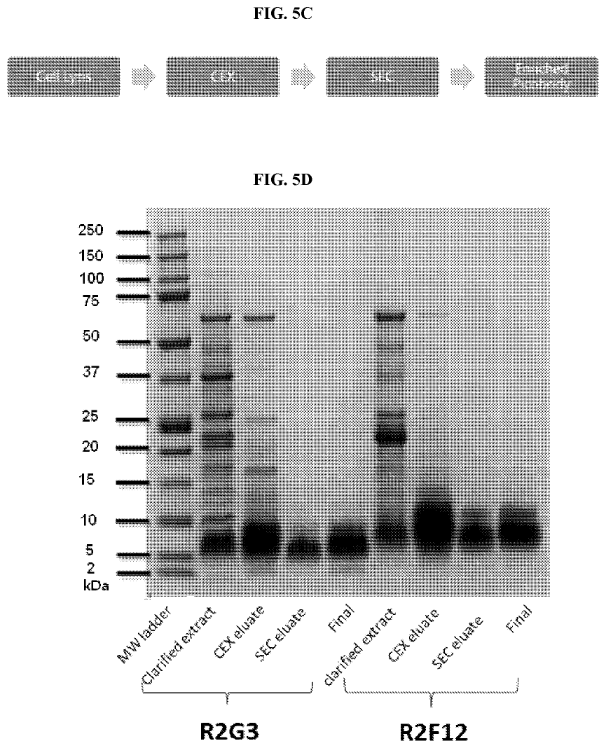

Host strains based on the plasmids and host strain combinations of C-terminal His tagged knobs R2G3-His and R2F12 His identified by expression at 96-well scale were evaluated at 2 L scale. The identified host strain and plasmid combinations are shown in Table 6. Expression results from 2 L scale are shown in Table 7. Two to four fermentation conditions were explored for each strain and soluble fractions of lysates from whole broth and cell-free-broth were accessed by binding assay to spike RBD. A truncated R2G3 (SEQ ID NO: 67, differing in amino acid sequence from R2G3-mini) was used as reference for titer estimation of R2G3-mini and R2F12-mini in BLI binding assay. FIG. 4 shows that induction temperature had an impact on knob production, and the most productive strains were PS830-003 for R2G3-mini and PS830-92 for R2F12-mini. Mature knob proteins from high-yield strains, PS830-003 and PS830-092, were purified by two column chromatography steps followed by a concentration step, and final products were characterized by analytical methods (Table 8A). Purification included cation exchange chromatography (CEX) and size exclusion chromatography (SEC). FIGS. 5A-B show chromatograms (FIG. 5A: R2G3-mini, and FIG. 5B: R2F12-mini) after the final chromatography step. Titers of both mature R2G3-mini and R2F12-mini were up to 1 g/L when expressed under fermentation induction conditions of 32° C., pH 6 and approximately half of the expressed mature knob appeared to be released from the periplasm into the culture medium (cell-free broth) for all strains evaluated (see Table 7). Expression strains used are listed in Table 9. The CEX-SEC purification process is illustrated by the flowchart shown in FIG. 5C. FIG. 5D shows SDS-PAGE analysis of chromatography fractions from R2G3-mini and R2F12-mini knob protein (picobody) purification from clarified lysate: clarified extract from cell free broth, eluates from CEX and SEC chromatography and the final sample following CEX column concentration chromatography.

### Purification of Mature Knob Proteins Using Ultrafiltration

Mature R2F12-mini (SEQ ID NO: 18) produced from PS830-003 was also purified using ultrafiltration. Ultrafiltration of R2F12-mini containing PS830-092 lysate was performed as a purification step to separate P. fluorescens host cell proteins from R2F12-mini and increase its purity prior to cation-exchange capture chromatography. Fermentation materials processed via ultrafiltration include the cell-free broth (CFB) and frozen cell paste components that are separated and frozen at the end of fermentation.

Frozen cell paste was resuspended to 20% (w/w) in 10 mM phosphate, pH 7.0 and mechanically lysed with one pass through a Microfluidics M110-P microfluidizer set to 15,000 psi with a cooling heat-exchanger on the instrument outlet to maintain a lysate temperature between 2-8° C. The lysate was clarified by batch centrifugation at 15,000×g for 45 minutes and subsequent filtration of the lysate supernatant with a 0.45/0.2 μm Sartobran P filter (Catalog #5231307H5-00-B).

Ultrafiltration was performed on the clarified lysate using a 50 cm² Millipore Pellicon XL Ultracel 30 kDa molecular weight cut off (MWCO) composite regenerated cellulose membrane in cassette format (Catalog #PXC030C50). Membrane challenge was 43.2 L/m². The general process involved permeate collection during two diavolumes of diafiltration followed by concentration of the retentate. Clarified lysate was recirculated through the membrane and retentate vessel at a crossflow rate of 528-600 LMH (same rate was maintained throughout the process). Less than 1 psi permeate backpressure was applied to prevent membrane fouling and no restriction was administered to the retentate line resulting in a Trans-Membrane Pressure between 9.0-10.9 psi. The retentate was exchanged against two diavolumes of 10 mM phosphate, pH 7.0 without permeate flux decay; after completion of the first diavolume, the permeate backpressure was removed. After completion of the second diavolume, the retentate was concentrated by a factor of 3.8.

SDS-PAGE analysis revealed significant transmission of target as evidenced by depletion in the concentrated retentate; due to retention of higher molecular weight impurities, R2F12-mini purity in the permeate was significantly higher than in the lysate.

The aforementioned process was repeated with a 50 cm² Millipore Pellicon XL Ultracel 10 kDa MWCO composite regenerated cellulose membrane in cassette format (Catalog #PXC010C50). Membrane challenge was 39.8 L/m². Trans-Membrane pressure during the process ranged from 6.9-9.1 psi. Reducing the MWCO from 30 to 10 kDa had the added benefit of removing additional host cell proteins from the clarified lysate although permeate flux rates were reduced.

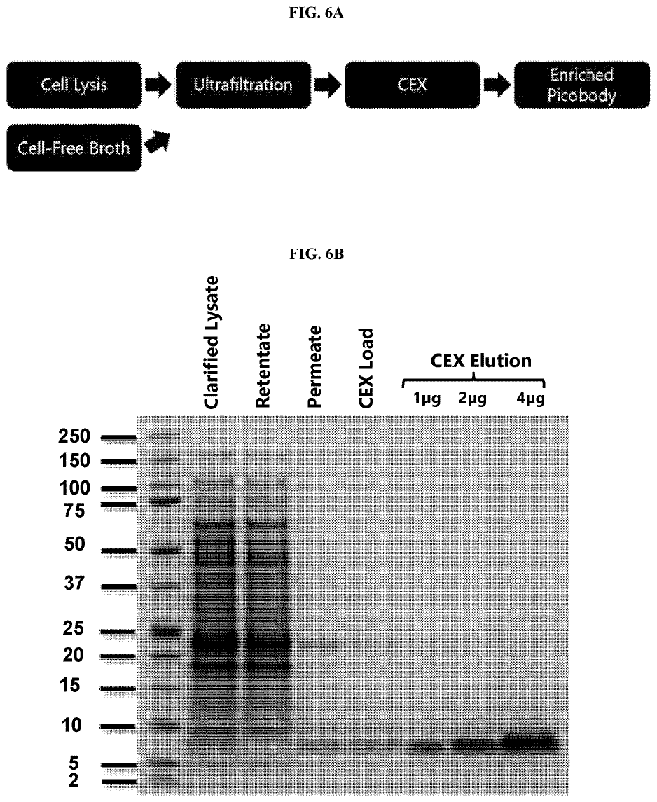

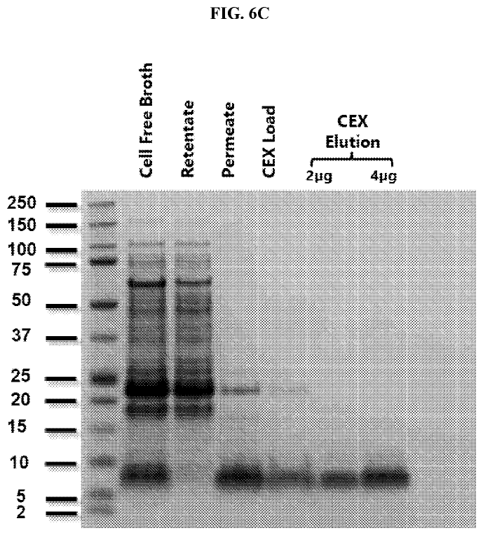

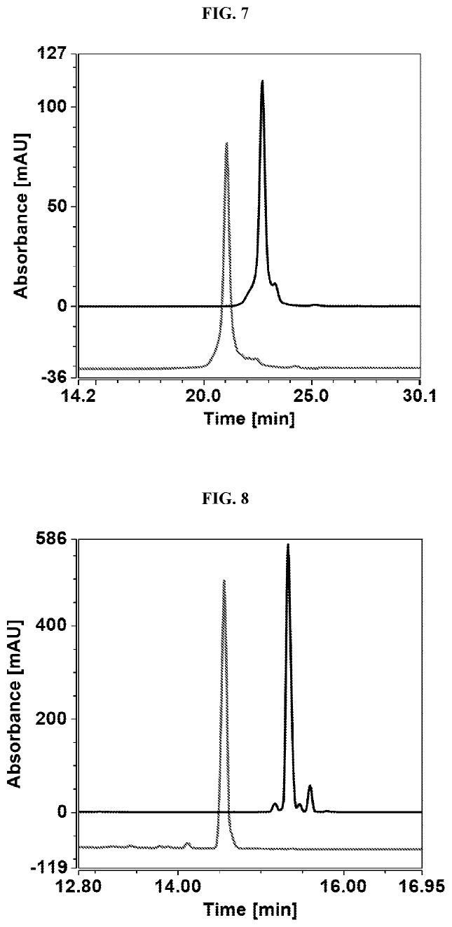

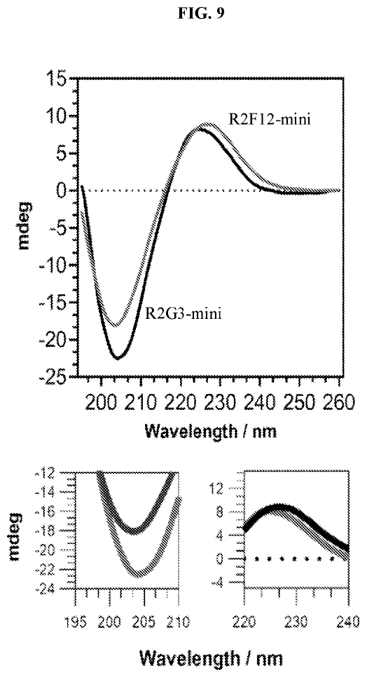

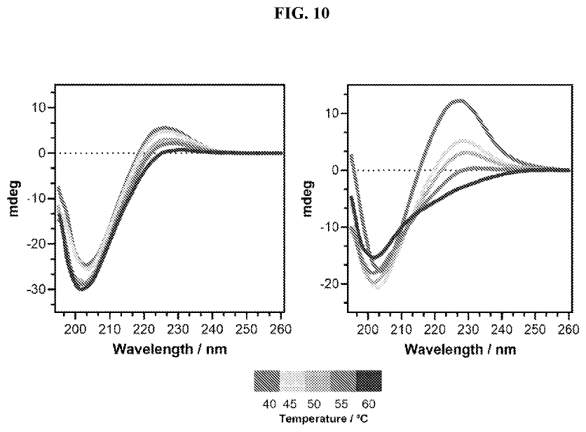

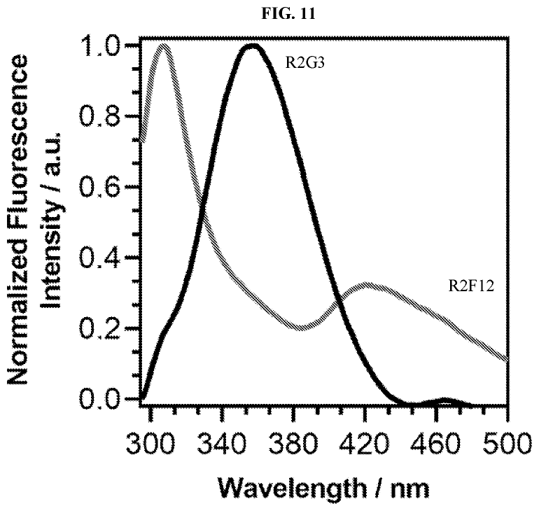

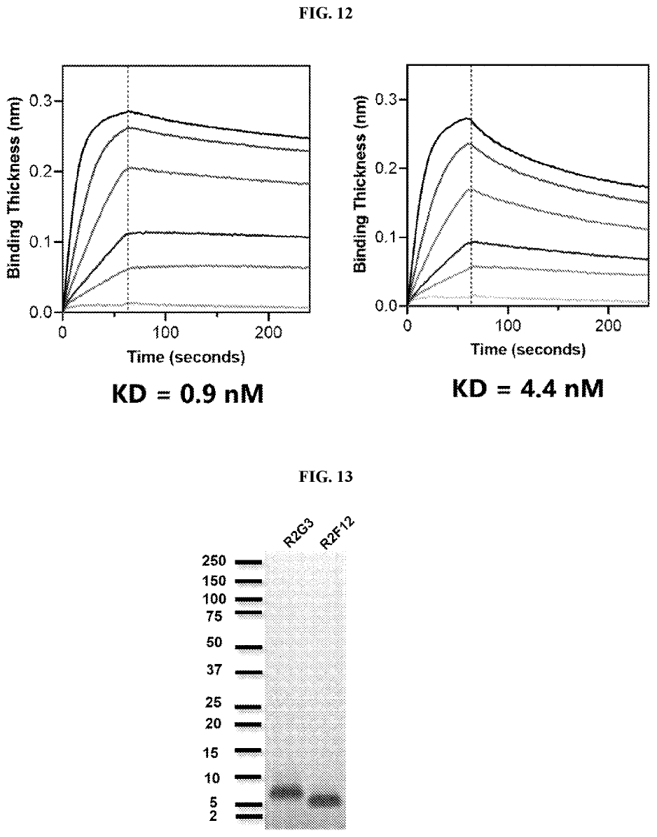

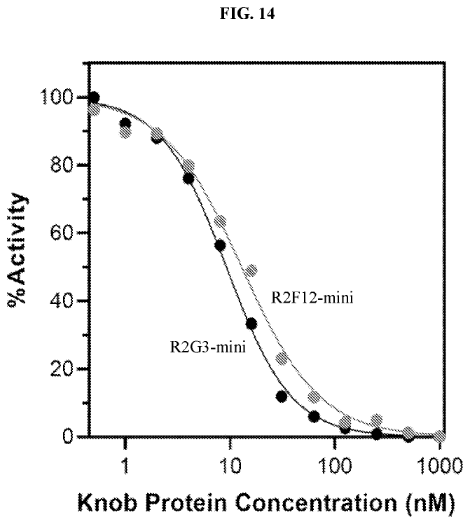

R2F12-mini was also purified from the cell-free broth portion of the same fermentation material via 10 kDa ultrafiltration. Frozen CFB was thawed and then clarified by batch centrifugation at 15,000×g for 45 minutes. The supernatant was filtered with a 0.45/0.2 μm Sartobran P filter (Catalog #5231307H5-00-B). Ultrafiltration of clarified CFB was then performed using the same parameters as described above with a 50 cm² Millipore Pellicon XL Ultracel 10 kDa MWCO composite regenerated cellulose. Membrane challenge was 13.6 L/m². The Trans-Membrane Pressure ranged between 8.4-10.0 psi. After diafiltration, the retentate was concentrated by a factor of 5.8. Process parameters for the ultrafiltration/diafiltration purification process are presented in Table 10A. Solutions used in the ultrafiltration (UF)/diafiltration (DF) process are presented in Table 10B. The UF/DF purification process is illustrated by the flowchart shown in FIG. 6A. FIG. 6B shows SDS-PAGE of the different process materials/solutions (clarified lysate, UF retentate, UF permeate, CEX load, and CEX eluate) obtained during knob protein purification from cell lysate using UF/DF purification process. FIG. 6C shows SDS-PAGE of the different process materials/solutions (cell free broth, UF retentate, UF permeate, CEX load, and CEX eluate) obtained during knob protein purification from cell free broth using UF/DF purification process. Results of the characterization of purified knob proteins produced using the mature knob expression strategy, performed as described below, are shown in FIG. 7 through FIG. 14.

### Characterization of Purified Knob Proteins

Intact Mass Analysis: Intact mass analysis was performed using a PLRP-S column (Agilent 5 μm, 4.6×50 mm), heated to 60° C., at a flow rate of 0.4 ml/min. Mobile phase A consisted of water with 0.1% (v/v) formic acid and mobile phase B acetonitrile with 0.1% (v/v) formic acid. A gradient elution of 3-70% mobile phase B in 20 minutes was used to elute the knob protein (R2G3-mini or R2F12-mini). For reduced analysis, 10 microliters of a 2 mg/mL solution of knob protein was mixed with 10 microliters of 8M guanidine solution, 2 microliters of 0.5M DTT and 2 microliters of 1M Tris buffer, pH 7.5 and the sample heated at 40° C. for 30 minutes prior to analysis. For non-reduced analysis, all samples were analyzed neat. Mass spectra was obtained on a Waters Xevo G2-XS TOF spectrometer calibrated in positive mode, with a capillary voltage of 3.0 kV, cone voltage of 50V, source temperature of 120° C., desolvation temperature of 280° C. and a desolvation gas flow of 600 L/Hr. Table 8B shows the results obtained from intact mass analysis of R2G3-mini, and R2F12-mini, as described in Examples 2 and 3 herein.

Size Exclusion Chromatography (SEC)-HPLC: Aggregation analysis was performed using a Sepax Zenix-C SEC-80 (II) (3 μm, 80Å, 7.8×300 mm, PN: 233081-7830) column fitted with a Sepax Zenix-C SEC-80 HPLC Pre-column filter kit, (PN: 102001-P356) and Sepax PEEK Refill Frits (0.5 μm, PN: 102000-P356). The UV wavelength used was 214 nm. The mobile phase flow was set to 0.4 mL/min and was composed of 100 mM sodium chloride, 0.05% v/v trifluoroacetic acid, 15% (v/v) methanol, and 15% (v/v) acetonitrile. The method time was 40 minutes. 10 micrograms of protein was injected for analysis. The knob proteins eluted between 20 and 25 minutes.

Reverse Phase (RP)-UPLC Analysis: RP-UPLC analysis was performed on a Phenomenex Aeris Peptide XB-C18 column (3 μm, 250×4.6 mm, PN: #00G-4507-E0) heated to 80° C. Mobile phase A consisted of water with 0.05% (v/v) trifluoroacetic acid and mobile phase B consisted of 90% acetonitrile (v/v), 10% water (v/v) and 0.05% (v/v) trifluoroacetic acid. An isocratic hold of 5% mobile phase B for 9 minutes was employed prior to a gradient increase of 5% to 29% mobile phase B in 3 minutes and then a 29% to 69% mobile phase B increase over 15 minutes. The UV detector was set to monitor a wavelength of 280 nm. 10-12 microgram of the knob proteins were injected for analysis.

Capillary isoelectric focusing (cIEF): 2 mg/mL knob protein samples were diluted 2-fold by combining 30 microliter of sample with 30 μL of Milli-Q water. 20 μL of the diluted sample was added to 180 μL of Master Mix comprised of: 35% of 1% Methyl cellulose, 10 mM of 0.5M Arginine, 10 mM of 0.25M Iminodiacetic acid, 4% of pI 3-10 Pharmalyte solution, 1% of pI 4.05 marker, 1% of pI 9.5 marker, 2M of 8M Urea, and Milli-Q water. The total amount of 200 μL was then aliquoted into the sample plate for iCIEF analysis. Separation of the analyte occurs in the Maurice capillary cIEF cartridge (Bio-techne PN: PS-MC02-C) set to 55 seconds of sample load, and 1 min at 1500 V then 15 min at 3000 V. Both absorbance and fluorescence detection settings were enabled. Absorbance exposure time was set to 0.005 seconds, while fluorescence exposure times were set to 3, 5, 10, 20 and 40 seconds. Data processing was performed by Compass for iCE software (Version: 2.1.0, Build ID: 0226) from Bio-techne.

Circular Dichroism (CD) Analysis: Circular dichroism were performed on a Jasco J-815 CD spectrophotometer fitted with a Peltier temperature controller and scanning emission monochromator. Knob protein samples were diluted to a final concentration of 0.25 mg/mL using ultra-pure water prior to analysis. A 1 mm pathlength cuvette was used for all analysis. Data was collected between 195 and 260 nm. For variable temperature experiments, the temperature controller was set to the desired temperature and allowed to equilibrate for 5 minutes prior to data acquisition.

KD Determination: KD determination of knob protein samples was performed utilizing biolayer interferometry. All sample and reagent dilutions and biosensor hydration were performed using 1× kinetics buffer (Sartorius, Cat #18-1105). Biotinylated SARS-CoV-2 Spike RBD (Sino Biologics, Cat #40592-V08H-B) at 94 nM was immobilized onto pre-hydrated SAX streptavidin biosensor tips (Sartorius, Cat #18-0037) for four minutes followed by baseline normalization in 1× kinetics buffer for 30 seconds. Association was evaluated by incubating the SARS-CoV-2 Spike RBD-loaded biosensor tips with a knob protein concentration gradient (125, 62.5, 31.3, 15.6, 7.8, 0 nM) for 60 seconds followed by dissociation in 1× kinetics buffer for three minutes. Data acquisition was performed using the Octet Red 96 instrument (Sartorius) using the Data Acquisition CFR11 v11.1 software (Sartorius). All data analysis was performed using the Data Analysis HT CFR v11.1 software (Sartorius).

SDS-PAGE Analysis: Knob protein samples were mixed at a 1:1 (v/v) ratio with 2× Laemmli sample buffer (BioRad, Cat #1610737) in the presence of 50 mM DTT (Sigma, Cat #A39255) and heated at 95° C. for five minutes. 1.5 μg of knob protein was loaded onto a 4-12% bis-tris gel (Invitrogen, Cat #NP0335). The gel was run at 175 V for 40 minutes using MES running buffer (Invitrogen, Cat #NP0002) and Precision Plus Protein Dual Xtra Standard (BioRad, Cat #1610377). The gel was stained using InstantBlue Coomassie Protein Stain (AbCam, Cat #ab119211) for 1 hour followed by de-staining in MilliQ water for 30 minutes. The gel was analyzed using the Image Lab Software v5.2.1 (BioRad).

IC50 determination: IC50 determination of knob protein samples was performed utilizing biolayer interferometry. All sample and reagent dilutions and biosensor hydration were performed using 1× kinetics buffer (Sartorius, Cat #18-1105). Biotinylated SARS-CoV-2 Spike RBD (Sino Biologics, Cat #40592-V08H-B) at 94 nM was immobilized onto pre-hydrated SAX streptavidin biosensor tips (Sartorius, Cat #18-0037) for four minutes followed by baseline normalization in 1× kinetics buffer for 30 seconds. The SARS-CoV-2 Spike RBD-loaded biosensor tips were then incubated with a concentration gradient of knob protein (1000, 500, 250, 125, 62.5, 31.3, 15.6, 7.8, 3.9, 2.0, 1.0, 0.5 and 0.25 nM) for 60 seconds followed by baseline normalization in 1× kinetics buffer for 30 seconds. Association of knob protein-bound SARS-CoV-2 Spike RBD to 91 nM ACE-2 receptor (Sino Biologics, Cat #10108-H05H) was performed for 60 seconds followed by dissociation in 1× kinetics buffer for three minutes. Data acquisition was performed using the Octet Red 96 instrument (Sartorius) using the Data Acquisition CFR11 v11.1 software (Sartorius). All data analysis was performed using the Data Analysis HT CFR v11.1 software (Sartorius). Response rates were analyzed and IC50 values calculated using non-linear regression in GraphPad Prism v8.4.2.

Reduced Peptide Mapping Analysis: 20 microliters of a 1 mg/mL solution of knob protein sample was added to 20 microliters of a solution of 2M urea, 50 mM TCEP and 50 mM Tris-Cl, pH 7.5. The solution was heated to 50° C. for 60 minutes. The digest mixture was diluted 2-fold with water, and 1 μg of trypsin added and the solution incubated at 40° C. for 2 hours. The digest solutions were injected onto an Agilent Advanced-Bio Peptide Column (2.7 μm, 3×150 mm), heated at 50° C., using a flow rate of 0.2 ml/min using Water and Acetonitrile with 0.1% formic acid as mobile phase A and B respectively. A gradient elution of 3-50% B in 26 minutes was used to resolve the peptides. Mass spectrometry analysis was performed using a Thermo Scientific Fusion Orbitrap. Data-dependent MS/MS was performed as follows for a three second cycle: the first event was a survey positive mass scan from 230 to 1700 m/z, followed by a data dependent collision induced fragmentation event (CID) with a collision energy of 35% and activation time of 10 ms. The next event was an electron transfer dissociation reaction (ETD) with an AGC target of 100%, with calibration dependent ETD parameters. Ions were generated using a sheath gas flow of 35, and auxiliary gas flow of 10, spray voltage of 3.5 kV, a capillary temperature of 325° C., and an S-lens RF level of 60%. Resolution was set at 120,000 (with an AGC target of 100%). The dynamic exclusion duration of 60 sec was used with a single repeat count. All data was analyzed using either BioPharmaFinder or Protein Metrics BYOS software.

Characterization results are summarized in Table 8A.

Conclusion: The mature knob protein expression strategy results in the production of equivalent or higher titers of active knob protein relative to expression of the knob protein as part of a fusion protein.

TABLE 6
Mature knob expression strains at 2 L scale up

| | Strain | Plasmid | Secretion | RBS | Host | | | Genomic |
|---|---|---|---|---|---|---|---|---|
| Protein | ID | ID | Leader | Strength | Strain | Phenotype | FMO | KO |
| R2G3-mini | PS830- | p830-402 | AnsB | Hi | DC1102 | PD/FMO | secB | degP2 |
| | 003 | | | | | | | |
| R2F12- | PS830- | P830-612 | TolB | Me | DC514.6 | PD/FMO | DsbA, | degP2 |
| mini | 092 | | | | | | DsbC, | |
| | | | | | | | Skp | |
| | PS830- | P830-602 | FlgI | Hi | DC1102 | PD/FMO | secB | degP2 |
| | 142 | | | | | | | |
| | PS830- | P830-612 | TolB | Me | DC1102 | PD/FMO | secB | degP2 |
| | 152 | | | | | | | |

TABLE 7
Mature knob expression at 2 L scale. BLI binding assay results of mature
knob expression strains. Titer measurement of active soluble knobs
was based on a standard curve using truncated R2G3 (SEQ ID NO: 67).

| | | Induction | | | Adjusted | |
|---|---|---|---|---|---|---|
| | | Temperature | Induction | | Binding Rate | Active Titer |
| Protein | Strain ID | (° C.) | pH | Fraction | (nm/sec) | by BLI (g/L) |
| R2G3- | PS830-003 | 26 | 6 | Whole Broth | 10.0 | 0.25 |
| mini | | | | Cell-free Broth | 6.5 | 0.16 |
| | | 32 | | Whole Broth | 42.7 | 1.24 |
| | | | | Cell-free Broth | 25.3 | 0.72 |
| R2F12- | PS830-092 | 26 | 6 | Whole Broth | 14.5 | 0.38 |
| mini | | | | Cell-free Broth | 10.8 | 0.10 |
| | | | 6.8 | Whole Broth | 10.0 | 0.27 |
| | | | | Cell-free Broth | 8.8 | 0.08 |
| | | 32 | 6 | Whole Broth | 45.9 | 1.12 |
| | | | | Cell-free Broth | 38.0 | 0.37 |
| | | | 6.8 | Whole Broth | 40.8 | 1.00 |
| | | | | Cell-free Broth | 76.6 | 0.62 |
| | PS830-142 | 26 | 6 | Whole Broth | 10.1 | 0.28 |
| | | | | Cell-free Broth | 4.4 | 0.13 |
| | | | 6.8 | Whole Broth | 6.7 | 0.20 |
| | | | | Cell-free Broth | 3.2 | 0.10 |
| | | 32 | 6 | Whole Broth | 29.7 | 0.72 |
| | | | | Cell-free Broth | 17.5 | 0.43 |
| | | | 6.8 | Whole Broth | 21.3 | 0.53 |
| | | | | Cell-free Broth | 15.9 | 0.39 |
| | PS830-152 | 26 | 6 | Whole Broth | 4.7 | 0.16 |
| | | | | Cell-free Broth | 2.5 | 0.08 |
| | | | 6.8 | Whole Broth | 2.4 | 0.09 |
| | | | | Cell-free Broth | 1.0 | 0.04 |
| | | 32 | 6 | Whole Broth | 16.1 | 0.41 |
| | | | | Cell-free Broth | 8.4 | 0.22 |
| | | | 6.8 | Whole Broth | 8.2 | 0.24 |
| | | | | Cell-free Broth | 6.0 | 0.16 |

TABLE 8A
Analytical characterization of enriched R2G3-mini and R2F12-mini knobs
produced at 2 L scale using mature knob expression strategy.

| | Results | |
|---|---|---|
| Analytical Test | R2G3-mini | R2F12-mini |
| Intact Mass | Mass matched to sequence | Mass matched to sequence |
| | 4 disulfide bonds present | 4 disulfide bonds present |
| Peptide Mapping | 100% Sequence Coverage, | 100% Sequence Coverage, |
| (Reduced) | Correct Primary Amino Acid | Correct Primary Amino Acid |
| | Sequence | Sequence |
| Purity by reverse phasc | 88.4% | 97.4% |
| Purity by size exclusion | 90.8% | 91.1% |
| Isoelectric Point (pI) | 7.37 (non-reduced) | 5.15 (non-reduced) |
| Determination by iCEF | | |
| Binding Activity | 111% | 97% |
| (Active conc by | | |
| BLI/A280) | | |
| KD | 0.9 nM | 4 nM |
| IC50 by ELISA | 9 nM | 13 nM |

TABLE 8B
Non-reduced and reduced mass spectrometry data characterizing
the monoisotopic mass for the R2G3-mini and R2F12-mini knob
proteins produced by mature expression strategy.

| | Expression Strategy | | | |
|---|---|---|---|---|
| | Mature Knob Expression | | | |
| | Protein | | | |
| | R2G3-mini | | R2F12-mini | |
| | Condition | | | |
| | Non-Reduced | Reduced | Non-Reduced | Reduced |
| Theoretical Isotopic | 3915.55 | 3923.61 | 4610.84 | 4618.90 |
| Mass (Da) | | | | |
| Observed Monoisotopic | 3915.56 | 3923.61 | 4610.85 | 4618.90 |
| Mass (Da) | | | | |
| Error (Da) | 0.01 | 0 | 0 | 0 |
| Error (ppm) | 2.4 | 0.4 | 1.4 | 0.7 |
| Δ Mass (Reduced-Non- | 8.05 | | 8.05 | |
| Reduced) | | | | |

TABLE 9
Expression strains used in Examples 1 and 2.

| | Protein | Strain | Plasmid | Secretion | | Host | | | FMO | |
|---|---|---|---|---|---|---|---|---|---|---|
| Protein | Group | ID | ID | Leader | RBS | Strain | Phenotype | FMO | Plasmid | Genotype |
| DsbA-R2G3 | Fusion | PS835b- | p835b- | AnsB | Me | DC454 | WT | N/A | N/A | ΔpyrF lsc::lacIQ1 |
| | | 007 | 207 | | | | | | | |
| DsbA-R2G3 | Fusion | PS835b- | p835b- | CupB2 | Hi | DC454 | WT | N/A | N/A | ΔpyrF lsc::lacIQ1 |
| | | 011 | 211 | | | | | | | |
| DsbA-R2G3 | Fusion | PS835b- | p835b- | Leader | Hi | DC454 | WT | N/A | N/A | ΔpyrF lsc::lacIQ1 |
| | | 029 | 229 | M | | | | | | |
| L-Asp-R2G3 | Fusion | PS836b- | p836b- | 8484 | Hi | DC454 | WT | N/A | N/A | ΔpyrF lsc::lacIQ1 |
| | | 004 | 244 | | | | | | | |
| L-Asp-R2G3 | Fusion | PS836b- | p836b- | Lao | Me | DC454 | WT | N/A | N/A | ΔpyrF lsc::lacIQ1 |
| | | 027 | 267 | | | | | | | |
| L-Asp-R2G3 | Fusion | PS836b- | p836b- | PorE | Me | DC454 | WT | N/A | N/A | ΔpyrF lsc::lacIQ1 |
| | | 036 | 276 | | | | | | | |
| R2G3-His | His-Tag | PS830b- | p830b- | 8484 | Hi | DC454 | WT | N/A | N/A | ΔpyrF lsc::lacIQ1 |
| | | 004 | 004 | | | | | | | |
| R2G3-His | His-Tag | PS830b- | p830b- | AnsB | Hi | DC454 | WT | N/A | N/A | ΔpyrF lsc::lacIQ1 |
| | | 006 | 006 | | | | | | | |
| R2G3-His | His-Tag | PS830b- | p830b- | FlgI | Hi | DC454 | WT | N/A | N/A | ΔpyrF lsc::lacIQ1 |
| | | 018 | 018 | | | | | | | |
| R2G3-His | His-Tag | PS830b- | p830b- | Lao | Hi | DC454 | WT | N/A | N/A | ΔpyrF lsc::lacIQ1 |
| | | 026 | 026 | | | | | | | |
| R2F12-His | His-Tag | PS831b- | p831b- | 8484 | Hi | DC454 | WT | N/A | N/A | ΔpyrF lsc::lacIQ1 |
| | | 004 | 044 | | | | | | | |
| R2F12-His | His-Tag | PS831b- | p831b- | FlgI | Hi | DC454 | WT | N/A | N/A | ΔpyrF lsc::lacIQ1 |
| | | 018 | 058 | | | | | | | |
| R2F12-His | His-Tag | PS831b- | p831b- | Ibp- | Hi | DC454 | WT | N/A | N/A | ΔpyrF lsc::lacIQ1 |
| | | 024 | 064 | S31A | | | | | | |
| R2F12-His | His-Tag | PS831b- | p831b- | TolB | Me | DC454 | WT | N/A | N/A | ΔpyrF lsc::lacIQ1 |
| | | 040 | 080 | | | | | | | |
| DsbA-R2G3 | Fusion | PS835b- | p835b- | AnsB | Me | PF1596 | PD | N/A | N/A | Δprc1 Δprc2 |
| | | 086 | 207 | | | | | | | ΔhslU ΔhslV |
| | | | | | | | | | | ΔmepM1 |
| | | | | | | | | | | ΔRXF04495.2 |
| | | | | | | | | | | (serralysin) ΔpyrF |
| | | | | | | | | | | lsc::lacIQ1 |
| DsbA-R2G3 | Fusion | PS835b- | p835b- | CupB2 | Hi | PF1596 | PD | N/A | N/A | Δprc1 Δprc2 |
| | | 096 | 211 | | | | | | | ΔhslU ΔhslV |
| | | | | | | | | | | ΔmepM1 |
| | | | | | | | | | | ΔRXF04495.2 |
| | | | | | | | | | | (serralysin) ΔpyrF |
| | | | | | | | | | | lsc::lacIQ1 |
| DsbA-R2G3 | Fusion | PS835b- | p835b- | Leader | Hi | PF1596 | PD | N/A | N/A | Δprc1 Δprc2 |
| | | 106 | 229 | M | | | | | | ΔhslU ΔhslV |
| | | | | | | | | | | ΔmepM1 |
| | | | | | | | | | | ΔRXF04495.2 |
| | | | | | | | | | | (serralysin) ΔpyrF |
| | | | | | | | | | | lsc::lacIQ1 |
| L-Asp-R2G3 | Fusion | PS836b- | p836b- | Lao | Me | PF1596 | PD | N/A | N/A | Δprc1 Δprc2 |
| | | 106 | 267 | | | | | | | ΔhslU ΔhslV |
| | | | | | | | | | | ΔmepM1 |
| | | | | | | | | | | ΔRXF04495.2 |
| | | | | | | | | | | (serralysin) ΔpyrF |
| | | | | | | | | | | lsc::lacIQ1 |
| R2G3-His | His-Tag | PS830b- | p830b- | AnsB | Hi | DC1102 | PD/ | secB | pDOW3702 | ΔpyrF ΔproC |
| | | 174 | 006 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | AnsB | Hi | DC514.6 | PD/ | dsbACSkp | pFNX3965 | ΔpyrF ΔproC |
| | | 177 | 006 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | FlgI | Hi | DC514.7 | PD/ | dsbCA | pFNX3966 | ΔpyrF ΔproC |
| | | 188 | 018 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | FlgI | Hi | PF1479 | FMO | FklB2- | pFNX7403 | ΔpyrF ΔproC |
| | | 189 | 018 | | | | | DsbC | | ΔbenA ΔbenB |
| | | | | | | | | | | ΔmtlD ΔmtlY |
| | | | | | | | | | | ΔmtlZ lsc::lacIQ1 |
| R2F12-His | His-Tag | PS831b- | p831b- | FlgI | Hi | DC514.6 | PD/ | dsbACSkp | pFNX3965 | ΔpyrF ΔproC |
| | | 177 | 058 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2F12-His | His-Tag | PS831b- | p831b- | FlgI | Hi | PF1479 | FMO | FklB2- | pFNX7403 | ΔpyrF ΔproC |
| | | 179 | 058 | | | | | DsbC | | ΔbenA ΔbenB |
| | | | | | | | | | | ΔmtlD ΔmtlY |
| | | | | | | | | | | ΔmtlZ lsc::lacIQ1 |
| R2G3-His | His-Tag | PS830b- | p830b- | AnsB | Hi | DC1102 | PD/ | secB | pDOW3702 | ΔpyrF ΔproC |
| | | 174 | 006 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | AnsB | Hi | DC514.6 | PD/ | dsbACSkp | pFNX3965 | ΔpyrF ΔproC |
| | | 177 | 006 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | FlgI | Hi | DC514.7 | PD/ | dsbCA | pFNX3966 | ΔpyrF ΔproC |
| | | 188 | 018 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2G3-His | His-Tag | PS830b- | p830b- | FlgI | Hi | PF1479 | FMO | FklB2- | pFNX7403 | ΔpyrF ΔproC |
| | | 189 | 018 | | | | | DsbC | | ΔbenA ΔbenB |
| | | | | | | | | | | ΔmtlD ΔmtlY |
| | | | | | | | | | | ΔmtlZ lsc::lacIQ1 |
| R2F12-His | His-Tag | PS831b- | p831b- | FlgI | Hi | DC514.6 | PD/ | dsbACSkp | pFNX3965 | ΔpyrF ΔproC |
| | | 177 | 058 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2F12-His | His-Tag | PS831b- | p831b- | FlgI | Hi | PF1479 | FMO | FklB2- | pFNX7403 | ΔpyrF ΔproC |
| | | 179 | 058 | | | | | DsbC | | ΔbenA ΔbenB |
| | | | | | | | | | | ΔmtlD ΔmtlY |
| | | | | | | | | | | ΔmtlZ lsc::lacIQ1 |
| R2G3_mini | Tagless | PS830- | p830- | AnsB | Hi | DC1102 | PD/ | secB | pDOW3702 | ΔpyrF ΔproC |
| | | 003 | 402 | | | | FMO | | | ΔbenA ΔbenB |
| | | | | | | | | | | ΔmtlD ΔmtlY |
| | | | | | | | | | | ΔmtIZ ΔdegP2 |
| | | | | | | | | | | lsc::lacIQ1 |
| R2G3_mini | Tagless | PS830- | p830- | FlgI | Hi | PF1479 | FMO | FklB2- | pFNX7403 | ΔpyrF ΔproC |
| | | 009 | 405 | | | | | DsbC | | ΔbenA ΔbenB |
| | | | | | | | | | | ΔmtlD ΔmtlY |
| | | | | | | | | | | ΔmtlZ lsc::lacIQ1 |
| R2G3_mini | Tagless | P830- | P830- | TolB | Me | DC514.6 | PD/ | dsbACSkp | pFNX3965 | ΔpyrF ΔproC |
| | | 092 | 612 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2F12_mini | Tagless | P830- | P830- | FlgI | Hi | DC1102 | PD/ | secB | pDOW3702 | ΔpyrF ΔproC |
| | | 142 | 602 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |
| R2F12_mini | Tagless | P830- | P830- | TolB | Me | DC1102 | PD/ | secB | pDOW3702 | ΔpyrF ΔproC |
| | | 152 | 612 | | | | FMO | | | ΔbenA ΔbenB |
| | | | | | | | | | | ΔmtlD ΔmtlY |
| | | | | | | | | | | ΔmtlZ ΔdegP2 |
| | | | | | | | | | | lsc::lacIQ1 |

TABLE 10A
UF/DF process parameters.

| | TMP | | | Retentate | Permeate | | |
|---|---|---|---|---|---|---|---|
| Step | (psi) | Solution | Mode* | Line | Line | Volume | End Condition |
| System | 6-11 | 0.5N NaOH | SPPO | Open | Open | ≥10 | ≥60 min recirculation |
| Sanitization | | | TRPO | | | L/m² | |
| System Rinse | | High Purity | SPPO | | | ≥20 | Permeate: |
| | | Water | | | | L/m² | Cond. ≤ 5 mS/cm |
| | | | | | | | pH 6.0-8.0 |
| System EQ | | DF Buffer | SPPO | | | ≥5 L/m² | Permeate within ± 0.1 pH |
| | | | TRPO | | | | and ± 0.5 mS/cm Cond. of |
| | | | | | | | EQ buffer |
| Diafiltration | | DF Buffer | Normal | | | Variable | 2 Diavolumes |
| Concentration | | Load | | | | Variable | Up to 6X VCF |
| Low Pressure | N/A | N/A | | Open | Closed | N/A | ≥5 min |
| Recirculation | | | | | | | |
| System | 3-11 | 0.5N NaOH | SPPO | Open | Open | ≥10 | ≥30 min recirculation |
| | | | | | | L/m² | |
| Cleaning | | | TRPO | | | NA | |
| | | High Purity | SPPO | | | ≥20 | |
| | | Water | | | | L/m² | |
| Storage | 3-11 | 0.1N NaOH | TRPO | Open | Open | ≥10 | Close Retentate and |
| | | | | | | L/m² | Permeate lines so 0.1N |
| | | | | | | | NaOH is retained in |
| | | | | | | | system. |

*Normal: Feed + retentate in retentate vessel, permeate to collection. SPPO: Single pass, permeate open. TRPO: Total recirculation, permeate open.

TABLE 10B
UF/DF solutions

| Buffer | Composition |
|---|---|
| Diafiltration Buffer | 10 mM phosphate pH 7.0 |
| Sanitization Buffer | 0.5N NaOH |
| Storage Buffer | 0.1N NaOH |

### Example 3. Fusion Expression Strategy: Recombinant Fusion Knob Protein Expression in a Pseudomonad Host Cell

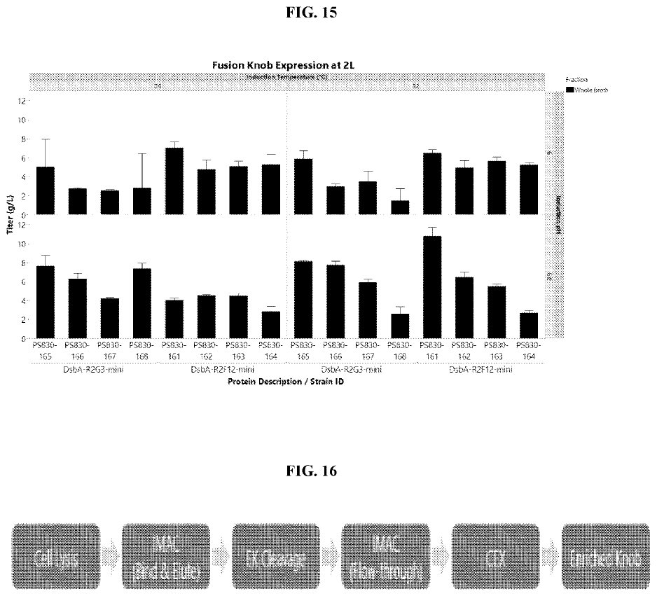

The expression of fusion knobs from constructs encoding DsbA-R2G3-mini, and DsbA-R2F12-mini (Table 11) was evaluated. The identified host strain and plasmid combinations based on DsbA-R2G3 fusion knob production at 96-well and 2 L scale are shown in Table 12. Expression results from 2 L scale are shown in Table 13. Four fermentation conditions were explored for each strain, and soluble fractions of lysates from whole broth were accessed by SDS-CGE under reducing conditions. Due to lack of fusion knob references, titers were estimated by CGE internal ladder for relative titer comparison. Estimated knob titers from fusion knob constructs based on mass contribution were up to 1.2 g/L from PS830-165 for R2G3-mini and 1.8 g/L from PS830-161 for R2F12-mini, respectively, at 32° C., pH 6.8. The estimated knob titers from the fusion protein constructs are comparable with mature knob expression strategy at 2 L scale; PS830-003 at 32° C., pH 6 produced 1.2 g/L of active R2G3-mini and PS830-092 at 32° C., pH 6.8 produced 1.1 g/L of active R2F12-mini measured by binding to RBD spike protein. FIG. 15 shows that induction temperature and pH had no significant impact on fusion knob production, and the most productive strains were PS830-165 for DsbA-R2G3-mini fusion and PS830-161 for DsbA-R2F12-mini fusion. Fusion knobs from high-yield strains PS830-165 and PS830-161 were subjected to enzymatic cleavage reactions to obtain processed knobs, which were purified using 3 column chromatography steps. The final products were characterized by multiple analytical methods (Table 15), as set forth in Example 2 and below.

A general, nonlimiting, purification process for knobs from the fusion knob proteins is shown in FIG. 16. Table 14 compares the overall purification yields for the knobs expressed as mature knobs or as fusion knobs. There is an apparent 20-30-fold difference in recovery for the mature knob over the fusion knob expression strategy [i.e., 30-fold and 22-fold increase in recovery for R2G3-mini and R2F12-mini, respectively]. This can be attributed to the requirement by the mature knob expression strategy for fewer processing steps that incur recovery losses, such as mechanical homogenization and enzymatic cleavage from the fusion protein, as well as the use of ultrafiltration in the early stage of processing to achieve several-fold purification that subsequently requires fewer chromatography steps (i.e., 1 vs. 3). The mature knob and fusion knob purification processes both produced purified mature knob proteins with comparable quality and activity as shown in Table 15.

Purification of Knob Proteins from Fusion Knobs: The frozen cell paste was resuspended to 20% (w/w) in 75 mM phosphate, 100 mM NaCl, pH 7.4 and mechanically lysed with one pass through a Microfluidics M110-P microfluidizer set to 15,000 psi with a cooling heat-exchanger on the instrument outlet to maintain a lysate temperature between 2-8° C. The lysate was clarified by batch centrifugation at 15,000×g for 45 minutes and subsequent filtration of the lysate supernatant with a 0.45/0.2 μm Sartobran P filter (Sartorius, Catalog #5231307H5-00-B).

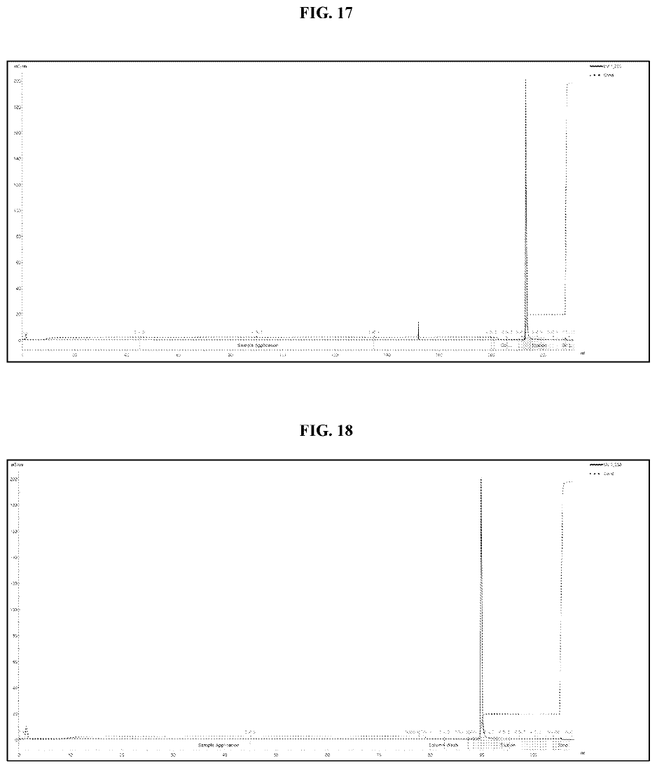

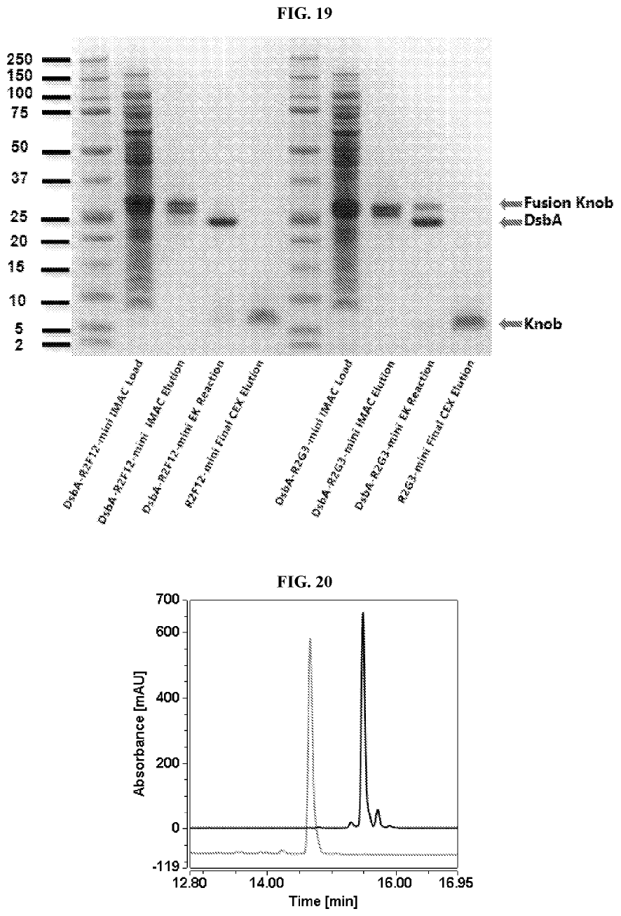

The fusion knob proteins were captured from the lysate by immobilized metal affinity chromatography (IMAC) on column. The knob fusion products were then concentrated, and buffer exchanged into a phosphate buffered saline solution using an Amicon 10 kD molecular weight cut-off (MWCO) centrifugal spin filter (Millipore, Catalog #UFC901096) prior to cleavage by bovine enterokinase (Bio Basic Inc., Catalog #RC572-12EH). Cleavage proceeded over 18-24 hours at 25° C. An additional IMAC step operated in flow-through mode removed EK reaction by-products before the knob proteins were polished and concentrated in two cation exchange chromatography (CEX) column steps with the role of the final CEX step to concentrate the purified product. Representative chromatograms of the CEX purification of the R2F12 mini knob and R2G3 mini knob are shown in FIG. 17 and FIG. 18, respectively. FIG. 19 shows SDS-PAGE analysis of the load material for each of the process intermediates along with the final CEX elution of both R2F12 mini knob and the R2G3 mini knob. Over-expressed putative fusion knob bands in both strain lysates migrate near 29 kDa and were successfully enriched by IMAC (Load and IMAC elution lanes, respectively). Knob proteins were cleaved from DsbA fusion partners via enterokinase digestion (EK reaction lanes). In the case of DsbA-R2G3-mini, the EK reaction was incomplete as evidenced by presence of unreacted fusion knob remaining in the reaction mix after 20 hours. Final knob proteins migrating near 7 kDa were polished and concentrated by CEX chromatography (CEX elution lanes). The purified knob proteins were further analyzed by reverse phase HPLC, with the main peak migrating as expected for each R2F12 mini knob protein and R2G3 mini knob protein.

Hydrogen-Deuterium Exchange Mass Spectrometry: All reagents were purchased from Thermo-Scientific, except for deuterium oxide, which was purchased from Sigma-Aldrich Chemical and the SARS-CoV-2-Spike receptor binding domain protein (RBD) which was purchased from Sino Biological. The RBD protein supplied as a lyophilized powder was reconstituted to a stock concentration of 1.0 mg/mL in Milli-Q water. Peptide sequence coverage analysis was performed on the RBD in water. HDX analysis of the R2G3-mini and R2F12-mini obtained from both fusion knob and mature knob expression binding to the RBD was performed in two states 1) in the presence of either the R2G3-mini or R2F12-mini with the RBD (RBD: knob protein, 1:6 molar ratio, 1 μg: 1 μg mass ratio) and second, in the absence of R2G3-mini or R2F12-mini.

For peptide sequence coverage analysis, 1 μL of RBD stock solution was mixed with 60 μL of water prior to analysis. To generate the RBD-knob solution, 5 μL of purified knob was added to 10 μL of 1 mg/ml RBD solution and the mixture incubated at room temperature for 20 minutes prior to deuterium labeling. For the RBD solution absent of knob protein, in the stock RBD solution the volume of knob protein was replaced with phosphate buffer solution (PBS).

For deuterium labeling experiments, 1 μL of either the RBD in the presence or absence of knob protein was diluted with 30 μL of deuterium oxide (D2O). The labeling reaction was incubated at 25° C. for 20 sec, 1, 5, 20 and 60-min timepoints prior to quenching. Each timepoint was performed in triplicate. The labeling reactions were quenched by adding an equal volume of 30 μL of a solution of ice cooled 2.0 M guanidine and 0.3 M TCEP in 0.2% Formic acid. Samples were flash frozen at −80° C. until analysis. HDX analysis was performed using a Xevo G2-XS Q-TOF Mass spectrometer with a nano Acquity HDX manager and Waters M Class Acquity μBinary Solvent manager and auxiliary solvent manager. Samples were manually injected into an ice cooled loop, digested online with a Waters Enzymate Pepsin Column (2.1×30 mm), and trapped onto a Acquity BEH C18 VanGuard (2.1×5.0 mm) trapping column. Digested peptides were eluted from the analytical Waters Acquity UPLC BEH C18 column (2.1×100 mm) with a linear gradient of 5 to 35% acetonitrile in 7 min at 40 μl/min. Mass spectrometry data was acquired in positive mode, with a capillary voltage of 3.0 kV, cone voltage of 30V, source temperature of 80° C., desolvation temperature of 150° C., a cone gas flow of 50 L/h and a desolvation gas flow of 600 L/h. Data analysis was performed using the Waters PLGS server and DynamX software.

To investigate the epitope of the R2G3 knob, hydrogen/deuterium exchange (HDX) LC-MS analysis was performed to map the interacting residues on RBD. In deuterium labelling experiments, compared to RBD alone, regions with reduced deuterium uptake in the RBD-R2G3 knob complex revealed potential binding residues that were protected by the knob (Table 15). HDX deuterium exchange analysis was superimposed onto the crystal structure of SARS-CoV-2 RBD (PDB ID: 6M0J). The amino acid sequence 158-184, located in the reference binding motif showed maximum protection against deuterium uptake in the presence of the R2G3 knob. Peptides 12-34, 63-86 and 105-123 showed reduced deuterium uptake in presence of 2G3 knob. The HDX data showed that R2G3 knob most likely interacted with the amino acid region 158-184 of RBD, where the knob had the maximal protection against deuterium uptake; this region is within the ACE2 receptor binding motif according to the published crystal structure. Thus, knobs produced by the mature expression strategy (“independently produced”) maintain a single disulfide bound structure as their parent Fab, and produce chemical shifts and slow deuterium uptake upon interaction with antigen, enabling elucidation of the binding residues.

TABLE 11
Constructs.

| Protein | Nucleic acid SEQ ID NO | Amino Acid SEQ ID NO |
|---|---|---|
| DsbA-R2G3-mini | 92 | 93 |
| DsbA-R2F12-mini | 90 | 91 |
| R2G3-mini | 11 | 12 |
| R2F12-mini | 17 | 18 |

TABLE 12
Fusion knob expression strains at 2 L scale up. Fusion protein and host strain were
selected for DsbA-R2G3-mini and DsbA-R2F12-mini constructs based on DsbA-R2G3 fusion
knob production at 2 L scale. Top plasmids were selected based on binding activity
to spike RBD from fusion knob host strain screening at 96-well scale.

| | Molecular | | | | | | | |
|---|---|---|---|---|---|---|---|---|
| | Weight | | Plasmid | Secretion | | Host | | Genomic |
| Protein | (kDa) | Strain ID | ID | Leader | RBS | Strain | Phenotype | Knockout |
| DsbA-R2G3- | 27.05 | PS830-165 | p830-625 | AnsB | Me | PF1596 | Protease | hslUV |
| mini | | PS830-166 | p830-626 | CupB2 | Hi | | deletion | prc 1 |
| | | PS830-167 | p830-627 | 8484 | Hi | | | prc 2 |
| | | PS830-168 | p830-628 | LAO | Me | | | MepM1 |
| DsbA-R2F12- | 27.75 | PS830-161 | p830-621 | AnsB | Me | | | serralysin |
| mini | | PS830-162 | p830-622 | CupB2 | Hi | | | |
| | | PS830-163 | p830-623 | 8484 | Hi | | | |
| | | PS830-164 | p830-624 | LAO | Me | | | |

TABLE 13
Fusion knob expression at 2 L scale up. Titer of fusion knob construct from soluble fraction
of whole broth at harvest (24 hours post-induction) was measured by SDS-CGE under reducing
conditions. Titer of knob protein was calculated by mass contribution.

| | | | | Soluble Fusion | | |
|---|---|---|---|---|---|---|
| | | Induction | | Knob Titer | % Mass of | Calculated Knob |
| Protein | Strain ID | Temp (° C.) | pH | (g/L) | Knob | Titer (g/L) |
| DsbA-R2G3- | PS830-165 | 26 | 6 | 5.0 | 14.5% | 0.7 |
| mini | | | 6.8 | 7.6 | | 1.1 |
| | | 32 | 6 | 5.9 | | 0.9 |
| | | | 6.8 | 8.1 | | 1.2 |
| | PS830-166 | 26 | 6 | 2.8 | | 0.4 |
| | | | 6.8 | 6.3 | | 0.9 |
| | | 32 | 6 | 3.0 | | 0.4 |
| | | | 6.8 | 7.7* | | 1.1 |
| | PS830-167 | 26 | 6 | 2.6 | | 0.4 |
| | | | 6.8 | 4.2 | | 0.6 |
| | | 32 | 6 | 3.5 | | 0.5 |
| | | | 6.8 | 5.9 | | 0.9 |
| | PS830-168 | 26 | 6 | 2.8 | | 0.4 |
| | | | 6.8 | 7.4 | | 1.1 |
| | | 32 | 6 | 1.5 | | 0.2 |
| | | | 6.8 | 2.6 | | 0.4 |
| DsbA- | PS830-161 | 26 | 6 | 7.0 | 16.7% | 1.2 |
| R2F12-mini | | | 6.8 | 4.0 | | 0.7 |
| | | 32 | 6 | 6.5 | | 1.1 |
| | | | 6.8 | 10.8* | | 1.8 |
| | PS830-162 | 26 | 6 | 4.8 | | 0.8 |
| | | | 6.8 | 4.5 | | 0.8 |
| | | 32 | 6 | 4.9 | | 0.8 |
| | | | 6.8 | 6.5 | | 1.1 |
| | PS830-163 | 26 | 6 | 5.1 | | 0.9 |
| | | | 6.8 | 4.5 | | 0.7 |
| | | 32 | 6 | 5.7 | | 0.9 |
| | | | 6.8 | 5.5 | | 0.9 |
| | PS830-164 | 26 | 6 | 5.3 | | 0.9 |
| | | | 6.8 | 2.8 | | 0.5 |
| | | 32 | 6 | 5.3 | | 0.9 |
| | | | 6.8 | 2.7 | | 0.4 |

*Fermentation pastes of fusion knobs at 2 L scale were processed to obtain knob proteins.

TABLE 14
Overall purification yields for mature knob and fusion knob expression strategies.

| Protein | Expression Strategy | Starting Material | Recovery (%) |
|---|---|---|---|
| R2G3-mini | Mature knob expression | Cell-free fermentation broth | 12 |
| | Fusion knob expression | Cell-free lysate | 0.4 |
| R2F12-mini | Mature knob expression | Cell-free fermentation broth | 38 |
| | Fusion knob expression | Cell-free lysate | 1.7 |

TABLE 15
Analytical characterization of purified knobs from mature knob and fusion knob expression strategies.

| | Mature Knob Expression | | Fusion Knob Expression | |
|---|---|---|---|---|
| Analytical Method | R2G3-mini | R2F12-mini | R2G3-mini | R2F12-mini |
| Peptide Mapping | 100% Sequence | 100% Sequence | 100% Sequence | 100% Sequence |
| | Coverage | Coverage | Coverage, Matches to | Coverage, Matches to |
| | | | Non-Fusion | Non-Fusion |
| | | | Expression | Expression |
| Retention Time (RT) | 15.327 | 14.557 | 15.482 | 14.661 |
| of knob proteins by | | | | |
| RP-LC | | | | |
| Relative Retention | NA¹ | NA¹ | 1.01 | 1.01 |
| Time (RRT) | | | | |
| Comparison | | | | |
| between Mature and | | | | |
| Fusion Knob | | | | |
| Expression | | | | |
| Circular Dichroism | Positive Maxima = | Positive Maxima = | N.D.² | Positive Maxima = |
| (CD) Analysis | 203.2 nm | 204.4 nm | | 204.3 nm |
| | Negative Maxima = | Negative Maxima = | | Negative Maxima = |
| | 227.0 nm | 224.6 nm | | 224.8 nm |
| SDS-PAGE | Single Band | Single Band | Single Band | Single Band |
| | migrating close to | migrating close to | migrating close to | migrating close to |
| | 5 kDa | 5 kDa | 5 kDa | 5 kDa |
| KD by BLI | KD = 0.9 nM | KD = 4.4 nM | KD = 1.1 nM | KD = 5.0 nM |
| Potency Analysis by | IC50 = 9 nM | IC50 = 13 nM | IC50 = 11 nM | IC50 = 15 nM |
| BLI | | | | |
| Amino Acid | 154-192 (Major | 154-194 (Major | 154-194 (Major | 154-192 (Major |
| Sequence of Binding | Epitope; 89-102 | Epitope); 89-104 | Epitope); 82-104 | Epitope); 83-104 |
| Epitopes to Spike | and 118-123 | and 118-134 | and 114-123 | and 118-134 |
| RBD Protein as | (Minor Epitopes) | (Minor Epitopes) | (Minor Epitopes) | (Minor Epitopes) |
| determined by HDX | | | | |
| Analysis | | | | |

TABLE 16
Analytical characterization of purified knobs from
mature knob and fusion knob expression strategies.

| | Expression Strategy | | | |
|---|---|---|---|---|
| | Mature Expression | | | |
| | Protein | | | |
| | R2G3-mini | | R2F12-mini | |
| | Condition | | | |
| | Non-Reduced | Reduced | Non-Reduced | Reduced |
| Theoretical Average | 3918.5 | 3926.5 | 4614.2 | 4622.2 |
| Mass (Da) | | | | |
| Observed Average Mass (Da) | 3918.4 | 3926.1 | 4613.9 | 4622.0 |
| Error (Da) | 0.1 | 0.4 | 0.3 | 0.2 |
| Error (ppm) | 20.4 | 112.1 | 56.3 | 49.8 |
| Δ Mass (Reduced-Non- | 7.7 | | 8.1 | |
| Reduced) | | | | |

TABLE 17
Non-Reduced and reduced mass spectrometry data characterizing
the monoisotopic mass for the R2G3-mini and R2F12-mini
knob proteins produced through fusion expression.

| | Expression Strategy | | | |
|---|---|---|---|---|
| | Fusion Knob Expression | | | |
| | Protein | | | |
| | R2G3-mini | | R2F12-mini | |
| | Condition | | | |
| | Non-Reduced | Reduced | Non-Reduced | Reduced |
| Theoretical Isotopic | 3915.55 | 3923.61 | 4610.84 | 4618.90 |
| Mass (Da) | | | | |
| Observed Monoisotopic | 3915.57 | 3923.11 | 4610.85 | 4618.89 |
| Mass (Da) | | | | |
| Error (Da) | 0.02 | 0.5 | 0.01 | 0.01 |
| Error (ppm) | 5.1 | 127.4 | 2.2 | 2.2 |
| Δ Mass (Reduced-Non- | 7.5 | | 8.04 | |
| Reduced) | | | | |

TABLE 18
Non-Reduced and reduced mass spectrometry data characterizing
the average mass for the R2G3-mini and R2F12-mini knob
proteins produced through fusion expression.

| | Expression Strategy | | | |
|---|---|---|---|---|
| | Fusion Knob Expression | | | |
| | Protein | | | |
| | R2G3-mini | | R2F12-mini | |
| | Condition | | | |
| | Non-Reduced | Reduced | Non-Reduced | Reduced |
| Theoretical Average | 3918.5 | 3926.5 | 4614.2 | 4622.2 |
| Mass (Da) | | | | |
| Observed Average Mass (Da) | 3918.2 | 3925.9 | 4613.6 | 4621.5 |
| Error (Da) | 0.3 | 0.6 | 0.6 | 0.7 |
| Error (ppm) | 71.5 | 163.0 | 121.4 | 157.9 |
| Δ Mass (Reduced-Non- | 7.7 | | 7.9 | |
| Reduced) | | | | |

TABLE 19
Table of Sequences.
Protein coding nucleic acid sequences are provided as examples of sequences that
can encode the corresponding amino acid (aa) sequence.

| SEQ ID | | |
|---|---|---|
| NO | Name | Sequence |
| 1 | DsbA-R2G3 | gcggacgtaccgctggaagcgggcaagacttacgtggagctcgcc |
| | (DNA) | aacccggtcccggtcgccgtgccgggcaaaatcgaagtggtggag |
| | | ctgttctggtacggctgccctcattgctacgcgttcgagcccacg |
| | | atcaacccctgggccgagaagctgcccaaagacgtcaacttccgt |
| | | cgcatccccgcgatgttcgggggtccctgggacgcccatggtcag |
| | | ctgtttctcaccttggaagccatgggcgtagagcacaaggtccac |
| | | aacgccgtgttcgaggccatccaaaagcaaggcaagcggctgacc |
| | | aagccggacgagatggccgattttgtggccactcaaggcgtggac |
| | | aaggataagtttctggcgaccttcaatagcttcgcgatccaaggc |
| | | cagatcaagcaagcgaaagaactggcccagaagtatggcgtgcag |
| | | ggcgtccccaccctgatcgtgaacggcaagtaccgcttcgacctg |
| | | ggttcgaccggtgggcccgaggcgaccctgaacgtggcggatcaa |
| | | ctcatcgcgaaggaacgcgcagccaagggtggcggtggctcgggc |
| | | ggtggcggtagccaccatcatcaccaccacgggggcgggggtagc |
| | | gacgatgacgataagggcggcggcggtgccatgggctcggaaggt |
| | | gacaagacgtgtccggacggctacgagcacacgtgtggctgcatc |
| | | gggggctgcggctgcaagcggagcgcgtgcattggcgccctctgc |
| | | tgccaggcctccctcggcggctggctgagcgacggtgagacttac |
| | | acc |
| 2 | DsbA-R2G3 (aa) | ADVPLEAGKTYVELANPVPVAVPGKIEVVELFWYGCPHCYAFEPT |
| | | INPWAEKLPKDVNFRRIPAMFGGPWDAHGQLFLTLEAMGVEHKVH |
| | | NAVFEAIQKQGKRLTKPDEMADFVATQGVDKDKFLATFNSFAIQG |
| | | QIKQAKELAQKYGVQGVPTLIVNGKYRFDLGSTGGPEATLNVADQ |
| | | LIAKERAAKGGGGSGGGGSHHHHHHGGGGSDDDDKGGGGAMGSEG |
| | | DKTCPDGYEHTCGCIGGCGCKRSACIGALCCQASLGGWLSDGETY |
| | | T |
| 3 | L-Asp-R2G3 | ctgcccaacatcaccatcctcgcgaccggtggcacgatcgcgggt |
| | (DNA) | ggcggggatagcgcgaccaagtcgaattacactgcgggcaaggta |
| | | ggcgtggagaacctcgtcaatgccgtcccgcagctgaaagatatt |
| | | gcgaacgtgaaaggcgagcaagtcgtgaacattggctcccaggat |
| | | atgaacgacgacgtgtggctgaccctggcgaaaaagatcaacact |
| | | gactgcgacaagacggatggcttcgtgatcacccatggcacggac |
| | | accatggaagaaaccgcctactttctggacctgaccgtgaagtgc |
| | | gacaagcccgtggtgatggtgggcgcgatgcgccccagcacctcg |
| | | atgtcggccgacgggcccttcaacctgtacaacgccgtggtcacc |
| | | gcggccgataaagcaagcgcgaatcggggcgtcctcgtcgtgatg |
| | | aacgatactgtcctggatggccgcgacgtgaccaagacgaatacg |
| | | actgacgtcgccaccttcaagagcgtaaactacggtcctctgggc |
| | | tacatccacaacggtaagatcgattaccaacggacccccgcccgc |
| | | aagcacaccagcgacacgccgttcgatgtcagcaagctgaacgaa |
| | | ctgccgaaggtaggcatcgtgtacaactatgccaacgccagcgac |
| | | ctccccgccaaggccctcgtggacgcgggctacgacggcatcgtg |
| | | tcggcgggtgtgggcaacgggaacctctacaagaccgtctttgac |
| | | actctggcgaccgccgccaagaacggtaccgcggtagtgcgctcg |
| | | tcgcgtgtacccactggtgcgacgacgcaagacgccgaagtggac |
| | | gatgccaagtacggcttcgtggcgtcagggacgctcaacccgcaa |
| | | aaggcccgggtgctgctgcagctggccctgacccagactaaggat |
| | | ccgcagcagatccaacaaatcttcaaccaatacggcggcggcggc |
| | | tcgggcggtggtggcagccatcatcaccaccaccacggtggcggt |
| | | ggcagcgacgacgacgacaagggcgggggcggtgcgatgggtagc |
| | | gagggcgacaagacttgcccggacggctacgagcatacctgtggc |
| | | tgcatcggcggctgcggctgtaagcgctccgcatgcatcggtgcc |
| | | ctctgctgccaagccagcttgggggctggctgagtgatggggag |
| | | acttacacc |
| 4 | L-Asp-R2G3 (aa) | LPNITILATGGTIAGGGDSATKSNYTAGKVGVENLVNAVPQLKDI |
| | | ANVKGEQVVNIGSQDMNDDVWLTLAKKINTDCDKTDGFVITHGTD |
| | | TMEETAYFLDLTVKCDKPVVMVGAMRPSTSMSADGPFNLYNAVVT |
| | | AADKASANRGVLVVMNDTVLDGRDVTKTNTTDVATFKSVNYGPLG |
| | | YIHNGKIDYQRTPARKHTSDTPFDVSKLNELPKVGIVYNYANASD |
| | | LPAKALVDAGYDGIVSAGVGNGNLYKTVFDTLATAAKNGTAVVRS |
| | | SRVPTGATTQDAEVDDAKYGFVASGTLNPQKARVLLQLALTQTKD |
| | | PQQIQQIFNQYGGGGSGGGGSHHHHHHGGGGSDDDDKGGGGAMGS |
| | | EGDKTCPDGYEHTCGCIGGCGCKRSACIGALCCQASLGGWLSDGE |
| | | TYT |
| 5 | R2G3-His (DNA) | ggcggcggtggcgccatgggcagcgagggcgacaagacttgcccc |
| | | gacggctacgagcatacgtgcgggtgcatcgggggctgcggctgc |
| | | aagcgcagcgcctgcatcggtgcgctgtgttgccaagcgagcctg |
| | | gggggctggctctcggatggcgaaacctacaccggtggcggtggc |
| | | tcgggtggtggcggctcccaccatcaccaccaccac |
| 6 | R2G3-His (aa) | GGGGAMGSEGDKTCPDGYEHTCGCIGGCGCKRSACIGALCCQASL |
| | | GGWLSDGETYTGGGGSGGGGSHHHHHH |
| 7 | R2F12-His | ggtggcggtggcgcgatgggcagcaagactaagaacgcgtgcccg |
| | (DNA) | gatgacttcgactaccggtgcagctgcatcggtggctgcgggtgt |
| | | gcccgcaagggctgcgtgggtccgctgtgctgccgcagcgacctg |
| | | ggcggctacctgaccgattcccccgcctacatctacctcggcggc |
| | | gggggctcgggcggtggcggttcgcaccaccatcaccaccat |
| 8 | R2F12-His (aa) | GGGGAMGSKTKNACPDDFDYRCSCIGGCGCARKGCVGPLCCRSDL |
| | | GGYLTDSPAYIYLGGGGSGGGGSHHHHHH |
| 9 | Prc1 (aa) | MKHLFPSTALAFFIGLGFASMSTNTFAANSWDNLQPDRDEVIASL |
| | P. fluorescens | NVVELLKRHHYSKPPLDDARSVIIYDSYLKLLDPSRSYFLASDIA |
| | | EFDKWKTQFDDFLKSGDLQPGFTIYKRYLDRVKARLDFALGELNK |
| | | GVDKLDFTQKETLLVDRKDAPWLTSTAALDDLWRKRVKDEVLRLK |
| | | IAGKEPKAIQELLTKRYKNQLARLDQTRAEDIFQAYINTFAMSYD |
| | | PHTNYLSPDNAENFDINMSLSLEGIGAVLQSDNDQVKIVRLVPAG |
| | | PADKTKQVAPADKIIGVAQADKEMVDVVGWRLDEVVKLIRGPKGS |
| | | VVRLEVIPHTNAPNDQTSKIVSITREAVKLEDQAVQKKVLNLKQD |
| | | GKDYKLGVIEIPAFYLDFKAFRAGDPDYKSTTRDVKKILTELQKE |
| | | KVDGVVIDLRNNGGGSLQEATELTSLFIDKGPTVLVRNADGRVDV |
| | | LEDENPGAFYKGPMALLVNRLSASASEIFAGAMQDYHRALIIGGQ |
| | | TFGKGTVQTIQPLNHGELKLTLAKFYRVSGQSTQHQGVLPDIDFP |
| | | SIIDTKEIGESALPEAMPWDTIRPAIKPASDPFKPFLAQLKADHD |
| | | TRSAKDAEFVFIRDKLALAKKLMEEKTVSLNEADRRAQHSSIENQ |
| | | QLVLENTRRKAKGEDPLKELKKEDEDALPTEADKTKPEDDAYLAE |
| | | TGRILLDYLKITKQVAKQ |
| 10 | Prc2 (aa) | MLHLSRLTSLALTIALVIGAPLAFADQAAPAAPATAATTKAPLPL |
| | P. fluorescens | DELRTFAEVMDRIKAAYVEPVDDKALLENAIKGMLSNLDPHSAYL |
| | | GPEDFAELQESTSGEFGGLGIEVGSEDGQIKVVSPIDDTPASKAG |
| | | IQAGDLIVKINGQPTRGQTMTEAVDKMRGKLGQKITLTLVRDGGN |
| | | PFDVTLARATITVKSVKSQLLESGYGYIRITQFQVKTGDEVAKAL |
| | | AKLRKDNGKKLNGIVLDLRNNPGGVLQSAVEVVDHFVTKGLIVYT |
| | | KGRIANSELRFSATGNDLSENVPLAVLINGGSASASEIVAGALQD |
| | | LKRGVLMGTTSFGKGSVQTVLPLNNERALKITTALYYTPNGRSIQ |
| | | AQGIVPDIEVRRAKITNEIDGEYYKEADLQGHLGNGNGGADQPTG |
| | | SRAKAKPMPQDDDYQLAQALSLLKGLSITRSR |
| 11 | R2G3-mini | ggtagcacttgccccgacggctacgagcacacgtgcgggtgcatc |
| | (DNA) | ggcggctgcggctgcaagcgctcggcctgcatcggtgcgctgtgc |
| | | tgtcaagcctccctcggcggctggctgagc |
| 12 | R2G3-mini (aa) | GSTCPDGYEHTCGCIGGCGCKRSACIGALCCQASLGGWLS |
| 13 | MepM1 (aa) | MTTEPSKAPPLYPKTHLLAASGIAALLSLALLVFPSSDVEAKRTS |
| | RXF01291 | LSLDLESPVEQLTQDQDASDAQQATNTATESPFAQIESTPEDTQQ |
| | P. fluorescens | AAQEAPAAAKSPQHREVIVGKGDTLSTLFEKVGLPAAAVNDVLAS |
| | | DKQAKQFTQLKRGQKLEFELTPDGQLNNLYTSISDLESISLSKGA |
| | | KGFAFNRITTKPVMRSAYVHGVINSSLSQSAARAGLSHSMTMDMA |
| | | SVFGYDIDFAQDIRQGDEFDVIYEQKVANGKVVGTGNILSARFTN |
| | | RGKTYTAVRYTNKQGNSSYYTADGNSMRKAFIRTPVDFARISSRF |
| | | SMGRKHPILNKIRAHKGVDYAAPRGTPIKAAGDGKVLLAGRRGGY |
| | | GNTVIIQHGNTYRTLYGHMQGFAKGVKTGGNVKQGQVIGYIGTTG |
| | | LSTGPHLHYEFQVNGVHVDPLGQKLPMADPIAKAERARFMQQSQP |
| | | LMARMDQERSTLLASAKR |
| 14 | First stalk- | C-X1-T-V-X2-Q; |
| | forming (aa) | X1 = any amino acid, X2 = any amino acid |
| 15 | First stalk- | CTTVHQ |
| | forming (aa) | |
| 16 | Serralysin | MHIPVRQSSYSRPSDKLQPDLSPDEHQVVLWANNKKSFTTDQAAK |
| | precursor; | HITRGGFKFHDRNNDGKIVVGYNFAGGFNAAQKERARQALQYWAD |
| | extracellular | VANIEFVENGPNTDGTISIKGVPGSAGVAGLPNKYNSNVQANIGT |
| | alkaline | QGGQNPAMGSHFLGLLIHELGHTLGLSHPGKYDGQGFNYDRAAEY |
| | metalloprotease | AQDTKARSVMSYWTETHQPGHNFAGRSPGAPMMDDIAAAQRLYGA |
| | (aa) | NTKTRNTDTTYGFNSNSGREAYSLKQGSDKPIFTVWDGGGNDTLD |
| | (RXF04495.2; | FSGFTQNQTINLKAESFSDVGGLRGNVSIAKGVSVENAIGGTGND |
| | PROKKA_01104) | TLTGNEGNNRLTGGKGADKLHGGAGADTFVYRRASDSTPQAPDII |
| | P. fluorescens | QDFQSGSDKIDLTGVVQEAGLKSLSFVEKFSGKAGEAVLGQDAKT |
| | | GRFTLAVDTTGNGTADLLVASQSQIKQADVIWNGQAPTVTPTPEP |
| | | TVVPVSDPVPTPTSEPTEPEPTPEPAPLPVPTPRPGGGFIGKIFS |
| | | SFKGFIKKVWSIFR |
| 17 | R2F12-mini | ggtggttccaacgcgtgccctgatgacttcgactatcgctgttcc |
| | (DNA) | tgtattggtggttgcggctgcgcccggaaggggtgcgtgggcccc |
| | | ctctgctgccgcagcgacctgggcggctacctgaccgattcgccg |
| | | gcc |
| 18 | R2F12-mini (aa) | GGSNACPDDFDYRCSCIGGCGCARKGCVGPLCCRSDLGGYLTDSP |
| | | A |
| 19 | DegP2 (aa) | MSIPRLKSYLSIVATVLVLGQALPAQAVELPDFTQLVEQASPAVV |
| | (Protease | NISTTQKLPDRKVSNQQMPDLEGLPPMLREFFERGMPQPRSPRGG |
| | RXF07210; | GGQREAQSLGSGFIISPDGYILINNHVIADADEILVRLADRSELK |
| | PROKKA_01390) | AKLIGTDPRSDVALLKIEGKDLPVLKLGKSQDLKAGQWVVAIGSP |
| | P. fluorescens | FGFDHTVTQGIVSAIGRSLPNENYVPFIQTDVPINPGNSGGPLFN |
| | | LAGEVVGINSQIYTRSGGFMGVSFAIPIDVAMDVSNQLKSGGKVS |
| | | RGWLGVVIQEVNKDLAESFGLDKPAGALVAQIQDNGPAAKGGLKV |
| | | GDVILSMNGQPIIMSADLPHLVGALKAGGKAKLEVIRDGKRQNVE |
| | | LTVGAIPEEGATLDALGNAKPGAERSSNRLGIAVVELTAEQKKTF |
| | | DLQSGVVIKEVQDGPAALIGLQPGDVITHLNNQAIDTTKEFADIA |
| | | KALPKNRSVSMRVLRQGRASFITFKLAE |
| 20 | HslU (aa) | MSMTPREIVHELNRHIIGQDDAKRAVAIALRNRWRRMQLPEELRV |
| | (RXF01957; | EVTPKNILMIGPTGVGKTEIARRLAKLANAPFIKVEATKFTEVGY |
| | PROKKA_01919) | VGRDVESIIRDLADAALKMLREQEVTKVSHRAEDAAEERILDALL |
| | P. fluorescens | PPARMGFNEDAAPATDSNTRQLFRKRLREGQLDDKEIEIEVAEVS |
| | | GVDISAPPGMEEMTSQLQNLFANMGKGKKKSRKLKVKEALKLVRD |
| | | EEAGRLVNEEELKAKALEAVEQHGIVFIDEIDKVAKRGNSGGVDV |
| | | SREGVQRDLLPLIEGCTVNTKLGMVKTDHILFIASGAFHLSKPSD |
| | | LVPELQGRLPIRVELKALTPGDFERILSEPHASLTEQYRELLKTE |
| | | GLGIEFQADGIKRLAEIAWQVNEKTENIGARRLHTLLERLLEEVS |
| | | FSAGDMAGAQNGEAIKIDADYVNSHLGELAQNEDLSRYIL |
| | High Ribosome | aggaggt |
| | Binding Site | |
| | (RBS) (DNA) | |
| | Medium | ggagcgt |
| | Ribosome | |
| | Binding Site | |
| | (RBS) (DNA) | |
| 23 | 8484 Signal | atgcgacaactatttttctgtttgatgctgatggtgtcgctcacg |
| | Peptide (DNA) | gcgcacgcc |
| 24 | 8484 Signal | MRQLFFCLMLMVSLTAHA |
| | Peptide (aa) | |
| 25 | AnsB Signal | atgaaatctgcattgaagaacgttattccgggcgccctggccctt |
| | Peptide (DNA) | ctgct gctattccccgtcgccgcccaggcc |
| 26 | AnsB Signal | MKSALKNVIPGALALLLLFPVAAQA |
| | Peptide (aa) | |
| 27 | CupB2 Signal | atgctttttcgcacattactggcgagccttacctttgctgtcatc |
| | Peptide (DNA) | gccggcttaccgtccacggcccacgcc |
| 28 | CupB2 Signal | MLFRTLLASLTFAVIAGLPSTAHA |
| | Peptide (aa) | |
| 29 | FlgI Signal | atgaagttcaaacagctgatggcgatggcgcttttgttggccttg |
| | Peptide (DNA) | agcgctgtggcccaggcc |
| 30 | FlgI Signal | MKFKQLMAMALLLALSAVAQA |
| | Peptide (aa) | |
| 31 | Ibp-S31A Signal | atgatccgtgacaaccgactcaagacatcccttctgcgcggcctg |
| | Peptide (DNA) | accctcaccctactcagcctgaccctgctctcgcccgcggcccat |
| | | gcc |
| 32 | Ibp-S31A Signal | MIRDNRLKTSLLRGLTLTLLSLTLLSPAAHA |
| | Peptide (aa) | |
| 33 | Lao Signal | atgcagaactataaaaaattccttctggccgcggccgtctcgatg |
| | Peptide (DNA) | gcgttcagcgccacggccatggcc |
| 34 | Lao Signal | MQNYKKELLAAAVSMAFSATAMA |
| | Peptide (aa) | |
| 35 | R2G3 knob | GGGGAMGSEGDKTCPDGYEHTCGCIGGCGCKRSACIGALCCQASL |
| | protein (aa) | GGWLSDGETYT |
| 36 | R2F12 knob | GGGGAMGSKTKNACPDDFDYRCSCIGGCGCARKGCVGPLCCRSDL |
| | protein (aa) | GGYLTDSPAYIYLGGGGGGGGS |
| 37 | PorE Signal | atgaagaagtccaccttggctgtggctgtaacgttgggcgcaatc |
| | Peptide (DNA) | gcccagcaagcaggcgcc |
| 38 | PorE Signal | MKKSTLAVAVTLGAIAQQAGA |
| | Peptide (aa) | |
| 39 | TolB Signal | atgagaaaccttcttcgaggaatgcttgtcgttatttgctgtatg |
| | Peptide (DNA) | gcagggatagcggcggcc |
| 40 | TolB Signal | MRNLLRGMLVVICCMAGIAAA |
| | Peptide (aa) | |
| 41 | SecB Folding | atgactgatcaacagaacaccgaagcagcgcaagaccaaggccca |
| | Modulator (DNA) | cagttctcgctgcagcggatctatgtgcgtgacctgtcgttcgaa |
| | | gcgccaaaaagcccggccatcttccgtcaggagtggaccccaagc |
| | | gttgcgctggacctgaacactcgtcagaaatccctggaaggtgac |
| | | ttccacgaagtggtgctgaccctgtcggtcaccgtcaagaatggt |
| | | gaagaagtcgctttcatcgctgaagtgcaacaggccggtatcttc |
| | | ctgatccagggcctggacgaagcgtccatgagccacaccctgggc |
| | | gcgttctgcccgaacatcctgttcccgtatgcccgtgagaccctg |
| | | gacagcctggtcacccgtggctcgttcccggcactgatgctggcg |
| | | ccggttaacttcgatgccctgtacgctcaagagctgcagcgcatg |
| | | caacaggaaggcgcgccgaccgttcag |
| 42 | SecB Folding | MTDQQNTEAAQDQGPQFSLQRIYVRDLSFEAPKSPAIFRQEWTPS |
| | Modulator | VALDLNTRQKSLEGDFHEVVLTLSVTVKNGEEVAFIAEVQQAGIF |
| | (pDOW3702) | LIQGLDEASMSHTLGAFCPNILFPYARETLDSLVTRGSFPALMLA |
| | (aa) | PVNFDALYAQELQRMQQEGAPTVQ |
| 43 | DsbA Folding | atgcgtaatctgatcctcagcgccgctctcgtcactgccagcctc |
| | Modulator (DNA) | ttcggcatgaccgcacaagctgccgatgtgccgcttgaagccggt |
| | (sequence | aaaacctatgttgagctggctaacccggttcccgttgcagtgccg |
| | encoding leader | ggcaagatcgaagtggtggagctgttctggtacggctgcccgcat |
| | underlined) | tgctacgccttcgagccgactatcaacccatgggctgaaaagctg |
| | | cccaaggacgttaacttccgtcgcattcccgccatgttcggtggc |
| | | ccatgggacgcccacggccagctgttcctgaccctggaagccatg |
| | | ggtgttgagcacaaggtccacaacgctgtcttcgaagcgatccag |
| | | aaacaaggcaagcgcctgaccaagccggacgaaatggctgacttc |
| | | gttgccactcagggtgtcgacaaggacaagttcctggcgaccttc |
| | | aactccttcgctatccagggccagatcaaacaggccaaggaactc |
| | | gcgcagaagtacggcgtgcaaggcgttccaaccctgatcgtcaac |
| | | ggcaaataccgtttcgacctgggcagcaccggtggtcctgaagcg |
| | | accctgaacgttgctgaccagctgattgccaaagaacgcgctgcc |
| | | aag |
| 44 | DsbA Folding | MRNLILSAALVTASLFGMTAQAADVPLEAGKTYVELANPVPVAVP |
| | Modulator (aa) | GKIEVVELFWYGCPHCYAFEPTINPWAEKLPKDVNFRRIPAMFGG |
| | (leader sequence | PWDAHGQLFLTLEAMGVEHKVHNAVFEAIQKQGKRLTKPDEMADF |
| | underlined) | VATQGVDKDKFLATFNSFAIQGQIKQAKELAQKYGVQGVPTLIVN |
| | | GKYRFDLGSTGGPEATLNVADQLIAKERAAK |
| 45 | DsbC Folding | atgcgcttgacccagattattgccgccgcagccattgcgttggtt |
| | Modulator (DNA) | tccacctttgcgctcgccgatgatgcggccgagcagaccatccgc |
| | | aagagcctggccaacctggcgctcgacacgcctatcgaaagcatt |
| | | agcgccagccccatggccggcctgtacgaagtcaagctcaagggc |
| | | agccgcgtgctgtacgccagtgccgatggccagtacatcgtccag |
| | | ggctacctgttccagctcaaggacggcaagccggtcaacctgacc |
| | | gagaaggccgagcgcctgggcgtgtccaagctgatcaacggcatc |
| | | ccggtggctgaaaccgtggtttacccggccattggcgaaaccaag |
| | | acccacatcaccgtgttcaccgacaccacctgcccgtactgccac |
| | | aagctgcacgctgaaatcccggcactgaacaagctgggcgtggaa |
| | | gtgcgctacgtcgcgttcccgcgccagggcctgggttcgccgggt |
| | | gacgagcagttgcaagccgtatggtgttcggccgacaaaaaggcg |
| | | gccatggacaagatggtcgacggcaaggaaatcaaatcggccaaa |
| | | tgcgccaacccggtttccaagcagttcgccctgggccagtccatt |
| | | ggtgtgaacggtacaccggccatcgttttggccgacggccaggtg |
| | | attccgggctaccagccggcgccgcaagttgccaaactggcactg |
| | | ggtgccaag |
| 46 | DsbC Folding | MRLTQIIAAAAIALVSTFALADDAAEQTIRKSLANLALDTPIESI |
| | Modulator (aa) | SASPMAGLYEVKLKGSRVLYASADGQYIVQGYLFQLKDGKPVNLT |
| | | EKAERLGVSKLINGIPVAETVVYPAIGETKTHITVFTDTTCPYCH |
| | | KLHAEIPALNKLGVEVRYVAFPRQGLGSPGDEQLQAVWCSADKKA |
| | | AMDKMVDGKEIKSAKCANPVSKQFALGQSIGVNGTPAIVLADGQV |
| | | IPGYQPAPQVAKLALGAK |
| 47 | Skp/OmpH | atgcgtaagttgactcaattggtcttgctggccactgtgctggtc |
| | Folding | accaccccggccttcgccgaaatgaaaatcgccgttctgaactat |
| | Modulator (DNA) | cagatggccctgctggaatccgatgcggccaagcgatacgccgtg |
| | | gatgccgagaagaagttcggtccgcaactgaccaagctcaagaca |
| | | ctggaaagcagcgccaaaggcatccaggaccgcctggtagccggt |
| | | ggcgacaagatgcagcaaggcgagcgcgagcgtctggagcttgaa |
| | | ttcaagcaaaaggcccgtgactaccagttccaatccaaggagctg |
| | | aacgaagccaaggctgtggccgaccgcgaaatgctcaagcagctc |
| | | aagcctaaattggacagcgctgtggaagaagtcatcaagaagggt |
| | | gcctttgacctggtgttcgagcgtggcgccgtgatcgacgtcaag |
| | | cctcaatacgacatcacccgccaggtgatcgagcgcatgaaccag |
| | | ctgaag |
| 48 | Skp/OmpH | MRKLTQLVLLATVLVTTPAFAEMKIAVLNYQMALLESDAAKRYAV |
| | Folding | DAEKKFGPQLTKLKTLESSAKGIQDRLVAGGDKMQQGERERLELE |
| | Modulator (aa) | FKQKARDYQFQSKELNEAKAVADREMLKQLKPKLDSAVEEVIKKG |
| | | AFDLVFERGAVIDVKPQYDITRQVIERMNQLK |
| 49 | FklB2 Folding | atgaaacagcatcggttggcggcggcggtggccctggttagcctg |
| | Modulator (DNA) | gtacttgcgggttgtgattcgcagaccagcgtagagctgaaaacc |
| | | ccggcgcaaaaggcctcctacggcatcggcctgaacatgggcaag |
| | | agccttgcccaagaaggcatggacgacctggactccaaagctgtt |
| | | gcccagggcatcgaagatgccgtcggcaagaaagagcagaagctc |
| | | aaggacgatgagctggttgaagcgtttgccgcactgcaaaagcgt |
| | | gctgaagaacgcatgaccaaaatgagcgaagagtcggcagccgct |
| | | ggcaagaaattcctcgaagacaacgccaagaaagacggtgtcgtc |
| | | accaccgcttccggcctgcagtacaagatcgtgaagaaggccgac |
| | | ggcgcccagcctaagccgaccgacgtggtgactgttcactacacc |
| | | ggcaagctcaccaacggcaccacctttgacagctccgtagatcgc |
| | | ggtagcccgatcgacctgccggtcagcggcgtgatcccgggttgg |
| | | gtcgaaggcctgcaactgatgcacgtgggcgagaaggttgagctg |
| | | tacatcccgtccgacctggcctacggcgcccagagcccgagcccg |
| | | gcgatcccagcgaactccgtgctggtattcgacctggaactgctg |
| | | ggcatcaaggacccagccaaggcagaagcggctgacgcacctgct |
| | | gcaccagccgccaagaag |
| 50 | FklB2 Folding | MKQHRLAAAVALVSLVLAGCDSQTSVELKTPAQKASYGIGLNMGK |
| | Modulator (aa) | SLAQEGMDDLDSKAVAQGIEDAVGKKEQKLKDDELVEAFAALQKR |
| | | AEERMTKMSEESAAAGKKFLEDNAKKDGVVTTASGLQYKIVKKAD |
| | | GAQPKPTDVVTVHYTGKLINGTTFDSSVDRGSPIDLPVSGVIPGW |
| | | VEGLQLMHVGEKVELYIPSDLAYGAQSPSPAIPANSVLVFDLELL |
| | | GIKDPAKAEAADAPAAPAAKK |
| 51 | RXF01037 prc2 | atgctgcatttgtcccgcctcacttcgctggccctgacgatcgcc |
| | (DNA) | ctggtgatcggcgcgcctctggcttttgccgaccaggccgcaccg |
| | | gctgcacccgccacggctgcgacgaccaaggcgccattgccgctg |
| | | gacgagctgcgtacctttgccgaggtcatggaccggatcaaggca |
| | | gcgtatgtcgaacccgtagacgacaaggccctgctggaaaatgcc |
| | | atcaagggcatgctcagcaacctcgacccgcactccgcctacctg |
| | | ggcccggaagatttcgccgagctgcaggaaagcaccagcggtgag |
| | | ttcggcggcctgggcatcgaagtgggctccgaagacggccagatc |
| | | aaagtggtctcgcctatcgacgacaccccggcgtccaaggccggt |
| | | atccaggccggcgacctgatcgtgaagatcaacggccagccaacc |
| | | cgcggccagaccatgaccgaagccgtcgacaagatgcgcggcaag |
| | | ctcggccagaagatcaccctgaccctggtacgcgacggcggcaac |
| | | ccgtttgacgtgaccctggcccgcgcgaccatcacggtcaagagc |
| | | gtgaaaagccagctgctggagtcgggctacggttatatccgtatc |
| | | acccagttccaggtcaagaccggcgacgaagtggccaaggccctg |
| | | gccaagctgcgcaaagacaacggcaagaagctcaacggcatcgtg |
| | | cttgacctgcgcaacaacccaggcggcgtgttgcagtcggcggtc |
| | | gaggtggtcgaccacttcgtcaccaagggcctgatcgtctacacc |
| | | aagggccgtatcgccaactcagagttgcgcttctcggccaccggc |
| | | aacgacctcagcgagaacgtgccactggcggtattgatcaacggt |
| | | ggcagcgcctcggcttcggaaatcgtcgccggtgccctgcaagac |
| | | ctcaagcgcggcgtgctgatgggcaccaccagcttcggcaaaggc |
| | | tcggtgcagaccgtattgccgctgaacaacgagcgtgcgctgaag |
| | | atcaccacggcgctgtactacacgcccaacggccgctcgatccag |
| | | gcccagggcatcgtgccggacatcgaagtacgccgcgccaagatc |
| | | accaacgagatcgacggcgaatactacaaagaggccgacctgcaa |
| | | ggtcacctgggcaatggcaacggcggtgccgaccagccaaccggc |
| | | agccgcgccaaggccaagccgatgccgcaggacgatgactaccaa |
| | | ctggcccaggcactcagcctgctcaagggcttgagcatcacccgc |
| | | agccgt |
| 52 | RXF01037 prc2 | MLHLSRLTSLALTIALVIGAPLAFADQAAPAAPATAATTKAPLPL |
| | (aa) | DELRTFAEVMDRIKAAYVEPVDDKALLENAIKGMLSNLDPHSAYL |
| | | GPEDFAELQESTSGEFGGLGIEVGSEDGQIKVVSPIDDTPASKAG |
| | | IQAGDLIVKINGQPTRGQTMTEAVDKMRGKLGQKITLTLVRDGGN |
| | | PFDVTLARATITVKSVKSQLLESGYGYIRITQFQVKTGDEVAKAL |
| | | AKLRKDNGKKLNGIVLDLRNNPGGVLQSAVEVVDHFVTKGLIVYT |
| | | KGRIANSELRFSATGNDLSENVPLAVLINGGSASASEIVAGALQD |
| | | LKRGVLMGTTSFGKGSVQTVLPLNNERALKITTALYYTPNGRSIQ |
| | | AQGIVPDIEVRRAKITNEIDGEYYKEADLQGHLGNGNGGADQPTG |
| | | SRAKAKPMPQDDDYQLAQALSLLKGLSITRSR |
| 53 | RXF01291 | atgtcattcatcttttccatttcatcttcaaagtcaaaattactt |
| | MepM1 (DNA) | atgaccactgaaccgtctaaagcgccgccgctttacccgaagacc |
| | | cacctgctcgccgcaagtggtatcgccgcccttctcagcctggca |
| | | ctgctggtattcccttccagtgacgttgaagccaaacgaacatcc |
| | | ctgagccttgatctggaaagcccagttgaacaactgacacaagat |
| | | caagacgcttccgacgctcaacaagccacaaacactgcaactgaa |
| | | tcacctttcgcccagatcgaaagcacacccgaagacacccagcaa |
| | | gccgcccaggaagcacctgcagcagccaagagtccccagcatcgc |
| | | gaagtcatcgtgggcaaaggcgacacactctcgaccctgttcgaa |
| | | aaagttgggttgcctgccgccgctgtaaatgacgtgctcgccagc |
| | | gataagcaagccaagcaattcactcagctcaaacgtggtcaaaag |
| | | cttgaatttgagctgacgccagacggccagttgaacaacctgtac |
| | | accagcatcagtgacttggaaagcatcagcctgagcaaaggcgcc |
| | | aaaggcttcgcattcaacagaatcaccaccaaacccgtcatgcgt |
| | | tccgcctacgtacatggcgtgatcaacagctccctgtcgcagtcg |
| | | gccgcgcgtgcgggcctgtcgcatagcatgaccatggacatggcc |
| | | agcgtatttggctacgacatcgacttcgcccaggacatccgtcaa |
| | | ggcgacgaattcgacgtgatctacgaacagaaagtagccaacgga |
| | | aaagtggtcggcactggcaacattctttctgcacgcttcacaaac |
| | | cgtggcaaaacctacaccgccgtgcgctacaccaacaaacaaggc |
| | | aacagcagctactacacggctgatggcaacagcatgcgtaaggcc |
| | | ttcatccgtacacccgttgactttgcccgtattagctcgcgtttc |
| | | tccatgggccgcaagcatccaattctgaacaaaattcgcgcacac |
| | | aagggcgtcgactatgccgcgccgcgtggcacgccaatcaaagca |
| | | gcgggcgacggcaaggtcttgttggcggggcgccgtggtggttac |
| | | ggcaatacggtgatcatccagcacggcaacacttaccgcacgctg |
| | | tacggccacatgcaagggttcgccaagggcgtcaagacaggcggc |
| | | aacgtgaaacagggccaagtgatcggctacatcggtaccaccggc |
| | | ctctccaccggcccgcacttgcactacgagttccaggtcaacggc |
| | | gtacacgtcgacccattgggccagaagctgccgatggccgacccg |
| | | attgccaaggccgaacgcgcgcgcttcatgcaacagagccagccg |
| | | ctgatggcacggatggatcaagagcgctccaccttgctggcttcg |
| | | gcgaagcgt |
| 54 | RXF01291 | MSFIFSISSSKSKLLMTTEPSKAPPLYPKTHLLAASGIAALLSLA |
| | MepM1 (aa) | LLVFPSSDVEAKRTSLSLDLESPVEQLTQDQDASDAQQATNTATE |
| | | SPFAQIESTPEDTQQAAQEAPAAAKSPQHREVIVGKGDTLSTLFE |
| | | KVGLPAAAVNDVLASDKQAKQFTQLKRGQKLEFELTPDGQLNNLY |
| | | TSISDLESISLSKGAKGFAFNRITTKPVMRSAYVHGVINSSLSQS |
| | | AARAGLSHSMTMDMASVEGYDIDFAQDIRQGDEFDVIYEQKVANG |
| | | KVVGTGNILSARFTNRGKTYTAVRYTNKQGNSSYYTADGNSMRKA |
| | | FIRTPVDFARISSRFSMGRKHPILNKIRAHKGVDYAAPRGTPIKA |
| | | AGDGKVLLAGRRGGYGNTVIIQHGNTYRTLYGHMQGFAKGVKTGG |
| | | NVKQGQVIGYIGTTGLSTGPHLHYEFQVNGVHVDPLGQKLPMADP |
| | | IAKAERARFMQQSQPLMARMDQERSTLLAS |
| | | AKR |
| 55 | RXF01957 hslU | atgtccatgactccccgcgaaatcgtccatgaactcaatcgccat |
| | (DNA) | atcatcggccaggacgatgccaagcgcgccgttgccattgcgctg |
| | | cgtaaccgctggcgccggatgcaactgccggaagaactgcgcgtt |
| | | gaagtaacgcccaagaacatcctgatgatcggccccaccggcgtg |
| | | ggtaaaaccgagatcgcccggcgcctggccaaactggccaatgca |
| | | ccgttcatcaaggtcgaagcgaccaagttcaccgaagtcggctat |
| | | gtgggccgcgatgtcgagtcgatcattcgtgacctggctgacgcc |
| | | gccctgaagatgctgcgcgaacaggaagtaaccaaggtcagccac |
| | | cgcgccgaagacgccgctgaagagcgcatcctcgacgccctgttg |
| | | ccaccggcacgcatgggtttcaacgaagacgccgcaccggctacc |
| | | gattccaacactcgccagctgttccgcaagcgcctgcgtgaaggc |
| | | cagctggatgacaaggaaatcgagatcgaagtggctgaagtgtcc |
| | | ggcgtggatatttctgccccgcctggcatggaagaaatgaccagc |
| | | cagctgcagaacctgttcgccaacatgggcaagggcaagaagaaa |
| | | agccgcaagctcaaggtgaaagaggcgctcaagctcgtgcgcgac |
| | | gaagaagccgggcgcctggtcaatgaggaagaactcaaggccaag |
| | | gccctggaagcggtcgagcaacatggcatcgtgtttatcgacgag |
| | | atcgacaaagtggccaagcgaggcaactcaggcggcgtggatgtg |
| | | tcccgcgaaggcgtgcagcgcgatttgctgccgctgatcgagggc |
| | | tgcacggtcaacaccaagctgggcatggtcaagactgaccacatc |
| | | ctgtttatcgcttccggtgctttccacctgagcaagcccagcgac |
| | | ctggtgcccgagctgcaaggccgcttgccgattcgggtggagctc |
| | | aaggcgctgacgccgggcgacttcgagcgcatcctcagcgagccg |
| | | catgcctcgctcaccgagcagtaccgcgagttgctgaaaaccgaa |
| | | gggctgggtatcgaattccaggcagacgggatcaagcgcctggcg |
| | | gagatcgcctggcaggtcaacgagaagaccgagaacatcggtgcc |
| | | cgtcgcctgcataccttgcttgagcgcctgctggaggaagtgtcc |
| | | ttcagtgccggcgacatggccggtgcgcagaatggcgaagcgatc |
| | | aagatcgatgctgattacgtcaacagccacttgggcgaattggcg |
| | | cagaacgaagatctgtctcgttatatcctg |
| 56 | RXF01957 hslU | MSMTPREIVHELNRHIIGQDDAKRAVAIALRNRWRRMQLPEELRV |
| | (aa) | EVTPKNILMIGPTGVGKTEIARRLAKLANAPFIKVEATKFTEVGY |
| | | VGRDVESIIRDLADAALKMLREQEVTKVSHRAEDAAEERILDALL |
| | | PPARMGFNEDAAPATDSNTRQLFRKRLREGQLDDKEIEIEVAEVS |
| | | GVDISAPPGMEEMTSQLQNLFANMGKGKKKSRKLKVKEALKLVRD |
| | | EEAGRLVNEEELKAKALEAVEQHGIVFIDEIDKVAKRGNSGGVDV |
| | | SREGVQRDLLPLIEGCTVNTKLGMVKTDHILFIASGAFHLSKPSD |
| | | LVPELQGRLPIRVELKALTPGDFERILSEPHASLTEQYRELLKTE |
| | | GLGIEFQADGIKRLAEIAWQVNEKTENIGARRLHTLLERLLEEVS |
| | | FSAGDMAGAQNGEAIKIDADYVNSHLGELAQNEDLSRYIL |
| 57 | RXF01961 hslV | ttgaccaccatcgtttcagtacgtcgccacggcaaagttgtcatg |
| | (DNA) | ggcggcgacggccaggtttccctgggcaacaccgtgatgaaaggc |
| | | aacgccaagaaagtgcgccgcctgtaccacggccaggtgcttgcc |
| | | ggcttcgcaggcgcaaccgccgacgcctttaccctgttcgagcgt |
| | | ttcgaaggccagcttgagaaacaccagggccacctggtgcgcgcc |
| | | gctgtggaactagccaaagaatggcgcaccgaccgctccctcagc |
| | | cgcctggaggccatgctcgcggttgcgaacaaagacgcttccctg |
| | | atcatcactggcaacggcgacgtggttgaacccgagcatggcctg |
| | | atcgccatgggttccggcggcggctacgcccaggctgcggccagc |
| | | gcgctgttgaagaaaaccgacctgtcggcccgtgaaatcgtcgag |
| | | accgccctgggtatcgctggcgatatctgcgtgttcaccaaccac |
| | | aaccagaccattgaggagcaggacctcgccgag |
| 58 | RXF01961 hslV | MTTIVSVRRHGKVVMGGDGQVSLGNTVMKGNAKKVRRLYHGQVLA |
| | (aa) | GFAGATADAFTLFERFEGQLEKHQGHLVRAAVELAKEWRTDRSLS |
| | | RLEAMLAVANKDASLIITGNGDVVEPEHGLIAMGSGGGYAQAAAS |
| | | ALLKKTDLSAREIVETALGIAGDICVFTNHNQTIEEQDLAE |
| 59 | RXF07210 degP2 | atgtcgataccacgtttgaagtcttacttatccatagtcgccaca |
| | (DNA) | gtgctggtgctgggtcaggccttacctgcgcaagcggtcgagttg |
| | | cctgacttcacccaactggtggagcaggcctcgcctgccgtggtg |
| | | aacatcagtaccacgcagaagctgccggatcgcaaagtctcgaac |
| | | cagcagatgcccgacctggaaggcttgccgcccatgctgcgcgag |
| | | ttcttcgaacgagggatgccgcaaccacgctccccccgtggcggc |
| | | ggtggccagcgcgaagcccaatccctgggctccggcttcatcatt |
| | | tcgcctgacggctatatcctcaccaacaaccacgtgattgccgat |
| | | gccgacgagattctcgtgcgcctggccgaccgcagtgaactcaag |
| | | gccaagctgattggcaccgatccacgttccgacgtggccttgctt |
| | | aaaatcgagggcaaggacttgccggtgcttaagctgggcaagtcc |
| | | caggacctgaaggccggtcagtgggtggtcgcgatcggttcgccg |
| | | ttcggctttgaccacaccgttacccaaggcatcgtcagcgccatc |
| | | ggtcgcagcctgccgaacgaaaactacgtaccgttcatccagacc |
| | | gacgtgccgatcaacccgggtaactccggtggcccgctgttcaac |
| | | ctggccggcgaagtggtggggatcaactcgcagatctacacccgc |
| | | tccggcggcttcatgggcgtgtctttcgcgatcccaatcgatgtg |
| | | gccatggacgtctccaatcagctcaaaagcggcggcaaggtcagc |
| | | cgcggctggttgggcgtggtaatccaggaagtgaacaaggacctg |
| | | gctgagtccttcggtctcgacaagccggccggtgccctggttgcg |
| | | cagattcaggacaatggccctgcggccaaaggcggcctgaaagtc |
| | | ggtgacgtcatcctgagcatgaacggccagccgatcatcatgtcg |
| | | gcagacttgcctcatttggtcggcgcgctcaaggccggcggcaaa |
| | | gccaagctggaagtgattcgtgatggcaagcgccagaacgtcgaa |
| | | ctgaccgtaggtgccatcccggaagaaggcgcgaccctggatgcc |
| | | ctgggcaacgccaagcccggtgccgagcgcagcagtaaccgcctg |
| | | ggtatcgccgtggttgaactgaccgccgagcagaagaaaaccttc |
| | | gacctgcaaagcggtgtggtgatcaaggaagttcaggacggccca |
| | | gccgccttgatcggcctgcaaccgggtgacgtgatcactcacttg |
| | | aacaaccaggcaatcgataccaccaaggaattcgccgacatcgcc |
| | | aaggcgttgccgaagaatcgctcggtgtcgatgcgcgtcctgcgt |
| | | caaggccgtgccagcttcattaccttcaagctggctgag |
| 60 | RXF07210 degP2 | MSIPRLKSYLSIVATVLVLGQALPAQAVELPDFTQLVEQASPAVV |
| | (aa) | NISTTQKLPDRKVSNQQMPDLEGLPPMLREFFERGMPQPRSPRGG |
| | | GGQREAQSLGSGFIISPDGYILINNHVIADADEILVRLADRSELK |
| | | AKLIGTDPRSDVALLKIEGKDLPVLKLGKSQDLKAGQWVVAIGSP |
| | | FGFDHTVTQGIVSAIGRSLPNENYVPFIQTDVPINPGNSGGPLEN |
| | | LAGEVVGINSQIYTRSGGFMGVSFAIPIDVAMDVSNQLKSGGKVS |
| | | RGWLGVVIQEVNKDLAESFGLDKPAGALVAQIQDNGPAAKGGLKV |
| | | GDVILSMNGQPIIMSADLPHLVGALKAGGKAKLEVIRDGKRQNVE |
| | | LTVGAIPEEGATLDALGNAKPGAERSSNRLGIAVVELTAEQKKTF |
| | | DLQSGVVIKEVQDGPAALIGLQPGDVITHLNNQAIDTTKEFADIA |
| | | KALPKNRSVSMRVLRQGRASFITFKLAE |
| 61 | RXF04495.2- | atgacggtggtgaaggtcttttcaatgtgggagctttatcgggct |
| | Serralysin (DNA) | gacaacggagcagtcggcatcggtaactcgcatatatggacggtt |
| | | aactttccactgttcagagtatcaaagcacatgcatatccctgtt |
| | | aggcagtcttcttactcgcgtccttcagataagttacagcccgat |
| | | ctttcacccgatgaacaccaagttgttctctgggccaacaataaa |
| | | aaatctttcaccacggatcaggccgcgaaacacatcacccgcggt |
| | | ggcttcaagtttcatgatcgcaacaatgatggaaaaatcgtcgtg |
| | | ggttataactttgcgggcggcttcaatgcggctcagaaagaacgg |
| | | gccaggcaagcccttcagtactgggcggatgttgctaatatcgaa |
| | | tttgttgaaaatggcccgaacacggatggcacaataagcatcaag |
| | | ggtgttccgggttcggcaggcgtcgcggggttgcccaacaaatat |
| | | aattcgaacgtccaggccaatataggcacccagggtgggcaaaac |
| | | ccggcgatgggcagtcacttcctgggcttattgatccatgaactg |
| | | gggcataccctggggctgagtcatccaggtaaatacgacggccag |
| | | ggtttcaattacgatcgggctgccgaatatgcccaggacaccaag |
| | | gctcgcagtgtcatgagctattggacggagactcatcagccgggg |
| | | cacaattttgccgggcgcagcccgggtgccccgatgatggacgat |
| | | atcgccgccgcccagcggctctacggcgccaacaccaaaacccgg |
| | | aataccgacaccacctacggcttcaattccaattcaggccgggag |
| | | gcttatagcctcaagcaggggagcgacaagccgatcttcaccgtc |
| | | tgggacggtggaggtaatgacacgctcgacttctccgggttcacc |
| | | cagaaccaaaccatcaacctcaaggctgagtcattctcggacgtg |
| | | gggggcttgcgaggaaatgtgtcgattgccaagggtgtgagtgtg |
| | | gaaaacgccattggcggtacaggcaacgataccttgacggggaac |
| | | gagggcaacaatcggctcacgggcggcaagggggccgataagctg |
| | | cacggcggagctggagcagacacgtttgtttaccgccgcgccagc |
| | | gattcaacgccgcaggcaccggacatcatccaggacttccagagc |
| | | gggagcgacaagatcgacctgaccggtgttgttcaggaggcgggg |
| | | ctcaagtcgctgagcttcgtcgagaaattcagcggcaaggcgggc |
| | | gaggccgtgctcggccaagacgcgaaaaccggccgtttcacgttg |
| | | gcggtggacacaacgggaaatggtacggcggatctactggttgcc |
| | | agccaaagccagatcaaacaggcggatgtgatctggaacggtcag |
| | | gcgccgacagtgacgccaacgcctgaacccactgtggtgcctgtg |
| | | tcagatcccgtgccgacccctacttcagagccgactgaacctgaa |
| | | cccacgcctgagcccgcccctttgcccgtcccgactccacggcct |
| | | ggaggagggtttatcgggaaaattttttcatcattcaaggggttc |
| | | ataaaaaaagtgtggtcgatattcagg |
| 62 | RXF04495.2- | MTVVKVFSMWELYRADNGAVGIGNSHIWTVNFPLFRVSKHMHIPV |
| | Serralysin (aa) | RQSSYSRPSDKLQPDLSPDEHQVVLWANNKKSFTTDQAAKHITRG |
| | | GFKFHDRNNDGKIVVGYNFAGGENAAQKERARQALQYWADVANIE |
| | | FVENGPNTDGTISIKGVPGSAGVAGLPNKYNSNVQANIGTQGGQN |
| | | PAMGSHELGLLIHELGHTLGLSHPGKYDGQGENYDRAAEYAQDTK |
| | | ARSVMSYWTETHQPGHNFAGRSPGAPMMDDIAAAQRLYGANTKTR |
| | | NTDTTYGFNSNSGREAYSLKQGSDKPIFTVWDGGGNDTLDFSGFT |
| | | QNQTINLKAESFSDVGGLRGNVSIAKGVSVENAIGGTGNDTLTGN |
| | | EGNNRLTGGKGADKLHGGAGADTFVYRRASDSTPQAPDIIQDFQS |
| | | GSDKIDLTGVVQEAGLKSLSFVEKFSGKAGEAVLGQDAKTGRETL |
| | | AVDTTGNGTADLLVASQSQIKQADVIWNGQAPTVTPTPEPTVVPV |
| | | SDPVPTPTSEPTEPEPTPEPAPLPVPTPRPGGGFIGKIFSSFKGF |
| | | IKKVWSIFR |
| 63 | RXF06586 prc1 | atgcgttatcaattgcccccgcgtcgaatcagcatgaagcatctg |
| | (DNA) | ttccccagcaccgccctcgcttttttcattggtctcggcttcgcg |
| | | tcgatgtcgaccaatacgttcgcagccaatagctgggacaacctt |
| | | cagcctgatcgcgatgaggtgattgccagccttaacgtcgtcgag |
| | | ttgcttaagcgccatcactacagcaagccgccgctggacgacgct |
| | | cgctcagtgatcatctacgacagctacctcaagctgctggacccg |
| | | tcgcgcagctacttcctggccagcgatatcgctgagttcgacaag |
| | | tggaagacgcaattcgacgacttcctcaagagcggcgacctgcag |
| | | cctggcttcaccatctacaagcgctacctagaccgcgtcaaagcg |
| | | cgtctggacttcgccctgggtgagctgaacaaaggcgtcgacaag |
| | | ctcgatttcacccagaaagaaacccttctggtggaccgcaaggac |
| | | gccccttggctgaccagcaccgcagccctagacgacctgtggcgc |
| | | aaacgcgtcaaggacgaagtgctgcgcttgaagatcgccggcaaa |
| | | gagcccaaggccattcaagagctgttgaccaagcgctacaaaaac |
| | | cagctggcgcgcctggaccagacccgtgccgaggatatcttccag |
| | | gcctacatcaacacctttgcgatgtcctacgacccgcacaccaat |
| | | tatctgtcgccagataacgcggaaaatttcgatatcaatatgagt |
| | | ctgtccctggaaggcatcggtgccgtcctgcaaagcgacaatgac |
| | | caggtgaagattgtacgtctggtgccggcaggcccggctgacaaa |
| | | accaagcaagtggcaccggccgacaagatcatcggcgtggcccag |
| | | gccgacaaagagatggtcgatgtggtcggctggcgcctggacgaa |
| | | gtggtcaagctgatccgtgggcctaaaggcagcgtggtgcgcctg |
| | | gaagtgattccgcacaccaatgcaccgaacgaccagaccagcaag |
| | | atcgtgtccatcacccgtgaagcggtgaagctcgaagaccaggcc |
| | | gtgcagaagaaagtcctcaacctcaagcaggatggcaaggactac |
| | | aagctgggggtgattgaaatcccggccttctacctggacttcaag |
| | | gcgttccgtgccggtgatccggactacaagtccaccacccgcgac |
| | | gtgaagaaaatcctcacagaactgcagaaagagaaagtcgacggc |
| | | gtggtcatcgacctgcgcaacaacggcggcggctccctgcaggaa |
| | | gccaccgagctgaccagcctgtttatcgacaagggcccgaccgtg |
| | | ttggtacgcaacgctgacggccgtgtcgacgtgctcgaagacgag |
| | | aacccgggggccttctacaaagggccgatggcgctgctggtcaac |
| | | cgcctctcggcctcggcctcggagattttcgccggtgccatgcag |
| | | gactaccaccgtgcactgatcatcggcggccagaccttcggcaaa |
| | | ggcaccgtgcagaccatccagccgctgaaccatggcgagcttaag |
| | | ctgacactggccaagttctaccgggtctccgggcagagcacccag |
| | | catcagggcgtactgccggatatcgatttcccgtcgatcatcgac |
| | | accaaggaaattggcgaaagcgccctgcctgaagccatgccgtgg |
| | | gacaccatccgccctgcgatcaagccggcgtcggatccgttcaag |
| | | ccgttcctggcacagctgaaggctgaccacgacacccgctctgcc |
| | | aaggatgccgagttcgtgtttatccgcgacaagctggccctggcc |
| | | aagaagctgatggaagagaagaccgtcagcctcaacgaagcggat |
| | | cgccgtgcacagcactccagcatcgagaatcagcaactggtgctg |
| | | gaaaacacccgccgcaaggccaaaggtgaagacccgctcaaagag |
| | | ctgaagaaagaagatgaagacgcgctgccgaccgaggcggataaa |
| | | accaagccggaagacgacgcctacttggccgagactggccggatc |
| | | ctgctggattacctgaagatcaccaagcaggtggccaagcag |
| 64 | RXF06586 prc1 | MRYQLPPRRISMKHLFPSTALAFFIGLGFASMSTNTFAANSWDNL |
| | (aa) | QPDRDEVIASLNVVELLKRHHYSKPPLDDARSVIIYDSYLKLLDP |
| | | SRSYFLASDIAEFDKWKTQFDDELKSGDLQPGFTIYKRYLDRVKA |
| | | RLDFALGELNKGVDKLDFTQKETLLVDRKDAPWLTSTAALDDLWR |
| | | KRVKDEVLRLKIAGKEPKAIQELLTKRYKNQLARLDQTRAEDIFQ |
| | | AYINTFAMSYDPHTN |
| | | YLSPDNAENFDINMSLSLEGIGAVLQSDNDQVKIVRLVPAGPADK |
| | | TKQVAPADKIIGVAQADKEMVDVVGWRLDEVVKLIRGPKGSVVRL |
| | | EVIPHTNAPNDQTSKIVSITREAVKLEDQAVQKKVLNLKQDGKDY |
| | | KLGVIEIPAFYLDFKAFRAGDPDYKSTTRDVKKILTELQKEKVDG |
| | | VVIDLRNNGGGSLQEATELTSLFIDKGPTVLVRNADGRVDVLEDE |
| | | NPGAFYKGPMALLVN |
| | | RLSASASEIFAGAMQDYHRALIIGGQTFGKGTVQTIQPLNHGELK |
| | | LTLAKFYRVSGQSTQHQGVLPDIDFPSIIDTKEIGESALPEAMPW |
| | | DTIRPAIKPASDPFKPFLAQLKADHDTRSAKDAEFVFIRDKLALA |
| | | KKLMEEKTVSLNEADRRAQHSSIENQQLVLENTRRKAKGEDPLKE |
| | | LKKEDEDALPTEADKTKPEDDAYLAETGRILLDYLKITKQVAKQ |
| 65 | EcpD-R2G3 | gccgtggtgatcaccggcacccggctggtctaccccgcggaccag |
| | (DNA) | aaagaaatcaccgtcaagctcaacaacaacggtacgctgccggca |
| | | ctcgtgcaatcatggatcgacactggctcggtcgagagcaccccg |
| | | actagctccaaggccccattcctgctctcgcctccggtggcccgc |
| | | atcgaccccaccaaggggcaaagcctgcgtgtactgttcacgggt |
| | | gcgcccttggcgcaagacaaggaatcggtgttctggctgaatgtc |
| | | ctggaaatcccgccgaagcccgaggccggtgccgatctgaacact |
| | | ctccagatggccttccgctcgcggatcaagctgttctaccgtccg |
| | | gtaggtctgcccggcaacccgaacgaagcggtcgagcaagtgcag |
| | | tggcaactggtgactgcccgcgacggccagggcctggcgctcaaa |
| | | gcgtacaatcccagcgccttccacgtatcgctcatcgagctggat |
| | | ctggtcgccggcaaccagcgctaccgcagcgaagatggcatggtg |
| | | ggtcccggcgagactcgccagtttgccctccccaccctcaaggcg |
| | | cggccgagcagccaggcccaagtggagttttcggcgatcaacgac |
| | | tacggtgcgttggtgccgacccggaacacgctgcaaccgggcggg |
| | | ggcgggagtggtggtggcggtagccatcatcaccaccatcacggc |
| | | ggcggtggcagcgacgacgatgacaagggtggcggtggcgcgatg |
| | | gggtccgagggcgacaagacctgtcccgatggctatgagcacacg |
| | | tgcggctgcatcggcggctgcggctgcaagcgcagcgcctgcatt |
| | | ggggccctgtgctgccaagcgagcctcggcggctggctgtcggac |
| | | ggcgaaacctacacc |
| 66 | EcpD-R2G3 (aa) | AVVITGTRLVYPADQKEITVKLNNNGTLPALVQSWIDTGSVESTP |
| | | TSSKAPFLLSPPVARIDPTKGQSLRVLFTGAPLAQDKESVFWLNV |
| | | LEIPPKPEAGADLNTLQMAFRSRIKLFYRPVGLPGNPNEAVEQVQ |
| | | WQLVTARDGQGLALKAYNPSAFHVSLIELDLVAGNQRYRSEDGMV |
| | | GPGETRQFALPTLKARPSSQAQVEFSAINDYGALVPTRNTLQPGG |
| | | GGSGGGGSHHHHHHGGGGSDDDDKGGGGAMGSEGDKTCPDGYEHT |
| | | CGCIGGCGCKRSACIGALCCQASLGGWLSDGETYT |
| 67 | R2G3 truncation | GGSEGDKTCPDGYEHTCGCIGGCGCKRSACIGALCCQASLGGWLS |
| | (aa) | D |
| 68 | HslV (aa) | MTTIVSVRRHGKVVMGGDGQVSLGNTVMKGNAKKVRRLYHGQVLA |
| | (RXF01961; | GFAGATADAFTLFERFEGQLEKHQGHLVRAAVELAKEWRTDRSLS |
| | PROKKA_01920) | RLEAMLAVANKDASLIITGNGDVVEPEHGLIAMGSGGGYAQAAAS |
| | P. fluorescens | ALLKKTDLSAREIVETALGIAGDICVFTNHNQTIEEQDLAE |
| 69 | CupC2 secretion | atgccgcctcgttctatcgccgcatgtctggggctgctgggcttg |
| | leader nucleic | ctcatggctacccaggccgccgcc |
| | acid sequence | |
| | (P. fluorescens) | |
| 70 | CupC2 secretion | MPPRSIAACLGLLGLLMATQAAA |
| | leader (aa) | |
| | (P. fluorescens) | |
| 71 | Azurin secretion | atgtttgccaaactcgttgctgtttccctgctgactctggcgagc |
| | leader nucleic | ggccagttgcttgct |
| | acid sequence | |
| | (P. fluorescens) | |
| 72 | Azurin (Azu) | MFAKLVAVSLLTLASGQLLA |
| | secretion leader | |
| | (aa) | |
| | (P. fluorescens) | |
| 73 | Pbp secretion | atgaaactgaaacgtttgatggcggcaatgacttttgtcgctgct |
| | leader nucleic | ggcgttgcgaccgccaacgcggtggcc |
| | acid sequence | |
| | (P. fluorescens) | |
| 74 | Pbp secretion | MKLKRLMAAMTFVAAGVATANAVA |
| | leader (aa) | |
| | (P. fluorescens) | |
| 75 | PbpA20V | atgaaactgaaacgtttgatggcggcaatgacttttgtcgctgct |
| | secretion leader | ggcgttgcgaccgtcaacgcggtggcc |
| | nucleic acid | |
| | sequence | |
| | (P. fluorescens) | |
| 76 | PbpA20V | MKLKRLMAAMTFVAAGVATVNAVA |
| | secretion leader | |
| | (aa) | |
| | (P. fluorescens) | |
| 77 | 5193 secretion | atgcaaagcctgccgttctctgcgttacgcctgctcggtgtgctg |
| | leader nucleic | gcagtcatggtctgcgtgctgttgacgacgccagcccgtgcc |
| | acid sequence | |
| | (P. fluorescens) | |
| 78 | 5193 secretion | MQSLPFSALRLLGVLAVMVCVLLTTPARA |
| | leader (aa) | |
| | (P. fluorescens) | |
| 79 | Ibp secretion | atgatccgtgacaaccgactcaagacatcccttctgcgcggcctg |
| | leader nucleic | accctcaccctactcagcctgaccctgctctcgcccgcggcccat |
| | acid sequence | tct |
| | (P. fluorescens) | |
| 80 | Ibp secretion | MIRDNRLKTSLLRGLTLTLLSLTLLSPAAHS |
| | leader (aa) | |
| | (P. fluorescens) | |
| 81 | A first stalk- | CATVHQ |
| | forming sequence | |
| | (aa) | |
| 82 | A first stalk- | CAIVQQ |
| | forming sequence | |
| | (aa) | |
| 83 | A first stalk- | CATVDQ |
| | forming sequence | |
| | (aa) | |
| | A second stalk- | Y-X3-Y-X4-Y |
| | forming sequence | X3 = any amino acid, X4 = any amino acid |
| | (aa) | |
| 85 | DegP2 (S219A) | MSIPRLKSYLSIVATVLVLGQALPAQAVELPDFTQLVEQASPAVV |
| | (aa) | NISTTQKLPDRKVSNQQMPDLEGLPPMLREFFERGMPQPRSPRGG |
| | P. fluorescens | GGQREAQSLGSGFIISPDGYILINNHVIADADEILVRLADRSELK |
| | (Do family serine | AKLIGTDPRSDVALLKIEGKDLPVLKLGKSQDLKAGQWVVAIGSP |
| | endopeptidase; | FGFDHTVTQGIVSAIGRSLPNENYVPFIQTDVPINPGNAGGPLFN |
| | NCBI Reference | LAGEVVGINSQIYTRSGGFMGVSFAIPIDVAMDVSNQLKSGGKVS |
| | Sequence | RGWLGVVIQEVNKDLAESFGLDKPAGALVAQIQDNGPAAKGGLKV |
| | WP_198833397.1 | GDVILSMNGQPIIMSADLPHLVGALKAGGKAKLEVIRDGKRQNVE |
| | with | LTVGAIPEEGATLDALGNAKPGAERSSNRLGIAVVELTAEQKKTF |
| | S219A included | DLQSGVVIKEVQDGPAALIGLQPGDVITHLNNQAIDTTKEFADIA |
| | as indicated by | KALPKNRSVSMRVLRQGRASFITFKLAE |
| | bold text) | |
| 86 | DegP2 (S219A) | ATGTCGATACCACGTTTGAAGTCTTACTTATCCATAGTCGCCACA |
| | example nucleic | GTGCTGGTGCTGGGTCAGGCCTTACCTGCGCAAGCGGTCGAGTTG |
| | acid encoding | CCTGACTTCACCCAACTGGTGGAGCAGGCCTCGCCTGCCGTGGTG |
| | SEQ ID NO: 85 | AACATCAGTACCACGCAGAAGCTGCCGGATCGCAAAGTCTCGAAC |
| | | CAGCAGATGCCCGACCTGGAAGGCTTGCCGCCCATGCTGCGCGAG |
| | | TTCTTCGAACGAGGGATGCCGCAACCACGCTCCCCCCGTGGCGGC |
| | | GGTGGCCAGCGCGAAGCCCAATCCCTGGGCTCCGGCTTCATCATT |
| | | TCGCCTGACGGCTATATCCTCACCAACAACCACGTGATTGCCGAT |
| | | GCCGACGAGATTCTCGTGCGCCTGGCCGACCGCAGTGAACTCAAG |
| | | GCCAAGCTGATTGGCACCGATCCACGTTCCGACGTGGCCTTGCTT |
| | | AAAATCGAGGGCAAGGACTTGCCGGTGCTTAAGCTGGGCAAGTCC |
| | | CAGGACCTGAAGGCCGGTCAGTGGGTGGTCGCGATCGGTTCGCCG |
| | | TTCGGCTTTGACCACACCGTTACCCAAGGCATCGTCAGCGCCATC |
| | | GGTCGCAGCCTGCCGAACGAAAACTACGTACCGTTCATCCAGACC |
| | | GACGTGCCGATCAACCCGGGTAACGCCGGTGGCCCGCTGTTCAAC |
| | | CTGGCCGGCGAAGTGGTGGGGATCAACTCGCAGATCTACACCCGC |
| | | TCCGGCGGCTTCATGGGCGTGTCTTTCGCGATCCCAATCGATGTG |
| | | GCCATGGACGTCTCCAATCAGCTCAAAAGCGGCGGCAAGGTCAGC |
| | | CGCGGCTGGTTGGGCGTGGTAATCCAGGAAGTGAACAAGGACCTG |
| | | GCTGAGTCCTTCGGTCTCGACAAGCCGGCCGGTGCCCTGGTTGCG |
| | | CAGATTCAGGACAATGGCCCTGCGGCCAAAGGCGGCCTGAAAGTC |
| | | GGTGACGTCATCCTGAGCATGAACGGCCAGCCGATCATCATGTCG |
| | | GCAGACTTGCCTCATTTGGTCGGCGCGCTCAAGGCCGGCGGCAAA |
| | | GCCAAGCTGGAAGTGATTCGTGATGGCAAGCGCCAGAACGTCGAA |
| | | CTGACCGTAGGTGCCATCCCGGAAGAAGGCGCGACCCTGGATGCC |
| | | CTGGGCAACGCCAAGCCCGGTGCCGAGCGCAGCAGTAACCGCCTG |
| | | GGTATCGCCGTGGTTGAACTGACCGCCGAGCAGAAGAAAACCTTC |
| | | GACCTGCAAAGCGGTGTGGTGATCAAGGAAGTTCAGGACGGCCCA |
| | | GCCGCCTTGATCGGCCTGCAACCGGGTGACGTGATCACTCACTTG |
| | | AACAACCAGGCAATCGATACCACCAAGGAATTCGCCGACATCGCC |
| | | AAGGCGTTGCCGAAGAATCGCTCGGTGTCGATGCGCGTCCTGCGT |
| | | CAAGGCCGTGCCAGCTTCATTACCTTCAAGCTGGCTGAG |
| 87 | Disulfide-bond | MIDDMRLGRERRFLVLLGIICLALIGGALYMQVVLGEAPCPLCIL |
| | isomerase DsbB | QRYALLLIALFAFIGAAMRTKGALTFFEGLVVLSALGGVAAAGHH |
| | (aa) | VYTQFFPQVSCGIDVLQPIVDDLPLAKVFPLGFQVDGFCSTPYPP |
| | (RXF03204.1) | ILGLSLAQWALVAFVLTAILVPLCIYRNRHPKA |
| 88 | Disulfide-bond | MRHLFTFLLVLFAGFAQAAPGSPFETKPDFLPVGKAFAFTSERLE |
| | isomerase DsbD | SGETQLFWQIADGYYLYQQRMKFDGLAEKPVLPEGEAHSDEFFGE |
| | (aa) | QQVYRQGLEVKIPAGTTGQVKLGWQGCADAGLCYPPQSITVDLGG |
| | (RXF04886.2) | NPAVAATAQAQDQSLASGLQQRSLGWSLLVFFGLGLLLAFAPCSL |
| | | PMLPILAGLVVGSGASPRRGFALAGSYVVCMALVYAALGVMAALL |
| | | GANLAALLQTPWILGSFAALFVLLALPMFGFFELQLPAFLRDRLD |
| | | NVSRQQSGGSLVGAGVLGALSGLLVGPCMTAPLAGALLYIAQSGN |
| | | ALHGGLILFAMGIGIGIPLLLLVTVGNRFLPKPGTWMNVLKGIFG |
| | | FLFLGTAVLMIRPVVGDSLWIGLWGALALVMAYCGWALARESGLA |
| | | AKVFGAGSLVLGLWGAVLVVGAAGGSDELWQPLKVYSGSRVADAP |
| | | SAHDAFTTVSDPAVLQSQLDSAKAQGQWVLLDYYADWCVSCKIME |
| | | KQVFGKPEVMDALKDVRLLRLDVTADNAASRELLGRYKVPGPPSF |
| | | VWIGPDGEERRAQRITGEVDAAAFLQRWTQTRDAR |
| 89 | Disulfide-bond | MPRLRHLLTLLPLTLAAALAQAEDLPAPIKQIEAKGAKIIGKFDA |
| | isomerase DsbG | PSGLTGYAAQYQNRGMALYLTADGKNVIAGNLYDAQGNDLSTAPL |
| | (aa) | EKLVYAPMAKEVWAKMENSSWIQDGDKNAPRTIYLFSDPNCPYCN |
| | (RXF04890.2) | MFWEQARPWVKAGKVQLRHIMVGIIREDSPGKSAALLAAKDPQKA |
| | | LQDHEAAGKGSKLKALEKIPAEVEAKLDANMKLMDELELSATPAI |
| | | FYLDDKGGLQQQQGAPSPDKLVKILGPK |
| 90 | DsbA-R2F12- | gcggacgtaccgctggaagcgggcaagacttacgtggagctcgcc |
| | mini (DNA) | aacccggtcccggtcgccgtgccgggcaaaatcgaagtggtggag |
| | | ctgttctggtacggctgccctcattgctacgcgttcgagcccacg |
| | | atcaacccctgggccgagaagctgcccaaagacgtcaacttccgt |
| | | cgcatccccgcgatgttcgggggtccctgggacgcccatggtcag |
| | | ctgtttctcaccttggaagccatgggcgtagagcacaaggtccac |
| | | aacgccgtgttcgaggccatccaaaagcaaggcaagcggctgacc |
| | | aagccggacgagatggccgattttgtggccactcaaggcgtggac |
| | | aaggataagtttctggcgaccttcaatagcttcgcgatccaaggc |
| | | cagatcaagcaagcgaaagaactggcccagaagtatggcgtgcag |
| | | ggcgtccccaccctgatcgtgaacggcaagtaccgcttcgacctg |
| | | ggttcgaccggtgggcccgaggcgaccctgaacgtggcggatcaa |
| | | ctcatcgcgaaggaacgcgcagccaagggtggcggtggctcgggc |
| | | ggtggcggtagccaccatcatcaccaccacgggggcgggggtagc |
| | | gacgatgacgataagggtggttccaacgcgtgccctgatgacttc |
| | | gactatcgctgttcctgtattggtggttgcggctgcgcccggaag |
| | | gggtgcgtgggccccctctgctgccgcagcgacctgggcggctac |
| | | ctgaccgattcgccggcc |
| 91 | DsbA-R2F12- | ADVPLEAGKTYVELANPVPVAVPGKIEVVELFWYGCPHCYAFEPT |
| | mini (aa) | INPWAEKLPKDVNFRRIPAMFGGPWDAHGQLFLTLEAMGVEHKVH |
| | underlined; | NAVFEAIQKQGKRLTKPDEMADFVATQGVDKDKFLATENSFAIQG |
| | enterokinase | QIKQAKELAQKYGVQGVPTLIVNGKYRFDLGSTGGPEATLNVADQ |
| | cleavage site | LIAKERAAKGGGGSGGGGSHHHHHHGGGGSDDDDKGGSNACPDDE |
| | italicized) | DYRCSCIGGCGCARKGCVGPLCCRSDLGGYLTDSPA |
| 92 | DsbA-R2G3-mini | gcggacgtaccgctggaagcgggcaagacttacgtggagctcgcc |
| | (DNA) (encodes | aacccggtcccggtcgccgtgccgggcaaaatcgaagtggtggag |
| | by SEQ ID NO: | ctgttctggtacggctgccctcattgctacgcgttcgagcccacg |
| | 93) | atcaacccctgggccgagaagctgcccaaagacgtcaacttccgt |
| | | cgcatccccgcgatgttcgggggtccctgggacgcccatggtcag |
| | | ctgtttctcaccttggaagccatgggcgtagagcacaaggtccac |
| | | aacgccgtgttcgaggccatccaaaagcaaggcaagcggctgacc |
| | | aagccggacgagatggccgattttgtggccactcaaggcgtggac |
| | | aaggataagtttctggcgaccttcaatagcttcgcgatccaaggc |
| | | cagatcaagcaagcgaaagaactggcccagaagtatggcgtgcag |
| | | ggcgtccccaccctgatcgtgaacggcaagtaccgcttcgacctg |
| | | ggttcgaccggtgggcccgaggcgaccctgaacgtggcggatcaa |
| | | ctcatcgcgaaggaacgcgcagccaagggtggcggtggctcgggc |
| | | ggtggcggtagccaccatcatcaccaccacgggggcgggggtagc |
| | | gacgatgacgataagggtagcacttgccccgacggctacgagcac |
| | | acgtgcgggtgcatcggcggctgcggctgcaagcgctcggcctgc |
| | | atcggtgcgctgtgctgtcaagcctccctcggcggctggctgagc |
| 93 | DsbA-R2G3-mini | ADVPLEAGKTYVELANPVPVAVPGKIEVVELFWYGCPHCYAFEPT |
| | (aa) (linker | INPWAEKLPKDVNFRRIPAMFGGPWDAHGQLFLTLEAMGVEHKVH |
| | underlined; | NAVFEAIQKQGKRLTKPDEMADFVATQGVDKDKFLATENSFAIQG |
| | enterokinase | QIKQAKELAQKYGVQGVPTLIVNGKYRFDLGSTGGPEATLNVADQ |
| | cleavage site | LIAKERAAKGGGGSGGGGSHHHHHHGGGGSDDDDKGSTCPDGYEH |
| | italicized) | TCGCIGGCGCKRSACIGALCCQASLGGWLS |
| 94 | DsbA Folding | gcggacgtaccgctggaagcgggcaagacttacgtggagctcgcc |
| | Modulator (DNA) | aacccggtcccggtcgccgtgccgggcaaaatcgaagtggtggag |
| | | ctgttctggtacggctgccctcattgctacgcgttcgagcccacg |
| | | atcaacccctgggccgagaagctgcccaaagacgtcaacttccgt |
| | | cgcatccccgcgatgttcgggggtccctgggacgcccatggtcag |
| | | ctgtttctcaccttggaagccatgggcgtagagcacaaggtccac |
| | | aacgccgtgttcgaggccatccaaaagcaaggcaagcggctgacc |
| | | aagccggacgagatggccgattttgtggccactcaaggcgtggac |
| | | aaggataagtttctggcgaccttcaatagcttcgcgatccaaggc |
| | | cagatcaagcaagcgaaagaactggcccagaagtatggcgtgcag |
| | | ggcgtccccaccctgatcgtgaacggcaagtaccgcttcgacctg |
| | | ggttcgaccggtgggcccgaggcgaccctgaacgtggcggatcaa |
| | | ctcatcgcgaaggaacgcgcagccaag |
| 95 | DsbA Folding | ADVPLEAGKTYVELANPVPVAVPGKIEVVELFWYGCPHCYAFEPT |
| | Modulator, no | INPWAEKLPKDVNFRRIPAMFGGPWDAHGQLFLTLEAMGVEHKVH |
| | secretion leader | NAVFEAIQKQGKRLTKPDEMADEVATQGVDKDKFLATENSFAIQG |
| | (aa) | QIKQAKELAQKYGVQGVPTLIVNGKYRFDLGSTGGPEATLNVADQ |
| | | LIAKERAAK |
| 96 | Cleavable linker | DDDDK |
| 97 | Linker | GGGGAMGS |

What is claimed is:

1. A method for producing a recombinant ultralong CDR3 knob peptide, the method comprising: culturing a Pseudomonadales host cell in a culture medium and expressing the recombinant ultralong CDR3 knob peptide in the periplasm of the Pseudomonadales host cell from an expression construct comprising a nucleic acid encoding the recombinant ultralong CDR3 knob peptide directly and operably linked to a periplasmic secretion leader; wherein the recombinant ultralong CDR3 knob peptide is produced by secretion into the periplasm of the Pseudomonadales host cell, and wherein the secreted recombinant ultralong CDR3 knob peptide is present in soluble form, active form, and/or in properly processed form.

2. The method of claim 1, wherein the recombinant ultralong CDR3 knob peptide is about 3 to about 8 kDa.

3. The method of claim 1 or 2, wherein the recombinant ultralong CDR3 knob peptide is about 25 to about 90 amino acids in length and comprises a cysteine motif, wherein the cysteine motif comprises 2-20 cysteine residues capable of forming 1-10 disulfide bonds.

4. The method of claim 3, wherein the recombinant ultralong CDR3 knob peptide further comprises a first stalk-forming amino acid sequence and a second stalk-forming amino acid sequence, wherein the first and second stalk-forming sequences are, respectively, first and second β-strands of about 1 to about 15 amino acids in length, wherein the first β-strands are anti-parallel to the second β-strands.

5. The method of claim 4, wherein the cysteine motif is positioned between the first and second stalk-forming amino acid sequences.

6. The method of any one of claims 1 to 5, wherein the periplasmic secretion leader has at least 85% identity to an amino acid sequence selected from SEQ ID NOS: 24, 26, 28, 30, 32, 34, 38, and 40.

7. The method of any one of claims 1 to 6, wherein the nucleic acid encoding the recombinant ultralong CDR3 knob peptide is operably linked to a ribosome binding site sequence (RBS), optionally wherein the sequence of the RBS is aggaggt or ggagcgt.

8. The method of any one of claims 1 to 7, wherein the sequence of the nucleic acid encoding the recombinant ultralong CDR3 knob peptide is not directly and/or operably linked to a nucleic acid sequence encoding a fusion partner, a cleavable linker, or both.

9. The method of any one of claims 1 to 8, wherein the produced recombinant ultralong CDR3 knob peptide is present in the periplasm in soluble form at a yield of about 0.5 g/L to about 8 g/L.

10. The method of any one of claims 1 to 9, further comprising: measuring the quality of an amount of the recombinant ultralong CDR3 knob peptide produced.

11. The method of claim 10, wherein the quality is measured by a target binding assay.

12. The method of any one of claims 1 to 11, wherein at least 80% of the produced recombinant ultralong CDR3 knob peptide present in the periplasm in soluble form is active.

13. The method of any one of claim 11 or 12, wherein the assay comprises comparison to a reference, wherein the reference may be a negative control or a corresponding recombinant ultralong CDR3 knob peptide produced from a fusion construct.

14. The method of any one of claims 1 to 13, wherein the nucleic acid encoding the recombinant ultralong CDR3 knob peptide is optimized for expression in the host cell.

15. The method of any one of claims 1 to 14, wherein the Pseudomonadales host cell is Pseudomonas fluorescens.

16. The method of any one of claims 1 to 15, wherein the Pseudomonadales host cell is deficient in expression of one or more protease, overexpresses one or more folding modulator, overexpresses one or more inactivated protease, or a combination thereof.

17. The method of claim 16, wherein the one or more protease is selected from: Lon, HslU, HslV, DegP1, DegP2, DegP2 S219A, Prc1, Prc2, MepM1, a serralysin, and AprA.

18. The method of claim 17, wherein the one or more protease comprises DegP2.

19. The method of claim 18, wherein the one or more protease is selected from: Prc1, Prc2, HslU, HslV, MepM1, and a serralysin.

20. The method of claim 19, wherein the serralysin is RXF04495.2.

21. The method of any one of claims 16 to 20, wherein the one or more folding modulator is selected from: SecB, DsbA, DsbC, Skp, and FklB2.

22. The method of any one of claims 16 to 21, wherein the host cell overexpresses: i) SecB; ii) DsbA, DsbC and Skp; iii) DsbA and DsbC; or iv) DsbC and FklB2.

23. The method of any one of claims 1 to 18, wherein the host cell is deficient in expression of DegP2, and overexpresses SecB.

24. The method of any one of claims 1 to 18, wherein the host cell is deficient in expression of DegP2, and overexpresses DsbA, DsbC, and Skp.

25. The method of any one of claims 1 to 18, wherein the host cell is deficient in expression of DegP2, and overexpresses DsbA and DsbC.

26. The method of any one of claims 1 to 18, wherein the host cell overexpresses DsbC and FklB2.

27. The method of any one of claims 1 to 16, wherein the host strain has a phenotype and genotype as set forth for any host strain in any one of Tables 3, 4, 6, and 9.

28. The method of any one of claims 1 to 16, wherein the host strain has a phenotype, genotype, and expression construct sequence elements as set forth for any host strain in any one of Tables 3, 4, 6, and 9.

29. The method of any one of claims 1 to 16, wherein the host strain is any as set forth in any one of Tables 3, 4, 6, and 9.

30. The method of any one of claims 1 to 18, wherein
a) the periplasmic secretion leader has at least 85% identity to an amino acid sequence SEQ ID NO: 26;
b) the host cell is deficient in expression of DegP2; and overexpresses SecB.

31. The method of any one of claims 1 to 18, wherein
a) the periplasmic secretion leader has at least 85% identity to an amino acid sequence SEQ ID NO: 40;
b) the host cell is deficient in expression of DegP2; and overexpresses i) SecB, or ii) DsbA, DsbC and Skp.

32. The method of any one of claims 1 to 18, wherein
a) the periplasmic secretion leader has at least 85% identity to an amino acid sequence SEQ ID NO: 30;
b) the host cell is deficient in expression of DegP2; and overexpresses SecB.

33. The method of any one of claims 1 to 32 wherein an amount of the ultralong CDR3 knob peptide secreted into the periplasm is released to the culture medium.

34. The method of any one of claims 1 to 33, further comprising purifying the produced recombinant ultralong CDR3 knob peptide.

35. The method of claim 34, wherein purifying the produced recombinant ultralong CDR3 knob peptide comprises: separating the cultured Pseudomonadales host cell expressing the recombinant ultralong CDR3 knob peptide from the culture medium to obtain separated Pseudomonadales host cell and separated culture medium; obtaining a cell lysate from the separated Pseudomonadales host cell; performing ultrafiltration of the cell lysate and/or the separated culture medium, to obtain an ultrafiltration permeate and an ultrafiltration concentrate; and performing chromatographic separation of the ultrafiltration permeate to obtain the purified recombinant ultralong CDR3 knob peptide.

36. The method of claim 35, wherein the ultrafiltration comprises passing the cell lysate and/or the separated culture medium through one or more molecular weight cut offs (MWCO) of about 5 to about 50 kDA.

37. The method of claim 35 or 36, wherein performing chromatographic separation of the ultrafiltration permeate comprises performing cation exchange chromatography on the ultrafiltration permeate.

38. The method of claim 35, wherein purifying the recombinant ultralong CDR3 knob peptide comprises: separating the cultured Pseudomonadales host cell expressing the recombinant ultralong CDR3 knob peptide from the culture medium to obtain separated Pseudomonadales host cell and separated culture medium; obtaining a cell lysate from the separated Pseudomonadales host cell; performing a first chromatographic separation of the cell lysate and/or the separated culture medium, to obtain a first eluate containing the recombinant ultralong CDR3 knob peptide; and performing a second chromatographic separation of the first eluate to obtain the purified recombinant ultralong CDR3 knob peptide.

39. The method of claim 38, wherein performing the first chromatographic separation of the cell lysate and/or the separated culture medium comprises performing cation exchange chromatography on the cell lysate and/or the separated culture medium.

40. The method of claim 38 or 39, wherein performing the second chromatographic separation of the first eluate comprises performing size exclusion chromatography on the first eluate.
